(19) United States
(12) Patent Application Publication
(10) Pub. No.: US 2012/0184542 A1
(43) Pub. Date: Jul. 19, 2012

(54) PYRIDO PYRIMIDINES

(76) Inventors: Kevin Anderson, Hackettstown, NJ (US); Yi Chen, Nutley, NJ (US); Zhi Chen, Livingston, NJ (US); Kin-Chun Luk, North Caldwell, NJ (US); Pamela Loreen Rossman, Nutley, NJ (US); Hongmao Sun, Germantown, MD (US); Peter Michael Wovkulich, Nutley, NJ (US)

(21) Appl. No.: 13/348,665

(22) Filed: Jan. 12, 2012

Related U.S. Application Data
(60) Provisional application No. 61/433,994, filed on Jan. 19, 2011.

(51) Int. Cl. A61K 31/519; A61K 31/5377; A61P 25/00; A61K 31/5386; A61P 35/00; A61P 25/28; C07D 471/04; C07D 519/00

(52) U.S. Cl. 514/230.5; 544/279; 514/264.1; 544/117; 514/234.2; 514/252.16; 544/105; 514/264.11

(57) ABSTRACT

Compounds of formula

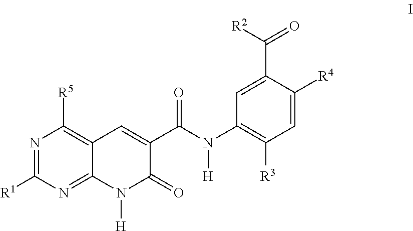

and pharmaceutically acceptable salts thereof are described, as well as the pharmaceutical compositions containing said compounds and their pharmaceutically acceptable salts, and the use of said compounds and pharmaceutical compositions for the treatment, control or amelioration of proliferative diseases, including cancer, Down syndrome or early onset Alzheimer's disease.

### PRIORITY TO RELATED APPLICATIONS

This application claims the benefit of U.S. Provisional Application No. 61/433,994, filed Jan. 19, 2011. The entire contents of the above-identified application is hereby incorporated by reference.

### FIELD OF THE INVENTION

The present invention relates to pyrido[2,3-d]pyrimidines which act as inhibitors of DYRK1B and/or DYRK1A and are useful in the amelioration, treatment or control of cancer, especially solid tumors, or in the amelioration, treatment or control of Down syndrome or early onset Alzheimer's disease.

### BACKGROUND OF THE INVENTION

Kinases are known to be important cellular enzymes that regulate cellular functions such as regulating cell division and proliferation. WO 2008/047307. Dual-specificity tyrosine-phosphorylation-regulated kinases (DYRKs) are a subfamily of protein kinases that have dual-specificity and are believed to play roles in cell proliferation and apoptosis induction. See, e.g., Kiyotsugu Yoshida, “Role for DYRK family kinases on regulation of apoptosis,” Biochemical Pharmacology 76 (2008) pp 1389-1394; Jinghun Gao et al., “Mirk/Dyrk1B, a novel therapeutic target, mediates cells survival in non-small cell lung cancer cells,” Cancer Biology & Therapy 8:17 (2009) pp. 1671-1679. DYRK1A is believed to be implicated in neural differentiation. Yoshida, id. at 1390. Over expression of this kinase is believed to be involved in Down syndrome and Alzheimer's disease. See Nam Kim, “Putative therapeutic agents for learning and memory deficits of people with Down syndrome,” Bioorganic & Medicinal Chemistry Letters,” 16 (2006) pp 3772-76 and Joongkyu Park et al, “Function and regulation of Dyrk1A: towards understanding Down syndrome,” Cell. Mol. Life. Sci 66 (2009) pgs. 3235-3240. Thus, inhibition of this kinase is believed to be of benefit in controlling or ameliorating the effects of Down syndrome and early onset Alzheimer's disease. See, e.g., Kim, id; Park, id, and Kyung Koo et al., “QSAR analysis of pyrazolidine-3,5-diones derivatives as Dyrk1A inhibitors,” Bioorganic & Medicinal Chemistry Letters 19 (2009) pp 2324-2328.

DYRK1B (also referred to as MIRK) mediates survival and differentiation in many tissues. It is believed to be implicated in certain cancers, particularly solid tumors. See, e.g., Gao, supra (lung cancer cells); Kangmoon Lee et al, “Mirk Protein Kinase is a Mitogen-activated Protein Kinase Substrate that Mediates Survival of Colon Cancer Cells”, Cancer Research 60 (2000):3631-3637 and Xiaobing Deng et al, “The Kinase Mirk/Dyrk1B Mediates Cell Survival in a Pancreatic Ductal Adenocarcinoma,” Cancer Res 66:8 (2006) pp 4149-58 (pancreatic cancer cells). Thus, inhibition of this kinase is believed to be of benefit in controlling or ameliorating cancer. See, Cao Yang et al, “The kinase Mirk is a potential therapeutic target in osteosarcoma,” Carcinogenesis 31:4 (2010) pp 552-558 and Eileen Friedman, “The Kinase Mirk/dyrk1B: A Possible Therapeutic Target in Pancreatic Cancer,” Cancers 2 (2010) 1492-1512.

### SUMMARY OF THE INVENTION

One aspect of the invention is a compound of formula I

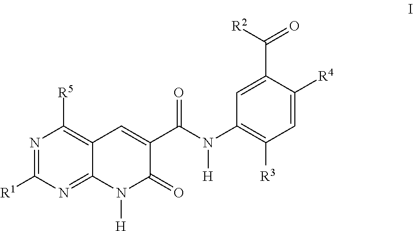

or a pharmaceutically acceptable salt thereof, wherein R¹ through R⁵ are as defined below.

The present invention also relates to pharmaceutical compositions comprising one or more compounds of the invention, or a pharmaceutically acceptable salt, and a pharmaceutically acceptable carrier or excipient.

The present invention further relates to a method of treating, ameliorating or controlling cancer, including specifically solid tumors, for example lung, pancreatic, colon, breast, bone and prostate cancers in a mammal, specifically a human, comprising administering to said mammal a therapeutically effective amount of a compound according to the invention or a pharmaceutically acceptable salt thereof.

The present invention further relates to a method of treating, ameliorating or controlling Down syndrome or Alzheimer's disease in a human, comprising administering to said human a therapeutically effective amount of a compound according to the invention or a pharmaceutically acceptable salt thereof.

### DETAILED DESCRIPTION OF THE INVENTION

### Definitions

As used herein, the following terms shall have the following definitions.

The terms “C₁₋₆ alkyl” or “C₁₋₄ alkyl” refer to straight- or branched-chain saturated hydrocarbon groups having from 1 to 6, or 1 to 4, carbon atoms, respectively. Examples of C₁₋₆ alkyl groups include, but are not limited to, methyl, ethyl, n-propyl, i-propyl, n-butyl, s-butyl, t-butyl, n-pentyl, and s-pentyl.

“Aryl” means a substituted or unsubstituted monovalent, monocyclic or bicyclic, aromatic carboxylic hydrocarbon radical, preferably a 6-10 member aromatic ring system. Preferred aryl groups include, but are not limited to, phenyl, naphthyl, tolyl, and xylyl.

“Cycloalkyl” means a substituted or unsubstituted stable monocyclic or polycyclic system which consists of carbon atoms only, all rings of which are saturated. Examples of cycloalkyls include, but are not limited to, cyclopropyl, cyclobutyl, cyclopentyl, cyclohexyl, cycloheptyl, adamantyl, cyclooctyl, bicycloalkyls, including bicyclooctanes such as [2.2.2]bicyclooctane or [3.3.0]bicyclooctane, bicyclononanes such as [4.3.0]bicyclononane, and bicyclodecanes such as [4.4.0]bicyclodecane (decalin), or spiro compounds.

“Halogen” means Cl, F and Br.

“Heteroaryl” means a substituted or unsubstituted aromatic heterocyclic ring system containing up to two rings. Preferred heteroaryl groups include, but are not limited to, thienyl (or thiophenyl), furyl, indolyl, pyrrolyl, pyridinyl, pyrazinyl, oxazolyl, thiaxolyl, quinolinyl, pyrimidinyl, imidazolyl, triazolyl and tetrazolyl.

In the case of a heteroaryl that is bicyclic it should be understood that one ring may be aryl while the other is heteroaryl and both may be independently substituted or unsubstituted.

“Hetero atom” means an atom selected from N, O and S.

“Heterocycle” or “heterocyclic ring” means a substituted or unsubstituted 5 to 10 membered, mono- or bicyclic, non-aromatic hydrocarbon, wherein 1 to 3 carbon atoms are replaced by a hetero atom selected from nitrogen, oxygen or sulfur atom. Examples include pyrrolidinyl, including pyrrolidin-1-yl, pyrrolidin-2-yl and pyrrolidin-3-yl; piperazinyl; piperidinyl; morpholinyl, including morpholin-4-yl; and the like, which in turn optionally may be substituted.

In the case of a heterocycle that is bicyclic it should be understood that one ring may be heterocycle while the other is cycloalkyl, and either or both may be independently substituted. An example of such a bicyclic heterocycle is 8-oxa-3-aza-bicyclo[3.2.1]octane.

Hydroxy or hydroxyl is a prefix indicating the presence of a monovalent —O—H group.

“IC₅₀” refers to the concentration of a particular compound required to inhibit 50% of a specific measured activity. IC₅₀ can be measured, inter alia, as is described subsequently in Examples 201 and 202.

“Pharmaceutically acceptable,” such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, trifluoroacetic acid and the like. Sample base-addition salts include those derived from ammonium, potassium, sodium and, quaternary ammonium hydroxides, such as for example, tetramethylammonium hydroxide. Chemical modification of a pharmaceutical compound (i.e. drug) into a salt is a technique well known to pharmaceutical chemists to obtain improved physical and chemical stability, hygroscopicity, flowability and solubility of compounds. See, e.g., Ansel et al., Pharmaceutical Dosage Forms and Drug Delivery Systems (1995) at pgs. 456-457.

“Substituted,” as in substituted alkyl, means that the substitution can occur at one or more positions and, unless otherwise indicated, that the substituents at each substitution site are independently selected from the specified options. The term “optionally substituted” refers to the fact that one or more hydrogen atoms of a chemical group (with one or more hydrogen atoms) can be, but does not necessarily have to be, substituted with another substituent.

In one embodiment, the present invention relates to compounds of formula I

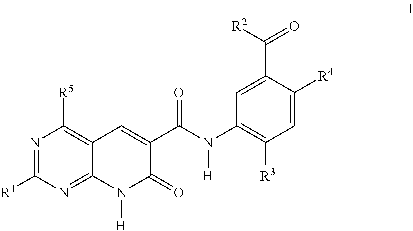

wherein

R¹ is selected from the group
(a) H,
(b) C₁₋₄ alkyl,
(c) C₁₋₄ alkyl substituted with up to 3 groups selected from cycloalkyl, heterocycle, OR⁶, NR⁶R⁷, and CN,
(d) OR⁶,
(e) NR⁶R⁷,
(f) heterocycle,
(g) heterocycle substituted with up to three groups selected from C₁₋₄ alkyl, OR⁸, NR⁸R⁹, CN and C(O)C₁₋₄ alkyl, and
(h) SR⁶;

R² is selected from the group
(a) NR¹⁶R¹¹, and
(b) OR¹²;

R³ is selected from the group
(a) H,
(b) CH₃,
(c) F,
(d) Cl, and
(f) Br;

R⁴ is selected from the group
(a) H, and
(b) F;

R⁵ is selected from the group
(a) H, and
(b) C₁₋₄ alkyl;

R⁶ and R⁷ are independently selected from the group
(a) H,
(b) C₁₋₄ alkyl,
(c) C₁₋₄ alkyl substituted with up to 3 groups selected from
OH,
OC₁₋₄ alkyl,
NR⁸R⁹,
CN,
heterocycle,
heterocycle substituted with up to three C₁₋₄ alkyl groups,
cycloalkyl, and
cycloalkyl substituted with up to 3 groups selected from OH, NH₂ and C₁₋₄ alkyl,
(d) cycloalkyl,
(e) cycloalkyl substituted with up to 3 groups selected from OH, NR⁸R⁹ and C₁₋₄ alkyl,
(f) heterocycle, and
(g) heterocycle substituted with up to three C₁₋₄ alkyl groups;

R⁸ and R⁹ are independently selected from the group
(a) H, and
(b) C₁₋₄ alkyl;

R¹⁰ and R¹¹ are independently selected from the group
(a) H,
(b) C₁₋₆ alkyl,
(c) C₁₋₆ alkyl substituted with up to 4 groups selected from
aryl,
aryl substituted with Cl, F, CH₃, or CF₃
heteroaryl,
cycloalkyl,
heterocycle,
F, provided that no more than two substituents are F at the same time,
OH,
OC₁₋₄ alkyl,
NR⁸R⁹,
CN, and
CONR⁸R⁹,
(d) heterocycle substituted with aryl that optionally is substituted with halogen or C₁₋₄ alkyl, and
(e) aryl optionally substituted with Cl, F or CH₃; and

R¹² is selected from the group
(a) C₁₋₆ alkyl,
(b) C₁₋₆ alkyl substituted with up to 4 groups selected from
aryl,
aryl substituted with Cl, F, CH₃, or CF₃
heteroaryl,
cycloalkyl,
heterocycle,
F, provided that no more than two substituents are F at the same time,
OH,
OC₁₋₄ alkyl,
NR⁸R⁹,
CN, and
CONR⁸R⁹,
(c) heterocycle substituted with aryl that is optionally substituted with halogen or C₁₋₄ alkyl, and
(d) aryl optionally substituted with Cl, F or CH₃;
   or a pharmaceutically acceptable salt thereof.

In another embodiment, the invention relates to compounds of formula I wherein R¹ is H, or a pharmaceutically acceptable salt thereof.

Another embodiment of the invention relates to compounds of Formula I wherein R¹ is OR⁶ and R⁶ is C₁₋₄ alkyl optionally substituted with OC₁₋₄ alkyl, heterocycle, or a pharmaceutically acceptable salt thereof. In one particular embodiment OR⁶ is OCH₃.

Another embodiment of the invention relates to compounds of Formula I wherein R¹ is SR⁶ and R⁶ is C₁₋₄ alkyl, or a pharmaceutically acceptable salt thereof. In one embodiment SR⁶ is SCH₃.

Another embodiment of the invention relates to compounds of Formula I wherein R¹ is a heterocycle optionally substituted with C₁₋₄ alkyl, NR⁸R⁹ or OR⁸, and R⁸ and R⁹ are independently H and methyl, or a pharmaceutically acceptable salt thereof. In one embodiment R¹ is heterocycle optionally substituted with CH₃, NH₂ or OH. In one embodiment the heterocycle is 8-oxa-3-aza-bycyclo[3.2.1]octane. In another embodiment R¹ is selected from piperidinyl, piperazinyl and morpholinyl each of which may optionally be substituted with CH₃.

Another embodiment of the invention relates to compounds of Formula I wherein R¹ is NR⁶R⁷ and R⁶ and R⁷ are independently selected from H, C₁₋₄ alkyl that is optionally substituted with OH or OC₁₋₄ alkyl, N-ethyl, heterocycle, cycloalkyl, and wherein each of the heterocycle and cycloalkyl by be substituted with CH₃, OH, NH₂, or a pharmaceutically acceptable salt thereof. In another embodiment NR⁶R⁷ is C₁₋₄ alkyl that is substituted with a heterocycle selected from piperidinyl, piperazinyl and morpholinyl each of which may optionally be substituted with CH₃.

Another embodiment of the invention relates to compounds wherein R¹ is as defined immediately above and R² is OR¹² and R¹² is C₁₋₄ alkyl optionally substituted with aryl or a pharmaceutically acceptable salt thereof. In one embodiment R¹² is methyl or ethyl. In another embodiment R¹² is C₁₋₄ substituted with phenyl which optionally is substituted with Cl.

Another embodiment of the invention relates to compounds of Formula I wherein R¹ is as defined above and R² is NR¹⁰R¹¹ and each of R¹⁰ and R¹¹ are independently selected from H, and C₁₋₆ alkyl that is optionally substituted with NH₂, OH, CONH₂, cycloalkyl, preferably cyclopentane, heterocycle, preferably piperidine, heteroaryl, preferably thiophene, or aryl, preferably phenyl that itself is optionally substituted with Cl, F or CF₃, or a pharmaceutically acceptable salt thereof.

In another embodiment, R² is NR¹⁰R¹¹ and one of R¹⁰ or R¹¹ is heterocycle optionally substituted with aryl, preferably phenyl, that is optionally substituted with Cl.

Another embodiment of the invention relates to compounds of Formula I wherein R¹ and R² are as defined immediately above and R³ is Cl, or a pharmaceutically acceptable salt thereof.

Another embodiment of the invention relates to compounds of Formula I wherein R¹ and R² are as defined immediately above and R³ is CH₃, or a pharmaceutically acceptable salt thereof.

Another embodiment of the invention relates to compounds of Formula I wherein R¹ and R² are as defined immediately above and R³ is H, Br or F, or a pharmaceutically acceptable salt thereof.

Another embodiment of the invention relates to compounds of Formula I wherein R¹, R² and R³ are as defined immediately above and R⁴ is H, or a pharmaceutically acceptable salt thereof.

Another embodiment of the invention relates to compounds of Formula I wherein R¹, R² and R³ are as defined immediately above and R⁴ is F, or a pharmaceutically acceptable salt thereof.

Another embodiment of the invention relates to compounds of Formula I wherein R¹, R², R³ and R⁴ are as defined immediately above and R⁵ is H, or a pharmaceutically acceptable salt thereof.

Another embodiment of the invention relates to compounds of Formula I wherein R¹, R², R³ and R⁴ are as defined immediately above and R⁵ is CH₃, or a pharmaceutically acceptable salt thereof.

Another embodiment of the invention relates to compounds of Formula I wherein R⁶ and R⁷ are independently selected from H, C₁₋₄ alkyl that is optionally substituted with OH or OC₁₋₄ alkyl and N-ethyl.

In another embodiment R⁶ and R⁷ are independently selected from heterocycle and cycloalkyl, wherein the cycloalkyl may be substituted with CH₃, OH, NH₂, and C₁₋₄ alkyl and the heterocyle optionally may be substituted with C₁₋₄ alkyl, or a pharmaceutically acceptable salt thereof.

Another embodiment of the invention relates to compounds of Formula I wherein R¹, R², R³, R⁴, R⁵, R⁶ and R⁷ are as defined immediately above and R⁸ and R⁹ are independently H or CH₃, or a pharmaceutically acceptable salt thereof.

Another embodiment of the invention relates to compounds of Formula I wherein R¹, R², R³, R⁴, R⁵, R⁶, R⁷, R⁸ and R⁹ are as defined immediately above and R¹⁰ and R¹¹ are independently selected from H and C₁₋₆ alkyl that is optionally substituted with aryl that is optionally substituted with Cl, F or CH₃, or a pharmaceutically acceptable salt thereof.

Another embodiment of the invention relates to compounds of Formula I wherein R¹, R², R³, R⁴, R⁵, R⁶, R⁷, R⁸ and R⁹ are as defined immediately above and R¹⁰ and R¹¹ are independently selected from H and C₁₋₆ alkyl that is optionally substituted with heterocycle that is optionally substituted with CH₃, or a pharmaceutically acceptable salt thereof.

Another embodiment of the invention relates to compounds of Formula I wherein R¹, R², R³, R⁴, R⁵, R⁶, R⁷, R⁸ and R⁹ are as defined immediately above and R¹⁰ and R¹¹ are independently selected from H and C₁₋₆ alkyl that is optionally substituted with heteroaryl. In one embodiment the heteroaryl is thiophenyl.

Another embodiment of the invention relates to compounds of Formula I wherein R¹, R², R³, R⁴, R⁵, R⁶, R⁷, R⁸ and R⁹ are as defined immediately above and R¹⁰ and R¹¹ are independently selected from H, OH, OCH₃, NH₂ and CONH₂, or a pharmaceutically acceptable salt thereof.

Another embodiment of the invention relates to compounds of Formula I wherein R¹, R², R³, R⁴, R⁵, R⁶, R⁷, R⁸ and R⁹ are as defined immediately above and R¹⁰ and R¹¹ are independently selected from H and aryl optionally substituted with Cl, or a pharmaceutically acceptable salt thereof.

Another embodiment of the invention relates to compounds of Formula I wherein R¹, R², R³, R⁴, R⁵, R⁶, R⁷, R⁸ and R⁹ are as defined immediately above, R¹⁰ is H and R¹¹ is C₁₋₆ alkyl that is substituted with up to three substituents selected from heteroaryl, NR⁸R⁹ and aryl which itself is optionally substituted with Cl, or a pharmaceutically acceptable salt thereof.

In another embodiment R¹⁰ and R¹¹ are both methyl.

Another embodiment of the invention relates to compounds of Formula I wherein R¹, R², R³, R⁴, R⁵, R⁶, R⁷, R⁸, R⁹, and R¹⁰ and R¹¹ are as defined immediately above and R¹² is selected from C₁₋₄ alkyl, preferably CH₃, or C₁₋₄ alkyl optionally substituted with aryl, preferably phenyl, that is optionally substituted with Cl, or a pharmaceutically acceptable salt thereof.

It is contemplated herein that salts of compounds of formula I such as hydrochloride or trifluoroacetic acid salts include salts with multiple conjugates such as mono HCl, di-HCl, etc.

Compounds according to the invention include:
3-[(2-Methoxy-7-oxo-7,8-dihydro-pyrido[2,3-d]pyrimidine-6-carbonyl)-amino]-4-methyl-benzoic acid methyl ester (Example 19);
4-Chloro-3-[(2-methoxy-7-oxo-7,8-dihydro-pyrido[2,3-d]pyrimidine-6-carbonyl)-amino]-benzoic acid methyl ester (Example 21);
4-Chloro-3-[(2-methoxy-7-oxo-7,8-dihydro-pyrido[2,3-d]pyrimidine-6-carbonyl)-amino]-benzoic acid (Example 22);
4-Methyl-3-[(2-methylsulfanyl-7-oxo-7,8-dihydro-pyrido[2,3-d]pyrimidine-6-carbonyl)-amino]-benzoic acid methyl ester (Example 25);
3-[(2-Methoxy-7-oxo-7,8-dihydro-pyrido[2,3-d]pyrimidine-6-carbonyl)-amino]-4-methyl-benzoic acid benzyl ester (Example 26);
2-Methoxy-7-oxo-7,8-dihydro-pyrido[2,3-d]pyrimidine-6-carboxylic acid (5-benzylcarbamoyl-2-methyl-phenyl)-amide (Example 27);
4-Methyl-3-[(2-morpholin-4-yl-7-oxo-7,8-dihydro-pyrido[2,3-d]pyrimidine-6-carbonyl)-amino]-benzoic acid methyl ester (Example 29);
4-Chloro-3-[(2-methylsulfanyl-7-oxo-7,8-dihydro-pyrido[2,3-d]pyrimidine-6-carbonyl)-amino]-benzoic acid methyl ester (Example 30);
2-Methoxy-7-oxo-7,8-dihydro-pyrido[2,3-d]pyrimidine-6-carboxylic acid (5-benzylcarbamoyl-2-chloro-phenyl)-amide (Example 31);
4-Chloro-3-[(2-morpholin-4-yl-7-oxo-7,8-dihydro-pyrido[2,3-d]pyrimidine-6-carbonyl)-amino]-benzoic acid methyl ester (Example 33);
4-Chloro-3-[(7-oxo-2-piperidin-1-yl-7,8-dihydro-pyrido[2,3-d]pyrimidine-6-carbonyl)-amino]-benzoic acid methyl ester (Example 34);
4-Chloro-3-{[2-(4-methyl-piperazin-1-yl)-7-oxo-7,8-dihydro-pyrido[2,3-d]pyrimidine-6-carbonyl]amino}-benzoic acid methyl ester (Example 35);
4-Chloro-3-[(7-oxo-2-piperazin-1-yl-7,8-dihydro-pyrido[2,3-d]pyrimidine-6-carbonyl)-amino]-benzoic acid methyl ester (Example 37);
4-Chloro-3-{[2-(2,3-dihydroxy-propylamino)-7-oxo-7,8-dihydro-pyrido[2,3-d]pyrimidine-6-carbonyl]amino}-benzoic acid methyl ester (Example 38);
3-({2-[Bis-(2-hydroxy-ethyl)-amino]-7-oxo-7,8-dihydro-pyrido[2,3-d]pyrimidine-6-carbonyl}-amino)-4-chloro-benzoic acid methyl ester (Example 39);
3-({2-[Bis-(2-ethoxy-ethyl)-amino]-7-oxo-7,8-dihydro-pyrido[2,3-d]pyrimidine-6-carbonyl}-amino)-4-chloro-benzoic acid methyl ester (Example 40);
4-Chloro-3-{[2-(2-hydroxy-ethylamino)-7-oxo-7,8-dihydro-pyrido[2,3-d]pyrimidine-6-carbonyl]amino}-benzoic acid methyl ester (Example 41);
4-Chloro-3-{[2-(2-ethoxy-ethylamino)-7-oxo-7,8-dihydro-pyrido[2,3-d]pyrimidine-6-carbonyl]-amino}-benzoic acid methyl ester (Example 42);
4-Fluoro-3-[(2-methoxy-7-oxo-7,8-dihydro-pyrido[2,3-d]pyrimidine-6-carbonyl)-amino]-benzoic acid methyl ester (Example 43);
4-Chloro-3-{[2-(2-ethoxy-ethoxy)-7-oxo-7,8-dihydro-pyrido[2,3-d]pyrimidine-6-carbonyl]amino}-benzoic acid methyl ester (Example 44);
4-Chloro-3-{[2-(2-diethylamino-ethoxy)-7-oxo-7,8-dihydro-pyrido[2,3-d]pyrimidine-6-carbonyl]amino}-benzoic acid methyl ester (Example 45);
2-Methoxy-7-oxo-7,8-dihydro-pyrido[2,3-d]pyrimidine-6-carboxylic acid [2-chloro-5-(4-chloro-benzylcarbamoyl)-phenyl]-amide (Example 46);
2-Methoxy-7-oxo-7,8-dihydro-pyrido[2,3-d]pyrimidine-6-carboxylic acid [2-chloro-5-(2-chloro-benzylcarbamoyl)-phenyl]-amide (Example 47);
4-Bromo-3-[(2-methoxy-7-oxo-7,8-dihydro-pyrido[2,3-d]pyrimidine-6-carbonyl)-amino]-benzoic acid methyl ester (Example 48);
2-Methoxy-7-oxo-7,8-dihydro-pyrido[2,3-d]pyrimidine-6-carboxylic acid (2-chloro-5-phenylcarbamoyl-phenyl)-amide (Example 49);
4-Chloro-3-[(2-methoxy-7-oxo-7,8-dihydro-pyrido[2,3-d]pyrimidine-6-carbonyl)-amino]-benzoic acid benzyl ester (Example 50);
4-Chloro-3-[(2-methoxy-7-oxo-7,8-dihydro-pyrido[2,3-d]pyrimidine-6-carbonyl)-amino]-benzoic acid 4-chloro-benzyl ester (Example 51);
4-Chloro-3-[(2-methoxy-7-oxo-7,8-dihydro-pyrido[2,3-d]pyrimidine-6-carbonyl)-amino]-benzoic acid 2-chloro-benzyl ester (Example 52);
4-Chloro-3-[(2-methoxy-7-oxo-7,8-dihydro-pyrido[2,3-d]pyrimidine-6-carbonyl)-amino]-benzoic acid 3-chloro-benzyl ester (Example 53);
2-Methoxy-7-oxo-7,8-dihydro-pyrido[2,3-d]pyrimidine-6-carboxylic acid [2-chloro-5-(3-chloro-benzylcarbamoyl)-phenyl]-amide (Example 54);
2-Methoxy-7-oxo-7,8-dihydro-pyrido[2,3-d]pyrimidine-6-carboxylic acid (2-chloro-5-dimethylcarbamoyl-phenyl)-amide (Example 55);
2-Methoxy-7-oxo-7,8-dihydro-pyrido[2,3-d]pyrimidine-6-carboxylic acid {2-chloro-5-[(thiophen-2-ylmethyl)-carbamoyl]-phenyl}-amide (Example 57);
2-Methoxy-7-oxo-7,8-dihydro-pyrido[2,3-d]pyrimidine-6-carboxylic acid {2-chloro-5-[(thiophen-3-ylmethyl)-carbamoyl]-phenyl}-amide (Example 58);
2-Methoxy-7-oxo-7,8-dihydro-pyrido[2,3-d]pyrimidine-6-carboxylic acid [2-chloro-5-(3-trifluoromethyl-benzylcarbamoyl)-phenyl]-amide (Example 59);
2-Methoxy-7-oxo-7,8-dihydro-pyrido[2,3-d]pyrimidine-6-carboxylic acid [5-(benzyl-methyl-carbamoyl)-2-chloro-phenyl]-amide (Example 60);
2-Methoxy-7-oxo-7,8-dihydro-pyrido[2,3-d]pyrimidine-6-carboxylic acid [5-(3-chloro-benzylcarbamoyl)-2-methyl-phenyl]-amide (Example 62);
2-Methoxy-7-oxo-7,8-dihydro-pyrido[2,3-d]pyrimidine-6-carboxylic acid {5-[(3-chloro-benzyl)-methyl-carbamoyl]-2-methyl-phenyl}-amide (Example 63);
7-oxo-2-piperidin-1-yl-7,8-dihydro-pyrido[2,3-d]pyrimidine-6-carboxylic acid {2-chloro-5-[(3-chloro-benzyl)-methyl-carbamoyl]-phenyl}-amide (Example 64);
2-Methoxy-7-oxo-7,8-dihydro-pyrido[2,3-d]pyrimidine-6-carboxylic acid {2-chloro-5-[(3-chloro-benzyl)-methyl-carbamoyl]-phenyl}-amide (Example 65);
2-Methoxy-7-oxo-7,8-dihydro-pyrido[2,3-d]pyrimidine-6-carboxylic acid {2-chloro-5-[(pyridin-2-ylmethyl)-carbamoyl]-phenyl}-amide (Example 66);
2-Methoxy-7-oxo-7,8-dihydro-pyrido[2,3-d]pyrimidine-6-carboxylic acid {2-chloro-5-[(pyridin-3-ylmethyl)-carbamoyl]-phenyl}-amide (Example 67);
2-Methoxy-7-oxo-7,8-dihydro-pyrido[2,3-d]pyrimidine-6-carboxylic acid {2-chloro-5-[(pyridin-4-ylmethyl)-carbamoyl]-phenyl}-amide (Example 68);
2-Methoxy-7-oxo-7,8-dihydro-pyrido[2,3-d]pyrimidine-6-carboxylic acid (2-chloro-5-phenethylcarbamoyl-phenyl)-amide (Example 70);
2-Methoxy-7-oxo-7,8-dihydro-pyrido[2,3-d]pyrimidine-6-carboxylic acid {2-chloro-5-[2-(3-chloro-phenyl)-ethylcarbamoyl]-phenyl}-amide (Example 71);
2-Methoxy-7-oxo-7,8-dihydro-pyrido[2,3-d]pyrimidine-6-carboxylic acid [5-(2-amino-1-phenyl-ethylcarbamoyl)-2-chloro-phenyl]-amide (Example 72);
2-Methylsulfanyl-7-oxo-7,8-dihydro-pyrido[2,3-d]pyrimidine-6-carboxylic acid [2-chloro-5-(3-chloro-benzylcarbamoyl)-phenyl]-amide (Example 74);
2-(4-Methyl-piperazin-1-yl)-7-oxo-7,8-dihydro-pyrido[2,3-d]pyrimidine-6-carboxylic acid [2-chloro-5-(3-chloro-benzylcarbamoyl)-phenyl]-amide (Example 75);
2-Methoxy-7-oxo-7,8-dihydro-pyrido[2,3-d]pyrimidine-6-carboxylic acid [5-(3-amino-1-phenyl-propylcarbamoyl)-2-chloro-phenyl]-amide (Example 76);
2-Methoxy-7-oxo-7,8-dihydro-pyrido[2,3-d]pyrimidine-6-carboxylic acid (2-chloro-5-methylcarbamoyl-phenyl)-amide (Example 78);
2-Methoxy-7-oxo-7,8-dihydro-pyrido[2,3-d]pyrimidine-6-carboxylic acid (2-chloro-5-isobutylcarbamoyl-phenyl)-amide (Example 79);
2-(3-Morpholin-4-yl-propoxy)-7-oxo-7,8-dihydro-pyrido[2,3-d]pyrimidine-6-carboxylic acid [2-chloro-5-(3-chloro-benzylcarbamoyl)-phenyl]-amide (Example 80);
2-(2-Morpholin-4-yl-ethoxy)-7-oxo-7,8-dihydro-pyrido[2,3-d]pyrimidine-6-carboxylic acid [2-chloro-5-(3-chloro-benzylcarbamoyl)-phenyl]-amide (Example 81);
4-Chloro-3-[(2-methoxy-7-oxo-7,8-dihydro-pyrido[2,3-d]pyrimidine-6-carbonyl)-amino]-benzoic acid ethyl ester (Example 82);
2-Methoxy-7-oxo-7,8-dihydro-pyrido[2,3-d]pyrimidine-6-carboxylic acid {5-[3-amino-1-(3-chloro-phenyl)-propylcarbamoyl]-2-chloro-phenyl}-amide (Example 84);
2-Methoxy-7-oxo-7,8-dihydro-pyrido[2,3-d]pyrimidine-6-carboxylic acid {5-[2-amino-1-(3-chloro-phenyl)-ethylcarbamoyl]-2-chloro-phenyl}-amide (Example 85);
2-Morpholin-4-yl-7-oxo-7,8-dihydro-pyrido[2,3-d]pyrimidine-6-carboxylic acid [2-chloro-5-(3-chloro-benzylcarbamoyl)-phenyl]-amide (Example 86);
2-[2-(4-Methyl-piperazin-1-yl)-ethoxy]-7-oxo-7,8-dihydro-pyrido[2,3-d]pyrimidine-6-carboxylic acid [2-chloro-5-(3-chloro-benzylcarbamoyl)-phenyl]amide (Example 87);
2-Methoxy-7-oxo-7,8-dihydro-pyrido[2,3-d]pyrimidine-6-carboxylic acid (5-carbamoyl-2-chloro-phenyl)-amide (Example 88);
7-Oxo-2-(2-pyrrolidin-1-yl-ethylamino)-7,8-dihydro-pyrido[2,3-d]pyrimidine-6-carboxylic acid [2-chloro-5-(3-chloro-benzylcarbamoyl)-phenyl]amide (Example 89);
2-(3-Morpholin-4-yl-propylamino)-7-oxo-7,8-dihydro-pyrido[2,3-d]pyrimidine-6-carboxylic acid [2-chloro-5-(3-chloro-benzylcarbamoyl)-phenyl]amide (Example 90);
2-(4-Hydroxy-piperidin-1-yl)-7-oxo-7,8-dihydro-pyrido[2,3-d]pyrimidine-6-carboxylic acid [2-chloro-5-(3-chloro-benzylcarbamoyl)-phenyl]-amide (Example 92);
2-(4-Hydroxy-cyclohexylamino)-7-oxo-7,8-dihydro-pyrido[2,3-d]pyrimidine-6-carboxylic acid [2-chloro-5-(3-chloro-benzylcarbamoyl)-phenyl]amide (Example 94);
2-(4-Hydroxy-cyclohexylamino)-7-oxo-7,8-dihydro-pyrido[2,3-d]pyrimidine-6-carboxylic acid [2-chloro-5-(3-chloro-benzylcarbamoyl)-phenyl]amide (Example 95);
2-Methoxy-7-oxo-7,8-dihydro-pyrido[2,3-d]pyrimidine-6-carboxylic acid {2-chloro-5-[1-(3-chloro-phenyl)-3-dimethylamino-propylcarbamoyl]-phenyl}-amide (Example 96);
2-(4-Amino-piperidin-1-yl)-7-oxo-7,8-dihydro-pyrido[2,3-d]pyrimidine-6-carboxylic acid [2-chloro-5-(3-chloro-benzylcarbamoyl)-phenyl]-amide (Example 97);
2-[2-(4-Methyl-piperazin-1-yl)-ethylamino]-7-oxo-7,8-dihydro-pyrido[2,3-d]pyrimidine-6-carboxylic acid [2-chloro-5-(3-chloro-benzylcarbamoyl)-phenyl]amide (Example 99);
N-(2-chloro-5-(3-chlorobenzylcarbamoyl)phenyl)-2-(2-(4-methylpiperazin-1-yl)ethylamino)-7-oxo-7,8-dihydropyrido[2,3-d]pyrimidine-6-carboxamide hydrochloride (Example 100);
2-[3-(4-Methyl-piperazin-1-yl)-propylamino]-7-oxo-7,8-dihydro-pyrido[2,3-d]pyrimidine-6-carboxylic acid [2-chloro-5-(3-chloro-benzylcarbamoyl)-phenyl]-amide (Example 101);
2-[3-(4-Methyl-piperazin-1-yl)-propylamino]-7-oxo-7,8-dihydro-pyrido[2,3-d]pyrimidine-6-carboxylic acid [2-chloro-5-(3-chloro-benzylcarbamoyl)-phenyl]-amide hydrochloride (Example 102);
N-(2-chloro-5-(3-chlorobenzylcarbamoyl)phenyl)-2-(2-hydroxyethylamino)-7-oxo-7,8-dihydropyrido[2,3-d]pyrimidine-6-carboxamide (Example 103);
N-(2-chloro-5-(3-chlorobenzylcarbamoyl)phenyl)-2-(2-(1-methylpiperidin-4-yl)ethylamino)-7-oxo-7,8-dihydropyrido[2,3-d]pyrimidine-6-carboxamide (Example 104);
N-(5-(2-amino-1-phenylethylcarbamoyl)-2-chlorophenyl)-2-(methylthio)-7-oxo-7,8-dihydropyrido[2,3-d]pyrimidine-6-carboxamide (Example 105);
2-Methylsulfanyl-7-oxo-7,8-dihydro-pyrido[2,3-d]pyrimidine-6-carboxylic acid [5-(3-amino-1-phenyl-propylcarbamoyl)-2-chloro-phenyl]-amide; hydrochloride (Example 107);
2-(4-Acetylpiperazin-1-yl)-N-(2-chloro-5-(3-chlorobenzylcarbamoyl)phenyl)-7-oxo-7,8-dihydropyrido[2,3-d]pyrimidine-6-carboxamide (Example 108);
N-(5-(3-Amino-1-phenylpropylcarbamoyl)-2-chlorophenyl)-2-methyl-7-oxo-7,8-dihydropyrido[2,3-d]pyrimidine-6-carboxamide hydrochloride (Example 112);
N-(5-(2-Amino-1-phenylethylcarbamoyl)-2-chlorophenyl)-2-methyl-7-oxo-7,8-dihydropyrido[2,3-d]pyrimidine-6-carboxamide hydrochloride (Example 114);
2-((1s,4s)-4-Aminocyclohexylamino)-N-(2-chloro-5-(3-chlorobenzylcarbamoyl)phenyl)-7-oxo-7,8-dihydropyrido[2,3-d]pyrimidine-6-carboxamide hydrochloride (Example 118);
N-(2-Chloro-5-(3-chlorobenzylcarbamoyl)phenyl)-7-oxo-2-(piperazin-1-yl)-7,8-dihydropyrido[2,3-d]pyrimidine-6-carboxamide hydrochloride (Example 119);
N-(5-(3-Amino-1-phenylpropylcarbamoyl)-2-chlorophenyl)-2-(3-(4-methylpiperazin-1-yl)propylamino)-7-oxo-7,8-dihydropyrido[2,3-d]pyrimidine-6-carboxamide (Example 120);
N-(5-(3-Amino-1-phenylpropylcarbamoyl)-2-chlorophenyl)-2-(3-(4-methylpiperazin-1-yl)propylamino)-7-oxo-7,8-dihydropyrido[2,3-d]pyrimidine-6-carboxamide hydrochloride (Example 121);
N-(5-(2-Amino-1-phenylethylcarbamoyl)-2-chlorophenyl)-2-(3-(4-methylpiperazin-1-yl)propylamino)-7-oxo-7,8-dihydropyrido[2,3-d]pyrimidine-6-carboxamide (Example 122);
N-(5-(2-Amino-1-phenylethylcarbamoyl)-2-chlorophenyl)-2-(3-(4-methylpiperazin-1-yl)propylamino)-7-oxo-7,8-dihydropyrido[2,3-d]pyrimidine-6-carboxamide hydrochloride (Example 123);
N-(2-Chloro-5-(3-chlorobenzylcarbamoyl)phenyl)-2-((2,2-dimethyl-1,3-dioxolan-4-yl)methylamino)-7-oxo-7,8-dihydropyrido[2,3-d]pyrimidine-6-carboxamide (Example 124);
N-(2-Chloro-5-(3-chlorobenzylcarbamoyl)phenyl)-7-oxo-2-(piperidin-4-ylamino)-7,8-dihydropyrido[2,3-d]pyrimidine-6-carboxamide hydrochloride (Example 125);
N-(2-Chloro-5-(3-chlorobenzylcarbamoyl)phenyl)-2-(2,3-dihydroxypropylamino)-7-oxo-7,8-dihydropyrido[2,3-d]pyrimidine-6-carboxamide (Example 126);
N-(2-Chloro-5-(3-chlorobenzylcarbamoyl)phenyl)-2-(2-hydroxy-2-methylpropylamino)-7-oxo-7,8-dihydropyrido[2,3-d]pyrimidine-6-carboxamide (Example 127);
N-(5-(3-Amino-1-phenylpropylcarbamoyl)-2-chlorophenyl)-2-(2-(4-methylpiperazin-1-yl)ethylamino)-7-oxo-7,8-dihydropyrido[2,3-d]pyrimidine-6-carboxamide hydrochloride (Example 132);
(S)—N-(5-(2-Amino-1-phenylethylcarbamoyl)-2-chlorophenyl)-2-methoxy-7-oxo-7,8-dihydropyrido[2,3-d]pyrimidine-6-carboxamide (Example 133);
2-Methoxy-7-oxo-7,8-dihydro-pyrido[2,3-d]pyrimidine-6-carboxylic acid {5-[(R)-2-amino-1-(3-chloro-phenyl)-ethylcarbamoyl]-2-chloro-phenyl}-amide (Example 134);
2-Methoxy-7-oxo-7,8-dihydro-pyrido[2,3-d]pyrimidine-6-carboxylic acid {5-[(S)-2-amino-1-(3-chloro-phenyl)-ethylcarbamoyl]-2-chloro-phenyl}-amide (Example 135);
2-Methoxy-7-oxo-7,8-dihydro-pyrido[2,3-d]pyrimidine-6-carboxylic acid {5-[(S)-3-amino-1-(3-chloro-phenyl)-propylcarbamoyl]-2-chloro-phenyl}-amide (Example 136);
N-(5-(3-Amino-1-phenylpropylcarbamoyl)-2-chlorophenyl)-2-(4-aminopiperidin-1-yl)-7-oxo-7,8-dihydropyrido[2,3-d]pyrimidine-6-carboxamide hydrochloride (Example 137);
N-(5-(2-Amino-1-phenylethylcarbamoyl)-2-chlorophenyl)-2-(4-aminopiperidin-1-yl)-7-oxo-7,8-dihydropyrido[2,3-d]pyrimidine-6-carboxamide hydrochloride (Example 138);
N-(5-(2-Amino-1-phenylethylcarbamoyl)-2-chlorophenyl)-2-(2-(4-methylpiperazin-1-yl)ethylamino)-7-oxo-7,8-dihydropyrido[2,3-d]pyrimidine-6-carboxamide hydrochloride (Example 139);
N-(2-Chloro-5-(2-hydroxy-1-phenylethylcarbamoyl)phenyl)-2-(methylthio)-7-oxo-7,8-dihydropyrido[2,3-d]pyrimidine-6-carboxamide (Example 140);
N-(2-Chloro-5-(3-hydroxy-1-phenylpropylcarbamoyl)phenyl)-2-(methylthio)-7-oxo-7,8-dihydropyrido[2,3-d]pyrimidine-6-carboxamide (Example 141);
2-Morpholin-4-yl-7-oxo-7,8-dihydro-pyrido[2,3-d]pyrimidine-6-carboxylic acid [2-chloro-5-(2,2-dimethyl-propylcarbamoyl)-phenyl]-amide (Example 142);
2-Morpholin-4-yl-7-oxo-7,8-dihydro-pyrido[2,3-d]pyrimidine-6-carboxylic acid {2-chloro-5-[1-(3-chloro-phenyl)-ethylcarbamoyl]-phenyl}-amide (Example 143);
2-Morpholin-4-yl-7-oxo-7,8-dihydro-pyrido[2,3-d]pyrimidine-6-carboxylic acid [2-chloro-5-(3-chloro-benzylcarbamoyl)-4-fluoro-phenyl]-amide (Example 144);
2-(4-Aminopiperidin-1-yl)-N-(2-chloro-5-(2-hydroxy-1-phenylethylcarbamoyl)phenyl)-7-oxo-7,8-dihydropyrido[2,3-d]pyrimidine-6-carboxamide hydrochloride (Example 147);
2-(4-Aminopiperidin-1-yl)-N-(2-chloro-5-(3-hydroxy-1-phenylpropylcarbamoyl)phenyl)-7-oxo-7,8-dihydropyrido[2,3-d]pyrimidine-6-carboxamide hydrochloride (Example 148);
N-(5-(2-Amino-1-phenylethylcarbamoyl)-2-chlorophenyl)-7-oxo-7,8-dihydropyrido[2,3-d]pyrimidine-6-carboxamide hydrochloride (Example 151);
N-(5-(3-Amino-1-phenylpropylcarbamoyl)-2-chlorophenyl)-7-oxo-7,8-dihydropyrido[2,3-d]pyrimidine-6-carboxamide hydrochloride (Example 152);
N-(2-Chloro-5-(2-hydroxy-1-phenylethylcarbamoyl)phenyl)-2-(2-hydroxy-2-methylpropylamino)-7-oxo-7,8-dihydropyrido[2,3-d]pyrimidine-6-carboxamide (Example 153);
N-(2-Chloro-5-(3-hydroxy-1-phenylpropylcarbamoyl)phenyl)-2-(2-hydroxy-2-methylpropylamino)-7-oxo-7,8-dihydropyrido[2,3-d]pyrimidine-6-carboxamide (Example 154);
N-(2-Chloro-5-(3-chlorobenzylcarbamoyl)phenyl)-2-(dimethylamino)-7-oxo-7,8-dihydropyrido[2,3-d]pyrimidine-6-carboxamide (Example 155);
2-Morpholin-4-yl-7-oxo-7,8-dihydro-pyrido[2,3-d]pyrimidine-6-carboxylic acid [2-chloro-5-((R)-1-phenyl-ethylcarbamoyl)-phenyl]-amide (Example 156);
2-Morpholin-4-yl-7-oxo-7,8-dihydro-pyrido[2,3-d]pyrimidine-6-carboxylic acid [2-chloro-5-((S)-1-phenyl-ethylcarbamoyl)-phenyl]amide (Example 157);
(R)—N-(2-Chloro-5-(3-hydroxy-1-phenylpropylcarbamoyl)phenyl)-2-methoxy-7-oxo-7,8-dihydropyrido[2,3-d]pyrimidine-6-carboxamide (Example 158);
(S)—N-(2-Chloro-5-(3-hydroxy-1-phenylpropylcarbamoyl)phenyl)-2-methoxy-7-oxo-7,8-dihydropyrido[2,3-d]pyrimidine-6-carboxamide (Example 159);
2-Morpholin-4-yl-7-oxo-7,8-dihydro-pyrido[2,3-d]pyrimidine-6-carboxylic acid {5-[3-amino-1-(3-chloro-phenyl)-propylcarbamoyl]-2-chloro-4-fluoro-phenyl}-amide (Example 160);
2-Morpholin-4-yl-7-oxo-7,8-dihydro-pyrido[2,3-d]pyrimidine-6-carboxylic acid [2-chloro-5-(3-fluoro-benzylcarbamoyl)-phenyl]-amide (Example 161);
2-Morpholin-4-yl-7-oxo-7,8-dihydro-pyrido[2,3-d]pyrimidine-6-carboxylic acid [2-chloro-4-fluoro-5-(3-fluoro-benzylcarbamoyl)-phenyl]-amide (Example 162);
N-(5-(3-Aminopropylcarbamoyl)-2-chlorophenyl)-2-methoxy-7-oxo-7,8-dihydropyrido[2,3-d]pyrimidine-6-carboxamide (Example 164);
N-(2-Chloro-5-(4-phenylpiperidin-4-ylcarbamoyl)phenyl)-2-methoxy-7-oxo-7,8-dihydropyrido[2,3-d]pyrimidine-6-carboxamide (Example 166);
N-(5-(3-Amino-3-oxopropylcarbamoyl)-2-chlorophenyl)-2-methoxy-7-oxo-7,8-dihydropyrido[2,3-d]pyrimidine-6-carboxamide (Example 167);
(R)—N-(5-(3-Amino-1-phenylpropylcarbamoyl)-2-chlorophenyl)-2-methyl-7-oxo-7,8-dihydropyrido[2,3-d]pyrimidine-6-carboxamide hydrochloride (Example 168);
N-(5-(3-Amino-1-phenylpropylcarbamoyl)-2-chlorophenyl)-2-methoxy-4-methyl-7-oxo-7,8-dihydropyrido[2,3-d]pyrimidine-6-carboxamide 2,2,2-trifluoroacetate (Example 169);
N-(5-(2-Amino-1-phenylethylcarbamoyl)-2-chlorophenyl)-2-methoxy-4-methyl-7-oxo-7,8-dihydropyrido[2,3-d]pyrimidine-6-carboxamide 2,2,2-trifluoroacetate (Example 170);
(R)—N-(5-(3-Amino-1-phenylpropylcarbamoyl)-2-chlorophenyl)-2-methoxy-7-oxo-7,8-dihydropyrido[2,3-d]pyrimidine-6-carboxamide (Example 171);
2-(4-Methyl-piperazin-1-yl)-7-oxo-7,8-dihydro-pyrido[2,3-d]pyrimidine-6-carboxylic acid (5-benzylcarbamoyl-2-chloro-phenyl)-amide (Example 172);
2-(4-Methyl-piperazin-1-yl)-7-oxo-7,8-dihydro-pyrido[2,3-d]pyrimidine-6-carboxylic acid [2-chloro-5-((S)-1-phenyl-ethylcarbamoyl)-phenyl]amide (Example 173);
2-(4-Methyl-piperazin-1-yl)-7-oxo-7,8-dihydro-pyrido[2,3-d]pyrimidine-6-carboxylic acid [2-chloro-5-((R)-1-phenyl-ethylcarbamoyl)-phenyl]amide (Example 174);
(S)—N-(5-(3-Amino-1-phenylpropylcarbamoyl)-2-chlorophenyl)-2-methoxy-7-oxo-7,8-dihydropyrido[2,3-d]pyrimidine-6-carboxamide (Example 176);
2-(4-Methyl-piperazin-1-yl)-7-oxo-7,8-dihydro-pyrido[2,3-d]pyrimidine-6-carboxylic acid [2-chloro-5-(3-fluoro-benzylcarbamoyl)-phenyl]amide (Example 177);
2-(4-Methyl-piperazin-1-yl)-7-oxo-7,8-dihydro-pyrido[2,3-d]pyrimidine-6-carboxylic acid [2-chloro-5-(2,5-difluoro-benzylcarbamoyl)-phenyl]amide (Example 178);
2-(1R,5S)-8-Oxa-3-aza-bicyclo[3.2.1]oct-3-yl-7-oxo-7,8-dihydro-pyrido[2,3-d]pyrimidine-6-carboxylic acid [2-chloro-5-(3-chloro-benzylcarbamoyl)-phenyl]amide (Example 179);
2-(1R,5S)-8-Oxa-3-aza-bicyclo[3.2.1]oct-3-yl-7-oxo-7,8-dihydro-pyrido[2,3-d]pyrimidine-6-carboxylic acid [2-chloro-5-(2-hydroxy-1-phenyl-ethylcarbamoyl)-phenyl]-amide (Example 181);
2-(1R,5S)-8-Oxa-3-aza-bicyclo[3.2.1]oct-3-yl-7-oxo-7,8-dihydro-pyrido[2,3-d]pyrimidine-6-carboxylic acid [2-chloro-5-(3-hydroxy-1-phenyl-propylcarbamoyl)-phenyl]-amide (Example 182);
2-(1R,5S)-8-Oxa-3-aza-bicyclo[3.2.1]oct-3-yl-7-oxo-7,8-dihydro-pyrido[2,3-d]pyrimidine-6-carboxylic acid [5-(3-amino-1-phenyl-propylcarbamoyl)-2-chloro-phenyl]-amide; hydrochloride (Example 183);
(R)—N-(5-(3-Amino-3-phenylpropylcarbamoyl)-2-chlorophenyl)-2-methoxy-7-oxo-7,8-dihydropyrido[2,3-d]pyrimidine-6-carboxamide (Example 184);
N-(5-(5-Amino-1-phenylpentylcarbamoyl)-2-chlorophenyl)-2-methoxy-7-oxo-7,8-dihydropyrido[2,3-d]pyrimidine-6-carboxamide (Example 186);
2-(4-Methyl-piperazin-1-yl)-7-oxo-7,8-dihydro-pyrido[2,3-d]pyrimidine-6-carboxylic acid {5-[(S)-1-aminomethyl-2-(3-fluoro-phenyl)-ethylcarbamoyl]-2-chloro-phenyl}-amide (Example 187);
N-(5-(4-Amino-1-phenylbutylcarbamoyl)-2-chlorophenyl)-2-methoxy-7-oxo-7,8-dihydropyrido[2,3-d]pyrimidine-6-carboxamide (Example 189);
N-(5-(2-Amino-1-cyclohexylethylcarbamoyl)-2-chlorophenyl)-2-methoxy-7-oxo-7,8-dihydropyrido[2,3-d]pyrimidine-6-carboxamide (Example 192);
N-(5-(3-Amino-1-cyclohexylpropylcarbamoyl)-2-chlorophenyl)-2-methoxy-7-oxo-7,8-dihydropyrido[2,3-d]pyrimidine-6-carboxamide 2,2,2-trifluoroacetate (Example 193);
N-(5-(3-amino-2,2-difluoro-1-phenylpropylcarbamoyl)-2-chlorophenyl)-2-methoxy-7-oxo-7,8-dihydropyrido[2,3-d]pyrimidine-6-carboxamide (Example 196);
2-Methoxy-7-oxo-7,8-dihydro-pyrido[2,3-d]pyrimidine-6-carboxylic acid [5-(3-amino-1-cyclopentyl-propylcarbamoyl)-2-chloro-phenyl]-amide (Example 198);
2-Methoxy-7-oxo-7,8-dihydro-pyrido[2,3-d]pyrimidine-6-carboxylic acid [5-(2-amino-1-cyclopentyl-ethylcarbamoyl)-2-chloro-phenyl]-amide (Example 200);
   and the pharmaceutically acceptable salts of the foregoing compounds.

The compounds of formula I, as well as their salts, that have at least one asymmetric carbon atom may be present as racemic mixtures or different stereoisomers. The various isomers can be isolated by known separation methods, e.g., chromatography.

Compounds disclosed herein and covered by formula I above may exhibit tautomerism or structural isomerism. It is intended that the invention encompasses any tautomeric or structural isomeric form of these compounds, or mixtures of such forms, and is not limited to any one tautomeric or structural isomeric form depicted in the formulas above.

### Dosages

The compounds of the present invention that are inhibitors of DYRK1B are useful in the treatment, amelioration or control of cell proliferative disorders, in particular chemoprevention of cancer. Chemoprevention is defined as inhibiting the development of invasive cancer by either blocking the initiating mutagenic event or by blocking the progression of pre-malignant cells that have already suffered an insult of inhibiting tumor relapse. These compounds and formulations containing said compounds are anticipated to be particularly useful in the treatment or control of solid tumors, such as, for example, lung, pancreas, colon, breast, bone and prostate tumors.

Compounds that are inhibitors of DYRK1A are useful in the treatment, amelioration or control of Down syndrome and Alzheimer's disease.

A “therapeutically effective amount” or “effective amount” of a compound in accordance with this invention means an amount of compound that is effective to alleviate, ameliorate or control symptoms of disease or prolong the survival of the subject being treated.

The therapeutically effective amount or dosage of a compound according to this invention can vary within wide limits. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration; it may be given as continuous infusion.

### Compositions/Formulations

In an alternative embodiment, the present invention includes pharmaceutical compositions comprising at least one compound of formula I, or a pharmaceutically acceptable salt thereof, and a pharmaceutically acceptable excipient and/or carrier.

These pharmaceutical compositions can be suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, as well as the particular mode of administration. The amount of active ingredient which can be combined with a carrier material to produce a single dosage form will generally be that amount of a formula I compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, sachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

The pharmaceutical preparations of the invention can also contain preserving agents, solubilizing agents, stabilizing agents, wetting agents, emulsifying agents, sweetening agents, coloring agents, flavoring agents, salts for varying the osmotic pressure, buffers, coating agents or antioxidants. They can also contain other therapeutically valuable substances, including additional active ingredients other than those of formula I.

### General Synthesis of the Compounds according to the Invention

The present invention also provides methods for the synthesis of the pyrido-pyrimidines of the invention.

The compounds of the invention can be prepared by processes known in the art. Suitable processes for synthesizing these compounds are also provided in the examples. Generally, compounds of formula I can be synthesized according to one of the below described synthetic routes.

The starting materials are either commercially available or can be synthesized by methods known to those of ordinary skill in the art. In general, according to Scheme 1 below, 2-substituted 4-amino-pyrimidine-5-carbaldehyde (1) is condensed with dialkyl malonate with base to give the corresponding pyrido[2,3-d]pyrimidine-6-carboxylic acid ester 2, which is hydrolyzed to the free acid 3. The acid 3 can be coupled with the appropriate 3-amino-benzoate (Scheme 2) to give compound 5. When desired, 5 can be hydrolyzed to the corresponding acid 6 (Scheme 3), which can be further esterified or coupled with an amine by standard methods (Scheme 4) to give compounds I of this invention.

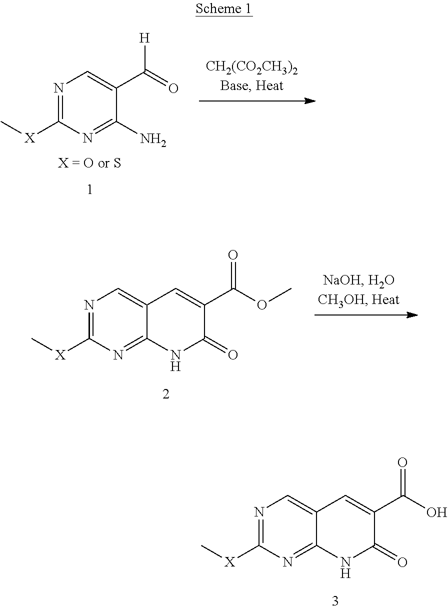

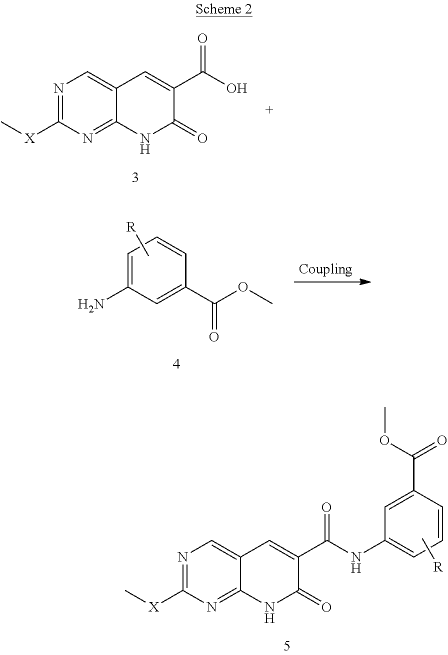

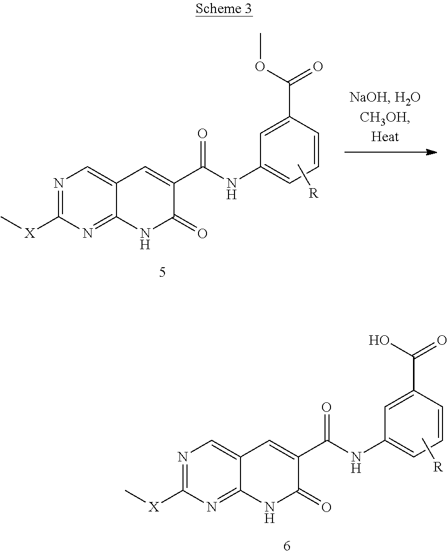

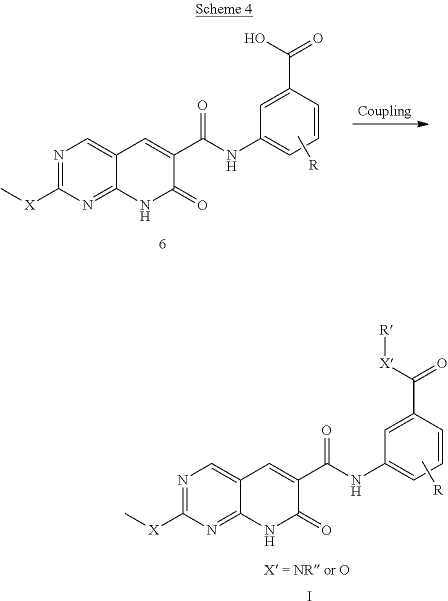

Alternately, the appropriate 3-amino-benzoic acid or 3-nitro benzoic acid can be modified by converting the acid to amide first (Schemes 5-6) to provide various starting materials and intermediates useful in the preparation of compounds I of this invention by standard amide formation reaction instead of the step-wise process of Schemes 2, 3 and 4.

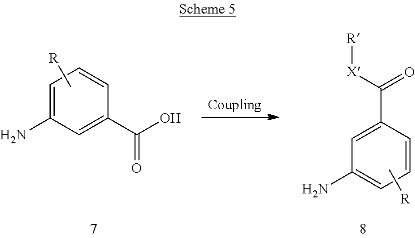

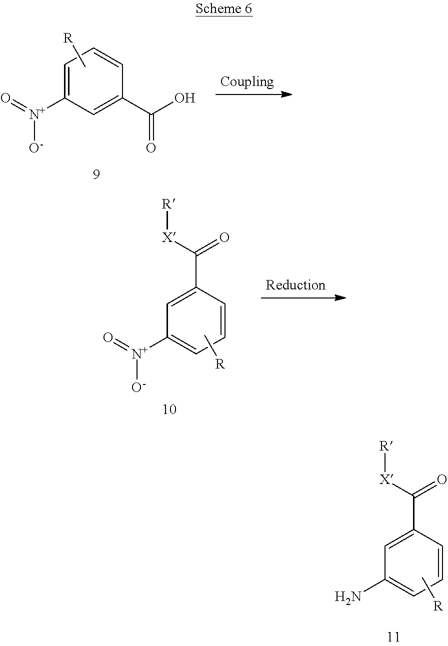

In the case when starting with 2-methylsulfanyl substituent, the sulfur can be oxidized to either the sulfoxide or sulfone. The sulfoxide or sulfone group can then be replaced by the appropriate amine or alcohol to give compounds I of this invention (Scheme 7).

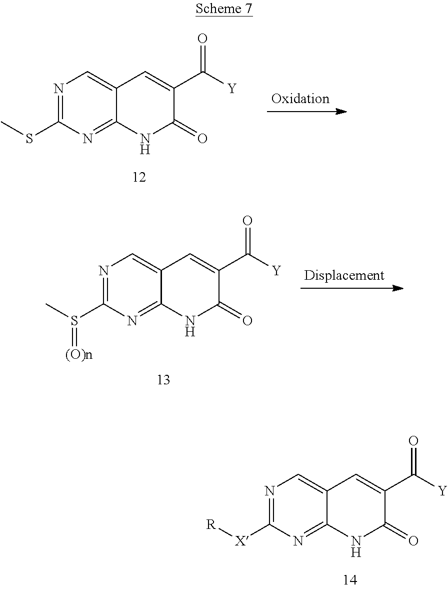

Alternately, according to Scheme 8, the appropriately substituted aniline 8 (Scheme 5) or 11 (Scheme 6) react with the mono acid chloride of methyl malonate to give the amide 16. Amide 16 can then be condensed with the appropriate amino-aldehyde to give compounds I of this invention.

In case of certain amines that contain additional functional groups, appropriate protecting groups (for example tert-butoxy-carbonyl group) may be employed to facilitate synthesis. If such protecting groups are employed, the removal of such protecting groups to generate the compounds of the invention can be accomplished by standard methods known to those skilled in the art of organic synthesis.

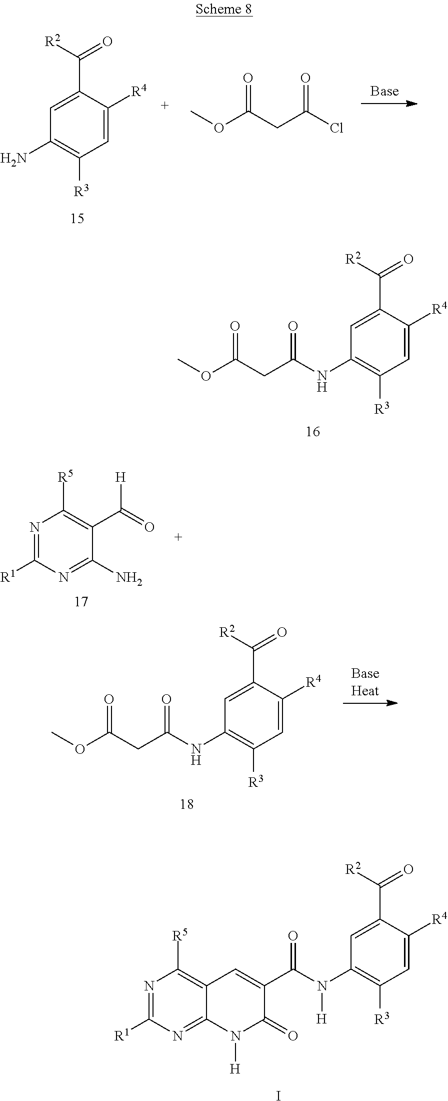

### Crystal Forms

When the compounds of the invention are solids, it is understood by those skilled in the art that these compounds, and their salts, may exist in different crystal or polymorphic forms, all of which are intended to be within the scope of the present invention and specified formulas.

### EXAMPLES

The compounds of the present invention may be synthesized according to known techniques. The following examples and references are provided to aid the understanding of the present invention. The examples are not intended, however, to limit the invention, the true scope of which is set forth in the appended claims. The names of the final products in the examples were generated using AutoNom 2000 Add-in v4.0 SP2, (function in ISIS Draw, Elsevier/MDL), or AutoNom 2000 TT v4.01.305 (Elsevier/MDL), or functions available in ChemDraw Pro Control 11.0.2 (CambridgeSoft Corp.).

### Abbreviations Used in the Examples

### HRMS: High Resolution Mass Spectrometry

### LC-MS: Liquid Chromatography Mass Spectrometry

RT (or rt): Room temperature

### Min: Minutes

### H: Hours

DCM: dichloromethane

### DIPEA: N,N-diisopropylethylamine

### DMF: N,N-dimethylformamide

HATU: O-(7-azabenzotriazol-1-yl)-N,N,N′,N′-tetramethyluronium hexafluorophosphate
HBTU: O-(benzotriazol-1-yl)-N,N,N′,N′-tetramethyluronium hexafluorophosphate
Boc₂O: di-tert-butyl dicarbonate
EtOAc: ethyl acetate
EtOH: ethanol
HCl: hydrogen chloride
HPLC: high performance liquid chromatography
LAH: lithium aluminum hydride
LiOH: lithium hydroxide
MeCN: acetonitrile
MeOH: methanol
MgSO₄: magnesium sulfate
NaHCO₃: sodium bicarbonate
NaOH: sodium hydroxide
TEA: triethylamine
TFA: trifluoroacetic acid
THF: tetrahydrofuran
TLC: thin layer chromatography

### Example 1

### 3-Amino-N-benzyl-4-methyl-benzamide

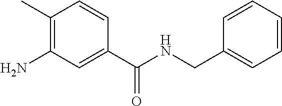

### Step A

### N-Benzyl-4-methyl-3-nitro-benzamide

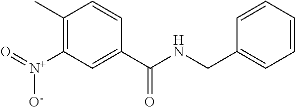

A solution of 4-methyl-3-nitrobenzoyl chloride (2.0 g, 10 mmol) (Aldrich) in ether (20 mL) was added dropwise to a solution of benzylamine (1.08 g, 10 mmol) (Aldrich), triethylamine (1.22 g, 12 mmol) and 4-dimethylamino-pyridine (60 mg, 0.5 mmol) (Fluka) in ether (80 mL) at room temperature with magnetic stirring. After 18 hours, mixture was diluted with water (100 mL). After thorough mixing, layers were separated. Organic layer was washed with 1 N aqueous hydrochloric acid, saturated aqueous sodium bicarbonate solution and brine (100 mL each). Aqueous layers were back washed with ether (100 mL). Ether layers were then combined, dried (MgSO₄), filtered and concentrated under reduced pressure to give N-benzyl-4-methyl-3-nitro-benzamide as white crystalline solid. (Yield 2.42 g, 89.5%).

### Step B

### 3-Amino-N-benzyl-4-methyl-benzamide

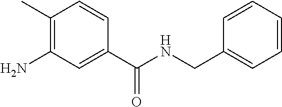

Iron powder (1.5 g, 26.9 mmol) was added slowly to a suspension of benzyl 4-methyl-3-nitro-benzamide (2.42 g, 8.95 mmol) in mixture of acetic acid (20 mL), ethanol (20 mL) and water (10 mL). Mixture was heated in an oil bath at 100° C. for 30 minutes with magnetic stirring. After cooling to room temperature, mixture was diluted with ethyl acetate (150 mL) and filtered through a pad of Celite. Filtrate was mixed thoroughly with mixture of conc. aqueous ammonium hydroxide (40 mL) and ice (100 g). Precipitate was filtered off and layers separated. Organic layer was washed with saturated aqueous sodium bicarbonate solution and brine (100 mL each). Aqueous layers were back washed with ethyl acetate (200 mL). Organic layers were then combined, dried (MgSO₄), filtered and concentrated to give 3-amino-N-benzyl-4-methyl-benzamide as yellow crystalline solid. This was used in next step without further purification. (Yield 2.27 g, 106%).

### Example 2

### 3-Amino-N-benzyl-4-chloro-benzamide

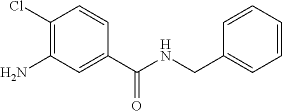

### Step A

### Benzyl 4-chloro-3-nitro-benzamide

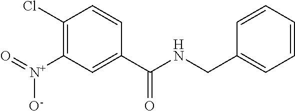

A solution of 4-chloro-3-nitrobenzoyl chloride (2.2 g, 10 mmol) (Aldrich) in ether (20 mL) was added dropwise to a solution of benzylamine (1.08 g, 10 mmol) (Aldrich), triethylamine (1.22 g, 12 mmol) and 4-dimethylamino-pyridine (60 mg, 0.5 mmol) (Fluka) in ether (80 mL) at room temperature with magnetic stirring. After 18 hours, mixture was diluted with water (100 mL). After thorough mixing, layers were separated. Organic layer was washed with 1 N aqueous hydrochloric acid, saturated aqueous sodium bicarbonate solution and brine (100 mL each). Aqueous layers were back washed with ether (100 mL). Ether layers were then combined, dried (MgSO₄), filtered and concentrated under reduced pressure to give benzyl 4-chloro-3-nitro-benzamide as white crystalline solid. (Yield 2.812 g, 96.7%).

### Step B

### 3-Amino-N-benzyl-4-chloro-benzamide

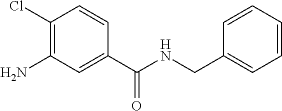

Iron powder (1.62 g, 29 mmol) was added slowly to a suspension of benzyl 4-chloro-3-nitro-benzamide (2.81 g, 9.7 mmol) in mixture of acetic acid (20 mL), ethanol (20 mL) and water (10 mL). Mixture was heated in an oil bath at 100° C. for 30 minutes with magnetic stirring. After cooling to room temperature, mixture was diluted with ethyl acetate (150 mL) and filtered through a pad of Celite. Filtrate was mixed thoroughly with mixture of conc. aqueous ammonium hydroxide (40 mL) and ice (100 g). Precipitate was filtered off and layers separated. Organic layer was washed with saturated aqueous sodium bicarbonate solution and brine (100 mL each). Aqueous layers were back washed with ethyl acetate (200 mL). Organic layers were then combined, dried (MgSO₄), filtered and concentrated to give crude product as yellow crystalline solid. This was re-crystallized fromethyl acetate-heptane to give 3-amino-N-benzyl-4-chloro-benzamide as pale yellow crystals. (Yield 2.26 g, 89.7%).

### Example 3

### (2-Amino-2-thiophen-3-yl-ethyl)-carbamic acid tert-butyl ester

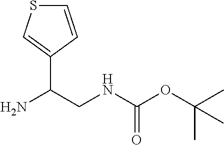

(2-Amino-2-thiophen-3-yl-ethyl)-carbamic acid tert-butyl ester was prepared in an analogous process according to the literature procedure of Seefeld, M. A.; Rouse, M. B.; Heerding, D. A.; Peace, S.; Yamashita, D. S.; McNulty, K. C. WO 2008/098104, Aug. 14, 2008.

### Step A

### Hydroxy-thiophen-3-yl-acetonitrile

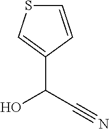

To a stirred suspension of KCN (18.6 g, 286 mmol) in methanol (100 mL) was added thiophene-3-carbaldehyde (20 mL, 178 mmol) at 0° C. under nitrogen atmosphere. Then acetic acid (4.4 mL) was added dropwise at 0° C. After 30 minutes, the mixture was warmed to 15° C. and stirred for 20 hours. NaHCO₃ (15 g) was added. The mixture was concentrated and extracted with ethyl acetate (200 mL). The organic mixture was washed with water (3×25 mL), brine (25 mL), dried over anhydrous sodium sulfate and concentrated to dryness. The residue was purified by column chromatography (ethyl acetate:petroleumether, 1:10) to afford hydroxy-thiophen-3-yl-acetonitrile. (Yield 15 g, 60%).

LC-MS: [M+Na]⁺ 162.

### Step B

### 2-Amino-1-thiophen-3-yl-ethanol

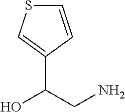

To a stirred suspension of LAH (8.7 g, 225 mmol) in dry THF (300 mL) was added a solution of hydroxy-thiophen-3-yl-acetonitrile (12.5 mL, 90 mmol) in dry THF (50 mL) dropwise at 0° C. under nitrogen atmosphere. Then the mixture was warmed to 25° C. and stirred overnight. After cooling to 10° C., H₂O (8.7 mL) was added to the solution, followed by NaOH solution (8.7 mL, 15%), then H₂O (26 mL). The reaction mixture was filtered and the filtration was concentrated to dryness to afford crude 2-amino-1-thiophen-3-yl-ethanol. (Yield 12.9 g, crude). LC-MS: [M+H]⁺ 144.

### Step C

### (2-Hydroxy-2-thiophen-3-yl-ethyl)-carbamic acid tert-butyl ester

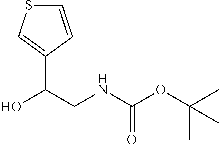

To a stirred solution of crude 2-amino-1-thiophen-3-yl-ethanol (12.9 g, crude) in THF (150 mL) was added Boc₂O (21.6 g, 99 mmol). After stirring for 1 hour, the mixture was concentrated to dryness which was purified by column chromatography (ethyl acetate:petroleumether, 1:5) to afford (2-hydroxy-2-thiophen-3-yl-ethyl)-carbamic acid tert-butyl ester. (Yield 15.3 g, 70%). LC-MS: [M+Na]⁺ 266.

### Step D

### [2-(1,3-Dioxo-1,3-dihydro-isoindol-2-yl)-2-thiophen-3-yl-ethyl]-carbamic acid tert-butyl ester

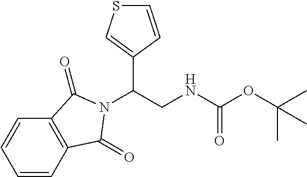

To a stirred solution of (2-hydroxy-2-thiophen-3-yl-ethyl)-carbamic acid tert-butyl ester (15.3 g, 63 mmol), pathalimide (9.5 g, 63 mmol), PPh₃ (21.4 g, 82 mmol) in THF (400 mL) was added DEAD (12.6 mL, 82 mmol) dropwise at 25° C. After 20 hours, the mixture was concentrated to dryness. The residue was purified by column chromatography (ethyl acetate:petroleum ether, 1:6) to afford [2-(1,3-dioxo-1,3-dihydro-isoindol-2-yl)-2-thiophen-3-yl-ethyl]-carbamic acid tert-butyl ester. (Yield 23 g, crude).

LC-MS: [M+Na]⁺ 395.

### Step E

### (2-Amino-2-thiophen-3-yl-ethyl)-carbamic acid tert-butyl ester

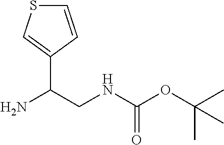

To a stirred solution of [2-(1,3-dioxo-1,3-dihydro-isoindol-2-yl)-2-thiophen-3-yl-ethyl]-carbamic acid tert-butyl ester (23 g, crude) in THF (100 mL) and methanol (100 mL) was added hydrazine hydrate (63 g, 1.26 mol). The mixture was heated at 60° C. for 2 hours and then cooled to 20° C. The reaction mixture was filtered and the filtration was concentrated to dryness. The residue was purified by column chromatography (methanol:dichloromethane, 1:50) to afford (2-amino-2-thiophen-3-yl-ethyl)-carbamic acid tert-butyl ester. (Yield 8.6 g, 57% for the two steps).

LC-MS: [M+H]⁺ 243.

### Example 4

### (2-Amino-2-phenyl-ethyl)-carbamic acid tert-butyl ester

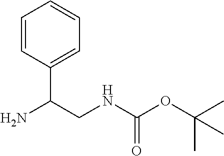

(2-Amino-2-phenyl-ethyl)-carbamic acid tert-butyl ester was prepared according to the literature procedure of Seefeld, M. A.; Rouse, M. B.; Heerding, D. A.; Peace, S.; Yamashita, D. S.; McNulty, K. C. WO 2008/098104, Aug. 14, 2008.

### Step A

### (2-Hydroxy-2-phenyl-ethyl)-carbamic acid tert-butyl ester

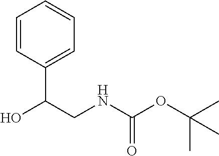

To a stirred solution of 2-amino-1-phenylethanol (20 g, 145.8 mmol) in THF (300 mL) was added the solution of Boc₂O (31.1 g, 153.1 mmol) in THF (100 mL) at 0° C. After addition, the mixture was stirred at room temperature for 0.5 hour. This mixture was concentrated to give the pure (2-hydroxy-2-phenyl-ethyl)-carbamic acid tert-butyl ester as a white solid. (Yield 34.4 g, 100%).

### Step B

### [2-(1,3-Dioxo-1,3-dihydro-isoindol-2-yl)-2-phenyl-ethyl]-carbamic acid tert-butyl ester

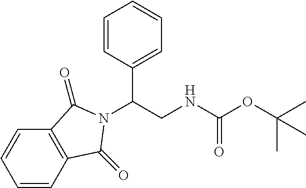

To a solution of (2-hydroxy-2-phenyl-ethyl)-carbamic acid tert-butyl ester (34.4 g, 145.0 mmol), phthalimide (21.3 g, 145 mmol), and PPh₃ (49.4 g, 188.5 mmol) was added drop-wise DEAD (32.8 g, 188.5 mmol) under stirring at 0° C. After addition, the mixture was stirred at room temperature for an additional 1 hour. The mixture was concentrated under reduced pressure. The residue was purified by column chromatography on silica gel (petroleumether:ethyl acetate, 20:1 to 5:1) to give [2-(1,3-dioxo-1,3-dihydro-isoindol-2-yl)-2-phenyl-ethyl]-carbamic acid tert-butyl ester as a white solid. (Yield 39 g, 74%).

¹H NMR (300 MHz, CDCl₃): δ 7.88-7.80 (m, 2H), 7.74-7.68 (m, 2H), 7.49-7.47 (m, 2H), 7.38-7.26 (m, 3H), 5.56-5.50 (m, 1H), 4.83 (brs, 1H), 4.28-4.22 (m, 1H), 3.93-3.87 (m, 1H), 1.35 (s, 9H). LC-MS: [M-Boc+H]⁺ 267.

### Step C

### (2-Amino-2-phenyl-ethyl)-carbamic acid tert-butyl ester

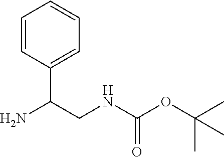

To a solution of [2-(1,3-dioxo-1,3-dihydro-isoindol-2-yl)-2-phenyl-ethyl]-carbamic acid tert-butyl ester (23 g, 63 mmol) in THF (180 mL) and MeOH (180 mL) was added 85% hydrazine hydrate (37 mL, 630 mmol) slowly. The resulting mixture was heated at 65° C. for 15 hours. The reaction mixture was cooled to room temperature, then concentrated to dryness. The residue was purified by column chromatography on silica gel (dichloromethane:MeOH, 100:1, 1% NH₃H₂O) to give (2-amino-2-phenyl-ethyl)-carbamic acid tert-butyl ester as a white solid. (Yield 7.4 g, 50%).

¹H NMR (300 MHz, CDCl₃): δ 7.35-7.24 (m, 5H), 4.81 (brs, 1H), 4.08-4.03 (m, 1H), 3.38-3.21 (m, 2H), 1.44 (s, 9H). LC-MS: [M+H]⁺ 237.

### Example 5

### ((S)-2-Amino-2-phenyl-ethyl)-carbamic acid tert-butyl ester

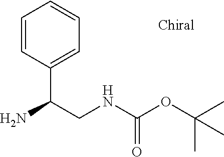

### Step A

### [(R)-2-Phenyl-propyl]-carbamic acid tert-butyl ester

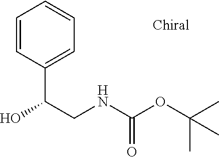

To a solution of R-(−)-2-amino-1-phenylethanol (6.52 g, 47.53 mmol) (Lancaster) in tetrahydrofuran (50 mL) was added di-tert-butyl dicarbonate (11.41 g, 52.78 mmol) (Aldrich) at room temperature with magnetic stirring. After 3 hours, mixture was concentrated and the residue was purified by flash chromatography eluting with 0-40% ethyl acetate in hexanes to give [(R)-2-phenyl-propyl]-carbamic acid tert-butyl ester. ((Yield 10.05 g, 89.1%).

### Step B

### [(S)-2-(1,3-Dioxo-indan-2-yl)-2-phenyl-ethyl]carbamic acid tert-butyl ester

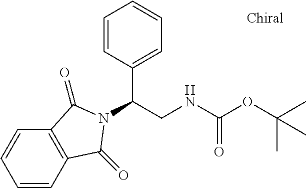

To a solution of [(R)-2-phenyl-propyl]-carbamic acid tert-butyl ester (10.05 g, 42.35 mmol), phthalimide (6.85 g, 46.59 mmol) (Aldrich) and triphenylphosphine (14.44 g, 55.06 mmol) (Aldrich) in tetrahydrofuran (150 mL) was added diisopropylazodicarboxylate (10.84 mL, 55.06 mmol) (Aldrich) dropwise at room temperature with magnetic stirring. After 18 hours, mixture was concentrated and the residue was purified by flash chromatography eluting with 15-30% ethyl acetate in hexanes to give [(S)-2-(1,3-dioxo-indan-2-yl)-2-phenyl-ethyl]carbamic acid tert-butyl ester. (Yield 15.04 g, 96.9%).

### Step C

### ((S)-2-Amino-2-phenyl-ethyl)-carbamic acid tert-butyl ester

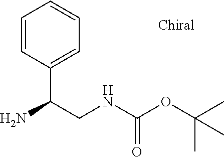

A solution of [(S)-2-(1,3-dioxo-indan-2-yl)-2-phenyl-ethyl]carbamic acid tert-butyl ester (5.9 g, 16.1 mmol) and anhydrous hydrazine (5.1 mL, 0.16 mol) (Aldrich) in methanol (20 mL) was heated at 60° C. for 18 hours. Solid was filtered off and washed with methanol. The solution was concentrate. The residue was diluted with dichloromethane and 1N NaOH solution. The aqueous phase was extracted with dichloromethane (1×). The combined organic phase was washed with brine, dried (MgSO₄) and concentrated. The residue was purified by flash chromatography eluting with 0-10% methanol in dichloromethane to give ((S)-2-amino-2-phenyl-ethyl)-carbamic acid tert-butyl ester. (Yield 1.84 g, 48.4%).

### Example 6

### (3-Amino-3-phenyl-propyl)-carbamic acid tert-butyl ester

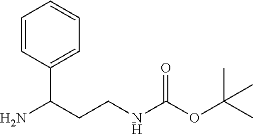

(3-Amino-3-phenyl-propyl)-carbamic acid tert-butyl ester was prepared according to the literature procedure of Seefeld, M. A.; Rouse, M. B.; Heerding, D. A.; Peace, S.; Yamashita, D. S.; McNulty, K. C. WO 2008/098104, Aug. 14, 2008.

### Step A

### 3-Amino-1-phenyl-propan-1-ol

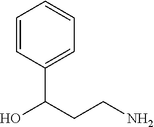

To a stirred suspension of LAH (20 g, 517 mmol) in dry THF (500 mL) was added a solution of 3-oxo-3-phenylpropanenitrile (30 g, 207 mmol) in dry THF (300 mL) drop-wise at 0° C. under nitrogen atmosphere. The mixture was warmed to 25° C. and then heated at 70° C. for 2 hours. After cooling to 0° C., a saturated solution of sodium hydroxide was added drop-wise and extracted with dichloromethane (200 mL). The organic solution was dried over anhydrous sodium sulfate and concentrated to dryness. The residue was purified by column chromatography (methanol:dichloromethane, 1:10) to afford 3-amino-1-phenyl-propan-1-ol. (Yield 30 g, crude).

LC-MS: [M+H]⁺ 152.

### Step B

### (3-Hydroxy-3-phenyl-propyl)-carbamic acid tert-butyl ester

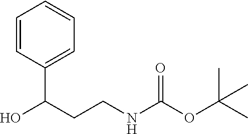

Et₃N (1.36 g, 14 mmol) was added to a solution of 3-amino-1-phenyl-propan-1-ol (1.7 g, 11.3 mmol) in THF (20 mL) under stirring. Boc₂O (3.0 g, 13.7 mmol) in THF (20 mL) was added dropwise to the solution at 0° C. Then the resulting mixture was warmed to room temperature and stirred for an additional 2 hours. The mixture was concentrated under reduced pressure. The residue was purified by column chromatography on silica gel (petroleumether:ethyl acetate, 3:1) to give (3-hydroxy-3-phenyl-propyl)-carbamic acid tert-butyl ester. (Yield 1.7 g, 60%). LC-MS: [M+23]⁺ 274.

### Step C

### [3-(1,3-Dioxo-1,3-dihydro-isoindol-2-yl)-3-phenyl-propyl]-carbamic acid tert-butyl ester

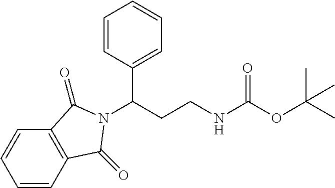

To a solution of (3-hydroxy-3-phenyl-propyl)-carbamic acid tert-butyl ester (10.4 g, 41.4 mmol), phthalimide (5.2 g, 36.6 mmol), and PPh₃ (14.6 g, 55.5 mmol) in THF (204 mL) was added dropwise DEAD (8.9 mL, 55 mmol) with stirring at 0° C. Then the resulting mixture was warmed to room temperature for an additional 2 hours. The mixture was concentrated under reduced pressure. The residue was purified by column chromatography on silica gel (petroleum ether:ethyl acetate, 3:1) to give [3-(1,3-dioxo-1,3-dihydro-isoindol-2-yl)-3-phenyl-propyl]-carbamic acid tert-butyl ester. (Yield 10.5 g, 66.8%).

¹H NMR (300 MHz, CDCl₃): δ 7.81-7.75 (m, 2H), 7.69-7.64 (m, 2H), 7.53-7.50 (m, 2H), 7.34-7.23 (m, 3H), 5.44-5.38 (m, 1H), 4.74 (brs, 1H), 3.29-3.07 (m, 2H), 2.83-2.75 (m, 1H), 2.51-2.42 (m, 1H), 1.42 (s, 9H). LC-MS: [M-Boc+H]⁺ 281.

### Step D

### (3-Amino-3-phenyl-propyl)-carbamic acid tert-butyl ester

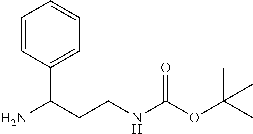

85% Hydrazine hydrate (5.1 mL, 74 mmol) was added to a solution of [3-(1,3-dioxo-1,3-dihydro-isoindol-2-yl)-3-phenyl-propyl]-carbamic acid tert-butyl ester (2.8 g, 7.4 mmol) in THF (25 mL) and MeOH (25 mL). The resulting mixture was heated at 65° C. for 6 hours. Then the precipitate was filtered, and the filtrate was concentrated under reduced pressure to give crude product which was purified by column chromatography on silica gel (dichloromethane:MeOH, 100:1, 1% NH₃H₂O) to give (3-amino-3-phenyl-propyl)-carbamic acid tert-butyl ester as an off-white solid. (Yield 1.7 g, 92%).

¹H NMR (300 MHz, CDCl₃): δ 7.31-7.18 (m, 5H), 6.82 (brs, 1H), 3.78-3.74 (m, 1H), 2.92 (brs, 2H), 1.82 (s, 2H), 1.63-1.61 (m, 2H), 1.37 (s, 9H). LC-MS: [M+H]⁺ 251.

### Example 7

### ((S)-3-Amino-3-phenyl-propyl)-carbamic acid tert-butyl ester

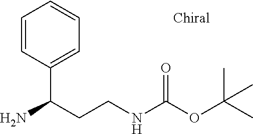

### Step A

### Racemic (3-hydroxy-3-phenyl-propyl)-carbamic acid tert-butyl ester

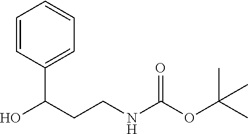

A solution of 3-oxo-3-phenylpropanenitrile (7.26 g, 50 mmol) in tetrahydrofuran (100 mL) was added, over 15 minutes, to an ice cooled solution of a 1.0 M solution of lithium aluminum hydride in tetrhydrofuran (100 mL). The mixture was stirred for 15 minutes at room temperature, then at 60° C. for 2 hours. The mixture was cooled in an ice bath and quenched by the dropwise addition of water (3.8 mL), followed by the dropwise addition of 4 M sodium hydroxide solution (3.8 mL), followed by the dropwise addition of water (11.4 mL). The mixture was stirred at room temperature for an additional 20 minutes, and the solids were then removed by suction filtration, rinsing the solids with tetrahydrofuran. The mixture was stirred overnight with BOC-anhydride (13 g, 59.6 mmol), concentrated under reduced pressure, and the residue purified by chromatography on silica gel, eluting with hexanes-ethyl acetate (70:30) to give racemic (3-hydroxy-3-phenyl-propyl)-carbamic acid tert-butyl ester. (Yield 7.9 g, 62.9%).

### Step B

### Racemic 3-(1,3-dioxo-1,3-dihydro-isoindol-2-yl)-3-phenyl-propyl]-carbamic acid tert-butyl ester

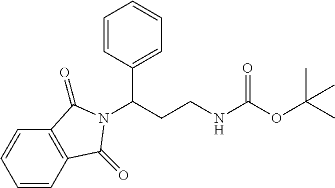

To a solution of racemic (3-hydroxy-3-phenyl-propyl)-carbamic acid tert-butyl ester (5.32 g, 36.1 mmol), and triphenylphosphine (9.48 g, 36.1 mmol) in tetrahydrofuran (200 mL) was added, over 10 minutes, diisopropyl azodicarboxylate (7.31 g, 36.1 mmol). After stirring at room temperature for 3 hours, the mixture was concentrated under reduced pressure and the residue purified by chromatography on silica gel, eluting with dichloromethane-ethyl acetate (90:10) to give pure racemic 3-(1,3-dioxo-1,3-dihydro-isoindol-2-yl)-3-phenyl-propyl-carbamic acid tert-butyl ester. (Yield 2.3 g, 16.7%).

### Step C

### Separation of (R)- and [(S)-3-(1,3-dioxo-1,3-dihydro-isoindol-2-yl)-3-phenyl-propyl]-carbamic acid tert-butyl ester

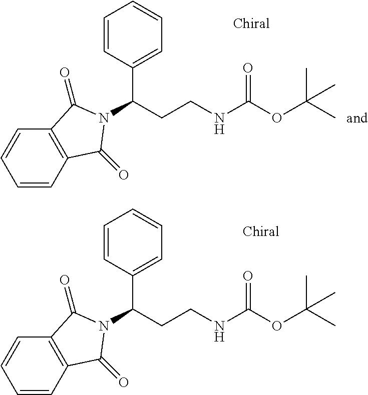

The separation of enantiomers of racemic [3-(1,3-dioxo-1,3-dihydro-isoindol-2-yl)-3-phenyl-propyl]-carbamic acid tert-butyl ester (2.3 g) was accomplished by chromatography under supercritical fluid chromatography (SFC), eluting with carbon dioxide as the carrier and 15% methanol as modifier (AD column), to give [(R)-3-(1,3-dioxo-1,3-dihydro-isoindol-2-yl)-3-phenyl-propyl]-carbamic acid tert-butyl ester as the first eluting peak (Yield 1.14 g), and [(S)-3-(1,3-dioxo-1,3-dihydro-isoindol-2-yl)-3-phenyl-propyl]-carbamic acid tert-butyl ester as the second eluting peak (Yield 1.13 g). Each enantiomer was obtained as a glass.

### Step D

### ((S)-3-Amino-3-phenyl-propyl)-carbamic acid tert-butyl ester

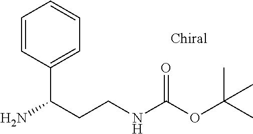

A solution of [(S)-3-(1,3-dioxo-1,3-dihydro-isoindol-2-yl)-3-phenyl-propyl]-carbamic acid tert-butyl ester (1.13 g, 2.98 mmol), hydrazine (0.93 mL, 29.8 mmol) in methanol (20 mL) was heated at reflux for 2 hours. The mixture was cooled, taken up in dichloromethane (400 mL) as was once with 1 M sodium hydroxide (85 mL), dried over anhydrous sodium sulfate plus a few pellets of sodium hydroxide, filtered and concentrated under reduced pressure to give ((S)-3-amino-3-phenyl-propyl)-carbamic acid tert-butyl ester, as an oil, which was used without further purification. (Yield 0.73 g, 92%).

### Example 8

### ((R)-3-Amino-3-phenyl-propyl)-carbamic acid tert-butyl ester

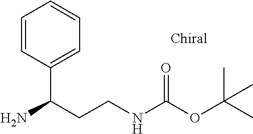

A solution of [(R)-3-(1,3-dioxo-1,3-dihydro-isoindol-2-yl)-3-phenyl-propyl]-carbamic acid tert-butyl ester (1.14 g, 3 mmol) (from Example 7 supra), hydrazine (0.94 mL, 30 mmol) in methanol (20 mL) was heated at reflux for 2 hours. The mixture was cooled, taken up in dichloromethane (400 mL) as was once with 1 M sodium hydroxide (85 mL), dried over anhydrous sodium sulfate plus a few pellets of sodium hydroxide, filtered and concentrated under reduced pressure to give ((R)-3-amino-3-phenyl-propyl)-carbamic acid tert-butyl ester, as an oil, which was used without further purification. (Yield 0.74 g, 92.6%).

### Example 9

### [3-Amino-3-(3-chloro-phenyl)-propyl]-carbamic acid tert-butyl ester

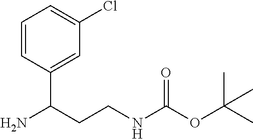

[3-Amino-3-(3-chloro-phenyl)-propyl]-carbamic acid tert-butyl ester was prepared in an analogous process according to the literature procedure of Seefeld, M. A.; Rouse, M. B.; Heerding, D. A.; Peace, S.; Yamashita, D. S.; McNulty, K. C. WO 2008/098104, Aug. 14, 2008.

### Step A

### 3-Amino-1-(3-chloro-phenyl)-propan-1-ol

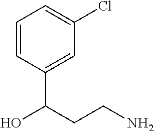

To a stirred suspension of LAH (16 g, 90 mmol) in dry THF (200 mL) was added a solution of 3-(3-chlorophenyl)-3-oxopropanenitrile (10.4 g, 270 mmol) in dry THF (200 mL) dropwise at 0° C. under nitrogen atmosphere. The mixture was warmed to 25° C. and then heated at 60° C. for 3 hours. After cooling to 0° C., a saturated solution of sodium hydroxide was added dropwise and extracted with ethyl acetate (200 mL). The solution was dried over anhydrous sodium sulfate and concentrated to dryness. The crude 3-amino-1-(3-chloro-phenyl)-propan-1-ol obtained was used in the next step without further purification. (Yield 14.5 g, crude).

LC-MS: [M+H]⁺ 186.

### Step B

### [3-(3-Chloro-phenyl)-3-hydroxy-propyl]carbamic acid tert-butyl ester

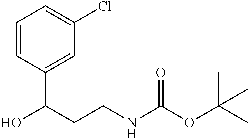

To a stirred solution of crude 3-amino-1-(3-chloro-phenyl)-propan-1-ol (29 g, 156 mmol) in THF (300 mL) was added Boc₂O (40.5 g, 187 mmol). After 0.5 hour, the mixture was concentrated to dryness. The residue was purified by column chromatography (ethyl acetate:petroleum ether, 1:20) to afford [3-(3-chloro-phenyl)-3-hydroxy-propyl]-carbamic acid tert-butyl ester. (Yield 23 g, 52%).

LC-MS: [M+Na]⁺ 308.

### Step C

### [3-(3-Chloro-phenyl)-3-(1,3-dioxo-1,3-dihydro-isoindol-2-yl)-propyl]-carbamic acid tert-butyl ester

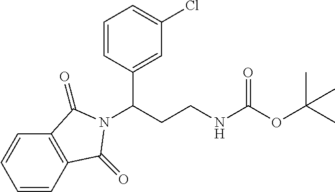

To a stirred solution of [3-(3-chloro-phenyl)-3-hydroxy-propyl]carbamic acid tert-butyl ester (12 g, 42 mmol), phthalimide (6.2 g, 42 mmol), and PPh₃ (14.3 g, 55 mmol) in THF (150 mL) was added DEAD (9.0 mL, 55 mmol) dropwise at about 5° C. After 1 hour, the mixture was concentrated to dryness. The residue was purified by column chromatography (ethyl acetate:petroleumether, 1:8) to afford [3-(3-chloro-phenyl)-3-(1,3-dioxo-1,3-dihydro-isoindol-2-yl)-propyl]-carbamic acid tert-butyl ester. (Yield 15.65 g, 90%).

LC-MS: [M+H]⁺ 415.

### Step D

### [3-Amino-3-(3-chloro-phenyl)-propyl]-carbamic acid tert-butyl ester

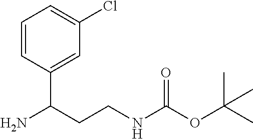

To a stirred solution of [3-(3-chloro-phenyl)-3-(1,3-dioxo-1,3-dihydro-isoindol-2-yl)-propyl]-carbamic acid tert-butyl ester (0.15 g, 0.36 mmol) in THF (2 mL) and methanol (2 mL) was added hydrazine hydrate (0.18 g, 3.6 mmol). The mixture was heated at 55° C. for 2 hours. Then the reaction mixture was concentrated and extracted with ethyl acetate (10 mL). The organic mixture was washed with water (3×1 mL), brine (1 mL), dried over anhydrous sodium sulfate and concentrated to dryness. The residue was purified by column chromatography (methanol:dichloromethane, 1:100) to afford [3-amino-3-(3-chloro-phenyl)-propyl]-carbamic acid tert-butyl ester. (Yield 0.061 g, 60%).

LC-MS: [M+H]⁺ 285.

### Example 10

### [(S)-3-Amino-3-(3-chloro-phenyl)-propyl]-carbamic acid tert-butyl ester

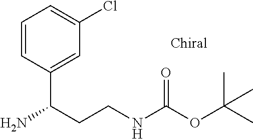

[3-Amino-3-(3-chloro-phenyl)-propyl]-carbamic acid tert-butyl ester (1 g, 3.51 mmol) (from Example Quin-007 supra) was purified by supercritical fluid chromatography [(R,R)-WHELK-O1 column, Regis Technologies, 20% methanol in carbon dioxide as solvent] to give [(S)-3-amino-3-(3-chloro-phenyl)-propyl]carbamic acid tert-butyl ester. (Yield 0.47 g).

### Example 11

### [2-Amino-2-(3-chloro-phenyl)-ethyl]carbamic acid tert-butyl ester

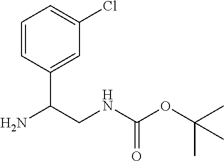

[2-Amino-2-(3-chloro-phenyl)-ethyl]-carbamic acid tert-butyl ester was prepared in an analogous process according to the literature procedure of Seefeld, M. A.; Rouse, M. B.; Heerding, D. A.; Peace, S.; Yamashita, D. S.; McNulty, K. C. WO 2008/098104, Aug. 14, 2008.

### Step A

### (3-Chloro-phenyl)-hydroxy-acetonitrile

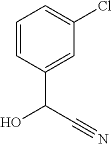

To a stirred suspension of KCN (5.04 g, 78 mmol) in methanol (20 mL) was added 3-chlorobenzaldehyde (7.0 g, 50 mmol) at 0° C. under nitrogen atmosphere. Then acetic acid (4.4 mL) was added dropwise at 0° C. After 30 minutes, the mixture was warmed to 15° C. and stirred for 5 hours. Then the reaction mixture was concentrated to dryness and extracted with ethyl acetate (200 mL). The organic solution was washed with water (3×25 mL), brine (25 mL), dried over anhydrous sodium sulfate and concentrated to dryness. The resulting residue was purified by column chromatography (ethyl acetate:petroleumether, 1:15) to afford (3-chloro-phenyl)-hydroxy-acetonitrile. (Yield 8.2 g, 97%).

LC-MS: [M+Na]⁺ 190.

### Step B

### 2-Amino-1-(3-chloro-phenyl)-ethanol

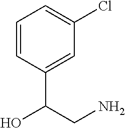

To a stirred suspension of LAH (2.36 g, 59 mmol) in dry THF (70 mL) was added a solution of (3-chloro-phenyl)-hydroxy-acetonitrile (4.0 g, 24 mmol) in dry THF (55 mL) dropwise at 0° C. under nitrogen atmosphere. The mixture was warmed to 25° C. and then heated at 60° C. for 2 hours. After cooling to 0° C., a saturated solution of sodium hydroxide was added dropwise and extracted with dichloromethane (200 mL). The organic solution was dried over anhydrous sodium sulfate and concentrated to dryness. The residue was purified by column chromatography (methanol:dichloromethane, 1:10) to afford 2-amino-1-(3-chloro-phenyl)-ethanol. (Yield 2.86 g, 70%). LC-MS: [M+H]⁺ 172.

### Step C

### [2-(3-Chloro-phenyl)-2-hydroxy-ethyl]-carbamic acid tert-butyl ester

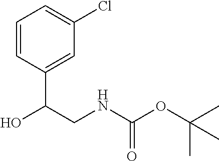

To a stirred solution of 2-amino-1-(3-chloro-phenyl)-ethanol (2.86 g, 16.7 mmol) in THF (100 mL) was added Boc₂O (4.3 g, 20 mmol). After 1 hour, the mixture was concentrated to dryness. The residue was purified by column chromatography (methanol:dichloromethane, 1:100) to afford [2-(3-chloro-phenyl)-2-hydroxy-ethyl]-carbamic acid tert-butyl ester. (Yield 3.9 g, 72%). LC-MS: [M+Na]⁺ 294.

### Step D

### [2-(3-Chloro-phenyl)-2-(1,3-dioxo-1,3-dihydro-isoindol-2-yl)-ethyl]carbamic acid tert-butyl ester

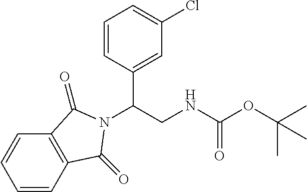

To a stirred solution of [2-(3-chloro-phenyl)-2-hydroxy-ethyl]-carbamic acid tert-butyl ester (20 g, 73.5 mmol), phthalimide (11.1 g, 73.5 mmol) and PPh₃ (25.1 g, 95.5 mmol) in THF (500 mL) was added DEAD (11.4 mL, 95.5 mmol) dropwise at −5 to 0° C. The reaction mixture was stirred at room temperature for 3 hours. Then the mixture was concentrated to dryness. The residue was purified by column chromatography (ethyl acetate:petroleumether, 1:10) to afford [2-(3-chloro-phenyl)-2-(1,3-dioxo-1,3-dihydro-isoindol-2-yl)-ethyl]carbamic acid tert-butyl ester. (Yield 20 g, 69%). LC-MS: [M+H]⁺ 401.

### Step E

### [2-Amino-2-(3-chloro-phenyl)-ethyl]carbamic acid tert-butyl ester

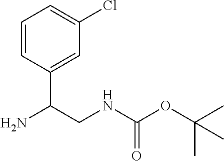

To a stirred solution of [2-(3-chloro-phenyl)-2-(1,3-dioxo-1,3-dihydro-isoindol-2-yl)-ethyl]carbamic acid tert-butyl ester (2.5 g, 6.2 mmol) in THF (10 mL) and methanol (10 mL) was added hydrazine hydrate (3.1 g, 62 mmol). The mixture was heated at 55° C. for 1 hour. Then it was concentrated to dryness, dissolved in H₂O (5 mL) and extracted with ethyl acetate (50 mL). The organic mixture was concentrated and purified by column chromatography (methanol:dichloromethane, 1:100) to afford [2-amino-2-(3-chloro-phenyl)-ethyl]-carbamic acid tert-butyl ester. (Yield 1.325 g, 79%).

LC-MS: [M+H]⁺ 271.

### Example 12

### [(R)-2-Amino-2-(3-chloro-phenyl)-ethyl]carbamic acid tert-butyl ester

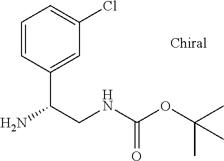

[2-Amino-2-(3-chloro-phenyl)-ethyl]-carbamic acid tert-butyl ester (1 g, 3.69 mmol) (from Example Quin-010 supra) was purified by supercritical fluid chromatography [(R,R)-WHELK-O1 column, Regis Technologies, 20% methanol in carbon dioxide as solvent] to give [(R)-2-amino-2-(3-chloro-phenyl)-ethyl]-carbamic acid tert-butyl ester. (Yield 0.47 g).

### Example 13

### [(S)-2-Amino-2-(3-chloro-phenyl)-ethyl]carbamic acid tert-butyl ester

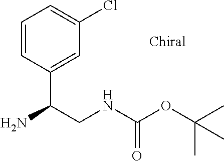

[2-Amino-2-(3-chloro-phenyl)-ethyl]-carbamic acid tert-butyl ester (1 g, 3.69 mmol) (from Example Quin-010 supra) was purified by supercritical fluid chromatography [(R,R)-WHELK-O1 column, Regis Technologies, 20% methanol in carbon dioxide as solvent] to give [(S)-2-Amino-2-(3-chloro-phenyl)-ethyl]-carbamic acid tert-butyl ester. (Yield 0.50 g).

### Example 14

### (3-Amino-3-thiophen-3-yl-propyl)-carbamic acid tert-butyl ester

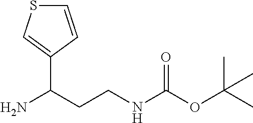

(3-Amino-3-thiophen-3-yl-propyl)-carbamic acid tert-butyl ester was prepared in an analogous process according to the literature procedure of Seefeld, M. A.; Rouse, M. B.; Heerding, D. A.; Peace, S.; Yamashita, D. S.; McNulty, K. C. WO 2008/098104, Aug. 14, 2008.

### Step A

### 3-Amino-1-thiophen-3-yl-propan-1-ol

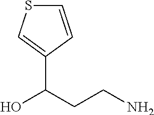

To a stirred suspension of LAH (1.45 g, 38.1 mmol) in dry THF (120 mL) was added a solution of 3-oxo-3-(thiophen-3-yl)propanenitrile (4.8 g, 31.8 mmol) in dry THF (40 mL) dropwise at 0° C. under nitrogen atmosphere. The mixture was warmed to 25° C. and then heated at 65° C. for 6 hours. After cooling to 0° C., a saturated solution of sodium hydroxide (2 mL) was added dropwise and the mixture was filtered. The filtrate was concentrated to dryness to give crude 3-amino-1-thiophen-3-yl-propan-1-ol which was used in next step without further purification.

¹H NMR (300 MHz, CDCl₃): δ 7.29-7.26 (m, 2H), 7.05 (dd, 1H, J₁=4.8 Hz, J₂=1.2 Hz), 5.04 (dd, 1H, J₁=8.1 Hz, J₂=3.0 Hz), 3.10-3.05 (m, 2H), 1.82-1.77 (m, 2H).

### Step B

### (3-Hydroxy-3-thiophen-3-yl-propyl)-carbamic acid tert-butyl ester

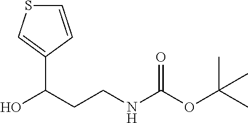

To a stirred solution of crude 3-amino-1-thiophen-3-yl-propan-1-ol (23 g) in THF (100 mL) was added Boc₂O (31.6 g, 146.3 mmol). The mixture was stirred at room temperature for 1 hour and then concentrated to dryness. The residue was purified by column chromatography (ethyl acetate:petroleumether, 1:10) to afford (3-hydroxy-3-thiophen-3-yl-propyl)-carbamic acid tert-butyl ester. (Yield 21.5 g, 51% for two steps).

¹H NMR (300 MHz, CDCl₃): δ 8.08-8.06 (m, 1H), 7.55-7.53 (m, 1H), 7.34-7.30 (m, 1H), 5.10 (s, 1H), 3.52-3.48 (m, 2H), 3.13-3.09 (m, 2H), 1.42 (s, 9H). LC-MS: [M+Na]⁺ 280.

### Step C

### 3-(1,3-Dioxo-1,3-dihydro-isoindol-2-yl)-3-thiophen-3-yl-propyl]-carbamic acid tert-butyl ester

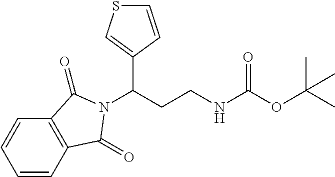

To a stirred solution of (3-hydroxy-3-thiophen-3-yl-propyl)-carbamic acid tert-butyl ester (21.5 g, 83.6 mmol), phthalimide (12.3 g, 83.6 mmol), and PPh₃ (28.5 g, 108.6 mmol) in THF (400 mL) was added DEAD (17.6 mL, 108.6 mmol) dropwise at 25° C. The mixture was stirred at room temperature for 14 hours, then concentrated. The residue was purified by column chromatography (ethyl acetate:petroleumether, 1:6) to afford 3-(1,3-dioxo-1,3-dihydro-isoindol-2-yl)-3-thiophen-3-yl-propyl]-carbamic acid tert-butyl ester. (Yield 12 g, 38%).

¹H NMR (300 MHz, CDCl₃): δ 7.82-7.77 (m, 2H), 7.72-7.68 (m, 2H), 7.36 (d, 1H, J=1.8 Hz), 7.26-7.18 (m, 2H), 5.50 (dd, 1H, J₁=9.6 Hz, J₂=6 Hz), 4.65 (brs, 1H), 3.24-3.07 (m, 2H), 2.72-2.67 (m, 1H), 2.47-2.40 (m, 1H), 1.40 (s, 9H). LC-MS: [M+H-Boc]⁺ 287.

### Step D

### (3-Amino-3-thiophen-3-yl-propyl)-carbamic acid tert-butyl ester

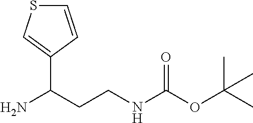

To a stirred solution of 3-(1,3-dioxo-1,3-dihydro-isoindol-2-yl)-3-thiophen-3-yl-propyl]-carbamic acid tert-butyl ester (12 g, 31.1 mmol) in methanol (150 mL) was added hydrazine hydrate (18 mL, 85% aqueous). The mixture was heated at reflux for 14 hours. After cooling to room temperature, the reaction mixture was filtered. The filtrate was concentrated and the residue was purified by column chromatography (methanol:dichloromethane, 1:50 to 1:20, 0.1% NH₃H₂O) to afford (3-amino-3-thiophen-3-yl-propyl)-carbamic acid tert-butyl ester. (Yield 7.6 g, 95%).

¹H NMR (300 MHz, CDCl₃): δ 7.49 (s, 1H), 7.25-7.08 (m, 2H), 6.82 (brs, 1H), 3.85 (t, 1H, J=6.0 Hz), 3.18-2.95 (m, 4H), 1.75-1.62 (m, 2H), 1.37 (s, 9H). LC-MS: [M+H]⁺ 257.

### Example 15

### 8-Oxa-3-aza-bicyclo[3.2.1]octane; hydrochloride

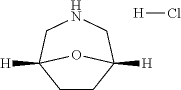

8-Oxa-3-aza-bicyclo[3.2.1]octane; hydrochloride was purchased from APAC Pharmaceutical LLC.

### Example 16

### 4-Amino-2-methoxy-pyrimidine-5-carbaldehyde

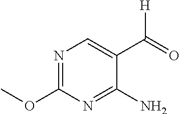

4-Amino-2-methoxy-pyrimidine-5-carbaldehyde was prepared according to the literature procedure of Perandones, F.; Soto, J. L. J. Heterocyclic Chem., 1998, 35, 413-419.

### Example 17

### 2-Methoxy-7-oxo-7,8-dihydro-pyrido[2,3-d]pyrimidine-6-carboxylic acid methyl ester

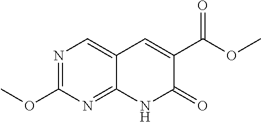

2-Methoxy-7-oxo-7,8-dihydro-pyrido[2,3-d]pyrimidine-6-carboxylic acid methyl ester was prepared according to the literature procedure of Perandones, F.; Soto, J. L. J. Heterocyclic Chem., 1998, 35, 413-419. HR-MS (ES⁺) m/z Calculated for C₁₀H₁₀N₃O₄ ([M+H]⁺): 236.0666.

Found: 236.0665.

### Example 18

### 2-Methoxy-7-oxo-7,8-dihydro-pyrido[2,3-d]pyrimidine-6-carboxylic acid

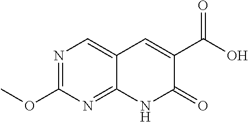

Aqueous sodium hydroxide solution (1 N, 36 mL, 36 mmol) was added to a suspension of 2-methoxy-7-oxo-7,8-dihydro-pyrido[2,3-d]pyrimidine-6-carboxylic acid methyl ester (2.50 g, 10.63 mmol) (from Example 17 supra) in methanol (36 mL) and water (18 mL). Mixture was heated at 65° C. for 18 hours. After cooling, mixture was diluted with water and concentrated under reduced pressure to remove most of the methanol. Clear solution was obtained. This was acidified with 2 N aqueous hydrochloric acid. White precipitate formed was collected by filtration and washed with water and dried in vacuum oven to give crude 2-methoxy-7-oxo-7,8-dihydro-pyrido[2,3-d]pyrimidine-6-carboxylic acid as white powder. (Yield 2.47 g, 105%).

HR-MS (ES⁺) m/z Calculated for C₉H₈N₃O₄ ([M+H]⁺): 222.0510. Found: 222.0510.

### Example 19

### 3-[(2-Methoxy-7-oxo-7,8-dihydro-pyrido[2,3-d]pyrimidine-6-carbonyl)-amino]-4-methyl-benzoic acid methyl ester

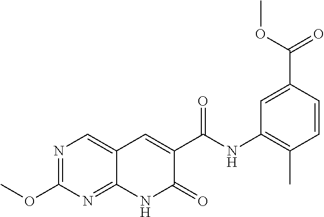

Triethylamine (0.12 g, 1.12 mmol) was added to a mixture of crude 2-methoxy-7-oxo-7,8-dihydro-pyrido[2,3-d]pyrimidine-6-carboxylic acid (0.22 g, 1 mmol) (from Example 18 supra) and HATU (0.43 g, 1.12 mmol) (Aldrich) in DMF (10 mL) at room temperature. Mixture was stirred until clear solution was obtained (light brown). 3-Amino-p-toluic acid methyl ester (0.2 g, 1.2 mmol) (TCI-US) was added. Mixture was stirred for another 18 hours. Precipitate was formed. Water (45 mL), saturated aqueous sodium bicarbonate solution (5 mL), and ethyl acetate (20 mL) were added. After thorough mixing, yellow precipitate was collected by filtration, washed with water and ethyl acetate and dried in vacuum oven. (Yield 0.25 g, 67.9%). Sample was recrystallized from DMF with trace amounts of water to give 3-[(2-methoxy-7-oxo-7,8-dihydro-pyrido[2,3-d]pyrimidine-6-carbonyl)-amino]-4-methyl-benzoic acid methyl ester as pale yellow crystals that contained trace amounts of DMF. (Yield 0.23 g, 62%).

LR-LC/MS (APCi⁺) m/z [M+H]⁺ 369.

### Example 20

### 3-[(2-Methoxy-7-oxo-7,8-dihydro-pyrido[2,3-d]pyrimidine-6-carbonyl)-amino]-4-methyl-benzoic acid

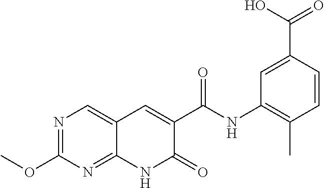

A solution of sodium hydroxide (0.088 g, 2.20 mmol) in water (5 mL) was added to a suspension of 3-[(2-methoxy-7-oxo-7,8-dihydro-pyrido[2,3-d]pyrimidine-6-carbonyl)-amino]-4-methyl-benzoic acid methyl ester (0.27 g, 0.73 mmol) (from Example 19 supra) in methanol (10 mL). Mixture was heated at 65° C. for 18 hours. After cooling, mixture was concentrated under reduced pressure to remove most of the methanol. The residue was diluted with water (10 mL) and acidified with concentrated hydrochloric acid. White precipitate formed was collected by filtration and washed with water and dried in vacuum oven to give crude 3-[(2-methoxy-7-oxo-7,8-dihydro-pyrido[2,3-d]pyrimidine-6-carbonyl)-amino]-4-methyl-benzoic acid as white powder. (Yield 0.26 g, 100%).

### Example 21

### 4-Chloro-3-[(2-methoxy-7-oxo-7,8-dihydro-pyrido[2,3-d]pyrimidine-6-carbonyl)-amino]-benzoic acid methyl ester

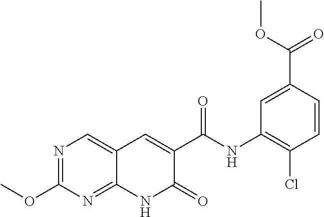

Triethylamine (0.57 g, 5.6 mmol) was added to a mixture of crude 2-methoxy-7-oxo-7,8-dihydro-pyrido[2,3-d]pyrimidine-6-carboxylic acid (1.11 g, 5 mmol) (from Example 18 supra) and HATU (2.13 g, 5.6 mmol) (Aldrich) in DMF (50 mL) at room temperature. Mixture was stirred until clear solution was obtained (light brown). Methyl 3-amino-4-chlorobenzoate (1.11 g, 6 mmol) (TCI-US) was added. Mixture was stirred for another 18 hours. Precipitate was formed. Water (25 mL), saturated sodium bicarbonate solution (25 mL), and ethyl acetate (100 mL) were added. After thorough mixing, yellow precipitate was collected by filtration, washed with water and ethyl acetate and dried in vacuum oven to give 3-[(2-methoxy-7-oxo-7,8-dihydro-pyrido[2,3-d]pyrimidine-6-carbonyl)-amino]-4-methyl-benzoic acid methyl ester. (Yield 1.17 g, 60.2%).

HR-MS (ES⁺) m/z Calculated for C₁₇H₁₄ClN₄O₅ ([M+H]⁺): 389.0647. Found: 389.0646.

### Example 22

### 4-Chloro-3-[(2-methoxy-7-oxo-7,8-dihydro-pyrido[2,3-d]pyrimidine-6-carbonyl)-amino]-benzoic acid

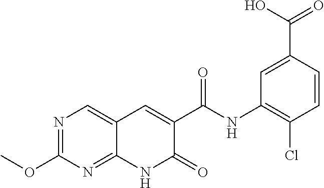

A sodium hydroxide solution (0.36 g, 8.95 mmol) in water (10 mL) was added to a suspension of 4-chloro-3-[(2-methoxy-7-oxo-7,8-dihydro-pyrido[2,3-d]pyrimidine-6-carbonyl)-amino]-benzoic acid methyl ester (1.16 g, 2.98 mmol) (from Example 21 supra) in methanol (20 mL). Mixture was heated at 65° C. for 18 hours. After cooling, mixture was concentrated under reduced pressure to remove most of the methanol. The residue was diluted with water (20 mL) and acidified with concentrated hydrochloric acid. White precipitate formed was collected by filtration and washed with water and dried in vacuum oven to give crude 4-chloro-3-[(2-methoxy-7-oxo-7,8-dihydro-pyrido[2,3-d]pyrimidine-6-carbonyl)-amino]-benzoic acid as white powder. (Yield 0.96 g, 85.7%).

HR-MS (ES⁺) m/z Calculated for C₁₆H₁₂ClN₄O₅ ([M+H]⁺): 375.0491. Found: 375.0489.

### Example 23

### 2-Methylsulfanyl-7-oxo-7,8-dihydro-pyrido[2,3-d]pyrimidine-6-carboxylic acid methyl ester

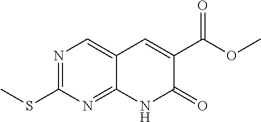

Potassium carbonate (2.15 g, 15.5 mmol) was added to a mixture of 4-amino-2-methylsulfanyl-pyrimidine-5-carbaldehyde (2.19 g, 12.9 mmol) (prepared according to the procedure of Arzeno, H. B.; Chen, J. J.; Dunn, J. P.; Goldstein, D. M.; Lim, J. A. WO 2002/18379, Mar. 7, 2002) and dimethyl malonate (2.28 g, 17.2 mmol) (Aldrich) in DMF (50 mL). Mixture was heated at 85° C. for 18 hours. TLC (silica gel, ethyl acetate) showed reaction not complete. Triethylamine (1.0 g, 9.8 mmol) was added and heating was continued for another 24 hours. After cooling, mixture was diluted with water (500 mL) and acidified with acetic acid and ethyl acetate (50 mL). Resulting precipitate was collected by filtration, washed with water. Aqueous layer was extracted with ethyl acetate (2×300 mL). Organic layers were washed with brine, then combined, dried (MgSO₄), filtered and concentrated. This residue was combined with the original precipitate and recrystallized from 95% ethanol to give 2-methylsulfanyl-7-oxo-7,8-dihydro-pyrido[2,3-d]pyrimidine-6-carboxylic acid methyl ester as off-white crystals. (Yield 2.71 g, 83.3%).

HR-MS (ES⁺) m/z Calculated for C₁₀H₁₀N₃O₃S ([M+H]⁺): 252.0438. Found: 252.0438.

### Example 24

### 2-Methylsulfanyl-7-oxo-7,8-dihydro-pyrido[2,3-d]pyrimidine-6-carboxylic acid

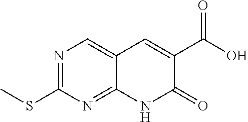

Aqueous sodium hydroxide solution (1 N, 30 mL, 30 mmol) was added to a suspension of 2-methylsulfanyl-7-oxo-7,8-dihydro-pyrido[2,3-d]pyrimidine-6-carboxylic acid methyl ester (2.51 g, 10 mmol) (from Example 23 supra) in methanol (30 mL) and water (15 mL). The mixture was heated at 65° C. for 18 hours. After cooling, the mixture was diluted with water and concentrated under reduced pressure to remove most of the methanol. Clear solution was obtained. This was acidified with 2 N aqueous hydrochloric acid. White precipitate formed was collected by filtration and washed with water and dried in vacuum oven to give crude product as white powder. LC/MS showed starting material still present. Material was redissolved in mixture of 1 N aqueous sodium hydroxide (30 mL) and water (15 mL) and heated at 65° C. for 18 hours. After cooling, mixture was acidified with 2 N aqueous hydrochloric acid. White precipitate formed was collected by filtration and washed with water and dried in vacuum oven to give crude 2-methylsulfanyl-7-oxo-7,8-dihydro-pyrido[2,3-d]pyrimidine-6-carboxylic acid as white powder. (Yield 2.52 g, 106%).

HR-MS (ES⁺) m/z Calculated for C₉H₈N₃O₃S ([M+H]⁺): 238.0281. Found: 238.0282.

### Example 25

### 4-Methyl-3-[(2-methylsulfanyl-7-oxo-7,8-dihydro-pyrido[2,3-d]pyrimidine-6-carbonyl)-amino]-benzoic acid methyl ester

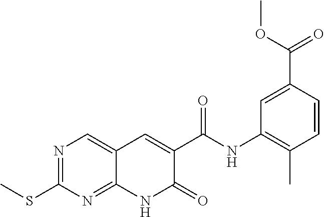

Triethylamine (0.12 g, 1.12 mmol) was added to a mixture of 2-methylsulfanyl-7-oxo-7,8-dihydro-pyrido[2,3-d]pyrimidine-6-carboxylic acid (0.24 g, 1 mmol) (from Example 24 supra) and HATU (0.43 g, 1.12 mmol) (Aldrich) in DMF (10 mL) at room temperature. Mixture was stirred until clear solution was obtained (light brown). 3-Amino-p-toluic acid methyl ester (0.2 g, 1.2 mmol) (TCI) was added. Mixture stirred for another 18 hours. Precipitate was formed. Water (50 mL) and ethyl acetate (20 mL) were added. After thorough mixing, yellow precipitate was collected by filtration, washed with water and ethyl acetate and dried in vacuum oven to give 4-methyl-3-[(2-methylsulfanyl-7-oxo-7,8-dihydro-pyrido[2,3-d]pyrimidine-6-carbonyl)-amino]-benzoic acid methyl ester. (Yield 0.32 g, 83.3%).

HR-MS (ES⁺) m/z Calculated for C₁₈H₁₇N₄O₄S ([M+H]⁺): 385.0965. Found: 385.0966.

### Example 26

### 3-[(2-Methoxy-7-oxo-7,8-dihydro-pyrido[2,3-d]pyrimidine-6-carbonyl)-amino]-4-methyl-benzoic acid benzyl ester

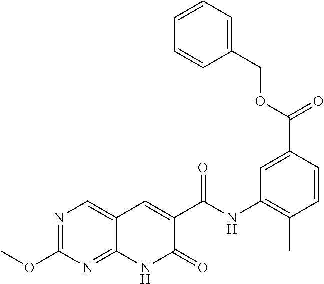

### Step A

### 4-Methyl-3-nitro-benzoic acid benzyl ester

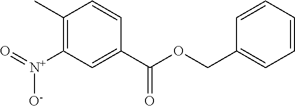

A solution of 4-methyl-3-nitrobenzoyl chloride (2.0 g, 10 mmol) (Aldrich) in ether (20 mL) was added dropwise to a solution of benzyl alcohol (1.08 g, 10 mmol) (Aldrich), triethylamine (1.22 g, 12 mmol) and 4-dimethylamino-pyridine (60 mg, 0.5 mmol) (Fluka) in ether (80 mL) at room temperature with magnetic stirring. After 18 hours, mixture was diluted with water (100 mL). After thorough mixing, layers were separated. Organic layer was washed with 1 N aqueous hydrochloric acid, saturated aqueous sodium bicarbonate solution and brine (100 mL each). Aqueous layers were back washed with ether (100 mL). Ether layers were then combined, dried (MgSO₄), filtered and concentrated under reduced pressure to give 4-methyl-3-nitro-benzoic acid benzyl ester as pale yellow oil which crystallized on standing. (Yield 2.73 g, 101%).

### Step B

### 3-Amino-4-methyl-benzoic acid benzyl ester

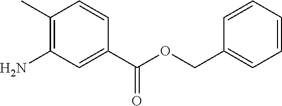

Iron powder (1.7 g, 30.2 mmol) was added slowly to a suspension of 4-methyl-3-nitro-benzoic acid benzyl ester (2.7 g, 10 mmol) in mixture of acetic acid (20 mL), ethanol (20 mL) and water (10 mL). Mixture was heated in an oil bath at 100° C. for 30 minutes with magnetic stirring. After cooling to room temperature, mixture was diluted with ethyl acetate (150 mL) and filtered through a pad of Celite. Filtrate was mixed thoroughly with mixture of conc. aqueous ammonium hydroxide (40 mL) and water (60 mL). Precipitate was filtered off and layers separated. Organic layer was washed with saturated aqueous sodium bicarbonate solution and brine (300 mL each). Aqueous layers were back washed with ethyl acetate (150 mL). Organic layers were then combined, dried (MgSO₄), filtered and concentrated to give crude 3-amino-4-methyl-benzoic acid benzyl ester as yellow crystalline solid. This was used in next step without further purification. (Yield 2.37 g, 97.6%).

### Step C

### 3-[(2-Methoxy-7-oxo-7,8-dihydro-pyrido[2,3-d]pyrimidine-6-carbonyl)-amino]-4-methyl-benzoic acid benzyl ester

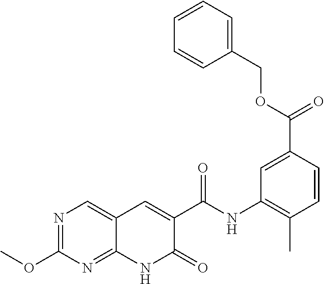

Triethylamine (0.07 g, 0.6 mmol) was added to a mixture of crude 2-methoxy-7-oxo-7,8-dihydro-pyrido[2,3-d]pyrimidine-6-carboxylic acid (0.12 g, 0.54 mmol) (from Example 18 supra) and HATU (0.23 g, 0.6 mmol) (Aldrich) in DMF (5 mL) at room temperature. Mixture was stirred until clear solution was obtained (light brown). 3-Amino-4-methyl-benzoic acid benzyl ester (0.13 g, 0.54 mmol) was added. Mixture was stirred for another 18 hours. Precipitate was formed. Water (45 mL), saturated sodium bicarbonate solution (5 mL), and ethyl acetate (20 mL) were added. After thorough mixing, yellow precipitate was collected by filtration, washed with water and ethyl acetate and dried in vacuum oven to give 3-[(2-methoxy-7-oxo-7,8-dihydro-pyrido[2,3-d]pyrimidine-6-carbonyl)-amino]-4-methyl-benzoic acid benzyl ester. (Yield 0.11 g, 46.2%).

HR-MS (ES⁺) m/z Calculated for C₂₄H₂₁N₄O₅ ([M+H]⁺): 445.1507. Found: 445.1508.

### Example 27

### 2-Methoxy-7-oxo-7,8-dihydro-pyrido[2,3-d]pyrimidine-6-carboxylic acid (5-benzylcarbamoyl-2-methyl-phenyl)-amide

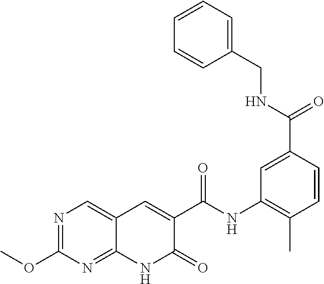

Triethylamine (0.07 g, 0.6 mmol) was added to a mixture of crude 2-methoxy-7-oxo-7,8-dihydro-pyrido[2,3-d]pyrimidine-6-carboxylic acid (0.12 g, 0.54 mmol) (from Example 18 supra) and HATU (0.23 g, 0.6 mmol) (Aldrich) in DMF (5 mL) at room temperature. Mixture was stirred until clear solution was obtained (light brown). 3-Amino-p-toluic acid benzyl amide (from Example 1 supra) (0.13 g, 0.54 mmol) was added. Mixture was stirred for another 18 hours. Precipitate was formed. Water (50 mL), saturated aqueous sodium bicarbonate solution (5 mL), and ethyl acetate (20 mL) were added. After thorough mixing, yellow precipitate was collected by filtration, washed with water and ethyl acetate and dried in vacuum oven to give 2-methoxy-7-oxo-7,8-dihydro-pyrido[2,3-d]pyrimidine-6-carboxylic acid (5-benzylcarbamoyl-2-methyl-phenyl)-amide. (Yield 0.19 g, 79.9%).

HR-MS (ES⁺) m/z Calculated for C₂₄H₂₂N₅O₄ ([M+H]⁺): 444.1667. Found: 444.1666.

### Example 28

### 3-[(2-Methanesulfonyl-7-oxo-7,8-dihydro-pyrido[2,3-d]pyrimidine-6-carbonyl)-amino]-4-methyl-benzoic acid methyl ester

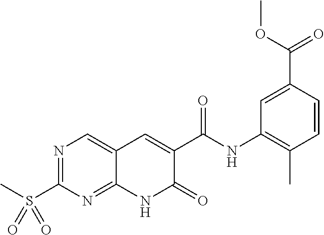

To a solution of 4-methyl-3-[(2-methylsulfanyl-7-oxo-7,8-dihydro-pyrido[2,3-d]pyrimidine-6-carbonyl)-amino]-benzoic acid methyl ester (0.15 g, 0.38 mmol) (from Example 23 supra) in dichloromethane (15 mL)/DMF (1 mL) at 0° C. was added 3-chloroperoxybenzoic acid (77% max., 0.2 g, 0.89 mmol) (Aldrich) portionwise. The mixture was stirred at room temperature for 3 days. The mixture was concentrated. The residue was washed with water and dried in vacuum oven to give 3-[(2-methanesulfonyl-7-oxo-7,8-dihydro-pyrido[2,3-d]pyrimidine-6-carbonyl)-amino]-4-methyl-benzoic acid methyl ester. (Yield 0.14 g, 88.6%).

HR-MS (ES⁺) m/z Calculated for C₁₈H₁₇N₄O₆S ([M+H]⁺): 417.0864. Found: 417.0863.

### Example 29

### 4-Methyl-3-[(2-morpholin-4-yl-7-oxo-7,8-dihydro-pyrido[2,3-d]pyrimidine-6-carbonyl)-amino]-benzoic acid methyl ester

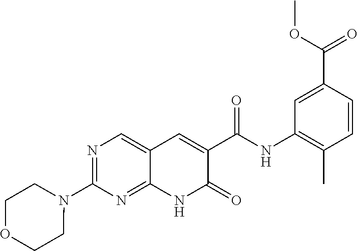

A mixture of 3-[(2-methanesulfonyl-7-oxo-7,8-dihydro-pyrido[2,3-d]pyrimidine-6-carbonyl)-amino]-4-methyl-benzoic acid methyl ester (0.05 g, 0.12 mmol) (from Example 28 supra) in morpholine (2 mL) was heated at 180° C. in a microwave reactor for 1 hour. The mixture was purified by HPLC eluting with MeCN/H₂O to give 4-methyl-3-[(2-morpholin-4-yl-7-oxo-7,8-dihydro-pyrido[2,3-d]pyrimidine-6-carbonyl)-amino]-benzoic acid methyl ester. (Yield 0.018 g, 35.4%).

HR-MS (ES⁺) m/z Calculated for C₂₁H₂₂N₅O₅ ([M+H]⁺): 424.1616. Found: 424.1612.

### Example 30

### 4-Chloro-3-[(2-methylsulfanyl-7-oxo-7,8-dihydro-pyrido[2,3-d]pyrimidine-6-carbonyl)-amino]-benzoic acid methyl ester

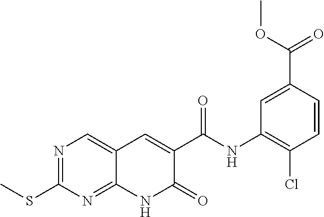

Triethylamine (0.48 g, 4.72 mmol) was added to a mixture of 2-methylsulfanyl-7-oxo-7,8-dihydro-pyrido[2,3-d]pyrimidine-6-carboxylic acid (1.0 g, 4.2 mmol) (from Example 24 supra) and HATU (1.80 g, 4.72 mmol) (Aldrich) in DMF (40 mL) at room temperature. Mixture was stirred until clear solution was obtained (light brown). Methyl 3-amino-4-chlorobenzoate (0.94 g, 5.1 mmol) (TCI) was added. Mixture was stirred for another 18 hours. Precipitate was formed. Water (200 mL) and ethyl acetate (50 mL) were added. After thorough mixing, yellow precipitate was collected by filtration, washed with water and ethyl acetate and dried in vacuum oven to give 4-chloro-3-[(2-methylsulfanyl-7-oxo-7,8-dihydro-pyrido[2,3-d]pyrimidine-6-carbonyl)-amino]-benzoic acid methyl ester. (Yield 1.04 g, 61.0%).

HR-MS (ES⁺) m/z Calculated for C₁₇H₁₄ClN₄O₄S ([M+H]⁺): 405.0419. Found: 405.0418.

### Example 31

### 2-Methoxy-7-oxo-7,8-dihydro-pyrido[2,3-d]pyrimidine-6-carboxylic acid (5-benzylcarbamoyl-2-chloro-phenyl)-amide

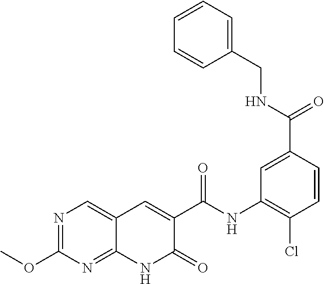

Triethylamine (0.07 g, 0.6 mmol) was added to a mixture of crude 2-methoxy-7-oxo-7,8-dihydro-pyrido[2,3-d]pyrimidine-6-carboxylic acid (0.12 g, 0.54 mmol) (from Example 18 supra) and HATU (0.23 g, 0.6 mmol) (Aldrich) in DMF (5 mL) at room temperature. The mixture was stirred until clear solution was obtained (light brown). 3-Amino-4-chlorobenzoic acid benzyl amide (from Example 2 supra) (0.14 g, 0.54 mmol) was added. The mixture was stirred for another 18 hours. Precipitate was formed. Water (50 mL), saturated aqueous bicarbonate solution (5 mL), and ethyl acetate (20 mL) were added. After thorough mixing, yellow precipitate was collected by filtration, washed with water and ethyl acetate and dried in vacuum oven to give 2-methoxy-7-oxo-7,8-dihydro-pyrido[2,3-d]pyrimidine-6-carboxylic acid

(5-benzylcarbamoyl-2-chloro-phenyl)-amide. (Yield 0.13 g, 52.3%).

HR-MS (ES⁺) m/z Calculated for C₂₃H₁₉ClN₅O₄ ([M+H]⁺): 464.1120. Found: 464.1119.

### Example 32

### 4-Chloro-3-[(2-methanesulfonyl-7-oxo-7,8-dihydro-pyrido-pyrido[2,3-d]pyrimidine-6-carbonyl)-amino]-benzoic acid methyl ester

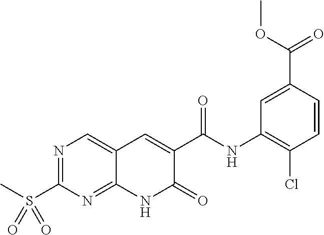

To a solution of 4-chloro-3-[(2-methylsulfanyl-7-oxo-7,8-dihydro-pyrido[2,3-d]pyrimidine-6-carbonyl)-amino]-benzoic acid methyl ester (0.60 g, 1.48 mmol) (from Example 30 supra) in dichloromethane (50 mL)/DMF (4 mL) at 0° C. was added 3-chloroperoxybenzoic acid (77% max., 0.8 g, 3.56 mmol) (Aldrich) portionwise. The mixture was stirred at room temperature for 2 days. The pricepitate was filtered, washed with methanol and dried in vacuum oven to give 4-chloro-3-[(2-methanesulfonyl-7-oxo-7,8-dihydro-pyrido-pyrido[2,3-d]pyrimidine-6-carbonyl)-amino]-benzoic acid methyl ester. (Yield 0.47 g, 72.3%).

### Example 33

### 4-Chloro-3-[(2-morpholin-4-yl-7-oxo-7,8-dihydro-pyrido[2,3-d]pyrimidine-6-carbonyl)-amino]-benzoic acid methyl ester

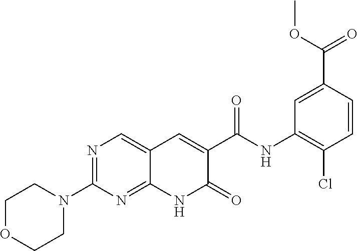

A mixture of 4-chloro-3-[(2-methanesulfonyl-7-oxo-7,8-dihydro-pyrido-pyrido[2,3-d]pyramid-ine-6-carbonyl)-amino]-benzoic acid methyl ester (0.04 g, 0.09 mmol) (from Example 32 supra) in morpholine (4 mL) was heated at 120° C. in a microwave reactor for 30 minutes. The mixture was purified by HPLC eluting with MeCN/H₂O to give 4-chloro-3-[(2-morpholin-4-yl-7-oxo-7,8-dihydro-pyrido[2,3-d]pyrimidine-6-carbonyl)-amino]-benzoic acid methyl ester. (Yield 0.04 g, 98.5%).

HR-MS (ES⁺) m/z Calculated for C₂₀H₁₉ClN₅O₅ ([M+H]⁺): 444.1069. Found: 444.1070.

### Example 34

### 4-Chloro-3-[(7-oxo-2-piperidin-1-yl-7,8-dihydro-pyrido[2,3-d]pyrimidine-6-carbonyl)-amino]-benzoic acid methyl ester

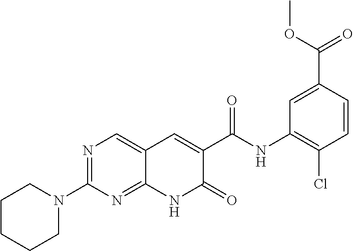

A mixture of 4-chloro-3-[(2-methanesulfonyl-7-oxo-7,8-dihydro-pyrido-pyrido[2,3-d]pyramid-ine-6-carbonyl)-amino]-benzoic acid methyl ester (0.04 g, 0.09 mmol) (from Example 32 supra) in piperidine (4 mL) was heated at 120° C. in a microwave reactor for 30 minutes. The mixture was purified by HPLC eluting with MeCN/H₂O to give 4-chloro-3-[(7-oxo-2-piperidin-1-yl-7,8-dihydro-pyrido[2,3-d]pyrimidine-6-carbonyl)-amino]-benzoic acid methyl ester. (Yield 0.02 g, 49.5%).

HR-MS (ES⁺) m/z Calculated for C₂₁H₂₁ClN₅O₄ ([M+H]⁺): 442.1277. Found: 442.1278.

### Example 35

### 4-Chloro-3-{[2-(4-methyl-piperazin-1-yl)-7-oxo-7,8-dihydro-pyrido[2,3-#]pyrimidine-6-carbonyl]-amino}-benzoic acid methyl ester

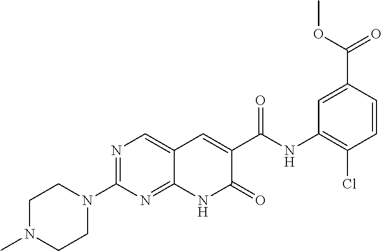

A mixture of 4-chloro-3-[(2-methanesulfonyl-7-oxo-7,8-dihydro-pyrido-pyrido[2,3-d]pyramid-ine-6-carbonyl)-amino]-benzoic acid methyl ester (0.04 g, 0.09 mmol) (from Example 32 supra) in 1-methylpiperazine (4 mL) was heated at 120° C. in a microwave reactor for 20 minutes. The mixture was concentrated and the residue was washed with hot methanol and dried in vacuum oven to give 4-chloro-3-{[2-(4-methyl-piperazin-1-yl)-7-oxo-7,8-dihydro-pyrido[2,3-d]pyrimidine-6-carbonyl]-amino}-benzoic acid methyl ester. (Yield 0.04 g, 95.7%).

HR-MS (ES⁺) m/z Calculated for C₂₁H₂₂ClN₆O₄ ([M+H]⁺): 457.1386. Found: 457.1385.

### Example 36

### 4-[6-(2-Chloro-5-methoxycarbonyl-phenylcarbamoyl)-7-oxo-7,8-dihydro-pyrido[2,3-d]pyrimidin-2-yl]-piperazine-1-carboxylic acid tert-butyl ester

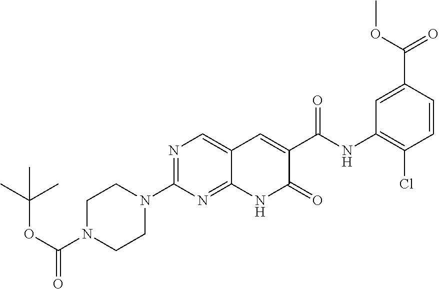

A mixture of 4-chloro-3-[(2-methanesulfonyl-7-oxo-7,8-dihydro-pyrido-pyrido[2,3-d]pyramid-ine-6-carbonyl)-amino]-benzoic acid methyl ester (0.04 g, 0.09 mmol) (from Example 32 supra) and 1-Boc-piperazine (0.34 g, 1.83 mmol) (Alfa Aesar) in DMF (4 mL) was heated at 120° C. in a microwave reactor for 20 minutes. The mixture was partitioned between dichloromethane and water. The aqueous phase was extracted with dichloromethane (2×). The combined organic phase was washed with water and brine, dried (magnesium sulfate), filtered and concentrated. The residue was purified by flash chromatography eluting with 40-95% ethyl acetate in hexanes to give 4-[6-(2-chloro-5-methoxycarbonyl-phenylcarbamoyl)-7-oxo-7,8-dihydro-pyrido[2,3-d]pyrimidin-2-yl]-piperazine-1-carboxylic acid tert-butyl ester. (Yield 0.027 g, 55.2%).

HR-MS (ES⁺) m/z Calculated for C₂₅H₂₈ClN₆O₆ ([M+H]⁺): 565.1573. Found: 565.1572.

### Example 37

### 4-Chloro-3-[(7-oxo-2-piperazin-1-yl-7,8-dihydro-pyrido[2,3-d]pyrimidine-6-carbonyl)-amino]-benzoic acid methyl ester

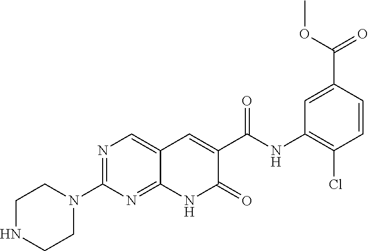

A mixture of 4-chloro-3-[(2-methanesulfonyl-7-oxo-7,8-dihydro-pyrido-pyrido[2,3-d]pyrimid-ine-6-carbonyl)-amino]-benzoic acid methyl ester (0.04 g, 0.09 mmol) (from Example 32 supra) and piperazine (0.16 g, 1.83 mmol) (Kodak) in DMF (4 mL) was heated at 120° C. in a microwave reactor for 20 minutes. The mixture was concentrated and the residue was washed with hot methanol and dried in vacuum oven to give 4-chloro-3-[(7-oxo-2-piperazin-1-yl-7,8-dihydro-pyrido[2,3-d]pyrimidine-6-carbonyl)-amino]-benzoic acid methyl ester. (Yield 0.03 g, 75.2%).

HR-MS (ES⁺) m/z Calculated for C₂₀H₂₀ClN₆O₄ ([M+H]⁺): 443.1229. Found: 443.1228.

### Example 38

### 4-Chloro-3-{[2-(2,3-dihydroxy-propylamino)-7-oxo-7,8-dihydro-pyrido[2,3-d]pyrimidine-6-carbonyl]-amino}-benzoic acid methyl ester

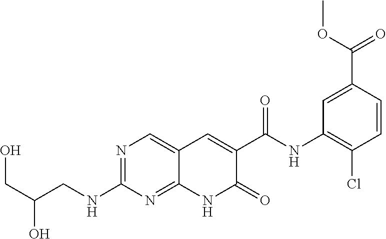

A mixture of 4-chloro-3-[(2-methanesulfonyl-7-oxo-7,8-dihydro-pyrido-pyrido[2,3-d]pyramid-ine-6-carbonyl)-amino]-benzoic acid methyl ester (0.04 g, 0.09 mmol) (from Example 32 supra) and 3-amino-1,2-propanediol (0.12 g, 1.35 mmol) (Aldrich) in DMF (4 mL) was heated at 120° C. in a microwave reactor for 20 minutes. The mixture was concentrated and the residue was washed with hot methanol and dried in vacuum oven to give 4-chloro-3-{[2-(2,3-dihydroxy-propylamino)-7-oxo-7,8-dihydro-pyrido[2,3-d]pyrimidine-6-carbonyl]-amino}-benzoic acid methyl ester. (Yield 0.023 g, 57.1%).

HR-MS (ES⁺) m/z Calculated for C₁₉H₁₉ClN₅O₆ ([M+H]⁺): 470.0838. Found: 470.0836.

### Example 39

### 3-({2-[Bis-(2-hydroxy-ethyl)-amino]-7-oxo-7,8-dihydro-pyrido[2,3-d]pyrimidine-6-carbonyl}-amino)-4-chloro-benzoic acid methyl ester

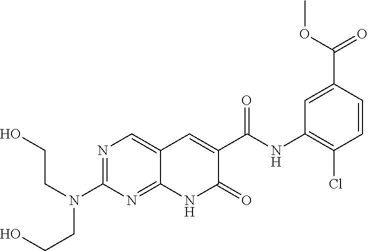

A mixture of 4-chloro-3-[(2-methanesulfonyl-7-oxo-7,8-dihydro-pyrido-pyrido[2,3-d]pyrimid-ine-6-carbonyl)-amino]-benzoic acid methyl ester (0.05 g, 0.11 mmol) (from Example 32 supra) and 2-(2-hydroxy-ethylamino)-ethanol (1 mL, 10.4 mmol) (Sigma) in DMF (3 mL) was heated at 120° C. in a microwave reactor for 20 minutes. The mixture was purified by HPLC eluting with MeCN/H₂O to give 3-({2-[bis-(2-hydroxy-ethyl)-amino]-7-oxo-7,8-dihydro-pyrido[2,3-d]pyrimidine-6-carbonyl}-amino)-4-chloro-benzoic acid methyl ester. (Yield 0.010 g, 19.7%).

HR-MS (ES⁺) m/z Calculated for C₂₀H₂₁ClN₅O₆ ([M+H]⁺): 462.1175. Found: 462.1175.

### Example 40

### 3-({2-[Bis-(2-ethoxy-ethyl)-amino]-7-oxo-7,8-dihydro-pyrido[2,3-d]pyrimidine-6-carbonyl}-amino)-4-chloro-benzoic acid methyl ester

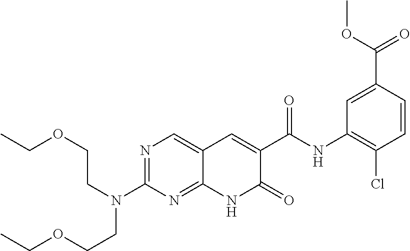

A mixture of 4-chloro-3-[(2-methanesulfonyl-7-oxo-7,8-dihydro-pyrido-pyrido[2,3-d]pyrimid-ine-6-carbonyl)-amino]-benzoic acid methyl ester (0.05 g, 0.11 mmol) (from Example 32 supra) and bis(2-ethoxyethyl)amine (1 mL, 6.84 mmol) (TCI-US) in DMF (3 mL) was heated at 120° C. in a microwave reactor for 20 minutes. The mixture was purified by HPLC eluting with MeCN/H₂O to give 3-({2-[bis-(2-ethoxy-ethyl)-amino]-7-oxo-7,8-dihydro-pyrido[2,3-d]pyrimidine-6-carbonyl}-amino)-4-chloro-benzoic acid methyl ester. (Yield 0.025 g, 43.9%).

HR-MS (ES⁺) m/z Calculated for C₂₄H₂₉ClN₅O₆ ([M+H]⁺): 518.1801. Found: 518.1801.

### Example 41

### 4-Chloro-3-{[2-(2-hydroxy-ethylamino)-7-oxo-7,8-dihydro-pyrido[2,3-d]pyrimidine-6-carbonyl]-amino}-benzoic acid methyl ester

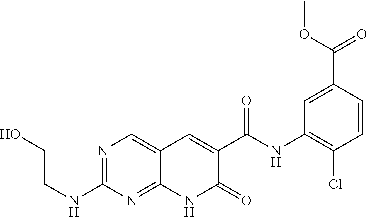

### Step A

### 2-(tert-Butyl-dimethyl-silanyloxy)-ethylamine

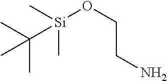

To a mixture of ethanolamine (0.916 g, 15 mmol) (Aldrich), tert-butyl-chloro-dimethyl-silane (2.48 g, 16.5 mmol) (Aldrich) and 4-dimethylamino-pyridine (0.183 g, 1.6 mmol) (Fluka) in 1,2-dichoroethane (15 mL) was added triethylamine (3.12 mL, 22.6 mmol) (Aldrich). The mixture was stirred at room temperature for 18 hours. The mixture was quenched with methanol (10 mL) and concentrated. The residue was diluted with ethyl acetate, the solid was filtered and the filtrate was washed with water, dried (MgSO₄), filtered and concentrated to give crude 2-(tert-butyl-dimethyl-silanyloxy)-ethylamine. This was used in next step without further purification. (Yield 1.74 g, 66.2%).

### Step B

### 3-{[2-(2-tert-Butyl-dimethyl-silanyloxy)-ethylamino]-7-oxo-7,8-dihydro-pyrido[2,3-d]pyrimidine-6-carbonyl}-amino}-4-chloro-benzoic acid methyl ester

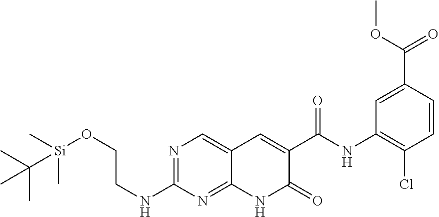

A mixture of 4-chloro-3-[(2-methanesulfonyl-7-oxo-7,8-dihydro-pyrido-pyrido[2,3-d]pyramid-ine-6-carbonyl)-amino]-benzoic acid methyl ester (from Example 32 supra) (0.05 g, 0.11 mmol) and 2-(tert-butyl-dimethyl-silanyloxy)-ethylamine (0.12 g, 0.68 mmol) in DMF (4 mL) was heated at 120° C. in a microwave reactor for 20 minutes. The mixture was diluted with ethyl acetate and the precipitate was filtered, washed with ethyl acetate and dried in vacuum oven to give 3-{[2-(2-tert-butyl-dimethyl-silanyloxy)-ethylamino]-7-oxo-7,8-dihydro-pyrido[2,3-d]pyrimidine-6-carbonyl}-amino}-4-chloro-benzoic acid methyl ester. (Yield 0.036 g, 61.5%).

### Step C

### 4-Chloro-3-{[2-(2-hydroxy-ethylamino)-7-oxo-7,8-dihydro-pyrido[2,3-d]pyrimidine-6-carbonyl]amino}-benzoic acid methyl ester

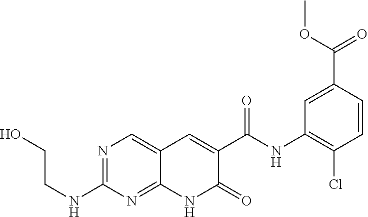

To a solution of 3-{[2-(2-tert-butyl-dimethyl-silanyloxy)-ethylamino]-7-oxo-7,8-dihydro-pyrido[2,3-d]pyrimidine-6-carbonyl}-amino}-4-chloro-benzoic acid methyl ester (0.034 g, 0.06 mmol) in THF (5 mL) was added 1M tetrabutylammonium fluoride in THF solution (0.19 mL, 0.19 mmol) (Aldrich). The mixture was heated at reflux for 3 hours. The mixture was concentrated and the residue was washed with methanol and dried in vacuum oven to give 4-chloro-3-{[2-(2-hydroxy-ethylamino)-7-oxo-7,8-dihydro-pyrido[2,3-d]pyrimidine-6-carbonyl]-amino}-benzoic acid methyl ester. (Yield 0.022 g, 88%).

HR-MS (ES⁺) m/z Calculated for C₁₈H₁₇ClN₅O₅ ([M+H]⁺): 440.0732. Found: 440.0731.

### Example 42

### 4-Chloro-3-{[2-(2-ethoxy-ethylamino)-7-oxo-7,8-dihydro-pyrido[2,3-d]pyrimidine-6-carbonyl]-amino}-benzoic acid methyl ester

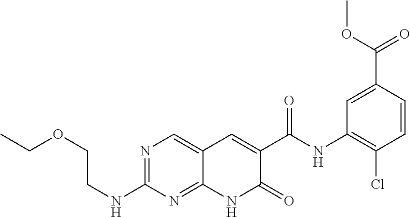

A mixture of 4-chloro-3-[(2-methanesulfonyl-7-oxo-7,8-dihydro-pyrido-pyrido[2,3-d]pyrimid-ine-6-carbonyl)-amino]-benzoic acid methyl ester (0.05 g, 0.11 mmol) (from Example 32 supra) and beta-ethoxyethylamine (1 mL, 9.53 mmol) (TCI-US) in DMF (3 mL) was heated at 120° C. in a microwave reactor for 20 minutes. The precipitate was filtered, washed with DMSO/methanol and dried in vacuum oven to give 4-chloro-3-{[2-(2-ethoxy-ethylamino)-7-oxo-7,8-dihydro-pyrido[2,3-d]pyrimidine-6-carbonyl]-amino}-benzoic acid methyl Ester. (Yield 0.032 g, 62.7%).

HR-MS (ES⁺) m/z Calculated for C₂₀H₂₁ClN₅O₅ ([M+H]⁺): 446.1226. Found: 446.1224.

### Example 43

### 4-Fluoro-3-[(2-methoxy-7-oxo-7,8-dihydro-pyrido[2,3-d]pyrimidine-6-carbonyl)-amino]-benzoic acid methyl ester

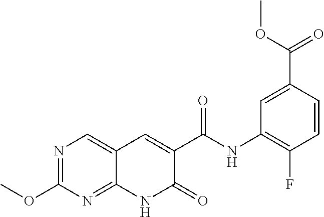

Triethylamine (61 mg, 0.60 mmol) was added to a mixture of crude 2-methoxy-7-oxo-7,8-dihydro-pyrido[2,3-d]pyrimidine-6-carboxylic acid (110 mg, 0.5 mmol) (from Example 18 supra) and HATU (0.23 g, 0.6 mmol) (Aldrich) in DMF (5.0 mL) at room temperature. The resulting mixture was stirred until clear solution was obtained (light brown). Methyl 3-amino-4-fluorobenzoate (0.110 g, 0.6 mmol) (Bionet) was added. The mixture was stirred for another 20 hours. Precipitate was formed. Water (50 mL), aqueous saturated sodium bicarbonate solution (10 mL), and ethyl acetate (30 mL) were added. After thorough mixing, pale yellow precipitate was collected by filtration, washed with water and ethyl acetate and dried in vacuum oven. Residue was recrystallized from DMF-ethyl acetate-hexanes to give 4-fluoro-3-[(2-methoxy-7-oxo-7,8-dihydro-pyrido[2,3-d]pyrimidine-6-carbonyl)-amino]-benzoic acid methyl ester as pale yellow needles. (Yield 0.10 g, 53.7%).

LR-LC/MS (APCi⁺) m/z [M+H]⁺ 373.

### Example 44

### 4-Chloro-3-{[2-(2-ethoxy-ethoxy)-7-oxo-7,8-dihydro-pyrido[2,3-d]pyrimidine-6-carbonyl]-amino}-benzoic acid methyl ester

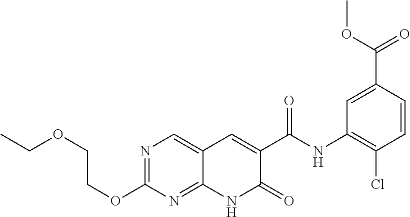

### Step A

### 4-Chloro-3-{[2-(2-ethoxy-ethoxy)-7-oxo-7,8-dihydro-pyrido[2,3-d]pyrimidine-6-carbonyl]-amino}-benzoic acid

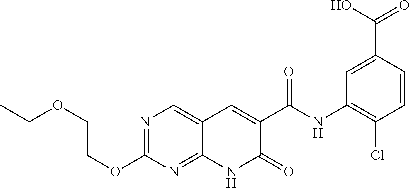

To a solution of 2-ethoxyethanol (0.07 mL, 0.7 mmol) (J T Baker) in DMF (5 mL) was added sodium hydride (0.028 g, 0.7 mmol). The mixture was stirred at room temperature for 30 minutes, and then 4-chloro-3-[(2-methanesulfonyl-7-oxo-7,8-dihydro-pyrido-pyrido[2,3-d]-pyrimidine-6-carbonyl)-amino]-benzoic acid methyl ester (0.044 g, 0.10 mmol) (from Example 32 supra) was added and the mixture was stirred at room temperature for 18 hours. The mixture was purified by HPLC eluting with MeCN/H₂O to give 4-chloro-3-{[2-(2-ethoxy-ethoxy)-7-oxo-7,8-dihydro-pyrido[2,3-d]pyrimidine-6-carbonyl]amino}-benzoic acid. (Yield 0.047 g, 109%).

### Step B

### 4-Chloro-3-{[2-(2-ethoxy-ethoxy)-7-oxo-7,8-dihydro-pyrido[2,3-d]pyrimidine-6-carbonyl]-amino}-benzoic acid methyl ester

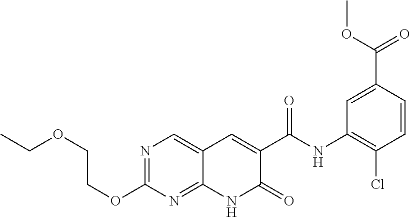

To a solution of 4-chloro-3-{[2-(2-ethoxy-ethoxy)-7-oxo-7,8-dihydro-pyrido[2,3-d]pyrimidine-6-carbonyl]amino}-benzoic acid (0.047 g, 0.11 mmol) in methanol (5 mL) was added conc. sulfuric acid (5 drops). The mixture was heated at reflux for 3 hours. The mixture was concentrated and the residue was purified by HPLC eluting with MeCN/H₂O to give 4-chloro-3-{[2-(2-ethoxy-ethoxy)-7-oxo-7,8-dihydro-pyrido[2,3-d]pyrimidine-6-carbonyl]-amino}-benzoic acid methyl ester. (Yield 0.016 g, 33.0%).

HR-MS (ES⁺) m/z Calculated for C₂₀H₂₀ClN₄O₆ ([M+H]⁺): 447.1066. Found: 447.1066.

### Example 45

### 4-Chloro-3-{[2-(2-diethylamino-ethoxy)-7-oxo-7,8-dihydro-pyrido[2,3-d]pyrimidine-6-carbonyl]-amino}-benzoic acid methyl ester

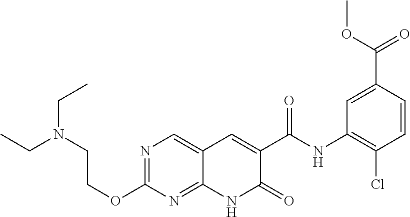

### Step A

### 4-Chloro-3-{[2-(2-diethylamino-ethoxy)-7-oxo-7,8-dihydro-pyrido[2,3-d]pyrimidine-6-carbonyl]amino}-benzoic acid

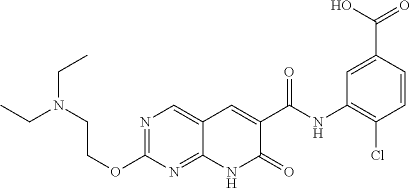

To a solution of N,N-diethylethanolamine (0.08 g, 0.7 mmol) (Aldrich) in DMF (5 mL) was added sodium hydride (0.028 g, 0.7 mmol). The mixture was stirred at room temperature for 30 minutes, and then 4-chloro-3-[(2-methanesulfonyl-7-oxo-7,8-dihydro-pyrido-pyrido[2,3-d]-pyrimidine-6-carbonyl)-amino]-benzoic acid methyl ester (0.044 g, 0.10 mmol) (from Example 32 supra) was added and the mixture was stirred at room temperature for 18 hours. The mixture was purified by HPLC eluting with MeCN/H₂O to give 4-chloro-3-{[2-(2-diethylamino-ethoxy)-7-oxo-7,8-dihydro-pyrido[2,3-d]pyrimidine-6-carbonyl]-amino}-benzoic acid. (Yield 0.030 g, 65.2%).

### Step B

### 4-Chloro-3-{[2-(2-diethylamino-ethoxy)-7-oxo-7,8-dihydro-pyrido[2,3-d]pyrimidine-6-carbonyl]-amino}-benzoic acid methyl ester

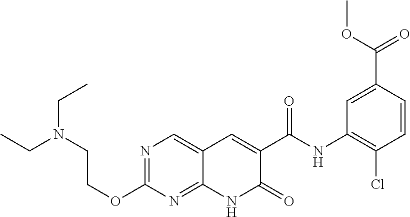

To a solution of 4-chloro-3-{[2-(2-diethylamino-ethoxy)-7-oxo-7,8-dihydro-pyrido[2,3-d]-pyramidine-6-carbonyl]amino}-benzoic acid (0.030 g, 0.11 mmol) in methanol (5 mL) was added conc. sulfuric acid (5 drops). The mixture was heated at reflux for 3 hours. The mixture was concentrated and the residue was purified by HPLC eluting with MeCN/H₂O to give 4-chloro-3-{[2-(2-diethylamino-ethoxy)-7-oxo-7,8-dihydro-pyrido[2,3-d]pyrimidine-6-carbonyl]-amino}-benzoic acid methyl ester. (Yield 0.009 g, 27.3%).

HR-MS (ES⁺) m/z Calculated for C₂₂H₂₅ClN₅O₅ ([M+H]⁺): 474.1539. Found: 474.1540.

### Example 46

### 2-Methoxy-7-oxo-7,8-dihydro-pyrido[2,3-d]pyrimidine-6-carboxylic acid [2-chloro-5-(4-chloro-benzylcarbamoyl)-phenyl]-amide

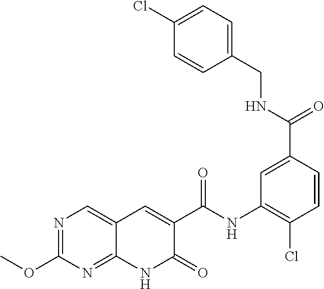

Diisopropylethylamine (0.07 mL, 0.40 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HBTU (0.074 g, 0.2 mmol) (Aldrich) in DMF (2.0 mL) at room temperature. The resulting mixture was stirred at room temperature for 30 minutes. 4-Choro-benzylamine (0.049 mL 0.4 mmol) (Aldrich) was added. Mixture was stirred for 1 day. The precipitate formed was collected by filtration, washed with methanol and dried in vacuum oven to give 2-methoxy-7-oxo-7,8-dihydro-pyrido[2,3-d]pyrimidine-6-carboxylic acid [2-chloro-5-(4-chloro-benzylcarbamoyl)-phenyl]-amide. (Yield 0.042 g, 64.6%).

HR-MS (ES⁺) m/z Calculated for C₂₃H₁₈Cl₂N₅O₄ ([M+H]⁺): 498.0731. Found: 498.0727.

### Example 47

### 2-Methoxy-7-oxo-7,8-dihydro-pyrido[2,3-d]pyrimidine-6-carboxylic acid [2-chloro-5-(2-chloro-benzylcarbamoyl)-phenyl]-amide

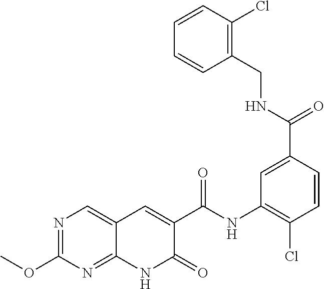

Diisopropylethylamine (0.07 mL, 0.40 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HBTU (0.074 g, 0.2 mmol) (Aldrich) in DMF (2.0 mL) at room temperature. The resulting mixture was stirred at room temperature for 30 minutes. 2-Choro-benzylamine (0.048 mL 0.4 mmol) (Aldrich) was added. The mixture was stirred for 1 day. The mixture was then purified by HPLC eluting with MeCN/H₂O to give 2-methoxy-7-oxo-7,8-dihydro-pyrido[2,3-d]pyrimidine-6-carboxylic acid [2-chloro-5-(2-chloro-benzylcarbamoyl)-phenyl]-amide. (Yield 0.020 g, 30.8%).

HR-MS (ES⁺) m/z Calculated for C₂₃H₁₈Cl₂N₅O₄ ([M+H]⁺): 498.0731. Found: 498.0731.

### Example 48

### 4-Bromo-3-[(2-methoxy-7-oxo-7,8-dihydro-pyrido[2,3-d]pyrimidine-6-carbonyl)-amino]-benzoic acid methyl ester

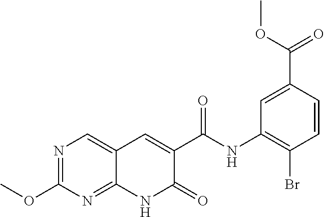

Triethylamine (61 mg, 0.60 mmol) was added to a mixture of crude 2-methoxy-7-oxo-7,8-dihydro-pyrido[2,3-d]pyrimidine-6-carboxylic acid (110 mg, 0.5 mmol) (from Example 18 supra) and HATU (0.23 g, 0.6 mmol) (Aldrich) in DMF (5.0 mL) at room temperature. The resulting mixture was stirred until clear solution was obtained (light brown). Methyl 3-amino-4-bromobenzoate (0.14 g, 0.6 mmol) (Oakwood) was added. The mixture was stirred for another 20 hours. Precipitate formed. Water (50 mL), aqueous saturated sodium bicarbonate solution (10 mL), and ethyl acetate (30 mL) were added. After thorough mixing, pale yellow precipitate was collected by filtration, washed with water and ethyl acetate and dried in vacuum oven. Residue was recrystallized from DMF-ethyl acetate-hexanes to give product as pale yellow crystals. (Yield 0.04 g, 18.5%).

HR-MS (ES⁺) m/z Calculated for C₁₇H₁₄BrN₄O₅ ([M+H]⁺): 433.0142. Found: 433.0143.

### Example 49

### 2-Methoxy-7-oxo-7,8-dihydro-pyrido[2,3-d]pyrimidine-6-carboxylic acid (2-chloro-5-phenylcarbamoyl-phenyl)-amide

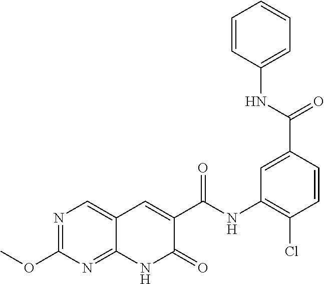

Diisopropylethylamine (0.07 mL, 0.40 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HBTU (0.074 g, 0.2 mmol) (Aldrich) in DMF (2.0 mL). The resulting mixture was stirred at room temperature for 30 minutes. Aniline (0.036 mL, 0.4 mmol) (Aldrich) was added. The mixture was stirred for 1 day. The mixture was purified by HPLC eluting with MeCN/H₂O to give 2-methoxy-7-oxo-7,8-dihydro-pyrido[2,3-d]pyrimidine-6-carboxylic acid [2-chloro-5-(2-chloro-benzylcarbamoyl)-phenyl]amide. (Yield 0.012 g, 20.5%).

HR-MS (ES⁺) m/z Calculated for C₂₂H₁₆ClN₅O₄ ([M+H]⁺): 450.0964. Found: 450.0962.

### Example 50

### 4-Chloro-3-[(2-methoxy-7-oxo-7,8-dihydro-pyrido[2,3-d]pyrimidine-6-carbonyl)-amino]-benzoic acid benzyl ester

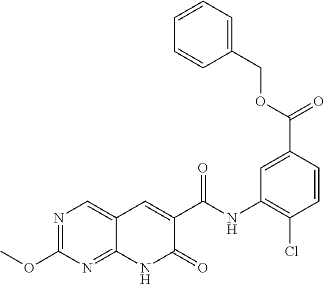

To a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and benzylbromide (0.024 mL, 0.2 mmol) in THF (10 mL) was added 1M tetrabutylammonium fluoride in THF solution (0.20 mL, 0.20 mmol) (Aldrich). The mixture was stirred at room temperature for 1 day. The mixture was concentrate and the residue was purified by HPLC eluting with MeCN/H₂O to give 4-chloro-3-[(2-methoxy-7-oxo-7,8-dihydro-pyrido[2,3-d]pyrimidine-6-carbonyl)-amino]-benzoic acid benzyl ester. (Yield 0.012 g, 19.9%).

HR-MS (ES⁺) m/z Calculated for C₂₃H₁₈ClN₄O₅ ([M+H]⁺): 465.0960. Found: 465.0960.

### Example 51

### 4-Chloro-3-[(2-methoxy-7-oxo-7,8-dihydro-pyrido[2,3-d]pyrimidine-6-carbonyl)-amino]-benzoic acid 4-chloro-benzyl ester

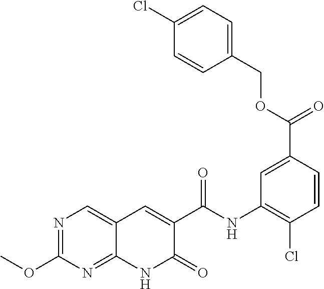

To a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and 4-chlorobenzylbromide (0.041 g, 0.2 mmol) in THF (10 mL) was added 1M tetrabutylammonium fluoride in THF solution (0.20 mL, 0.20 mmol) (Aldrich). The mixture was stirred at room temperature for 1 day. The mixture was concentrated and the residue was purified by HPLC eluting with MeCN/H₂O to give 4-chloro-3-[(2-methoxy-7-oxo-7,8-dihydro-pyrido[2,3-d]pyrimidine-6-carbonyl)-amino]-benzoic acid 4-chloro-benzyl ester. (Yield 0.005 g, 7.7%).

HR-MS (ES⁺) m/z Calculated for C₂₃H₁₆Cl₂N₄O₅ ([M+H]⁺): 499.0571. Found: 499.0572.

### Example 52

### 4-Chloro-3-[(2-methoxy-7-oxo-7,8-dihydro-pyrido[2,3-d]pyrimidine-6-carbonyl)-amino]-benzoic acid 2-chloro-benzyl ester

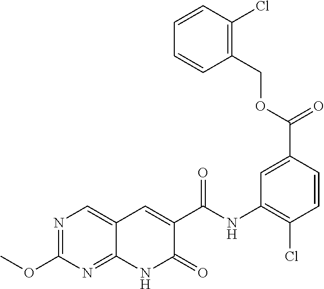

To a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and 2-chlorobenzylbromide (0.026 mL, 0.2 mmol) in THF (10 mL) was added 1M tetrabutylammonium fluoride in THF solution (0.20 mL, 0.20 mmol) (Aldrich). The resulting was mixture was stirred at room temperature for 1 day. The mixture was concentrated and the residue was purified by HPLC eluting with MeCN/H₂O to give 4-chloro-3-[(2-methoxy-7-oxo-7,8-dihydro-pyrido[2,3-d]pyrimidine-6-carbonyl)-amino]-benzoic acid 2-chloro-benzyl ester. (Yield 0.013 g, 20%).

HR-MS (ES⁺) m/z Calculated for C₂₃H₁₇Cl₂N₄O₅ ([M+H]⁺): 499.0571. Found: 499.0569.

### Example 53

### 4-Chloro-3-[(2-methoxy-7-oxo-7,8-dihydro-pyrido[2,3-d]pyrimidine-6-carbonyl)-amino]-benzoic acid 3-chloro-benzyl ester

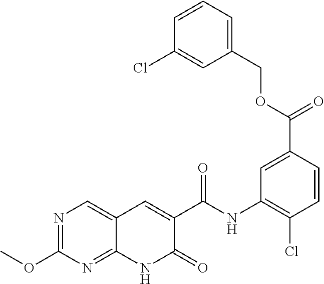

To a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and 3-chlorobenzylbromide (0.026 mL, 0.2 mmol) in THF (10 mL) was added 1M tetrabutylammonium fluoride in THF solution (0.20 mL, 0.20 mmol) (Aldrich). The mixture was stirred at room temperature for 1 day. The mixture was concentrate and the residue was purified by HPLC eluting with MeCN/H₂O to give 4-chloro-3-[(2-methoxy-7-oxo-7,8-dihydro-pyrido[2,3-d]pyrimidine-6-carbonyl)-amino]-benzoic acid 3-chloro-benzyl ester. (Yield 0.008 g, 12.3%).

HR-MS (ES⁺) m/z Calculated for C₂₃H₁₇Cl₂N₄O₅ ([M+H]⁺): 499.0571. Found: 499.0569.

### Example 54

### 2-Methoxy-7-oxo-7,8-dihydro-pyrido[2,3-d]pyrimidine-6-carboxylic acid [2-chloro-5-(3-chloro-benzylcarbamoyl)-phenyl]-amide

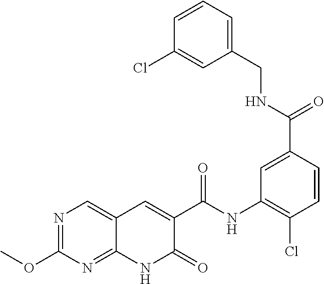

Diisopropylethylamine (0.07 mL, 0.40 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HBTU (0.074 g, 0.2 mmol) (Aldrich) in DMF (2.0 mL). Mixture was stirred at room temperature for 30 minutes. 3-Chlorobenzylamine (0.049 mL, 0.4 mmol) (Aldrich) was added. The resulting mixture was stirred for 1 day. Water was added and precipitate was collected by filtration, washed with water, ethyl acetate and methanol and dried in vacuum oven to give 2-methoxy-7-oxo-7,8-dihydro-pyrido[2,3-d]pyrimidine-6-carboxylic acid [2-chloro-5-(3-chloro-benzylcarbamoyl)-phenyl]amide. (Yield 0.022 g, 33.8%).

HR-MS (ES⁺) m/z Calculated for C₂₃H₁₇Cl₂N₅O₄ ([M+H]⁺): 498.0731. Found: 498.0730.

### Example 55

### 2-Methoxy-7-oxo-7,8-dihydro-pyrido[2,3-d]pyrimidine-6-carboxylic acid (2-chloro-5-dimethylcarbamoyl-phenyl)-amide

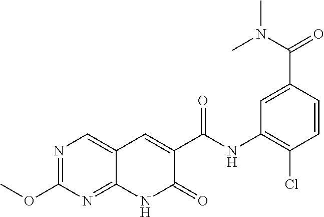

### Step A

### N,N-Dimethyl-3-nitro-4-chloro-benzamide

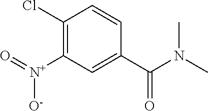

A solution of 4-chloro-3-nitrobenzoyl chloride (11.0 g, 50 mmol) (Aldrich) in ether (100 mL) was added dropwise to a mixture of dimethylamine (2.0 M in THF, 50 mL, 100 mmol) (Aldrich), sodium bicarbonate (8.5 g, 101 mmol), ether (50 mL) and water (100 mL) with cooling in ice bath and magnetic stirring. The resulting mixture was allowed to warm up to room temperature slowly. After stirring at room temperature for 2 hours, layers were separated. The organic layer was washed with water, 1 N aqueous hydrochloric acid and brine. The aqueous layers were back washed with ether. The ether layers were combined, dried (MgSO₄), filtered, and concentrated to give crude N,N-dimethyl-3-nitro-4-chloro-benzamide which was used without further purification. Yield was not determined.

### Step B

### 3-Amino-4-chloro-N,N-dimethyl-benzamide

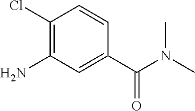

N,N-Dimethyl-3-nitro-4-chloro-benzamide (6.6 g, 28.9 mmol) was dissolved in mixture of ethanol (75 mL) and acetic acid (750 mL). Water (45 mL) was then added followed by conc. hydrochloric acid (0.18 g). Iron powder (6.06 g, 108.6 mmol) was added with vigorous stirring and the mixture was heated at reflux for 30 minutes. The mixture was filtered through Celite while hot. The filter cake was washed with ethanol. The combined filtrate and washing was diluted with water (300 mL) and extracted with ethyl acetate (3×300 mL). The organic layers were washed with saturated aqueous sodium bicarbonate solution (2×200 mL), water (200 mL) and brine (200 mL) and then combined. The combined organic layer was dried (MgSO₄), filtered, and concentrated to give crude 3-amino-4-chloro-N,N-dimethyl-benzamide as pale yellow oil which crystallized on standing. (Yield 5.50 g, 95.9%).

### Step C

### 2-Methoxy-7-oxo-7,8-dihydro-pyrido[2,3-d]pyrimidine-6-carboxylic acid (2-chloro-5-dimethylcarbamoyl-phenyl)-amide

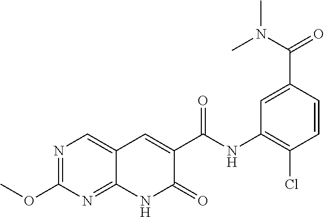

Triethylamine (0.1 g, 0.96 mmol) was added to a mixture of crude 2-methoxy-7-oxo-7,8-dihydro-pyrido[2,3-d]pyrimidine-6-carboxylic acid (180 mg, 0.8 mmol) (from Example 18 supra) and HATU (0.37 g, 0.96 mmol) (Aldrich) in DMF (9.0 mL) at room temperature. The resulting mixture was stirred until clear solution was obtained (light brown). 3-Amino-4-chloro-N,N-dimethyl-benzamide (0.19 g, 0.96 mmol) (TCI) was added. The mixture was stirred for another 20 hours. Precipitate was formed. Water (90 mL), aqueous saturated sodium bicarbonate solution (10 mL), and ethyl acetate (30 mL) were added. After thorough mixing and standing for 3 days, pale yellow precipitate was collected by filtration, washed with water and ethyl acetate. The residue was suspended in methanol and stirred at room temperature. Insoluble material was collected by filtration and dried in vacuum oven to give 2-methoxy-7-oxo-7,8-dihydro-pyrido[2,3-d]pyrimidine-6-carboxylic acid (2-chloro-5-dimethylcarbamoyl-phenyl)-amide as pale yellow crystals. (Yield 0.08 g, 24.9%).

HR-MS (ES⁺) m/z Calculated for C₁₈H₁₇ClN₅O₄ ([M+H]⁺): 402.0964. Found: 402.0964.

### Example 56

### 7-Oxo-2-piperidin-1-yl-7,8-dihydro-pyrido[2,3-d]pyrimidine-6-carboxylic acid

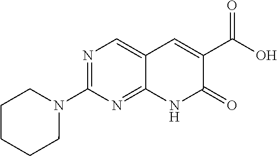

### Step A

### 7-oxo-2-piperidin-1-yl-7,8-dihydro-pyrido[2,3-d]pyrimidine-6-carboxylic acid methyl ester

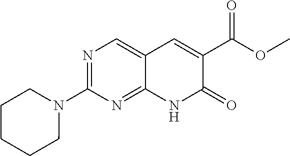

A mixture of 4-amino-2-methoxy-pyrimidine-5-carbaldehyde (6.89 g, 45 mmol) (from Example 16 supra), dimethyl malonate (16.5 g, 124.6 mmol) (Aldrich), piperidine (10.8 g, 126.9 mmol) (Aldrich), and acetic acid (0.3 g, 5 mmol) in methanol (180 mL) was heated at 60° C. for 24 hours. After cooling, the crystals formed were collected and washed with methanol to give 7-oxo-2-piperidin-1-yl-7,8-dihydro-pyrido[2,3-d]pyrimidine-6-carboxylic acid methyl ester as yellow crystals. The filtrate was concentrated to give a thick brown oil. This was suspended in small amount of methanol and the precipitate was collected by filteration and washing with methanol to give second crop of 7-oxo-2-piperidin-1-yl-7,8-dihydro-pyrido[2,3-d]pyrimidine-6-carboxylic acid methyl ester. (Combined yield 3.20 g, 24.7%).

### Step B

### 7-oxo-2-piperidin-1-yl-7,8-dihydro-pyrido[2,3-d]pyrimidine-6-carboxylic acid

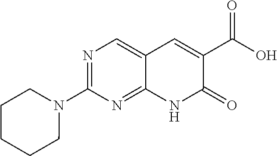

Aqueous sodium hydroxide solution (1 N, 41 mL, 41 mmol) was added to a suspension of 7-oxo-2-piperidin-1-yl-7,8-dihydro-pyrido[2,3-d]pyrimidine-6-carboxylic acid methyl ester (3.20 g, 13.61 mmol) in methanol (45 mL) and water (29 mL). The mixture was heated at 65° C. for 18 hours. After cooling, the mixture was diluted with water and concentrated under reduced pressure to remove most of the methanol. Clear solution was obtained which was washed with ether (2×50 mL). The aqueous layer was then acidified with concentrated aqueous hydrochloric acid. White precipitate that formed was collected by filtration and washed with water and dried in vacuum oven to give crude 7-oxo-2-piperidin-1-yl-7,8-dihydro-pyrido[2,3-d]pyrimidine-6-carboxylic acid as off-white powder. (Yield 2.37 g, 78.8%).

HR-MS (ES⁺) m/z Calculated for C₁₃H₁₅N₄O₃ ([M+H]⁺): 275.1139. Found: 275.1139.

### Example 57

### 2-Methoxy-7-oxo-7,8-dihydro-pyrido[2,3-d]pyrimidine-6-carboxylic acid {2-chloro-5-[(thiophen-2-ylmethyl)-carbamoyl]-phenyl}-amide

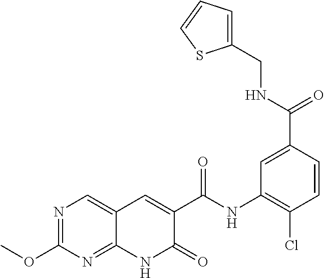

Diisopropylethylamine (0.07 mL, 0.40 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.076 g, 0.2 mmol) (Aldrich) in DMF (2.0 mL). The resulting mixture was stirred at room temperature for 30 minutes. 2-Thiophene-methylamine (0.041 mL, 0.4 mmol) (Aldrich) was added. The mixture was stirred for 18 hours. The mixture was partitioned between ethyl acetate and water and precipitate was collected by filtration, washed with water, ethyl acetate and methanol and dried in vacuum oven to give 2-methoxy-7-oxo-7,8-dihydro-pyrido[2,3-d]pyrimidine-6-carboxylic acid {2-chloro-5-[(thiophen-2-ylmethyl)-carbamoyl]-phenyl}-amide. (Yield 0.039 g, 63.9%).

HR-MS (ES⁺) m/z Calculated for C₂₁H₁₇ClN₅O₄S ([M+H]⁺): 470.0685. Found: 470.0684.

### Example 58

### 2-Methoxy-7-oxo-7,8-dihydro-pyrido[2,3-d]pyrimidine-6-carboxylic acid {2-chloro-5-[(thiophen-3-ylmethyl)-carbamoyl]-phenyl}-amide

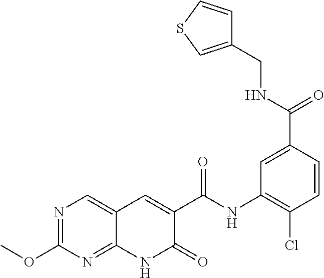

Diisopropylethylamine (0.07 mL, 0.40 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.076 g, 0.2 mmol) (Aldrich) in DMF (2.0 mL). The resulting mixture was stirred at room temperature for 30 minutes. 3-Thiophene-methylamine (0.045 g, 0.4 mmol) (MayBridge) was added. The mixture was stirred for 18 hours. The mixture was partitioned between ethyl acetate and water and precipitate was collected by filtration, washed with water, ethyl acetate and methanol and dried in vacuum oven to give 2-methoxy-7-oxo-7,8-dihydro-pyrido[2,3-d]pyrimidine-6-carboxylic acid {2-chloro-5-[(thiophen-3-ylmethyl)-carbamoyl]-phenyl}-amide. (Yield 0.057 g, 93.4%).

HR-MS (ES⁺) m/z Calculated for C₂₁H₁₇ClN₅O₄S ([M+H]⁺): 470.0685. Found: 470.0684.

### Example 59

### 2-Methoxy-7-oxo-7,8-dihydro-pyrido[2,3-d]pyrimidine-6-carboxylic acid [2-chloro-5-(3-trifluoromethyl-benzylcarbamoyl)-phenyl]-amide

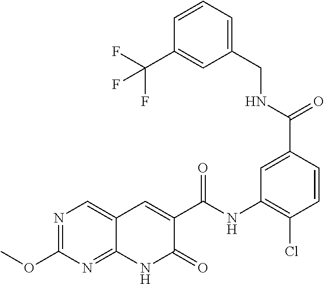

Diisopropylethylamine (0.07 mL, 0.40 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.076 g, 0.2 mmol) (Aldrich) in DMF (2.0 mL). The resulting mixture was stirred at room temperature for 30 minutes. 3-(Trifluoromethyl)benzylamine (0.06 mL, 0.4 mmol) (Aldrich) was added. The mixture was stirred for 18 hours. The precipitate was collected by filtration, washed with methanol and dried in vacuum oven to give 2-methoxy-7-oxo-7,8-dihydro-pyrido[2,3-d]pyrimidine-6-carboxylic acid [2-chloro-5-(3-trifluoromethyl-benzylcarbamoyl)-phenyl]amide. (Yield 0.022 g, 31.9%).

HR-MS (ES⁺) m/z Calculated for C₂₄H₁₈ClF₃N₅O₄ ([M+H]⁺): 532.0994. Found: 532.0995.

### Example 60

### 2-Methoxy-7-oxo-7,8-dihydro-pyrido[2,3-d]pyrimidine-6-carboxylic acid [5-(benzyl-methyl-carbamoyl)-2-chloro-phenyl]-amide

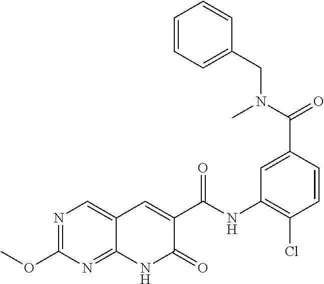

Diisopropylethylamine (0.07 mL, 0.40 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.076 g, 0.2 mmol) (Aldrich) in DMF (2.0 mL) at room temperature. The resulting mixture was stirred at room temperature for 30 minutes. N-Benzylmethyl-amine (0.05 mL, 0.4 mmol) (Aldrich) was added. The mixture was stirred for 18 hours. The mixture was partitioned between ethyl acetate and water. The aqueous phase was extracted with ethyl acetate (3×). The combined organic phase was washed with water and brine, dried (magnesium sulfate), filtered and concentrated. The residue was recrystallized from dichloromethane to give 2-methoxy-7-oxo-7,8-dihydro-pyrido[2,3-d]pyrimidine-6-carboxylic acid [5-(benzyl-methyl-carbamoyl)-2-chloro-phenyl]-amide. (Yield 0.040 g, 64.5%).

HR-MS (ES⁺) m/z Calculated for C₂₄H₂₁ClN₅O₄ ([M+H]⁺): 478.1277. Found: 478.1276.

### Example 61

### 4-Chloro-3-[(7-oxo-2-piperidin-1-yl-7,8-dihydro-pyrido[2,3-d]pyrimidine-6-carbonyl)-amino]-benzoic acid

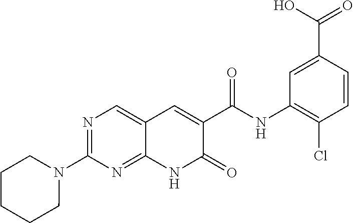

A solution of sodium hydroxide (0.16 g, 4.1 mmol) in water (10 mL) was added to a suspension of 4-chloro-3-[(7-oxo-2-piperidin-1-yl-7,8-dihydro-pyrido[2,3-d]pyrimidine-6-carbonyl)-amino]-benzoic acid methyl ester (0.32 g, 0.72 mmol) (from Example 34 supra) in methanol (10 mL). The resulting mixture was heated at 65° C. for 18 hours. After cooling, the mixture was concentrated under reduced pressure to remove most of the methanol. The residue was diluted with water (10 mL) and acidified with concentrated hydrochloric acid. A white precipitate formed which was then collected by filtration and washed with water and dried in vacuum oven to give crude 4-chloro-3-[(7-oxo-2-piperidin-1-yl-7,8-dihydro-pyrido[2,3-d]pyrimidine-6-carbonyl)-amino]-benzoic acid as white powder. (Yield 0.28 g, 90.3%).

HR-MS (ES⁺) m/z Calculated for C₂₀H₁₉ClN₅O₄ ([M+H]⁺): 428.1120. Found: 428.1121.

### Example 62

### 2-Methoxy-7-oxo-7,8-dihydro-pyrido[2,3-d]pyrimidine-6-carboxylic acid [5-(3-chloro-benzylcarbamoyl)-2-methyl-phenyl]-amide

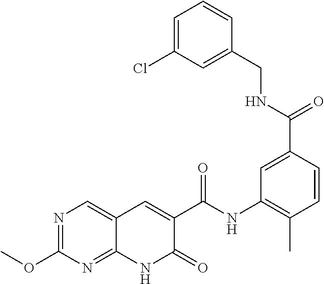

Diisopropylethylamine (0.073 mL, 0.42 mmol) was added to a mixture of 3-[(2-methoxy-7-oxo-7,8-dihydro-pyrido[2,3-d]pyrimidine-6-carbonyl)-amino]-4-methyl-benzoic acid (0.05 g, 0.14 mmol) (from Example 20 supra) and HATU (0.080 g, 0.21 mmol) (Aldrich) in DMF (2.0 mL). The resulting mixture was stirred at room temperature for 30 minutes. 3-Chlorobenzylamine (0.051 mL, 0.42 mmol) (Aldrich) was added. The mixture was stirred for 18 hours. The mixture was partitioned between ethyl acetate and water. The precipitate was collected by filtration and washed with ethyl acetate and water and dried in vacuum oven to give 2-methoxy-7-oxo-7,8-dihydro-pyrido[2,3-d]pyrimidine-6-carboxylic acid [5-(3-chloro-benzylcarbamoyl)-2-methyl-phenyl]-amide. (Yield 0.031 g, 46.3%).

HR-MS (ES⁺) m/z Calculated for C₂₄H₂₁ClN₅O₄ ([M+H]⁺): 500.1096. Found: 500.1096.

### Example 63

### 2-Methoxy-7-oxo-7,8-dihydro-pyrido[2,3-d]pyrimidine-6-carboxylic acid {5-[(3-chloro-benzyl)-methyl-carbamoyl]-2-methyl-phenyl}-amide

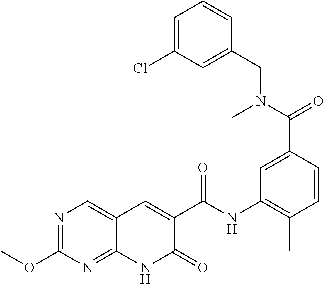

Diisopropylethylamine (0.073 mL, 0.42 mmol) was added to a mixture of 3-[(2-methoxy-7-oxo-7,8-dihydro-pyrido[2,3-d]pyrimidine-6-carbonyl)-amino]-4-methyl-benzoic acid (0.05 g, 0.14 mmol) (from Example 20 supra) and HATU (0.080 g, 0.21 mmol) (Aldrich) in DMF (2.0 mL). The resulting mixture was stirred at room temperature for 30 minutes. 3-Chloro-N-methylbenzylamine (0.061 mL, 0.42 mmol) (Aldrich) was added. The mixture was stirred for 18 hours. The mixture was partitioned between ethyl acetate and water. The precipitate formed was collected by filtration and washed with methanol and dried in vacuum oven to give 2-methoxy-7-oxo-7,8-dihydro-pyrido[2,3-d]pyrimidine-6-carboxylic acid {5-[(3-chloro-benzyl)-methyl-carbamoyl]-2-methyl-phenyl}-amide. (Yield 0.018 g, 26.1%).

HR-MS (ES⁺) m/z Calculated for C₂₅H₂₃ClN₅O₄ ([M+H]⁺): 514.1252. Found: 514.1251.

### Example 64

### 7-Oxo-2-piperidin-1-yl-7,8-dihydro-pyrido[2,3-d]pyrimidine-6-carboxylic acid {2-chloro-5-[(3-chloro-benzyl)-methyl-carbamoyl]-phenyl}-amide

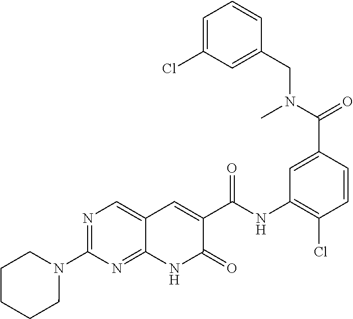

Diisopropylethylamine (0.07 mL, 0.40 mmol) was added to a mixture of 4-chloro-3-[(7-oxo-2-piperidin-1-yl-7,8-dihydro-pyrido[2,3-d]pyrimidine-6-carbonyl)-amino]-benzoic acid (0.05 g, 0.11 mmol) (from Example 61 supra) and HATU (0.076 g, 0.20 mmol) in DMF (2.0 mL). The resulting mixture was stirred at room temperature for 30 minutes. 3-Chloro-N-methylbenzylamine (Aldrich) (0.06 mL, 0.40 mmol) was added. The mixture was stirred for 18 hours and then partitioned between ethyl acetate and water. The precipitate was collected by filtration and washed with methanol and dried in vacuum oven to give 7-oxo-2-piperidin-1-yl-7,8-dihydro-pyrido[2,3-d]pyrimidine-6-carboxylic acid {2-chloro-5-[(3-chloro-benzyl)-methyl-carbamoyl]-phenyl}-amide. (Yield 0.040 g, 62.5%).

HR-MS (ES⁺) m/z Calculated for C₂₈H₂₇Cl₂N₆O₃ ([M+H]⁺): 587.1335. Found: 587.1340.

### Example 65

### 2-Methoxy-7-oxo-7,8-dihydro-pyrido[2,3-d]pyrimidine-6-carboxylic acid {2-chloro-5-[(3-chloro-benzyl)-methyl-carbamoyl]-phenyl}-amide

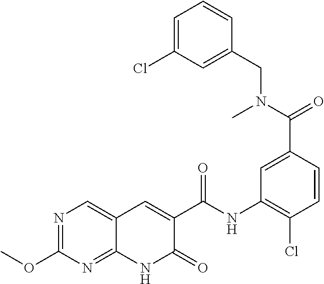

Diisopropylethylamine (0.07 mL, 0.40 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.076 g, 0.2 mmol) (Aldrich) in DMF (2.0 mL). The resulting mixture was stirred at room temperature for 30 minutes. 3-Chloro-N-methylbenzylamine (0.06 mL, 0.4 mmol) (Aldrich) was added. The mixture was stirred for 18 hours and then partitioned between ethyl acetate and water. The aqueous phase was extracted with ethyl acetate (3×). The combined organic phase was washed with water and brine, dried (magnesium sulfate), filtered, and concentrated. The residue was purified by flash chromatography eluting with 0-50% ethyl acetate in dichloromethane to give 2-methoxy-7-oxo-7,8-dihydro-pyrido[2,3-d]pyrimidine-6-carboxylic acid {2-chloro-5-[(3-chloro-benzyl)-methyl-carbamoyl]-phenyl}-amide.(Yield 0.017 g, 28.3%).

HR-MS (ES⁺) m/z Calculated for C₂₄H₂₀Cl₂N₅O₄ ([M+H]⁺): 512.0887. Found: 512.0888.

### Example 66

### 2-Methoxy-7-oxo-7,8-dihydro-pyrido[2,3-d]pyrimidine-6-carboxylic acid {2-chloro-5-[(pyridin-2-ylmethyl)-carbamoyl]-phenyl}-amide

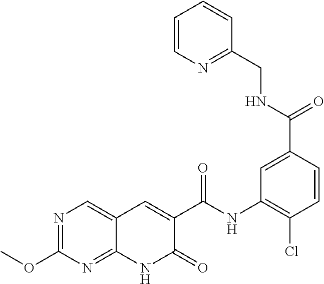

Triethylamine (0.056 mL, 0.40 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.076 g, 0.2 mmol) (Aldrich) in DMF (2.0 mL). The resulting mixture was stirred at room temperature for 30 minutes. 2-(Aminomethyl)-pyridine (0.041 mL, 0.4 mmol) (Aldrich) was added. Mixture was stirred for 18 hours and then partitioned between ethyl acetate and water. The aqueous phase was extracted with ethyl acetate (3×). The combined organic phase was washed with water and brine, dried (magnesium sulfate), filtered, and concentrated. The residue was collected by filtration and washed with ethyl acetate and water and dried in vacuum oven to give 2-methoxy-7-oxo-7,8-dihydro-pyrido[2,3-d]pyrimidine-6-carboxylic acid {2-chloro-5-[(pyridin-2-ylmethyl)-carbamoyl]-phenyl}-amide. (Yield 0.007 g, 11.3%).

HR-MS (ES⁺) m/z Calculated for C₂₂H₁₈ClN₆O₄ ([M+H]⁺): 465.1073. Found: 465.1074.

### Example 67

### 2-Methoxy-7-oxo-7,8-dihydro-pyrido[2,3-d]pyrimidine-6-carboxylic acid {2-chloro-5-[(pyridin-3-ylmethyl)-carbamoyl]-phenyl}-amide

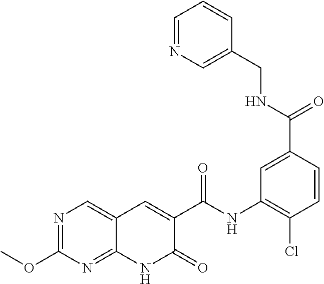

Triethylamine (0.056 mL, 0.40 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.076 g, 0.2 mmol) (Aldrich) in DMF (2.0 mL). The resulting mixture was stirred at room temperature for 30 minutes. 3-(Aminomethyl)-pyridine (0.041 mL, 0.4 mmol) (Aldrich) was added. The mixture was stirred for 18 hours and then partitioned between ethyl acetate and water. The aqueous phase was extracted with ethyl acetate (3×). The combined organic phase was washed with water and brine, dried (magnesium sulfate), filtered, and concentrated. The residue was collected by filtration and washed with ethyl acetate and water and dried in vacuum oven to give 2-methoxy-7-oxo-7,8-dihydro-pyrido[2,3-d]pyrimidine-6-carboxylic acid {2-chloro-5-[(pyridin-3-ylmethyl)-carbamoyl]-phenyl}-amide. (Yield 0.042 g, 67.7%).

HR-MS (ES⁺) m/z Calculated for C₂₂H₁₈ClN₆O₄ ([M+H]⁺): 465.1073. Found: 465.1073.

### Example 68

### 2-Methoxy-7-oxo-7,8-dihydro-pyrido[2,3-d]pyrimidine-6-carboxylic acid {2-chloro-5-[(pyridin-4-ylmethyl)-carbamoyl]-phenyl}-amide

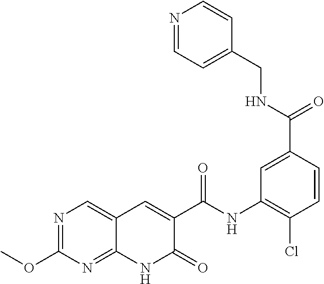

Triethylamine (0.056 mL, 0.40 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.076 g, 0.2 mmol) (Aldrich) in DMF (2.0 mL). The resulting mixture was stirred at room temperature for 30 minutes. 4-(Aminomethyl)-pyridine (0.041 mL, 0.4 mmol) (Aldrich) was added. The mixture was stirred for 18 hours and then partitioned between ethyl acetate and water. The aqueous phase was extracted with ethyl acetate (3×). The combined organic phase was washed with water and brine, dried (magnesium sulfate), filtered, and concentrated. The residue was collected by filtration and washed with ethyl acetate and water and dried in vacuum oven to give 2-methoxy-7-oxo-7,8-dihydro-pyrido[2,3-d]pyrimidine-6-carboxylic acid {2-chloro-5-[(pyridin-4-ylmethyl)-carbamoyl]-phenyl}-amide. (Yield 0.036 g, 58.1%).

HR-MS (ES⁺) m/z Calculated for C₂₂H₁₈ClN₆O₄ ([M+H]⁺): 465.1073. Found: 465.1073.

### Example 69

### (2-{4-Chloro-3-[(2-methoxy-7-oxo-7,8-dihydro-pyrido[2,3-d]pyrimidine-6-carbonyl)-amino]-benzoylamino}-2-phenyl-ethyl)-carbamic acid tert-butyl ester

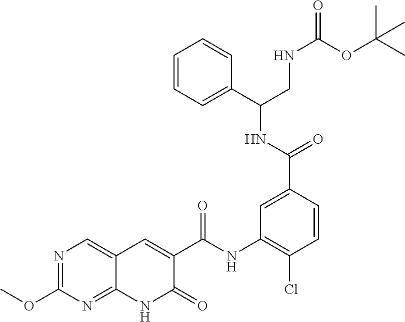

Triethylamine (0.042 mL, 0.30 mmol) was added to a mixture of 4-chloro-3-[(2-methoxy-7-oxo-7,8-dihydro-pyrido[2,3-d]pyrimidine-6-carbonyl)-amino]-benzoic acid (0.1 g, 0.27 mmol) (from Example 22 supra) and HATU (0.11 g, 0.3 mmol) (Aldrich) in DMF (4.0 mL). The resulting mixture was stirred at room temperature for 30 minutes. (2-Amino-2-phenyl-ethyl)-carbamic acid tert-butyl ester (0.08 g, 0.32 mmol) (from Example 4 supra) was added. The mixture was stirred for 18 hours. The mixture was partitioned between ethyl acetate and water. The aqueous phase was extracted with ethyl acetate (3×). The combined organic phase was washed with water and brine, dried (magnesium sulfate) and concentrated. The residue was collected by filtration and washed with ethyl acetate and water and dried in vacuum oven to give (2-{4-chloro-3-[(2-methoxy-7-oxo-7,8-dihydro-pyrido[2,3-d]pyrimidine-6-carbonyl)-amino]-benzoylamino}-2-phenyl-ethyl)-carbamic acid tert-butyl ester. (Yield 0.070 g, 46.7%).

HR-MS (ES⁺) m/z Calculated for C₂₉H₃₀ClN₆O₆ ([M+H]⁺): 615.1729. Found: 615.1732.

### Example 70

### 2-Methoxy-7-oxo-7,8-dihydro-pyrido[2,3-d]pyrimidine-6-carboxylic acid (2-chloro-5-phenethylcarbamoyl-phenyl)-amide

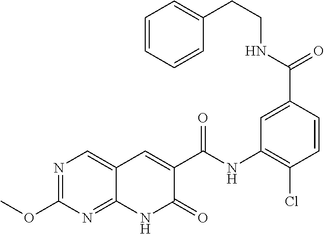

Triethylamine (0.021 mL, 0.15 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.056 g, 0.15 mmol) (Aldrich) in DMF (2.0 mL). The resulting mixture was stirred at room temperature for 30 minutes. 2-Phenethylamine (0.020 mL, 0.16 mmol) was added. The mixture was stirred for 18 hours and then partitioned between ethyl acetate and water. The aqueous phase was extracted with ethyl acetate (3×). The combined organic phase was washed with water and brine, dried (magnesium sulfate) and concentrated. The residue was collected by filtration and washed with ethyl acetate and water and dried in vacuum oven to give 2-methoxy-7-oxo-7,8-dihydro-pyrido[2,3-d]pyrimidine-6-carboxylic acid (2-chloro-5-phenethylcarbamoyl-phenyl)-amide. (Yield 0.051 g, 79.7%).

HR-MS (ES⁺) m/z Calculated for C₂₄H₂₁ClN₅O₄ ([M+H]⁺): 478.1277. Found: 478.1277.

### Example 71

### 2-Methoxy-7-oxo-7,8-dihydro-pyrido[2,3-d]pyrimidine-6-carboxylic acid {2-chloro-5-[2-(3-chloro-phenyl)-ethylcarbamoyl]-phenyl}-amide

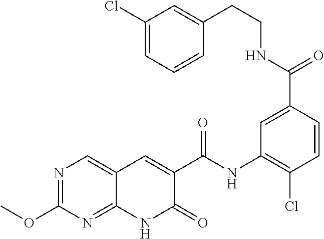

Triethylamine (0.021 mL, 0.15 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.056 g, 0.15 mmol) (Aldrich) in DMF (2.0 mL). The resulting mixture was stirred at room temperature for 30 minutes. 2-(3-Chlorophenyl)ethylamine (0.025 g, 0.16 mmol) was added. The mixture was stirred for 18 hours and then partitioned between ethyl acetate and water. The aqueous phase was extracted with ethyl acetate (3×). The combined organic phase was washed with water and brine, dried (magnesium sulfate) and concentrated. The residue was collected by filtration and washed with ethyl acetate and water and dried in vacuum oven to give 2-methoxy-7-oxo-7,8-dihydro-pyrido[2,3-d]pyrimidine-6-carboxylic acid {2-chloro-5-[2-(3-chloro-phenyl)-ethylcarbamoyl]-phenyl}-amide. (Yield 0.053 g, 77.9%).

HR-MS (ES⁺) m/z Calculated for C₂₄H₂₀Cl₂N₅O₄ ([M+H]⁺): 512.0887. Found: 512.0887.

### Example 72

### 2-Methoxy-7-oxo-7,8-dihydro-pyrido[2,3-d]pyrimidine-6-carboxylic acid [5-(2-amino-1-phenyl-ethylcarbamoyl)-2-chloro-phenyl]-amide

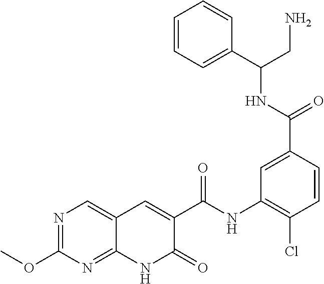

To a solution of (2-{4-chloro-3-[(2-methoxy-7-oxo-7,8-dihydro-pyrido[2,3-d]pyrimidine-6-carbonyl)-amino]-benzoylamino}-2-phenyl-ethyl)-carbamic acid tert-butyl ester (0.051 g, 0.09 mmol) (from Example 69 supra) in dichloromethane (5 mL) was added a mixture of 2,2,2-trifluoroacetic acid (2.5 mL) and dichloromethane (2.5 mL). The resulting mixture was stirred at 0° C. for 3 hours and room temperature for 18 hours. The mixture was concentrated and then partitioned between dichloromethane and saturated sodium carbonate solution. The aqueous phase was concentrated and the inorganic salt was removed by filtration and washed with methanol and the filtrate was purified by HPLC eluting with MeCN/H₂O to give 2-methoxy-7-oxo-7,8-dihydro-pyrido[2,3-d]pyrimidine-6-carboxylic acid [5-(2-amino-1-phenyl-ethylcarbamoyl)-2-chloro-phenyl]-amide. (Yield 0.007 g, 16.7%).

HR-MS (ES⁺) m/z Calculated for C₂₄H₂₂ClN₆O₄ ([M+H]⁺): 493.1386. Found: 493.1388.

### Example 73

### (3-{4-Chloro-3-[(2-methoxy-7-oxo-7,8-dihydro-pyrido[2,3-d]pyrimidine-6-carbonyl)-amino]-benzoylamino}-3-phenyl-propyl)-carbamic acid tert-butyl ester

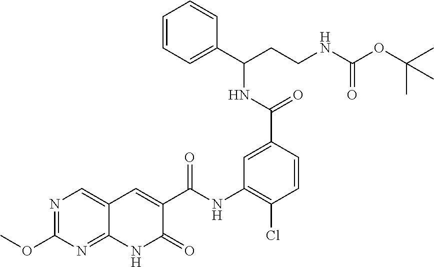

Triethylamine (0.042 mL, 0.30 mmol) was added to a mixture of 4-chloro-3-[(2-methoxy-7-oxo-7,8-dihydro-pyrido[2,3-d]pyrimidine-6-carbonyl)-amino]-benzoic acid (0.1 g, 0.27 mmol) (from Example 22 supra) and HATU (0.11 g, 0.3 mmol) (Aldrich) in DMF (4.0 mL). The resulting mixture was stirred at room temperature for 30 minutes. (3-Amino-3-phenyl-propyl)-carbamic acid tert-butyl ester (0.08 g, 0.32 mmol) (from Example 6 supra) was added. The mixture was stirred for 18 hours and then partitioned between ethyl acetate and water. The aqueous phase was extracted with ethyl acetate (3×). The combined organic phase was washed with water and brine, dried (magnesium sulfate) and concentrated. The residue was purified by flash chromatography eluting with 20-50% ethyl acetate in dichloromethane to give (3-{4-chloro-3-[(2-methoxy-7-oxo-7,8-dihydro-pyrido[2,3-d]pyrimidine-6-carbonyl)-amino]-benzoylamino}-3-phenyl-propyl)-carbamic acid tert-butyl ester. (Yield 0.083 g, 51.9%).

HR-MS (ES⁺) m/z Calculated for C₃₀H₃₂ClN₆O₆ ([M+H]⁺): 629.1888. Found: 629.1886.

### Example 74

### 2-Methylsulfanyl-7-oxo-7,8-dihydro-pyrido[2,3-d]pyrimidine-6-carboxylic acid [2-chloro-5-(3-chloro-benzylcarbamoyl)-phenyl]-amide

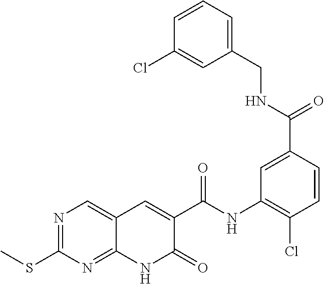

### Step A

### 4-Chloro-3-[(2-methylsulfanyl-7-oxo-7,8-dihydro-pyrido[2,3-d]pyrimidine-6-carbonyl)-amino]-benzoic acid methyl ester

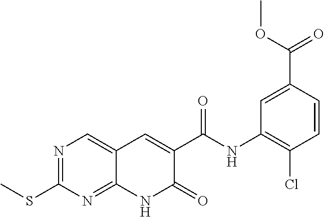

To a 250 mL round bottom flask equipped with a stir bar was added 2-methylsulfanyl-7-oxo-7,8-dihydro-pyrido[2,3-d]pyrimidine-6-carboxylic acid (4.0 g, 17.0 mmol) (from Example 24 supra), 3-amino-4-chloro-benzoic acid methyl ester (4.6 g, 25.0 mmol), N,N-dimethylformamide (45 mL) and triethylamine (4.9 mL, 35.0 mmol), after which HATU (9.5 g, 25.0 mmol) was added all at once. The reaction was allowed to stir at room temperature for 18 hours after which the reaction was diluted with ethyl acetate and the precipitate was filtered, rinsed with water, ethyl acetate and then dried under high vacuum to provide 4-chloro-3-[(2-methylsulfanyl-7-oxo-7,8-dihydro-pyrido[2,3-d]pyrimidine-6-carbonyl)-amino]-benzoic acid methyl ester as an off-white solid. (Yield 5.27 g, 76%).

### Step B

### 4-Chloro-3-[(2-methylsulfanyl-7-oxo-7,8-dihydro-pyrido[2,3-d]pyrimidine-6-carbonyl)-amino]-benzoic acid

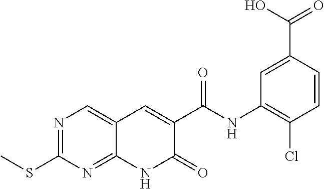

To a 250 mL round bottom flask equipped with a stir bar was added 4-chloro-3-[(2-methylsulfanyl-7-oxo-7,8-dihydro-pyrido[2,3-d]pyrimidine-6-carbonyl)-amino]-benzoic acid methyl ester (5.27 g, 13.0 mmol), lithium hydroxide (0.782 g, 32.6 mmol), methanol (50 mL), THF (20 mL) and water (30 mL). The flask was heated at 65° C. with stirring for 12 hours, after which it was concentrated to dryness under reduced pressure. The crude material was carefully acidified with aqueous hydrochloric acid (2.0 M) and the precipitated white solid was collected by filtration, rinsed with water and dried in an vacuum oven (80° C.) overnight to provide 4-chloro-3-[(2-methylsulfanyl-7-oxo-7,8-dihydro-pyrido[2,3-d]pyrimidine-6-carbonyl)-amino]-benzoic acid. (Yield 4.8 g, 94%).

### Step C

### 2-Methylsulfanyl-7-oxo-7,8-dihydro-pyrido[2,3-d]pyrimidine-6-carboxylic acid [2-chloro-5-(3-chloro-benzylcarbamoyl)-phenyl]-amide

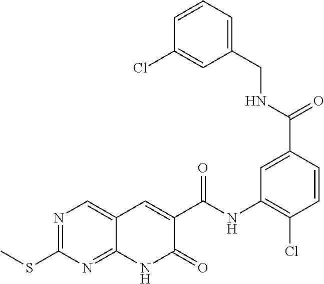

To a 250 mL round bottom flask equipped with a stir bar was added 4-chloro-3-[(2-methylsulfanyl-7-oxo-7,8-dihydro-pyrido[2,3-d]pyrimidine-6-carbonyl)-amino]-benzoic acid (4.8 g, 12.3 mmol), DMF (50 mL), triethylamine (8.3 mL, 60.0 mmol), 3-chlorobenyzl amine (2.2 mL, 18.0 mmol) and then HATU (6.84 g, 18.0 mmol). The reaction was allowed to stir at room temperature for 12 hours, after which it was diluted with ethyl acetate, filtered and then successively rinsed with water and ethyl acetate. The collected solid was dried in an vacuum oven (70° C.) overnight to provide 2-methylsulfanyl-7-oxo-7,8-dihydro-pyrido[2,3-d]pyrimidine-6-carboxylic acid [2-chloro-5-(3-chloro-benzylcarbamoyl)-phenyl]amide. (Yield 5.5 g, 87%).

HR-MS (ES⁺) m/z Calculated for C₂₃H₁₈Cl₂N₅O₃S ([M+H]⁺): 514.0502. Found: 514.0503.

### Example 75

### 2-(4-Methyl-piperazin-1-yl)-7-oxo-7,8-dihydro-pyrido[2,3-d]pyrimidine-6-carboxylic acid [2-chloro-5-(3-chloro-benzylcarbamoyl)-phenyl]amide

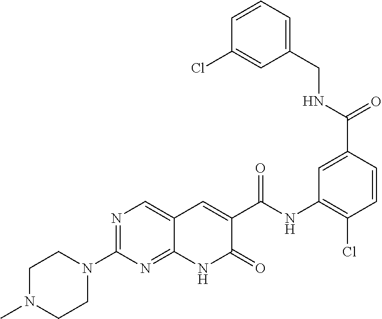

### Step A

### 2-Methanesulfonyl-7-oxo-7,8-dihydro-pyrido[2,3-d]pyrimidine-6-carboxylic acid [2-chloro-5-(3-chloro-benzylcarbamoyl)-phenyl]-amide

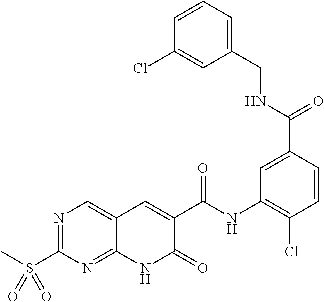

To a 1 L round bottom flask with stir bar was added 2-methylsulfanyl-7-oxo-7,8-dihydro-pyrido[2,3-d]pyrimidine-6-carboxylic acid [2-chloro-5-(3-chloro-benzylcarbamoyl)-phenyl]-amide (4.6 g, 16.7 mmol) (from Example 74 supra), THF-methanol (1:1, 150 mL), water (150 mL) and then oxone (22.0 g, 35.7 mmol). The reaction was allowed to stir at room temperature until the oxidation was complete (˜24 h). The solid was filtered and then rinsed successively with water and diethyl ether (3×), dried in a vacuum oven (80° C.) to provide 2-methanesulfonyl-7-oxo-7,8-dihydro-pyrido[2,3-d]pyrimidine-6-carboxylic acid [2-chloro-5-(3-chloro-benzylcarbamoyl)-phenyl]-amide as a yellow solid. (Yield 6.45 g, 70%).

### Step B

### 2-(4-Methyl-piperazin-1-yl-7-oxo-7,8-dihydro-pyrido[2,3-d]pyrimidine-6-carboxylic acid [2-chloro-5-(3-chloro-benzylcarbamoyl)-phenyl]-amide

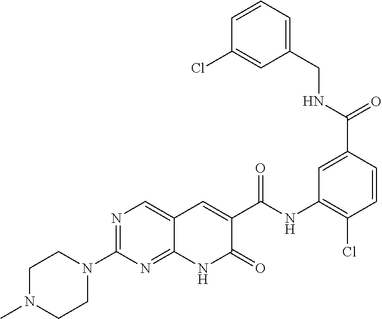

To a disposable tube equipped with a stir bar and Teflon-lined screw cap was added 2-methane-sulfonyl-7-oxo-7,8-dihydro-pyrido[2,3-d]pyrimidine-6-carboxylic acid [2-chloro-5-(3-chloro-benzylcarbamoyl)-phenyl]-amide (0.050 g, 0.093 mmol), N-methylpiperazine (0.052 mL, 0.47 mmol) and DMF (0.100 mL). The reaction was heated at 80° C. in a pre-heated oil bath for 3 hours, after which it was cooled to room temperature. The reaction was diluted with water and the precipitated solid was filtered, rinsed with water and ethyl acetate to provide 2-(4-methyl-piperazin-1-yl)-7-oxo-7,8-dihydro-pyrido[2,3-d]pyrimidine-6-carboxylic acid [2-chloro-5-(3-chloro-benzylcarbamoyl)-phenyl]-amide. (Yield 0.016 g, 30%).

HR-MS (ES⁺) m/z Calculated for C₂₇H₂₆Cl₂N₇O₃ ([M+H]⁺): 566.1469. Found: 566.1472.

### Example 76

### 2-Methoxy-7-oxo-7,8-dihydro-pyrido[2,3-d]pyrimidine-6-carboxylic acid [5-(3-amino-1-phenyl-propylcarbamoyl)-2-chloro-phenyl]-amide

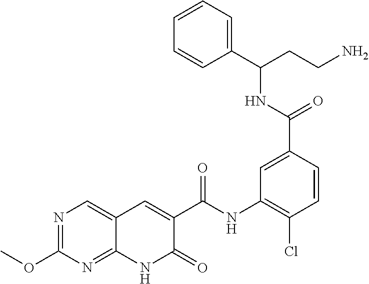

To a solution of (3-{4-chloro-3-[(2-methoxy-7-oxo-7,8-dihydro-pyrido[2,3-d]pyrimidine-6-carbonyl)-amino]-benzoylamino}-3-phenyl-propyl)-carbamic acid tert-butyl ester (0.060 g, 0.1 mmol) (from Example 73 supra) in dichloromethane (2 mL) was added a mixture of 2,2,2-trifluoroacetic acid (2.0 mL) and dichloromethane (2.0 mL). The mixture was stirred at room temperature for 3 hours. The mixture was concentrated and then partitioned between dichloromethane and saturated sodium carbonate solution. The precipitate was collected by filtration and washed with water and dried in vacuum oven to give 2-methoxy-7-oxo-7,8-dihydro-pyrido[2,3-d]pyrimidine-6-carboxylic acid [5-(3-amino-1-phenyl-propylcarbamoyl)-2-chloro-phenyl]-amide. (Yield 0.020 g, 40%).

HR-MS (ES⁺) m/z Calculated for C₂₅H₂₄ClN₆O₄ ([M+H]⁺): 507.1542. Found: 507.1546.

### Example 77

### [3-{4-Chloro-3-[(2-methoxy-7-oxo-7,8-dihydro-pyrido[2,3-d]pyrimidine-6-carbonyl)-amino]-benzoylamino}-3-(3-chloro-phenyl)-propyl]-carbamic acid tert-butyl ester

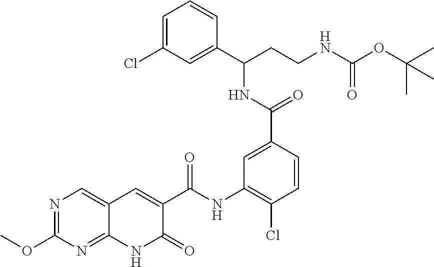

Triethylamine (0.042 mL, 0.30 mmol) was added to a mixture of 4-chloro-3-[(2-methoxy-7-oxo-7,8-dihydro-pyrido[2,3-d]pyrimidine-6-carbonyl)-amino]-benzoic acid (0.1 g, 0.27 mmol) (from Example 22 supra) and HATU (0.11 g, 0.3 mmol) (Aldrich) in DMF (4.0 mL). The mixture was stirred at room temperature for 30 minutes. [3-Amino-3-(3-chloro-phenyl)-propyl]-carbamic acid tert-butyl ester (0.09 g, 0.32 mmol) (from Example 9 supra) was added. The resultant mixture was stirred for 18 hours and then partitioned between ethyl acetate and water. The aqueous phase was extracted with ethyl acetate (3×). The combined organic phase was washed with water and brine, dried (magnesium sulfate) and concentrated. The residue was recrystallized fromethyl acetate and hexanes to give [3-{4-chloro-3-[(2-methoxy-7-oxo-7,8-dihydro-pyrido[2,3-d]pyrimidine-6-carbonyl)-amino]-benzoylamino}-3-(3-chloro-phenyl)-propyl]-carbamic acid tert-butyl ester. (Yield 0.11 g, 64.7%).

HR-MS (ES⁺) m/z Calculated for C₃₀H₃₁Cl₂N₆O₆ ([M+H]⁺): 663.1496. Found: 663.1497.

### Example 78

### 2-Methoxy-7-oxo-7,8-dihydro-pyrido[2,3-d]pyrimidine-6-carboxylic acid (2-chloro-5-methylcarbamoyl-phenyl)-amide

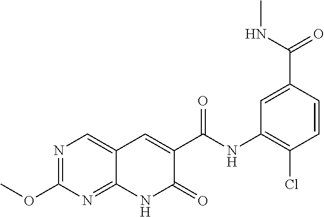

Triethylamine (0.021 mL, 0.15 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.056 g, 0.15 mmol) (Aldrich) in DMF (2.0 mL). The resulting mixture was stirred at room temperature for 30 minutes. 2M Methylamine in THF solution (0.08 mL, 0.16 mmol) was added. The mixture was stirred for 18 hours and then partitioned between ethyl acetate and water. The aqueous phase was extracted with ethyl acetate (3×). The combined organic phase was washed with water and brine, dried (magnesium sulfate) and concentrated. The residue was recrystallized fromethyl acetate and hexanes to give 2-methoxy-7-oxo-7,8-dihydro-pyrido[2,3-d]pyrimidine-6-carboxylic acid (2-chloro-5-methylcarbamoyl-phenyl)-amide. (Yield 0.019 g, 36.5%).

HR-MS (ES⁺) m/z Calculated for C₁₇H₁₅ClN₅O₄ ([M+H]⁺): 410.0626. Found: 410.0629.

### Example 79

### 2-Methoxy-7-oxo-7,8-dihydro-pyrido[2,3-d]pyrimidine-6-carboxylic acid (2-chloro-5-isobutylcarbamoyl-phenyl)-amide

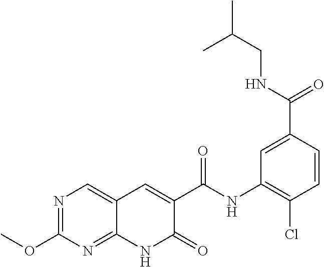

Triethylamine (0.021 mL, 0.15 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.056 g, 0.15 mmol) (Aldrich) in DMF (2.0 mL). The resultant mixture was stirred at room temperature for 30 minutes. Isobutylamine (0.016 mL, 0.16 mmol) was added. The mixture was stirred for 18 hours and then partitioned between ethyl acetate and water. The aqueous phase was extracted with ethyl acetate (3×). The combined organic phase was washed with water and brine, dried (magnesium sulfate) and concentrated. The residue was collected by filtration and washed with ethyl acetate and water and dried in vacuum oven to give 2-methoxy-7-oxo-7,8-dihydro-pyrido[2,3-d]pyrimidine-6-carboxylic acid (2-chloro-5-isobutylcarbamoyl-phenyl)-amide. (Yield 0.038 g, 66.7%).

HR-MS (ES⁺) m/z Calculated for C₂₀H₂₁ClN₅O₄ ([M+H]⁺): 430.1277. Found: 430.1277.

### Example 80

### 2-(3-Morpholin-4-yl-propoxy)-7-oxo-7,8-dihydro-pyrido[2,3-d]pyrimidine-6-carboxylic acid [2-chloro-5-(3-chloro-benzylcarbamoyl)-phenyl]-amide

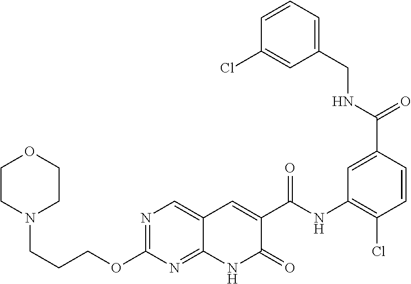

To a 25 mL round bottom flask equipped with a stir bar was added 3-morpholin-4-yl-propan-1-ol (0.094 g, 0.65 mmol) and DMF (5 mL) and the solution was cooled to 0° C. in an ice-water bath after which sodium hydride (0.026 g, 0.65 mmol, 60% oil dispersion) was added. The solution was allowed to stir at 0° C. for 20 minutes after which 2-methanesulfonyl-7-oxo-7,8-dihydro-pyrido[2,3-d]pyrimidine-6-carboxylic acid [2-chloro-5-(3-chloro-benzylcarbamoyl)-phenyl]-amide (0.050 g, 0.093 mmol) (from Example 75 supra) was added all at once. The reaction was allowed to warm to room temperature and stir for 16 hours. The reaction was filtered and then purified by reverse-phase HPLC (Gilson, C-18 Polaris column; eluting with 30-100% acetonitrile/water with 0.1% TFA) to provide, after basification to remove the TFA, 2-(3-morpholin-4-yl-propoxy)-7-oxo-7,8-dihydro-pyrido[2,3-d]pyrimidine-6-carboxylic acid [2-chloro-5-(3-chloro-benzylcarbamoyl)-phenyl]-amide. (Yield 0.018 g, 31%).

HR-MS (ES⁺) m/z Calculated for C₂₉H₂₉Cl₂N₆O₅ ([M+H]⁺): 611.1571. Found: 611.1570.

### Example 81

### 2-(2-Morpholin-4-yl-ethoxy)-7-oxo-7,8-dihydro-pyrido[2,3-d]pyrimidine-6-carboxylic acid [2-chloro-5-(3-chloro-benzylcarbamoyl)-phenyl]amide

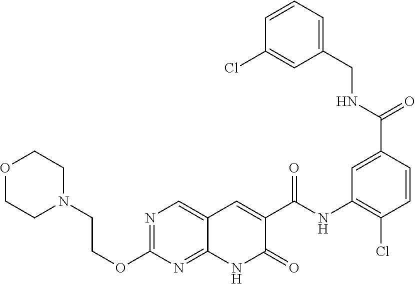

To a 25 mL round bottom flask equipped with a stir bar was added 2-morpholin-4-yl-ethanol (0.079 mL, 0.65 mmol) and DMF (5 mL) and the solution was cooled to 0° C. in an ice-water bath after which sodium hydride (0.026 g, 0.65 mmol, 60% oil dispersion) was added. The solution was allowed to stir at 0° C. for 20 minutes after which 2-methanesulfonyl-7-oxo-7,8-dihydro-pyrido[2,3-d]pyrimidine-6-carboxylic acid [2-chloro-5-(3-chloro-benzylcarbamoyl)-phenyl]-amide (0.050 g, 0.093 mmol) (from Example 75 supra) was added all at once. The reaction was allowed to warm to room temperature and stirred for 16 hours. The reaction was filtered and then purified by reverse-phase HPLC (Gilson, C-18 Polaris column; eluting with 30-100% acetonitrile/water with 0.1% TFA) to provide, after basification to remove the TFA, 2-(2-morpholin-4-yl-ethoxy)-7-oxo-7,8-dihydro-pyrido[2,3-d]pyrimidine-6-carboxylic acid [2-chloro-5-(3-chloro-benzylcarbamoyl)-phenyl]amide. (Yield 0.015 g, 27%).

HR-MS (ES⁺) m/z Calculated for C₂₈H₂₇Cl₂N₆O₅ ([M+H]⁺): 597.1415. Found: 597.1416.

### Example 82

### 4-Chloro-3-[(2-methoxy-7-oxo-7,8-dihydro-pyrido[2,3-d]pyrimidine-6-carbonyl)-amino]-benzoic acid ethyl ester

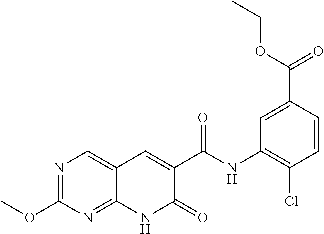

### Step A

### 3-Amino-4-chloro-benzoic acid ethyl ester

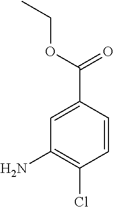

Zinc powder (5.69 g, 87.1 mmol) was added to a solution of ethyl-4-chloro-3-nitrobenzoate (1.0 g, 4.36 mmol) (Alfa Aesar) in acetonitrile (20.0 mL), ethanol (20.0 mL) and 1N aqueous hydrochloric acid (12 mL). The mixture was stirred at room temperature for 18 hours. Solid was filtered and the filterate was concentrated. The residue was partitioned between ethyl acetate and saturated sodium carbonate solution. The precipitate was collected by filtration and washed with water and ethyl acetate and dried in vacuum oven to give crude 3-amino-4-chloro-benzoic acid ethyl ester. (Yield 0.087 g, 100%).

### Step B

### 4-Chloro-3-[(2-methoxy-7-oxo-7,8-dihydro-pyrido[2,3-d]pyrimidine-6-carbonyl)-amino]-benzoic acid ethyl ester

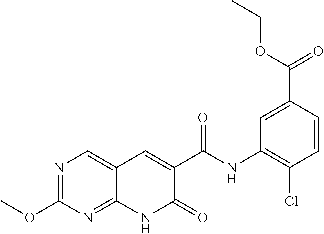

Triethylamine (0.036 mL, 0.26 mmol) was added to a mixture of 2-methoxy-7-oxo-7,8-dihydro-pyrido[2,3-d]pyrimidine-6-carboxylic acid (0.05 g, 0.23 mmol) (from Example 18 supra) and HATU (0.098 g, 0.26 mmol) in DMF (2.0 mL). The resultant mixture was stirred at room temperature for 30 minutes. 3-Amino-4-chloro-benzoic acid ethyl ester (0.06 g, 0.28 mmol) was added. The mixture was stirred for 18 hours and then partitioned between ethyl acetate and water. The aqueous phase was extracted with ethyl acetate (3×). The combined organic phase was washed with water and brine, dried (magnesium sulfate) and concentrated. The residue was re-crystallized fromethyl acetate and DMF to give 4-chloro-3-[(2-methoxy-7-oxo-7,8-dihydro-pyrido[2,3-d]pyrimidine-6-carbonyl)-amino]-benzoic acid ethyl ester. (Yield 0.026 g, 28.1%).

HR-MS (ES⁺) m/z Calculated for C₁₈H₁₆ClN₄O₅ ([M+H]⁺): 403.0804. Found: 403.0805.

### Example 83

### [2-{4-Chloro-3-[(2-methoxy-7-oxo-7,8-dihydro-pyrido[2,3-d]pyrimidine-6-carbonyl)-amino]-benzoylamino}-2-(3-chloro-phenyl)-ethyl]-carbamic acid tert-butyl ester

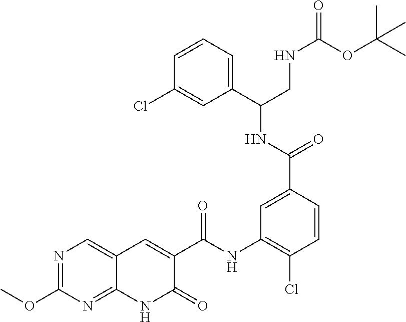

Triethylamine (0.042 mL, 0.30 mmol) was added to a mixture of 4-chloro-3-[(2-methoxy-7-oxo-7,8-dihydro-pyrido[2,3-d]pyrimidine-6-carbonyl)-amino]-benzoic acid (0.10 g, 0.27 mmol) (from Example 22 supra) and HATU (0.11 g, 0.30 mmol) (Aldrich) in DMF (4.0 mL). The mixture was stirred at room temperature for 30 minutes. [2-Amino-2-(3-chloro-phenyl)-ethyl]-carbamic acid tert-butyl ester (0.087 mL, 0.32 mmol) (from Example 11 supra) was added. The mixture was stirred for 18 hours and then partitioned between ethyl acetate and water. The precipitate was collected by filtration and washed with ethyl acetate and water and dried in vacuum oven to give [2-{4-chloro-3-[(2-methoxy-7-oxo-7,8-dihydro-pyrido[2,3-d]pyrimidine-6-carbonyl)-amino]-benzoylamino}-2-(3-chloro-phenyl)-ethyl]-carbamic acid tert-butyl ester. (Yield 0.125 g, 74.4%).

HR-MS (ES⁺) m/z Calculated for C₂₉H₂₉Cl₂N₆O₆ ([M+H]⁺): 649.1339. Found: 649.1339.

### Example 84

### 2-Methoxy-7-oxo-7,8-dihydro-pyrido[2,3-d]pyrimidine-6-carboxylic acid {5-[3-amino-1-(3-chloro-phenyl)-propylcarbamoyl]-2-chloro-phenyl}-amide

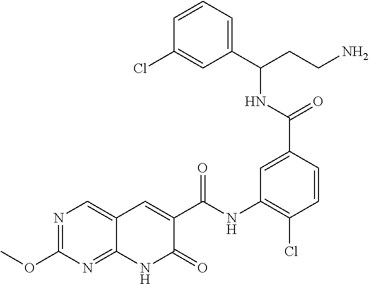

To a solution of [3-{4-chloro-3-[(2-methoxy-7-oxo-7,8-dihydro-pyrido[2,3-d]pyrimidine-6-carbonyl)-amino]-benzoylamino}-3-(3-chloro-phenyl)-propyl]-carbamic acid tert-butyl ester (0.060 g, 0.09 mmol) (from Example 77 supra) in dichloromethane (2 mL) was added a mixture of 2,2,2-trifluoroacetic acid (2.0 mL) and dichloromethane (2.0 mL). The mixture was stirred at room temperature for 4 hours. The mixture was concentrated and then partitioned between dichloromethane and saturated sodium carbonate solution. The precipitate was collected by filtration and washed with ethyl acetate and water and dried in vacuum oven to give 2-methoxy-7-oxo-7,8-dihydro-pyrido[2,3-d]pyrimidine-6-carboxylic acid {5-[3-amino-1-(3-chloro-phenyl)-propylcarbamoyl]-2-chloro-phenyl}-amide. (Yield 0.042 g, 83.0%).

HR-MS (ES⁺) m/z Calculated for C₂₅H₂₃Cl₂N₆O₄ ([M+H]⁺): 541.1153. Found: 541.1155.

### Example 85

### 2-Methoxy-7-oxo-7,8-dihydro-pyrido[2,3-d]pyrimidine-6-carboxylic acid {5-[2-amino-1-(3-chloro-phenyl)-ethylcarbamoyl]-2-chloro-phenyl}-amide

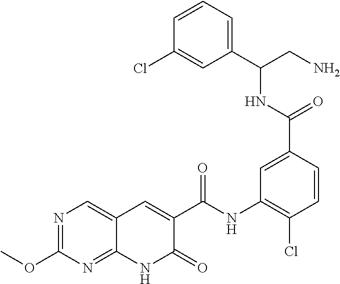

To a solution of [2-{4-chloro-3-[(2-methoxy-7-oxo-7,8-dihydro-pyrido[2,3-d]pyrimidine-6-carbonyl)-amino]-benzoylamino}-2-(3-chloro-phenyl)-ethyl]carbamic acid tert-butyl ester (0.083 g, 0.09 mmol) (from Example 83 supra) in dichloromethane (4 mL) was added a mixture of 2,2,2-trifluoroacetic acid (2.0 mL) and dichloromethane (2.0 mL). The mixture was stirred at room temperature for 4 hours. The mixture was concentrated and then partitioned between dichloromethane and saturated sodium carbonate solution. The precipitate was collected by filtration and washed with ethyl acetate and water and dried in vacuum oven to give 2-methoxy-7-oxo-7,8-dihydro-pyrido[2,3-d]pyrimidine-6-carboxylic acid [5-(3-amino-propionylamino)-2-chloro-phenyl]-amide. (Yield 0.058 g, 83.2%).

HR-MS (ES⁺) m/z Calculated for C₂₄H₂₁Cl₂N₆O₄ ([M+H]⁺): 527.0994. Found: 527.0996.

### Example 86

### 2-Morpholin-4-yl-7-oxo-7,8-dihydro-pyrido[2,3-d]pyrimidine-6-carboxylic acid [2-chloro-5-(3-chloro-benzylcarbamoyl)-phenyl]-amide

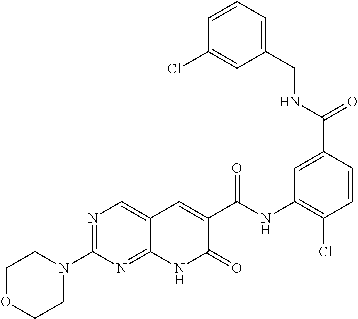

To a disposable tube equipped with a stir bar and Teflon-lined screw cap was added 2-methane-sulfonyl-7-oxo-7,8-dihydro-pyrido[2,3-d]pyrimidine-6-carboxylic acid [2-chloro-5-(3-chloro-benzylcarbamoyl)-phenyl]-amide d (0.050 g, 0.093 mmol) (from Example 75 supra), morpholine (0.200 mL, 2.3 mmol) and DMF (0.100 mL). The reaction was heated at 80° C. in a pre-heated oil bath for 1 hour, after which it was cooled to room temperature. The reaction was diluted with water and the precipitated solid was filtered, rinsed with water and ethyl acetate to provide 2-morpholin-4-yl-7-oxo-7,8-dihydro-pyrido[2,3-d]pyrimidine-6-carboxylic acid [2-chloro-5-(3-chloro-benzylcarbamoyl)-phenyl]-amide. (Yield 0.023 g, 44%).

HR-MS (ES⁺) m/z Calculated for C₂₆H₂₃Cl₂N₆O₄ ([M+H]⁺): 553.1153. Found: 553.1154.

### Example 87

### 2-[2-(4-Methyl-piperazin-1-yl)-ethoxy]-7-oxo-7,8-dihydro-pyrido[2,3-d]pyrimidine-6-carboxylic acid [2-chloro-5-(3-chloro-benzylcarbamoyl)-phenyl]amide

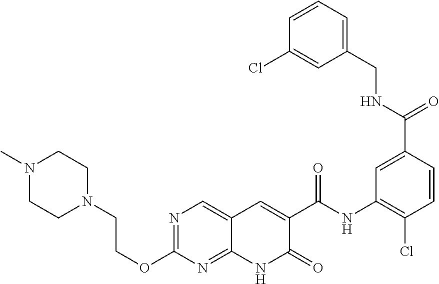

To a 25 mL round bottom flask equipped with a stir bar was added 2-(4-methyl-piperazin-1-yl)-ethanol (0.079 mL, 0.65 mmol) and DMF (5 mL) and the solution was cooled to 0° C. in an ice-water bath after which sodium hydride (0.026 g, 0.65 mmol, 60% oil dispersion) was added. The solution was allowed to stir at 0° C. for 20 minutes after which 2-methanesulfonyl-7-oxo-7,8-dihydro-pyrido[2,3-d]pyrimidine-6-carboxylic acid [2-chloro-5-(3-chloro-benzylcarbamoyl)-phenyl]-amide (0.050 g, 0.093 mmol) (from Example 75 supra) was added all at once. The reaction was allowed to warm to room temperature and stir for 16 hours. The reaction was filtered and then purified by reverse-phase HPLC (Gilson, C-18 Polaris column; eluting with 30-100% acetonitrile/water with 0.1% TFA) to provide, after basification to remove the TFA, 2-[2-(4-methyl-piperazin-1-yl)-ethoxy]-7-oxo-7,8-dihydro-pyrido[2,3-d]pyrimidine-6-carboxylic acid [2-chloro-5-(3-chloro-benzylcarbamoyl)-phenyl]amide. (Yield 0.010 g, 18%). LR-MS: [M+H]⁺: 610.

### Example 88

### 2-Methoxy-7-oxo-7,8-dihydro-pyrido[2,3-d]pyrimidine-6-carboxylic acid (5-carbamoyl-2-chloro-phenyl)-amide

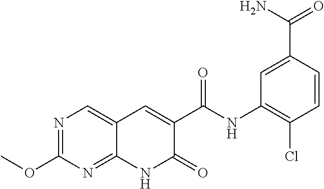

Triethylamine (0.021 mL, 0.15 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.06 g, 0.15 mmol) (Aldrich) in DMF (2.0 mL). The resultant mixture was stirred at room temperature for 30 minutes Ammonium chloride (9.0 mg, 0.16 mmol) (Fisher) was added. The mixture was stirred for 18 hours and then partitioned between ethyl acetate and water. The aqueous phase was extracted with ethyl acetate (3×). The combined organic phase was washed with water and brine, dried (magnesium sulfate), filtered, and concentrated. The residue was collected by filtration and washed with ethyl acetate and water and dried in vacuum oven to give 2-methoxy-7-oxo-7,8-dihydro-pyrido[2,3-d]pyrimidine-6-carboxylic acid (5-carbamoyl-2-chloro-phenyl)-amide. (Yield 0.030 g, 60%).

HR-MS (ES⁺) m/z Calculated for C₁₆H₁₃ClN₅O₄ ([M+H]⁺): 374.0651. Found: 374.0651.

### Example 89

### 7-Oxo-2-(2-pyrrolidin-1-yl-ethylamino)-7,8-dihydro-pyrido[2,3-d]pyrimidine-6-carboxylic acid [2-chloro-5-(3-chloro-benzylcarbamoyl)-phenyl]-amide

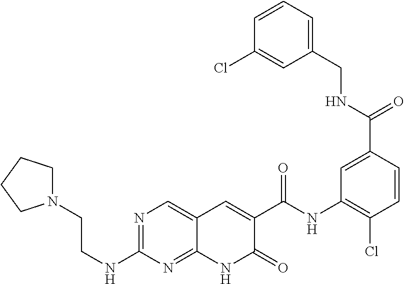

To a disposable tube equipped with a stir bar and Teflon-lined screw cap was added 2-methane-sulfonyl-7-oxo-7,8-dihydro-pyrido[2,3-d]pyrimidine-6-carboxylic acid [2-chloro-5-(3-chloro-benzylcarbamoyl)-phenyl]-amide (0.100 g, 0.18 mmol) (from Example 75 supra), 2-pyrrolidin-1-yl-ethylamine (104 μg, 0.92 mmol) and DMF (0.200 mL). The reaction was heated at 80° C. in a pre-heated oil bath for 3 hours, after which it was cooled to room temperature. The reaction was diluted with water and the precipitated solid was filtered, rinsed with 0.5 M aqueous sodium hydroxide, water and diethyl ether to provide 7-oxo-2-(2-pyrrolidin-1-yl-ethylamino)-7,8-dihydro-pyrido[2,3-d]pyrimidine-6-carboxylic acid [2-chloro-5-(3-chloro-benzylcarbamoyl)-phenyl]-amide. (Yield 29 mg, 28%).

LR-MS: [M+H]⁺: 580.

### Example 90

### 2-(3-Morpholin-4-yl-propylamino)-7-oxo-7,8-dihydro-pyrido[2,3-d]pyrimidine-6-carboxylic acid [2-chloro-5-(3-chloro-benzylcarbamoyl)-phenyl]-amide

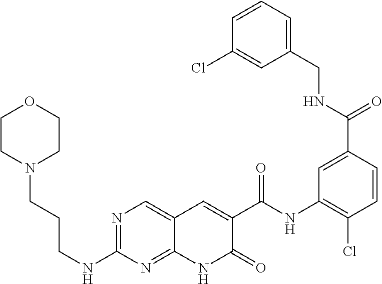

To a disposable tube equipped with a stir bar and Teflon-lined screw cap were added 2-methane-sulfonyl-7-oxo-7,8-dihydro-pyrido[2,3-d]pyrimidine-6-carboxylic acid [2-chloro-5-(3-chloro-benzylcarbamoyl)-phenyl]-amide (0.100 g, 0.18 mmol) (from Example 75 supra), 3-morpholin-4-yl-propylamine (0.132 mg, 0.92 mmol) and DMF (0.200 mL). The reaction was heated at 80° C. in a pre-heated oil bath for 3 hours, after which it was cooled to room temperature. The reaction was diluted with water and the precipitated solid was filtered, rinsed with 0.5 M aqueous sodium hydroxide, water and diethyl ether to provide 2-(3-morpholin-4-yl-propylamino)-7-oxo-7,8-dihydro-pyrido[2,3-d]pyrimidine-6-carboxylic acid [2-chloro-5-(3-chloro-benzylcarbamoyl)-phenyl]-amide. (Yield 15 mg, 14%).

HR-MS (ES⁺) m/z Calculated for C₂₉H₃₀Cl₂N₇O₄ ([M+H]⁺): 610.1731. Found: 610.1733.

### Example 91

### 4-{6-[2-Chloro-5-(3-chloro-benzylcarbamoyl)-phenylcarbamoyl]-7-oxo-7,8-dihydro-pyrido[2,3-d]pyrimidin-2-ylamino}-piperidine-1-carboxylic acid tert-butyl ester

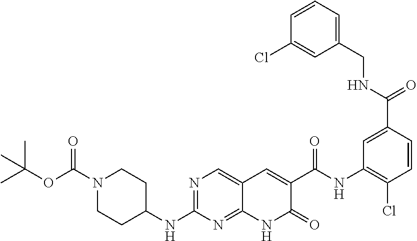

To a disposable tube equipped with a stir bar and Teflon-lined screw cap was added 2-methane-sulfonyl-7-oxo-7,8-dihydro-pyrido[2,3-d]pyrimidine-6-carboxylic acid [2-chloro-5-(3-chloro-benzylcarbamoyl)-phenyl]-amide (0.05 g, 0.09 mmol) (from Example 75 supra), 4-amino-piperidine-1-carboxylic acid tert-butyl ester (65 mg, 0.4 mmol) and DMF (0.200 mL). The reaction was heated at 80° C. in a pre-heated oil bath for 3 hours, after which it was cooled to room temperature. The reaction was diluted with water and the precipitated solid was filtered, rinsed with 0.5 M aqueous sodium hydroxide, water and diethyl ether to provide after drying 4-{6-[2-chloro-5-(3-chloro-benzylcarbamoyl)-phenylcarbamoyl]-7-oxo-7,8-dihydro-pyrido[2,3-d]pyrimidin-2-ylamino}-piperidine-1-carboxylic acid tert-butyl ester. (Yield 12 mg, 20%).

HR-MS (ES⁺) m/z Calculated for C₃₂H₃₄Cl₂N₇O₅ ([M+H]⁺): 666.1993. Found: 666.1991.

### Example 92

### 2-(4-Hydroxy-piperidin-1-yl)-7-oxo-7,8-dihydro-pyrido[2,3-d]pyrimidine-6-carboxylic acid [2-chloro-5-(3-chloro-benzylcarbamoyl)-phenyl]amide

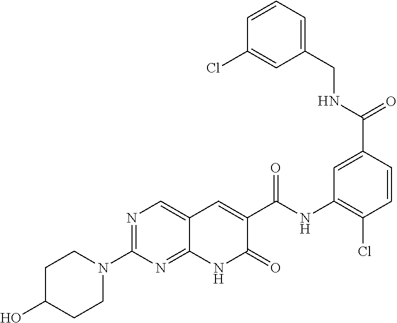

To a disposable tube equipped with a stir bar and Teflon-lined screw cap was added 2-methane-sulfonyl-7-oxo-7,8-dihydro-pyrido[2,3-d]pyrimidine-6-carboxylic acid [2-chloro-5-(3-chloro-benzylcarbamoyl)-phenyl]-amide (0.100 g, 0.18 mmol) (from Example 75 supra), piperidin-4-ol (92 mg, 0.92 mmol) and DMF (0.500 mL). The reaction was heated at 80° C. in a pre-heated oil bath for 3 hours, after which it was cooled to room temperature. The reaction was diluted with water and the precipitated solid was filtered, rinsed with 0.5 M aqueous sodium hydroxide, water and diethyl ether to provide after drying 2-(4-hydroxy-piperidin-1-yl)-7-oxo-7,8-dihydro-pyrido[2,3-d]pyrimidine-6-carboxylic acid [2-chloro-5-(3-chloro-benzylcarbamoyl)-phenyl]-amide. (Yield 44 mg, 43%).

HR-MS (ES⁺) m/z Calculated for C₂₇H₂₅Cl₂N₆O₄ ([M+H]⁺): 567.1309. Found: 567.1308.

### Example 93

### (1-{6-[2-Chloro-5-(3-chloro-benzylcarbamoyl)-phenylcarbamoyl]-7-oxo-7,8-dihydro-pyrido[2,3-d]pyrimidin-2-yl}-piperidin-4-yl)-carbamic acid tert-butyl ester

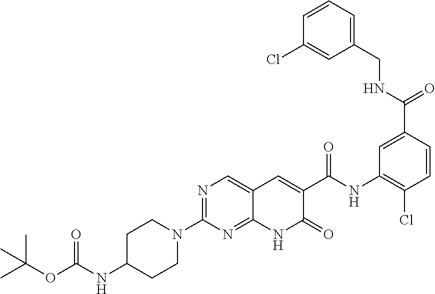

To a disposable tube equipped with a stir bar and Teflon-lined screw cap was added 2-methane-sulfonyl-7-oxo-7,8-dihydro-pyrido[2,3-d]pyrimidine-6-carboxylic acid [2-chloro-5-(3-chloro-benzylcarbamoyl)-phenyl]-amide (0.100 g, 0.18 mmol) (from Example 75 supra), piperidin-4-yl-carbamic acid tert-butyl ester (184 mg, 0.92 mmol) and DMF (0.200 mL). The reaction was heated at 80° C. in a pre-heated oil bath for 3 hours, after which it was cooled to room temperature. The reaction was diluted with water and the precipitated solid was filtered, rinsed with 0.5 M aqueous sodium hydroxide, water and diethyl ether to provide after drying (1-{6-[2-Chloro-5-(3-chloro-benzylcarbamoyl)-phenylcarbamoyl]-7-oxo-7,8-dihydro-pyrido[2,3-d]pyrimidin-2-yl}-piperidin-4-yl)-carbamic acid tert-butyl ester. (Yield 92 mg, 76%).

HR-MS (ES⁺) m/z Calculated for C₃₂H₃₄Cl₂N₇O₅ ([M+H]⁺): 666.1993. Found: 666.1995.

### Example 94

### 2-(4-Hydroxy-cyclohexylamino)-7-oxo-7,8-dihydro-pyrido[2,3-d]pyrimidine-6-carboxylic acid [2-chloro-5-(3-chloro-benzylcarbamoyl)-phenyl]-amide

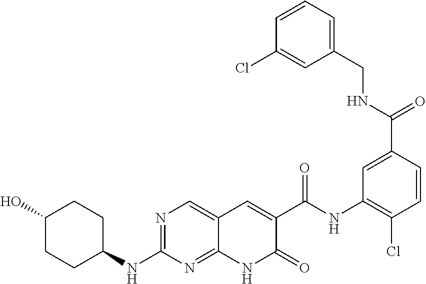

To a disposable tube equipped with a stir bar and Teflon-lined screw cap was added 2-methane-sulfonyl-7-oxo-7,8-dihydro-pyrido[2,3-d]pyrimidine-6-carboxylic acid [2-chloro-5-(3-chloro-benzylcarbamoyl)-phenyl]-amide (0.100 g, 0.18 mmol) (from Example 75 supra), trans-4-amino-cyclohexanol (95 mg, 0.92 mmol) and DMF (0.200 mL). The reaction was heated at 80° C. in a pre-heated oil bath for 3 hours, after which it was cooled to room temperature. The reaction was diluted with water and the precipitated solid was filtered, rinsed with 0.5 M aqueous sodium hydroxide, water and diethyl ether to provide after drying 2-(4-hydroxy-cyclohexylamino)-7-oxo-7,8-dihydro-pyrido[2,3-d]pyrimidine-6-carboxylic acid [2-chloro-5-(3-chloro-benzylcarbamoyl)-phenyl]-amide. (Yield 38 mg, 36%).

HR-MS (ES⁺) m/z Calculated for C₂₈H₂₇Cl₂N₆O₄ ([M+H]⁺): 581.1466. Found: 581.1466.

### Example 95

### 2-(4-Hydroxy-cyclohexylamino)-7-oxo-7,8-dihydro-pyrido[2,3-d]pyrimidine-6-carboxylic acid [2-chloro-5-(3-chloro-benzylcarbamoyl)-phenyl]-amide

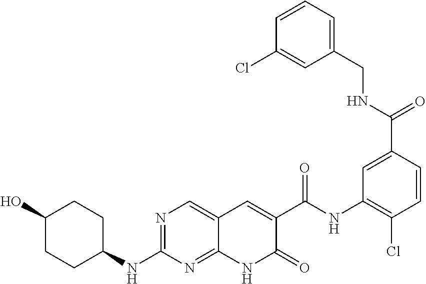

To a disposable tube equipped with a stir bar and Teflon-lined screw cap was added 2-methane-sulfonyl-7-oxo-7,8-dihydro-pyrido[2,3-d]pyrimidine-6-carboxylic acid [2-chloro-5-(3-chloro-benzylcarbamoyl)-phenyl]-amide (0.100 g, 0.18 mmol) (from Example 75 supra), cis-4-amino-cyclohexanol-HCl (139 mg, 0.92 mmol), triethylamine (0.100 mL, 0.72 mmol) and DMF (0.300 mL). The reaction was heated at 80° C. in a pre-heated oil bath for 3 hours, after which it was cooled to room temperature. The reaction was diluted with water and the precipitated solid was filtered, rinsed with 0.5 M aqueous sodium hydroxide, water and diethyl ether to provide after drying 2-(4-hydroxy-cyclohexylamino)-7-oxo-7,8-dihydro-pyrido[2,3-d]pyrimidine-6-carboxylic acid [2-chloro-5-(3-chloro-benzylcarbamoyl)-phenyl]amide. (Yield 33 mg, 32%).

HR-MS (ES⁺) m/z Calculated for C₂₈H₂₇Cl₂N₆O₄ ([M+H]⁺): 581.1466. Found: 581.1464.

### Example 96

### 2-Methoxy-7-oxo-7,8-dihydro-pyrido[2,3-d]pyrimidine-6-carboxylic acid {2-chloro-5-[1-(3-chloro-phenyl)-3-dimethylamino-propylcarbamoyl]-phenyl}-amide

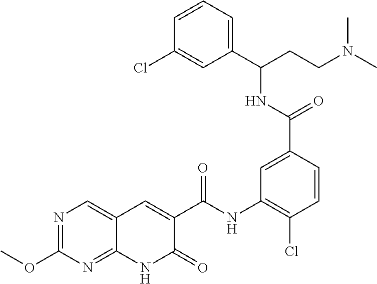

### Step A

### 1-(3-Chloro-phenyl)-3-dimethylamino-propan-1-one; hydrochloride

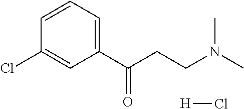

A mixture of 3-chloroacetophenone (17.9 g, 116 mmol), paraformaldehyde (6.9 g, 232 mmol), dimethylamine hydrochloride (14.1 g, 177 mmol), concentrated hydrochloric acid ((0.38 mL), and ethanol (30 mL) was heated at reflux for 5 hours. After cooling, the precipitated material was collected by filtration and dried under reduced pressure to give 1-(3-chloro-phenyl)-3-dimethylamino-propan-1-one; hydrochloride. (Yield 23.5 g, 82%).

LC-MS m/z ([M+H]⁺): 212.2.

### Step B

### 1-(3-Chloro-phenyl)-3-dimethylamino-propan-1-ol

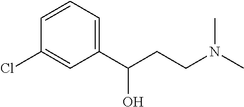

Lithium aluminum hydride (2 M in THF, 27 mL, 54 mmol) was added slowly to a solution of 1-(3-chloro-phenyl)-3-dimethylamino-propan-1-one; hydrochloride (10.5 g, 42.6 mmol) in THF (500 mL) at room temperature. The solution was stirred for 2 hours at room temperature. A saturated aqueous solution of NaHCO₃ was then added slowly and the mixture was extracted twice with ethyl acetate (300 mL each). The combined organic phase was washed with water (200 mL) and brine (200 mL). This solution was concentrated to give 1-(3-chloro-phenyl)-3-dimethylamino-propan-1-ol as a light yellow solid. (Yield 8.5 g, 94%).

LC-MS m/z ([M+H]⁺): 214.2.

### Step C

### N-[1-(3-Chloro-phenyl)-3-dimethylamino-propyl]-acetamide

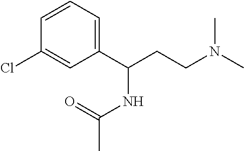

Concentrated sulfuric acid (20 mL) was added to a solution of 1-(3-chloro-phenyl)-3-dimethylamino-propan-1-ol (4.5 g, 23 mmol) in acetonitrile (9 mL) with cooling at −15° C. After stirring for 18 hours, the solution was added to water (45 mL) slowly, and the mixture was made basic to pH 14 using sodium hydroxide pellets. This mixture was then extracted with ethyl acetate (2×100 mL). The combined organic phase was washed with water (100 mL) and brine (100 mL) and concentrated to give crude N-[1-(3-chloro-phenyl)-3-dimethylamino-propyl]-acetamide. (Yield 5.8 g, 100%).

LC-MS m/z ([M+H]⁺): 255.2.

### Step D

### 1-(3-Chloro-phenyl)-3-N,N-dimethyl-propane-1,3-diamine

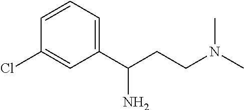

A solution of N-[1-(3-chloro-phenyl)-3-dimethylamino-propyl]-acetamide (5.8 g, 23 mmol) in 6M hydrochloric acid (50 mL) was heated at reflux for 18 hours. After cooling, the mixture was made basic to pH 14 using NaOH pellets and then extracted with ethyl acetate (2×100 mL). The combined organic phase was washed with water (100 mL) and brine (100 mL) and then concentrated to give 1-(3-chloro-phenyl)-3-N,N-dimethyl-propane-1,3-diamine. (Yield 4.6 g, 94%). LC-MS m/z ([M+H]⁺): 213.2.

### Step E

### 2-Methoxy-7-oxo-7,8-dihydro-pyrido[2,3-d]pyrimidine-6-carboxylic acid {2-chloro-5-[1-(3-chloro-phenyl)-3-dimethylamino-propylcarbamoyl]-phenyl}-amide

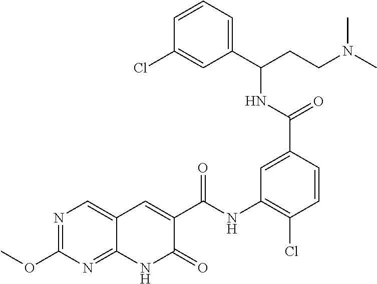

Triethylamine (0.021 mL, 0.15 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.06 g, 0.15 mmol) (Aldrich) in DMF (2.0 mL). Mixture was stirred at room temperature for 30 minutes. 1-(3-Chloro-phenyl)-3-N,N-dimethyl-propane-1,3-diamine (34 mg, 0.16 mmol) was added. Mixture was stirred for 18 hours and then partitioned between ethyl acetate and water. The aqueous phase was extracted with ethyl acetate (3×). The combined organic phase was washed with water and brine, dried (magnesium sulfate), filtered, and concentrated. The residue was collected by filtration and washed with ethyl acetate and water and dried in vacuum oven to give 2-methoxy-7-oxo-7,8-dihydro-pyrido[2,3-d]pyrimidine-6-carboxylic acid {2-chloro-5-[1-(3-chloro-phenyl)-3-dimethylamino-propylcarb-amoyl]-phenyl}-amide. (Yield 0.017 g, 13.2%).

HR-MS (ES⁺) m/z Calculated for C₂₇H₂₇Cl₂N₆O₄ ([M+H]⁺): 569.1466. Found: 569.1465.

### Example 97

### 2-(4-Amino-piperidin-1-yl)-7-oxo-7,8-dihydro-pyrido[2,3-d]pyrimidine-6-carboxylic acid [2-chloro-5-(3-chloro-benzylcarbamoyl)-phenyl]amide

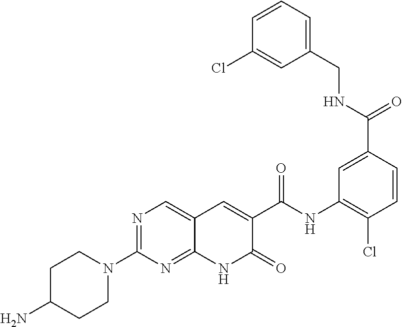

In a 10 mL round bottom flask equipped with stir bar was added (1-{6-[2-chloro-5-(3-chloro-benzylcarbamoyl)-phenylcarbamoyl]-7-oxo-7,8-dihydro-pyrido[2,3-d]pyrimidin-2-yl}-piperidin-4-yl)-carbamic acid tert-butyl ester (0.06 g, 0.09 mmol) (from Example 93 supra) and then hydrochloric acid in 1,4-dioxane (3.0 mL, 4M, 12 mmol) and the solution was allowed to stir at room temperature until completion. The reaction was concentrated to remove all solvents, diluted with 1.0N aqueous NaOH (2 mL) and the solid was then filtered, rinsed with 1.0N aqueous NaOH, water, and diethyl ether providing after drying 2-(4-Amino-piperidin-1-yl)-7-oxo-7,8-dihydro-pyrido[2,3-d]pyrimidine-6-carboxylic acid [2-chloro-5-(3-chloro-benzylcarbamoyl)-phenyl]-amide. (Yield 30 mg, 60%).

LC-MS ([M+H]⁺): 566.

### Example 98

### (2-{4-Chloro-3-[(2-methylsulfanyl-7-oxo-7,8-dihydro-pyrido[2,3-d]pyrimidine-6-carbonyl)-amino]-benzoylamino}-2-phenyl-ethyl)-carbamic acid tert-butyl ester

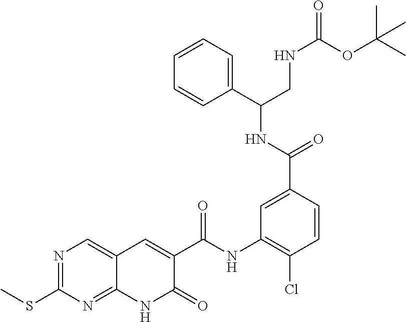

### Step A

### 4-Chloro-3-[(2-methylsulfanyl-7-oxo-7,8-dihydro-pyrido[2,3-d]pyrimidine-6-carbonyl)-amino]-benzoic acid

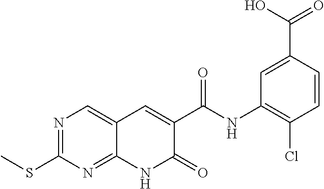

To a 250 mL round bottom flask equipped with a stir bar was added 4-chloro-3-[(2-methyl-sulfanyl-7-oxo-7,8-dihydro-pyrido[2,3-d]pyrimidine-6-carbonyl)-amino]-benzoic acid methyl ester (1.7 g, 4.2 mmol) (from Example 30 supra), lithium hydroxide (0.252 g, 10.5 mmol), methanol (16 mL), THF (8 mL) and water (12 mL). The flask was heated at 65° C. with stirring for 2 hours, after which it was concentrated to dryness under reduced pressure. The crude material was carefully acidified with aqueous hydrochloric acid (2.0 M) and the precipitated white solid was collected by filtration, rinsed with water and dried in an oven vacuum (80° C.) overnight to provide 4-chloro-3-[(2-methylsulfanyl-7-oxo-7,8-dihydro-pyrido[2,3-d]pyrimidine-6-carbonyl)-amino]-benzoic acid. (Yield 1.64 g, 105%).

### Step B

### (2-{4-Chloro-3-[(2-methylsulfanyl-7-oxo-7,8-dihydro-pyrido[2,3-d]pyrimidine-6-carbonyl)-amino]-benzoylamino}-2-phenyl-ethyl)-carbamic acid tert-butyl ester

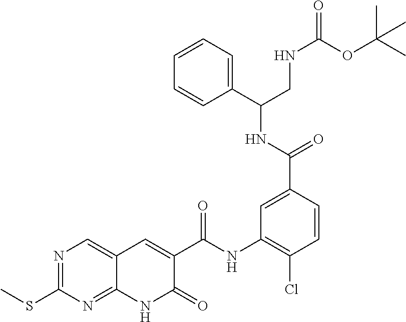

In a 25 mL round bottom flask equipped with a stir bar was added 4-chloro-3-[(2-methyl-sulfanyl-7-oxo-7,8-dihydro-pyrido[2,3-d]pyrimidine-6-carbonyl)-amino]-benzoic acid (0.751 g, 1.9 mmol), (2-amino-2-phenyl-ethyl)-carbamic acid tert-butyl ester (0.500 g, 2.11 mmol) (from Example 4 supra), DMF (10 mL) and then triethylamine (1.32 mL, 9.5 mmol). After 5 minutes of stirring at room temperature, HATU (0.36 g, 2.2 mmol) was added all at once. The reaction was allowed to stir at room temperature overnight. The reaction was diluted with water and the solid was collected by filtration, rinsed with water and ethyl acetate, dried in an oven vacuum (65° C.) overnight to provide (2-{4-chloro-3-[(2-methylsulfanyl-7-oxo-7,8-dihydro-pyrido[2,3-d]pyrimidine-6-carbonyl)-amino]-benzoylamino}-2-phenyl-ethyl)-carbamic acid tert-butyl ester. (Yield 0.874 g, 75%).

HR-MS (ES⁺) m/z Calculated for C₂₉H₂₉ClN₆O₅SNa ([M+Na]⁺): 631.1501. Found: 631.1496.

### Example 99

### 2-[2-(4-Methyl-piperazin-1-yl)-ethylamino]-7-oxo-7,8-dihydro-pyrido[2,3-d]pyrimidine-6-carboxylic acid [2-chloro-5-(3-chloro-benzylcarbamoyl)-phenyl]amide

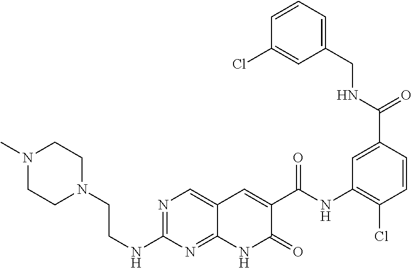

To a disposable tube equipped with a stir bar and Teflon-lined screw cap was added 2-methane-sulfonyl-7-oxo-7,8-dihydro-pyrido[2,3-d]pyrimidine-6-carboxylic acid [2-chloro-5-(3-chloro-benzylcarbamoyl)-phenyl]-amide (0.544 g, 1.0 mmol) (from Example 75 supra), 2-(4-methyl-piperazin-1-yl)-ethylamine (0.782 g, 5.0 mmol) and DMF (2.0 mL). The reaction was heated at 80° C. in a pre-heated oil bath for 2 hours, after which it was cooled to room temperature. The reaction was diluted with water and the precipitated solid was filtered, rinsed with 0.5 M aqueous sodium hydroxide, water and diethyl ether to provide after drying 2-[2-(4-methyl-piperazin-1-yl)-ethylamino]-7-oxo-7,8-dihydro-pyrido[2,3-d]pyrimidine-6-carboxylic acid [2-chloro-5-(3-chloro-benzylcarbamoyl)-phenyl]-amide. (Yield 353 mg, 58%).

HR-MS (ES⁺) m/z Calculated for C₂₉H₃₁Cl₂N₈O₃ ([M+H]⁺): 609.1891. Found: 609.1889.

### Example 100

### N-(2-chloro-5-(3-chlorobenzylcarbamoyl)phenyl)-2-(2-(4-methylpiperazin-1-yl)ethylamino)-7-oxo-7,8-dihydropyrido[2,3-d]pyrimidine-6-carboxamide hydrochloride

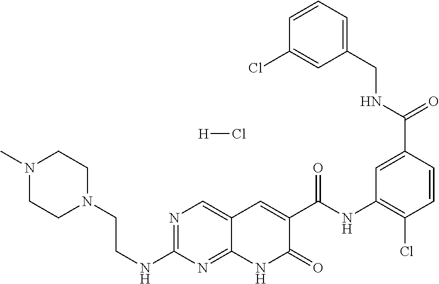

To a disposable tube with stir bar was added 2-[2-(4-methyl-piperazin-1-yl)-ethylamino]-7-oxo-7,8-dihydro-pyrido[2,3-d]pyrimidine-6-carboxylic acid [2-chloro-5-(3-chloro-benzylcarbamoyl)-phenyl]-amide (100 mg, 0.164 mmol) (from Example 99 supra), hydrochloric acid in 1,4-dioxane (2.00 mL, 8.00 mmol, 4M). The solution was allowed to stir for 5 minutes, after which the solvents were concentrated under reduced pressure and then freeze-dried from acetonitrile-water to provide N-(2-chloro-5-(3-chlorobenzylcarbamoyl)phenyl)-2-(2-(4-methylpiperazin-1-yl)ethylamino)-7-oxo-7,8-dihydropyrido[2,3-d]pyrimidine-6-carboxamide hydrochloride as solid. (Yield 95 mg, 85%).

HR-MS (ES⁺) m/z Calculated for C₂₉H₃₀Cl₂N₈O₃.2HCl ([M+H]⁺): 609.1891. Found: 609.1888.

### Example 101

### 2-[3-(4-Methyl-piperazin-1-yl)-propylamino]-7-oxo-7,8-dihydro-pyrido[2,3-d]pyrimidine-6-carboxylic acid [2-chloro-5-(3-chloro-benzylcarbamoyl)-phenyl]amide

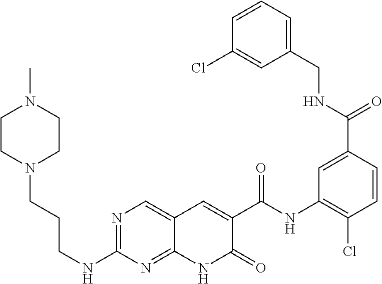

To a disposable tube equipped with a stir bar and Teflon-lined screw cap was added 2-methane-sulfonyl-7-oxo-7,8-dihydro-pyrido[2,3-d]pyrimidine-6-carboxylic acid [2-chloro-5-(3-chloro-benzylcarbamoyl)-phenyl]-amide (0.54 g, 1.0 mmol) (from Example 75 supra), 2-(4-methyl-piperazin-1-yl)-propylamine (1.0 mL, 5.0 mmol) and DMF (2.5 mL). The reaction was heated at 80° C. in a pre-heated oil bath for 2 hours, after which it was cooled to room temperature. The reaction was diluted with water and the precipitated solid was filtered, rinsed with 0.5 M aqueous sodium hydroxide, water and diethyl ether to provide after drying 2-[3-(4-methyl-piperazin-1-yl)-propylamino]-7-oxo-7,8-dihydro-pyrido[2,3-d]pyrimidine-6-carboxylic acid [2-chloro-5-(3-chloro-benzylcarbamoyl)-phenyl]-amide. (Yield 234 mg, 38%).

HR-MS (ES⁺) m/z Calculated for C₃₀H₃₃Cl₂N₈O₃ ([M+H]⁺): 623.2047. Found: 623.2048.

### Example 102

### 2-[3-(4-Methyl-piperazin-1-yl)-propylamino]-7-oxo-7,8-dihydro-pyrido[2,3-d]pyrimidine-6-carboxylic acid [2-chloro-5-(3-chloro-benzylcarbamoyl)-phenyl]-amide hydrochloride

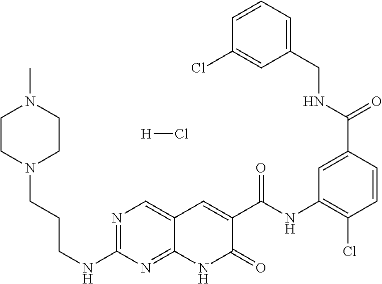

To 2-[3-(4-methyl-piperazin-1-yl)-propylamino]-7-oxo-7,8-dihydro-pyrido[2,3-d]pyrimidine-6-carboxylic acid [2-chloro-5-(3-chloro-benzylcarbamoyl)-phenyl]amide (75 mg, 120 μmol) (from Example 101 supra) was added 1.0 N hydrochloric acid (120 μmol, Eq: 1.00). The solution was concentrated. Then 4.0 N HCl in 1,4-dioxane (120 μmol, Eq: 1.00) was added, after which it was concentrated and freeze dried from acetonitrile-water to provide 2-[3-(4-methyl-piperazin-1-yl)-propylamino]-7-oxo-7,8-dihydro-pyrido[2,3-d]pyrimidine-6-carboxylic acid [2-chloro-5-(3-chloro-benzylcarbamoyl)-phenyl]amide hydrochloride as a solid. (Yield 67 mg, 76%).

HR-MS (ES⁺) m/z Calculated for C₃₀H₃₃Cl₂N₈O₃ ([M+H]⁺): 623.2047. Found: 623.2048

### Example 103

### N-(2-chloro-5-(3-chlorobenzylcarbamoyl)phenyl)-2-(2-hydroxyethylamino)-7-oxo-7,8-dihydropyrido[2,3-d]pyrimidine-6-carboxamide

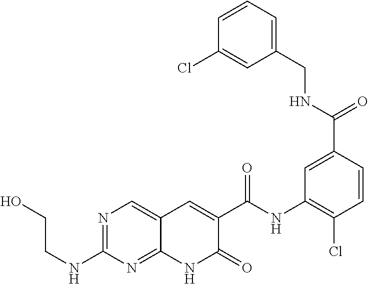

To a disposable tube with stir bar was added 2-methanesulfonyl-7-oxo-7,8-dihydro-pyrido[2,3-d]pyrimidine-6-carboxylic acid [2-chloro-5-(3-chloro-benzylcarbamoyl)-phenyl]-amide (100 mg, 183 μmol, Eq: 1.00) (from Example 75 supra) and N,N-dimethylformamide (0.5 mL). To the reaction flask was added 2-aminoethanol (506 mg, 8.28 mmol, Eq: 45.3) and the reaction was heated at 85° C. in a pre-heated oil bath for 2.5 hours. The solid was filtered and rinsed with 0.5 N aqueous NaOH, water, EtOAc and diethylether to provide N-(2-chloro-5-(3-chlorobenzylcarbamoyl)phenyl)-2-(2-hydroxyethylamino)-7-oxo-7,8-dihydropyrido[2,3-d]pyrimidine-6-carboxamide. (Yield 45 mg, 42%).

HR-MS (ES⁺) m/z Calculated for C₂₄H₂₁Cl₂N₆O₄ ([M+H]⁺): 527.0996. Found 527.0995.

### Example 104

### N-(2-chloro-5-(3-chlorobenzylcarbamoyl)phenyl)-2-(2-(1-methylpiperidin-4-yl)ethylamino)-7-oxo-7,8-dihydropyrido[2,3-d]pyrimidine-6-carboxamide

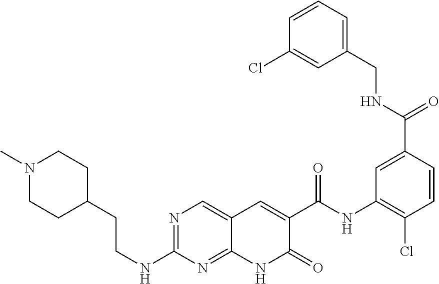

To a disposable tube with stir bar equipped with a Teflon-lined screw cap was added 2-methane-sulfonyl-7-oxo-7,8-dihydro-pyrido[2,3-d]pyrimidine-6-carboxylic acid [2-chloro-5-(3-chloro-benzylcarbamoyl)-phenyl]-amide (100 mg, 183 μmol, Eq: 1.00) (from Example 75 supra), N,N-dimethylformamide (200 μL) and 2-(1-methylpiperidin-4-yl)ethanamine (1000.0 mg, 7.03 mmol, Eq: 38.4). The reaction was then heated at 85° C. in a pre-heated oil bath for 2.3 hours. The reaction was cooled to room temperature, diluted with water (1 mL) and aq. NaOH (1N, 1 mL). After cooling, the precipitated solid was collected by filtration, rinsed with water and Et₂O to provide after drying N-(2-chloro-5-(3-chlorobenzylcarbamoyl)phenyl)-2-(2-(1-methylpiperidin-4-yl)ethylamino)-7-oxo-7,8-dihydropyrido[2,3-d]pyrimidine-6-carboxamide. (Yield 70 mg, 63%).

HR-MS (ES⁺) m/z Calculated for C₃₀H₃₂Cl₂N₇O₃ ([M+H]⁺): 608.1938. Found: 608.1940.

### Example 105

### N-(5-(2-amino-1-phenylethylcarbamoyl)-2-chlorophenyl)-2-(methylthio)-7-oxo-7,8-dihydropyrido[2,3-d]pyrimidine-6-carboxamide

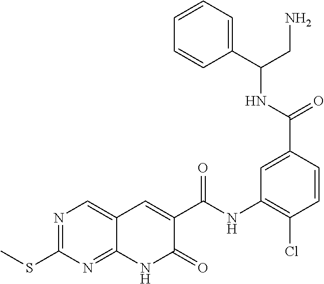

To a round bottom flask with stir bar was added tert-butyl 2-(4-chloro-3-(2-(methylthio)-7-oxo-7,8-dihydropyrido[2,3-d]pyrimidine-6-carboxamido)benzamido)-2-phenylethylcarbamate (50 mg, 82.1 μmol, Eq: 1.00) (from Example 98 supra) and 4.0 N hydrogen chloride in 1,4-dioxane (8.00 mmol, Eq: 97.5). The reaction was stirred at room temperature for 12 hours. The reaction mixture was concentrated under reduced pressure, after which 1.0 N NaOH was added (˜1 mL). The solid precipitate was collected by filtration, rinsed with water and then ethyl acetate and diethyl ether to provide after drying N-(5-(2-amino-1-phenylethylcarbamoyl)-2-chlorophenyl)-2-(methylthio)-7-oxo-7,8-dihydropyrido[2,3-d]pyrimidine-6-carboxamide. (Yield 22 mg, 53%).

HR-MS (ES⁺) m/z Calculated for C₂₄H₂₂ClN₆O₃S ([M+H]⁺): 509.1157. Found: 509.1156.

### Example 106

### tert-Butyl 3-(4-chloro-3-(7-(methylthio)-2-oxo-1,2-dihydroquinoline-3-carboxamido)benzamido)-3-phenylpropylcarbamate

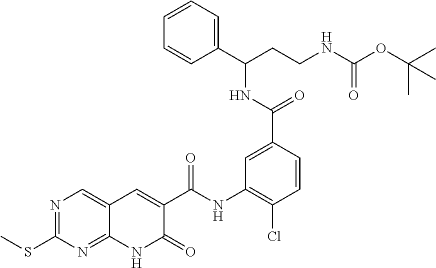

To 100 mL flask with stir bar was added 4-chloro-3-[(2-methylsulfanyl-7-oxo-7,8-dihydro-pyrido[2,3-d]pyrimidine-6-carbonyl)-amino]-benzoic acid (750 mg, 1.92 mmol, Eq: 1.00) (from Example 98 supra), tert-butyl 3-amino-3-phenylpropylcarbamate (528 mg, 2.11 mmol, Eq: 1.1) (from Example 6 supra), N,N-dimethylformamide (10 mL) and then triethylamine (971 mg, 9.6 mmol, Eq: 5). After 5 minutes of stirring at room temperature, HATU (839 mg, 2.21 mmol, Eq: 1.15) was added all at once. The reaction was allowed to stir at room temperature for 18 hours. The reaction was diluted with water and ethyl acetate. The solid was filtered off and rinsed with water and ethyl acetate. The collected solid was dried in a vacuum oven (65° C.) providing tert-butyl 3-(4-chloro-3-(7-(methylthio)-2-oxo-1,2-dihydroquinoline-3-carboxamido)-benzamido)-3-phenylpropylcarbamate. (Yield 990 mg, 83%).

HR-MS (ES⁺) m/z Calculated for C₃₀H₃₂ClN₆O₅S ([M+H]⁺): 623.1838. Found: 623.1836.

### Example 107

### 2-Methylsulfanyl-7-oxo-7,8-dihydro-pyrido[2,3-d]pyrimidine-6-carboxylic acid [5-(3-amino-1-phenyl-propylcarbamoyl)-2-chloro-phenyl]-amide; hydrochloride

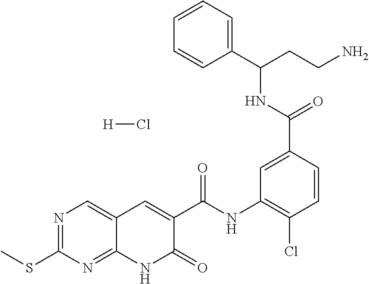

To a 10 mL reaction flask was added tert-butyl 3-(4-chloro-3-(2-(methylthio)-7-oxo-7,8-dihydro-pyrido[2,3-d]pyrimidine-6-carboxamido)benzamido)-3-phenylpropylcarbamate (50 mg, 80.2 μmol, Eq: 1.00) (from Example 106 supra), 1,4-dioxane (2 mL) and hydrochloric acid in 1,4-dioxane (2.4 g, 8.00 mmol, Eq: 99.7). The mixture was stirred at room temperature overnight. The reaction was concentrated to remove solvent and after drying provided 2-methylsulfanyl-7-oxo-7,8-dihydro-pyrido[2,3-d]pyrimidine-6-carboxylic acid [5-(3-amino-1-phenyl-propylcarbamoyl)-2-chloro-phenyl]-amide; hydrochloride. (Yield 35 mg, 78%).

HR-MS (ES⁺) m/z Calculated for C₂₅H₂₄ClN₆O₃S ([M+H]⁺): 523.1314. Found: 523.1313.

### Example 108

### 2-(4-Acetylpiperazin-1-yl)-N-(2-chloro-5-(3-chlorobenzylcarbamoyl)phenyl)-7-oxo-7,8-dihydropyrido[2,3-d]pyrimidine-6-carboxamide

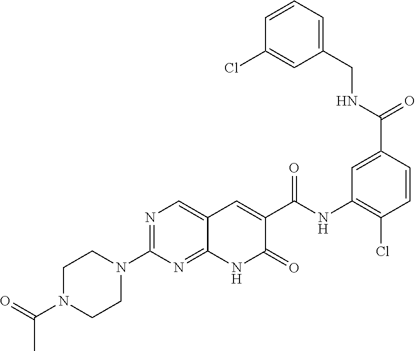

To a disposable tube with stir bar and equipped with a Teflon-lined screw cap was added 2-methane-sulfonyl-7-oxo-7,8-dihydro-pyrido[2,3-d]pyrimidine-6-carboxylic acid [2-chloro-5-(3-chloro-benzylcarbamoyl)-phenyl]-amide (100 mg, 183 μmol, Eq: 1.00) (from Example 75 supra), 1-(piperazin-1-yl)ethanone (469 mg, 3.66 mmol, Eq: 20) and N,N-dimethylformamide (500 μL). The reaction was heated at 85° C. in a pre-heated oil bath overnight. The reaction was cooled to room temperature and then diluted with water and concentrated to dryness. The crude material was suspended in DMSO and water. The solid was collected by filtration and then rinsed with water, ethyl acetate and diethyl ether to provide after drying 2-(4-acetylpiperazin-1-yl)-N-(2-chloro-5-(3-chloro-benzylcarbamoyl)phenyl)-7-oxo-7,8-dihydropyrido[2,3-d]pyrimidine-6-carboxamide. (Yield 42 mg, 39%).

HR-MS (ES⁺) m/z Calculated for C₂₈H₂₆Cl₂N₇O₄ ([M+H]⁺): 594.1418. Found: 594.1419.

### Example 109

### tert-Butyl 4-(6-(2-chloro-5-(3-chlorobenzylcarbamoyl)phenylcarbamoyl)-7-oxo-7,8-dihydropyrido[2,3-d]pyrimidin-2-yl)piperazine-1-carboxylate

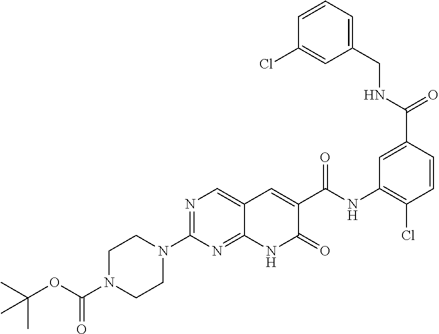

To a disposable tube with stir bar and equipped with a Teflon-lined screw cap was added 2-methane-sulfonyl-7-oxo-7,8-dihydro-pyrido[2,3-d]pyrimidine-6-carboxylic acid [2-chloro-5-(3-chloro-benzylcarbamoyl)-phenyl]-amide (150 mg, 275 μmol, Eq: 1.00) (from Example 75 supra), tert-butyl piperazine-1-carboxylate (1.02 g, 5.49 mmol, Eq: 20) and N,N-dimethylformamide (500 μL). The tube was then heated at 80° C. in a pre-heated oil bath with stirring overnight. The reaction was cooled to room temperature and diluted with water, after which it was basified with a 1.0 N NaOH aqueous solution. The reaction was concentrated under reduced pressure, after which water was added and the precipitated solid was collected by filtration. The solid was rinsed with EtOAc and water and dried to provide tert-butyl 4-(6-(2-chloro-5-(3-chlorobenzylcarbamoyl)phenylcarbamoyl)-7-oxo-7,8-dihydropyrido[2,3-d]pyrimidin-2-yl)piperazine-1-carboxylate. (Yield 70 mg, 39%).

HR-MS (ES⁺) m/z Calculated for C₃₁H₃₁Cl₂N₇O₅Na ([M+Na]⁺): 674.1656. Found: 674.1657.

### Example 110

### tert-Butyl (1s,4s)-4-(6-(2-chloro-5-(3-chlorobenzylcarbamoyl)phenylcarbamoyl)-7-oxo-7,8-dihydropyrido[2,3-d]pyrimidin-2-ylamino)cyclohexylcarbamate

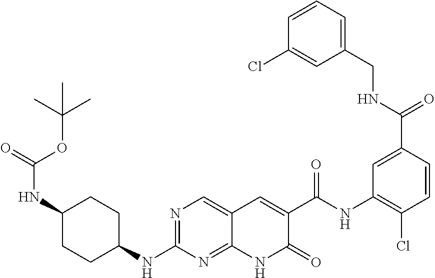

To a disposable tube with stir bar equipped with a Teflon-lined screw cap was added 2-methane-sulfonyl-7-oxo-7,8-dihydro-pyrido[2,3-d]pyrimidine-6-carboxylic acid [2-chloro-5-(3-chloro-benzylcarbamoyl)-phenyl]-amide (150 mg, 275 μmol, Eq: 1.00) (from Example 75 supra), tert-butyl (1S,4S)-4-aminocyclohexylcarbamate (1.18 g, 5.49 mmol, Eq: 20) and N,N-dimethyl-formamide (500 μL). The tube was then heated at 80° C. in a pre-heated oil bath with stirring overnight. The reaction was cooled to room temperature and then basified with 1.0 N NaOH (aq) solution after which the mixture was concentrated under reduced pressure. The crude material was diluted with water and ethyl acetate and the precipitated solid was collected, rinsed with water, ethyl acetate and diethyl ether to provide after drying tert-butyl (1s,4s)-4-(6-(2-chloro-5-(3-chlorobenzylcarbamoyl)phenylcarbamoyl)-7-oxo-7,8-dihydropyrido[2,3-d]pyrimidin-2-ylamino)cyclohexylcarbamate. (Yield 75 mg, 40%).

HR-MS (ES⁺) m/z Calculated for C₃₃H₃₅Cl₂N₇NaO₅ ([M+Na]⁺): 702.1969. Found: 702.1967.

### Example 111

### tert-Butyl 3-(4-chloro-3-(2-methyl-7-oxo-7,8-dihydropyrido[2,3-d]pyrimidine-6-carboxamido)benzamido)-3-phenylpropylcarbamate

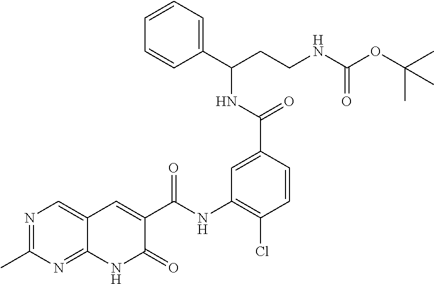

### Step A

### 4-Amino-2-methylpyrimidine-5-carbonitrile

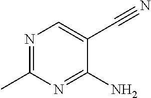

Acetimidamide hydrochloride (3.87 g, 40.9 mmol) was suspended in ethanol (10 mL). A solution of sodiumethoxide in ethanol (13.3 g, 40.9 mmol) was added and the mixture was stirred at room temperature for 30-40 minutes. The insoluble material was removed by filtration and washed with small volumes of ethanol. The filtrate was treated with 2-(ethoxymethylene)-malononitrile (2.50 g, 20.5 mmol). A thick precipitate rapidly dropped out of solution which stopped stirring. The mixture was diluted with additional ethanol (3-4 mL). Stirring resumed but was still inefficient. After one hour, the solid was collected by filtration, washed with small volumes of ethanol and dried with suction under house vacuum to give 4-amino-2-methylpyrimidine-5-carbonitrile. (Yield 2.05 g, 15.3 mmol, 74.7%).

### Step B

### 4-Amino-2-methylpyrimidine-5-carbaldehyde

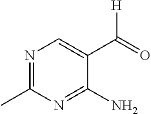

4-Amino-2-methylpyrimidine-5-carbonitrile (500 mg, 3.73 mmol) and 10% palladium on carbon (40.8 mg, 38.3 μmol) were combined in water (4 mL) and sulfuric acid (800 μL). The mixture was hydrogenated at room temperature and 30-50 psi for 75 minutes. The reaction mixture was filtered through Celite and washed with water. The filtrate was combined with the washes and treated with concentrated ammonium hydroxide. Solid precipitated out of solution, with heat given off. The mixture was chilled prior to collecting the solid by filtration. The filtered solid was washed with water and then dried under house vacuum to give 4-amino-2-methylpyrimidine-5-carbaldehyde. (Yield 299.2 mg, 2.18 mmol, 58.5%).

### Step C

### Ethyl 2-Methyl-7-oxo-7,8-dihydropyrido[2,3-d]pyrimidine-6-carboxylate

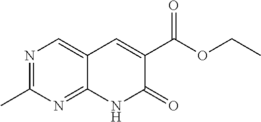

4-Amino-2-methylpyrimidine-5-carbaldehyde (0.293 g, 2.14 mmol), diethyl malonate (686 mg, 4.28 mmol), potassium carbonate (443 mg, 3.2 mmol) and triethylamine (218 mg, 2.15 mmol) were combined in DMF (9.5 mL). The reaction mixture was heated at 85° C. and stirred under argon overnight. The reaction mixture was filtered and the solid was washed with EtOAc. The material was purified by flash chromatography in two runs (silica gel, SF15-24 g and SF25-40 g, 0% to 100% MeOH in dichloromethane). Product-containing fractions from each run were combined, concentrated and dried under house vacuum to give ethyl 2-methyl-7-oxo-7,8-dihydropyrido[2,3-d]pyrimidine-6-carboxylate. (Yield 340 mg, 1.38 mmol, 64.8%).

### Step D

### 2-Methyl-7-oxo-7,8-dihydropyrido[2,3-d]pyrimidine-6-carboxylic acid

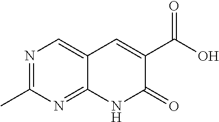

In a 25 mL pear-shaped flask, ethyl 2-methyl-7-oxo-7,8-dihydropyrido[2,3-d]pyrimidine-6-carboxylate (0.336 g, 1.37 mmol) was combined with water (2.0 mL) and methanol (4.0 mL) to give a yellow suspension. Lithium hydroxide (81.9 mg, 3.42 mmol) was added. Solid went into solution and the solution was heated at 65° C. for 4 hours after which the reaction was cooled to room temperature and concentrated. The solid residue was diluted with water and treated with 6N HCl. After 10-15 minutes the solid was collected by filtration, washed with water and dried under house vacuum with suction to give 2-methyl-7-oxo-7,8-dihydropyrido[2,3-d]pyrimidine-6-carboxylic acid. (Yield 265.4 mg, 1.29 mmol, 94.5%).

### Step E

### Methyl 4-chloro-3-(2-methyl-7-oxo-7,8-dihydropyrido[2,3-d]pyrimidine-6-carboxamido-benzoate

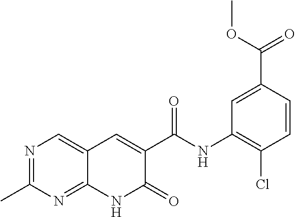

In a 25 mL pear-shaped flask, 2-methyl-7-oxo-7,8-dihydropyrido[2,3-d]pyrimidine-6-carboxylic acid (0.260 g, 1.27 mmol) and methyl 3-amino-4-chlorobenzoate (353 mg, 1.9 mmol) were combined with DMF (4.0 mL) to give a yellow suspension. Triethylamine (639 mg, 6.31 mmol) was added and the solid went into solution. HATU (723 mg, 1.9 mmol) was then added. The solution quickly began to darken from a yellow solution to orange and then deep red. Solid also began to precipitate out of solution. After 5 hours, the reaction was diluted with EtOAc. The solid was collected by filtration, washed with EtOAc and dried under house vacuum to give methyl 4-chloro-3-(2-methyl-7-oxo-7,8-dihydropyrido[2,3-d]pyrimidine-6-carboxamido)-benzoate. (Yield 0.212 g, 569 μmol, 44.9%).

### Step F

### 4-Chloro-3-(2-methyl-7-oxo-7,8-dihydropyrido[2,3-d]pyrimidine-6-carboxamido)benzoic acid

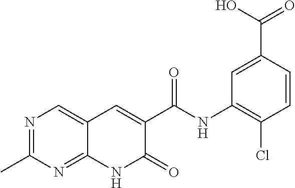

Methyl 4-chloro-3-(2-methyl-7-oxo-7,8-dihydropyrido[2,3-d]pyrimidine-6-carboxamido)-benzoate (0.246 g, 660 μmol) was suspended in methanol (2.0 mL) and water (1.0 mL). Lithium hydroxide (39.5 mg, 1.65 mmol) was added and mixture was heated at 60° C. The material remained insoluble. The reaction mixture was diluted with THF (4 mL), methanol (2 mL) and water (1 mL) to dissolve the solid. After three hours, the reaction was found to be incomplete. Additional LiOH (˜40 mg) was added and heating continued at 65° C. for another 2 hours. The reaction was then concentrated. The residue was triturated with water and EtOAc. The solid was collected and dried under house vacuum to give 4-chloro-3-(2-methyl-7-oxo-7,8-dihydropyrido[2,3-d]pyrimidine-6-carboxamido)benzoic acid. (Yield 0.234 g, 652 μmol, 98.8%).

### Step G

### tert-Butyl 3-(4-chloro-3-(2-methyl-7-oxo-7,8-dihydropyrido[2,3-d]pyrimidine-6-carboxamido)-benzamido)-3-phenylpropylcarbamate

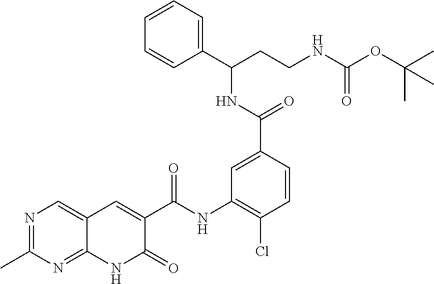

In a 25 mL pear-shaped flask, 4-chloro-3-(2-methyl-7-oxo-7,8-dihydropyrido[2,3-d]pyrimidine-6-carboxamido)benzoic acid (109.7 mg, 306 μmol), tert-butyl 3-amino-3-phenylpropylcarbamate (84.2 mg, 336 μmol) (from Example 6 supra) and triethylamine (152 mg, 1.51 mmol) were combined with DMF (1.53 mL) to give a red solution. HATU (134 mg, 352 μmol) was added and reaction was stirred at room temperature under an argon atmosphere. After 3 hours the reaction was diluted with water. The solid which precipitated was collected and washed with water and EtOAc and then dried under house vacuum with suction to give tert-butyl 3-(4-chloro-3-(2-methyl-7-oxo-7,8-dihydropyrido-[2,3-d]pyrimidine-6-carboxamido)benzamido)-3-phenylpropylcarbamate. (Yield 135.8 mg, 230 μmol, 75.1%).

HR-MS (ES⁺) m/z Calculated for C₃₀H₃₂ClN₆O₅ ([M+H]⁺): 591.2117. Found: 591.2122.

### Example 112

### N-(5-(3-Amino-1-phenylpropylcarbamoyl)-2-chlorophenyl)-2-methyl-7-oxo-7,8-dihydropyrido[2,3-d]pyrimidine-6-carboxamide hydrochloride

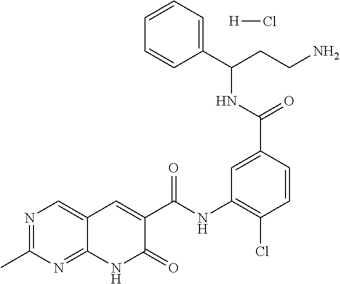

tert-Butyl 3-(4-chloro-3-(2-methyl-7-oxo-7,8-dihydropyrido[2,3-d]pyrimidine-6-carboxamido)benzamido)-3-phenylpropylcarbamate (119.9 mg, 203 μmol) (from Example 111 supra) was suspended in dioxane (2.0 mL). A dioxane solution of hydrochloric acid (4.0M, 2.0 mL, 2.4 g, 8.00 mmol) was added dropwise. A clear solution resulted briefly before material began to precipitate out of solution again. The mixture was stirred at room temperature for 2 hours and then was concentrated to a small volume and diluted with EtOAc. The solid was collected and washed with EtOAc. The solid was then dissolved in water-acetonitrile, filtered to remove any insoluble particles and freeze-dried to give N-(5-(3-amino-1-phenylpropylcarbamoyl)-2-chlorophenyl)-2-methyl-7-oxo-7,8-dihydropyrido[2,3-d]pyrimidine-6-carboxamide hydrochloride. (Yield 103.4 mg, 194 μmol, 95.7%).

HR-MS (ES⁺) m/z Calculated for C₂₅H₂₄ClN₆O₃ ([M+H]⁺): 491.1593. Found: 491.1591.

### Example 113

### tert-Butyl 2-(4-chloro-3-(2-methyl-7-oxo-7,8-dihydropyrido[2,3-d]pyrimidine-6-carboxamido)benzamido)-2-phenylethylcarbamate

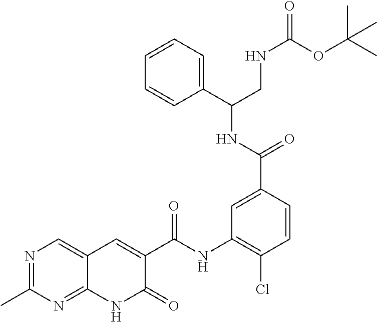

4-Chloro-3-(2-methyl-7-oxo-7,8-dihydropyrido[2,3-d]pyrimidine-6-carboxamido)benzoic acid (117.7 mg, 328 μmol) (from Example 111 supra), tert-butyl 2-amino-2-phenylethylcarbamate (85.3 mg, 361 μmol) (from Example 4 supra) and HATU (143 mg, 377 μmol, Eq: 1.15) were combined in DMF (1.64 mL). Material remained insoluble. Triethylamine (166 mg, 1.64 mmol) was added and the mixture was stirred at room temperature under an argon atmosphere. A clear red solution initially resulted but after a few minutes solid began to precipitate out of solution. After 6 hours the reaction mixture was diluted with water and stirred vigorously for 10-15 minutes. The solid was then collected, washed with water and dried under house vacuum to give tert-butyl 2-(4-chloro-3-(2-methyl-7-oxo-7,8-dihydropyrido[2,3-d]pyrimidine-6-carboxamido)benzamido)-2-phenylethylcarbamate. (Yield 161.3 mg, 280 μmol, 85.2%).

HR-MS (ES⁺) m/z Calculated for C₂₉H₃₀ClN₆O₅ ([M+H]⁺): 577.1961. Found: 577.1960.

### Example 114

### N-(5-(2-Amino-1-phenylethylcarbamoyl)-2-chlorophenyl)-2-methyl-7-oxo-7,8-dihydropyrido[2,3-d]pyrimidine-6-carboxamide hydrochloride

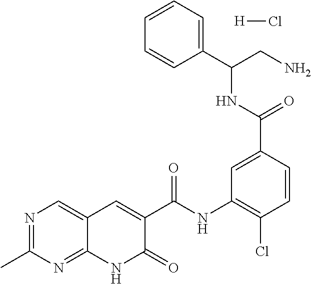

tert-Butyl 2-(4-chloro-3-(2-methyl-7-oxo-7,8-dihydropyrido[2,3-d]pyrimidine-6-carboxamido)-benzamido)-2-phenylethylcarbamate (0.155 g, 269 μmol) (from Example 113 supra) was suspended in dioxane (2.0 mL). Hydrogen chloride (4M solution in dioxane) (2.0 mL, 8.00 mmol) was added and stirring continued at room temperature under an argon atmosphere. Most of the material went into solution and a new precipitate then dropped out of solution. After 3 hours, the reaction mixture was concentrated to a smaller volume and diluted with EtOAc. The solid was collected by filtration and washed with EtOAc. The filtered solid was then dissolved in water and a small volume of acetonitrile, filtered to remove any insoluble particles and freeze dried to give N-(5-(2-amino-1-phenylethylcarbamoyl)-2-chlorophenyl)-2-methyl-7-oxo-7,8-dihydropyrido[2,3-d]pyrimidine-6-carboxamide hydrochloride. (Yield 124 mg, 242 μmol, 89.9%).

HR-MS (ES⁺) m/z Calculated for C₂₄H₂₂ClN₆O₃ ([M+H]⁺): 477.1437. Found: 477.1433.

### Example 115

### ((S)-2-{4-Chloro-3-[(2-methoxy-7-oxo-7,8-dihydro-pyrido[2,3-d]pyrimidine-6-carbonyl)-amino]-benzoylamino}-2-phenyl-ethyl)-carbamic acid tert-butyl ester

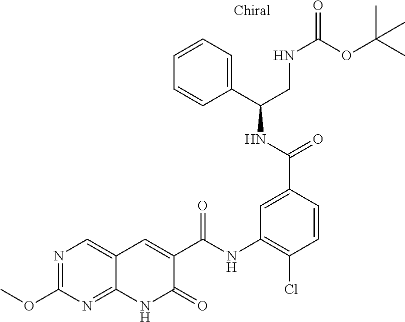

Triethylamine (0.042 mL, 0.30 mmol) was added to a mixture of 4-chloro-3-[(2-methoxy-7-oxo-7,8-dihydro-pyrido[2,3-d]pyrimidine-6-carbonyl)-amino]-benzoic acid (0.10 g, 0.27 mmol) (from Example 22 supra) and HATU (0.11 g, 0.30 mmol) (Aldrich) in DMF (4.0 mL). Mixture was stirred at room temperature for 30 minutes. ((R)-2-Amino-2-phenyl-ethyl)-carbamic acid tert-butyl ester (80 mg, 0.32 mmol) (from Example 5 supra) was added. Mixture was stirred for 18 hours and then partitioned between ethyl acetate and water. The aqueous phase was extracted with ethyl acetate (3×). The combined organic phase was washed with water and brine, dried (magnesium sulfate), filtered, and concentrated. The residue was collected by filtration and washed with ethyl acetate and water and dried in vacuum oven to give ((S)-2-{4-chloro-3-[(2-methoxy-7-oxo-7,8-dihydro-pyrido[2,3-d]pyrimidine-6-carbonyl)-amino]-benzoylamino}-2-phenyl-ethyl)-carbamic acid tert-butyl ester. (Yield 0.017 g, 13.2%).

HR-MS (ES⁺) m/z Calculated for C₂₉H₃₀ClN₆O₆ ([M+H]⁺): 593.1906. Found: 593.1910.

### Example 116

### tert-Butyl 3-(4-chloro-3-(2-(3-(4-methylpiperazin-1-yl)propylamino)-7-oxo-7,8-dihydropyrido[2,3-d]pyrimidine-6-carboxamido)benzamido)-3-phenylpropylcarbamate

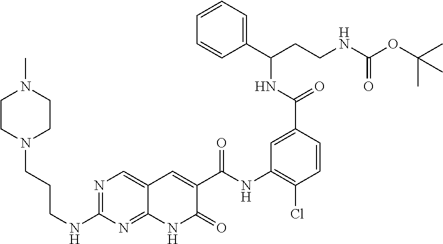

### Step A

### tert-Butyl 3-(4-chloro-3-(2-(methylsulfonyl)-7-oxo-7,8-dihydropyrido[2,3-d]pyrimidine-6-carboxamido)benzamido)-3-phenylpropylcarbamate

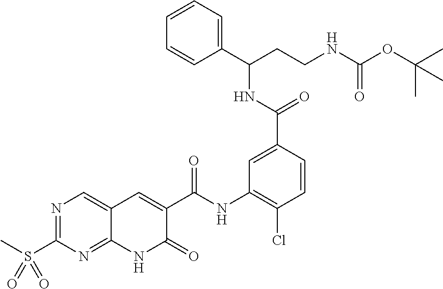

To a round bottom flask with stir bar was added tert-butyl 3-(4-chloro-3-(2-(methylthio)-7-oxo-7,8-dihydropyrido[2,3-d]pyrimidine-6-carboxamido)benzamido)-3-phenylpropylcarbamate (450 mg, 722 μmol, Eq: 1.00) (from Example 106 supra), MeOH (5 mL), THF (10 mL) and water (7 mL). The reaction was stirred at room temperature for 5 minutes after which Oxone (1.11 g, 1.81 mmol, Eq: 2.5) was added all at once. After 3 hours, another portion of oxone (1.11 g, 1.81 mmol, Eq: 2.5) was added. The reaction was allowed to stir at room temperature overnight. The precipitated solid was filtered off and rinsed with plenty of water to get rid of excess oxone. The solid was then dried in a vacuum oven at 65° C. to provide tert-butyl 3-(4-chloro-3-(2-(methylsulfonyl)-7-oxo-7,8-dihydropyrido[2,3-d]pyrimidine-6-carboxamido)benzamido)-3-phenylpropylcarbamate as a solid. (Yield 381 mg, 80%).

### Step B

### tert-Butyl 3-(4-chloro-3-(2-(3-(4-methylpiperazin-1-yl)propylamino)-7-oxo-7,8-dihydropyrido[2,3-d]pyrimidine-6-carboxamido)benzamido)-3-phenylpropylcarbamate

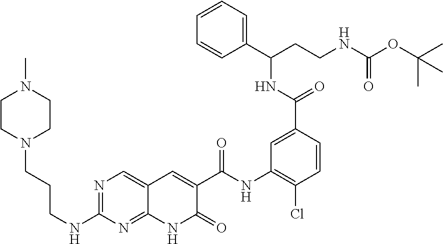

To a disposable tube with stir bar was added tert-butyl 3-(4-chloro-3-(2-(methylsulfonyl)-7-oxo-7,8-dihydropyrido[2,3-d]pyrimidine-6-carboxamido)benzamido)-3-phenylpropylcarbamate (100 mg, 153 μmol, Eq: 1.00), DMF (0.5 mL) and then 3-(4-methylpiperazin-1-yl)propan-1-amine (1.0 g, 6.36 mmol, Eq: 41.7). The reaction was then heated at 80° C. in a pre-heated oil bath for 2 hours. The reaction was cooled to room temperature, after which water was added and the solid was collected and rinsed with water, diethyl ether, ethyl acetate and water providing tert-butyl 3-(4-chloro-3-(2-(3-(4-methylpiperazin-1-yl)propylamino)-7-oxo-7,8-dihydropyrido[2,3-d]pyrimidine-6-carboxamido)benzamido)-3-phenylpropylcarbamate. (Yield 95 mg, 85%).

LR-MS [M+H]⁺: 732.

### Example 117

### tert-Butyl 2-(4-chloro-3-(2-(3-(4-methylpiperazin-1-yl)propylamino)-7-oxo-7,8-dihydropyrido[2,3-d]pyrimidine-6-carboxamido)benzamido)-2-phenylethylcarbamate

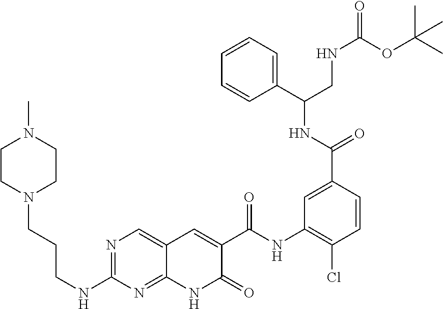

### Step A

### tert-Butyl 2-(4-chloro-3-(2-(methylsulfonyl)-7-oxo-7,8-dihydropyrido[2,3-d]pyrimidine-6-carboxamido)benzamido)-2-phenylethylcarbamate

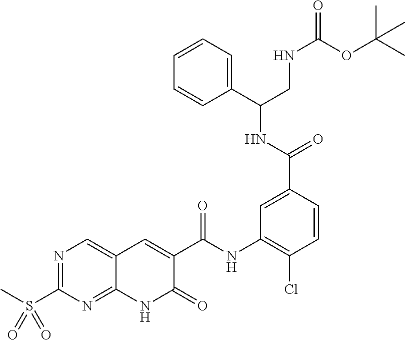

To a round bottom flask with stir bar was added tert-butyl 2-(4-chloro-3-(2-(methylthio)-7-oxo-7,8-dihydropyrido[2,3-d]pyrimidine-6-carboxamido)benzamido)-2-phenylethylcarbamate (300 mg, 493 μmol, Eq: 1.00) (from Example 98 supra), MeOH (3.5 mL), water (7 mL) and THF (3.5 mL). After stirring at room temperature for 5 minutes Oxone (757 mg, 1.23 mmol, Eq: 2.5) was added all at once. After stirring at room temperature for 49 hours another equivalent of oxone was added to the reaction mixture together with THF (1 mL) and MeOH (1 mL). Mixture was stirred at room temperature overnight. Solid was collected by filtration and rinsed with water, and dried in oven vacuum at 65° C. to provide tert-butyl 2-(4-chloro-3-(2-(methyl-sulfonyl)-7-oxo-7,8-dihydropyrido[2,3-d]pyrimidine-6-carboxamido)benzamido)-2-phenylethyl-carbamate as a solid. (Yield 279 mg, 88%).

### Step B

### tert-Butyl 2-(4-chloro-3-(2-(3-(4-methylpiperazin-1-yl)propylamino)-7-oxo-7,8-dihydro-pyrido[2,3-d]pyrimidine-6-carboxamido)benzamido)-2-phenylethylcarbamate

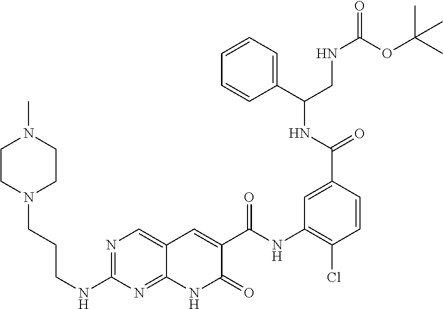

To a disposable tube with stir bar were added tert-butyl 2-(4-chloro-3-(2-(methylsulfonyl)-7-oxo-7,8-dihydropyrido[2,3-d]pyrimidine-6-carboxamido)benzamido)-2-phenylethylcarbamate (100 mg, 156 μmol, Eq: 1.00), DMF (0.5 mL) and then 3-(4-methylpiperazin-1-yl)propan-1-amine (1 g, 6.36 mmol, Eq: 40.8). The reaction vessel was then heated at 80° C. in a pre-heated oil bath for 2 hours. The reaction was cooled to room temperature, after which water was added. The precipitated solid was filtered off, rinsed with water, ethyl acetate, water and then diethyl ether to provide after drying tert-butyl 2-(4-chloro-3-(2-(3-(4-methylpiperazin-1-yl)propylamino)-7-oxo-7,8-dihydropyrido[2,3-d]pyrimidine-6-carboxamido)benzamido)-2-phenylethylcarbamate. (Yield 83 mg, 74%).

LR-MS (ES⁺) [M+H]⁺: 718.

### Example 118

### 2-((1s,4 s)-4-Aminocyclohexylamino)-N-(2-chloro-5-(3-chlorobenzylcarbamoyl)phenyl)-7-oxo-7,8-dihydropyrido[2,3-d]pyrimidine-6-carboxamide hydrochloride

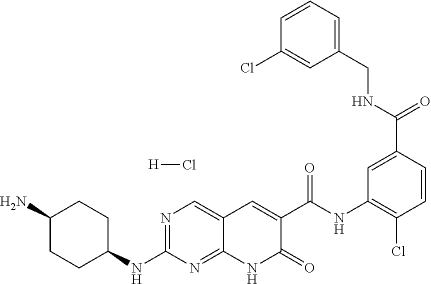

To a 50 mL round bottom flask with stir bar were added tert-butyl (1S,4S)-4-(6-(2-chloro-5-(3-chlorobenzylcarbamoyl)phenylcarbamoyl)-7-oxo-7,8-dihydropyrido[2,3-d]pyrimidin-2-ylamino)cyclohexylcarbamate (50 mg, 73.5 μmol, Eq: 1.00) (from Example 110 supra), 1,4-dioxane (2 mL) and then hydrochloric acid in 1,4-dioxane (8.00 mmol, Eq: 109). The reaction was allowed to stir at room temperature for 4 hours. The reaction was concentrated under reduced pressure to provide after drying 2-((1S,4S)-4-aminocyclohexylamino)-N-(2-chloro-5-(3-chlorobenzylcarbamoyl)phenyl)-7-oxo-7,8-dihydropyrido[2,3-d]pyrimidine-6-carboxamide hydrochloride. (Yield 40 mg, 88%).

HR-MS (ES⁺) m/z Calculated for C₂₈H₂₈Cl₂N₇O₃ ([M+H]⁺): 580.1625. Found: 580.1626.

### Example 119

### N-(2-Chloro-5-(3-chlorobenzylcarbamoyl)phenyl)-7-oxo-2-(piperazin-1-yl)-7,8-dihydropyrido[2,3-d]pyrimidine-6-carboxamide hydrochloride

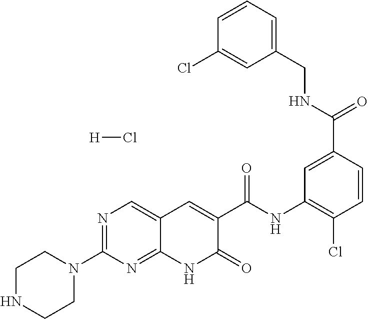

To a 50 mL round bottom flask with stir bar were added tert-butyl 4-(6-(2-chloro-5-(3-chlorobenzylcarbamoyl)phenylcarbamoyl)-7-oxo-7,8-dihydropyrido[2,3-d]pyrimidin-2-yl)piperazine-1-carboxylate (50 mg, 76.6 μmol, Eq: 1.00) (from Example 109 supra), 1,4-dioxane (2 mL) and then 4M hydrochloric acid in 1,4-dioxane (8.00 mmol, Eq: 104). The reaction was allowed to stir at room temperature for 3 hours. The reaction mixture was concentrated under reduced pressure to provide after drying N-(2-chloro-5-(3-chlorobenzylcarbamoyl)-phenyl)-7-oxo-2-(piperazin-1-yl)-7,8-dihydropyrido[2,3-d]pyrimidine-6-carboxamide hydrochloride. (Yield 41 mg, 91%).

HR-MS (ES⁺) m/z Calculated for C₂₆H₂₄Cl₂N₇O₃ ([M+H]⁺): 552.1312. Found: 552.1310.

### Example 120

### N-(5-(3-Amino-1-phenylpropylcarbamoyl)-2-chlorophenyl)-2-(3-(4-methylpiperazin-1-yl)propylamino)-7-oxo-7,8-dihydropyrido[2,3-d]pyrimidine-6-carboxamide

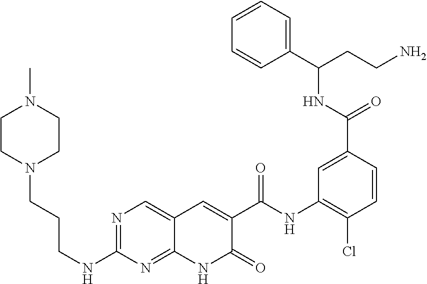

To a disposable vial with stir bar were added tert-butyl 3-(4-chloro-3-(2-(3-(4-methylpiperazin-1-yl)propylamino)-7-oxo-7,8-dihydropyrido[2,3-d]pyrimidine-6-carboxamido)benzamido)-3-phenylpropylcarbamate (70 mg, 95.6 μmol, Eq: 1.00) (from Example 116 supra), 1,4-dioxane (1.5 mL) and then 4M hydrochloric acid in 1,4-dioxane (8.00 mmol, Eq: 83.7). The reaction was stirrred at room temperature overnight, after which water was added. The crude solution was basified with 2.0 N aqueous NaOH and then concentrated under reduced pressure. The crude material was purified using a reverse phase C-18 column eluting with 0-100% MeOH/H₂O solvents system to provide after drying N-(5-(3-amino-1-phenylpropylcarbamoyl)-2-chlorophenyl)-2-(3-(4-methylpiperazin-1-yl)propylamino)-7-oxo-7,8-dihydropyrido[2,3-d]pyrimidine-6-carboxamide. (Yield 45 mg, 75%).

HR-MS (ES⁺) m/z Calculated for C₃₂H₃₉ClN₉O₃ ([M+H]⁺): 632.2859. Found: 632.2861.

### Example 121

### N-(5-(3-Amino-1-phenylpropylcarbamoyl)-2-chlorophenyl)-2-(3-(4-methylpiperazin-1-yl)propylamino)-7-oxo-7,8-dihydropyrido[2,3-d]pyrimidine-6-carboxamide hydrochloride

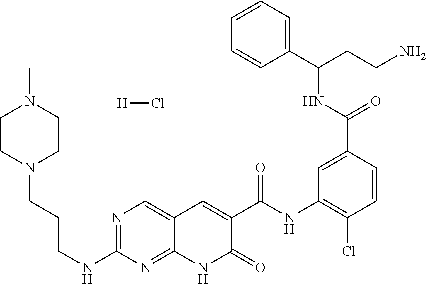

In a 5 mL vial containing N-(5-(3-amino-1-phenylpropylcarbamoyl)-2-chlorophenyl)-2-(3-(4-methylpiperazin-1-yl)propylamino)-7-oxo-7,8-dihydropyrido[2,3-d]pyrimidine-6-carboxamide (20 mg, 0.032 mmol) (from Example 116 supra) was added 0.5 M aqueous hydrochloric acid (1 mL). After stirring for 5 minutes, the solution was concentrated under reduced pressure and further dried in a vacuum oven (80° C.) providing N-(5-(3-amino-1-phenylpropylcarbamoyl)-2-chlorophenyl)-2-(3-(4-methylpiperazin-1-yl)propylamino)-7-oxo-7,8-dihydropyrido[2,3-d]pyrimidine-6-carboxamide hydrochloride. (Yield 23 mg, 98%).

HR-MS (ES⁺) m/z Calculated for C₃₂H₃₉ClN₉O₃ ([M+H]⁺): 632.2859. Found: 632.2856.

### Example 122

### N-(5-(2-Amino-1-phenylethylcarbamoyl)-2-chlorophenyl)-2-(3-(4-methylpiperazin-1-yl)propylamino)-7-oxo-7,8-dihydropyrido[2,3-d]pyrimidine-6-carboxamide

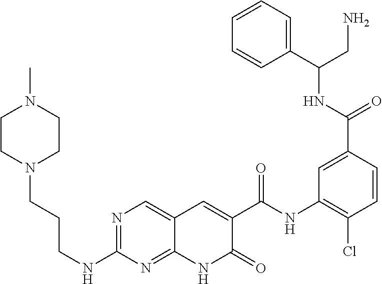

To a disposable vial with stir bar were added tert-butyl 2-(4-chloro-3-(2-(3-(4-methylpiperazin-1-yl)propylamino)-7-oxo-7,8-dihydropyrido[2,3-d]pyrimidine-6-carboxamido)benzamido)-2-phenylethylcarbamate (70 mg, 97.5 μmol, Eq: 1.00) (from Example 117 supra), 1,4-dioxane (1.5 mL) and then 4M hydrochloric acid in 1,4-dioxane (8.00 mmol, Eq: 82.1). The mixture was stirred at room temperature overnight, after which the reaction was basified with 1.0 N aq NaOH and dissolved in MeOH. The reaction was then concentrated under reduced pressure and the crude material was purified using a C-18 column eluting with 0-100% MeOH/H₂O to provide after drying N-(5-(2-amino-1-phenylethylcarbamoyl)-2-chlorophenyl)-2-(3-(4-methylpiperazin-1-yl)propylamino)-7-oxo-7,8-dihydropyrido[2,3-d]pyrimidine-6-carboxamide. (Yield 53 mg, 88%).

HR-MS (ES⁺) m/z Calculated for C₃₁H₃₇ClN₉O₃ ([M+H]⁺): 618.2703. Found: 618.2701.

### Example 123

### N-(5-(2-Amino-1-phenylethylcarbamoyl)-2-chlorophenyl)-2-(3-(4-methylpiperazin-1-yl)propylamino)-7-oxo-7,8-dihydropyrido[2,3-d]pyrimidine-6-carboxamide hydrochloride

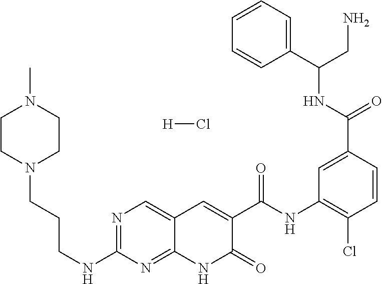

In a 5 mL vial containing N-(5-(2-amino-1-phenylethylcarbamoyl)-2-chlorophenyl)-2-(3-(4-methylpiperazin-1-yl)propylamino)-7-oxo-7,8-dihydropyrido[2,3-d]pyrimidine-6-carboxamide (30 mg, 0.049 mmol) (from Example 122 supra) was added 0.5 M aqueous hydrochloric acid (1 mL). After stirring for 5 minutes, the solution was concentrated under reduced pressure and further dried in a vacuum oven (80° C.) providing N-(5-(2-amino-1-phenylethylcarbamoyl)-2-chlorophenyl)-2-(3-(4-methylpiperazin-1-yl)propylamino)-7-oxo-7,8-dihydropyrido[2,3-d]pyrimidine-6-carboxamide hydrochloride. (Yield 20 mg, 57%).

LR-MS: [M+H]⁺: 618.

### Example 124

### N-(2-Chloro-5-(3-chlorobenzylcarbamoyl)phenyl)-2-((2,2-dimethyl-1,3-dioxolan-4-yl)methylamino)-7-oxo-7,8-dihydropyrido[2,3-d]pyrimidine-6-carboxamide

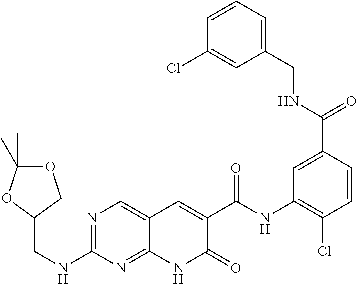

To a disposable tube with stir bar were added 2-methanesulfonyl-7-oxo-7,8-dihydro-pyrido[2,3-d]pyrimidine-6-carboxylic acid [2-chloro-5-(3-chloro-benzylcarbamoyl)-phenyl]-amide (100 mg, 183 μmol, Eq: 1.00) (from Example 75 supra), DMF (0.5 mL) and (2,2-dimethyl-1,3-dioxolan-4-yl)methanamine (506 mg, 3.86 mmol, Eq: 21.1). The reaction was heated at 75° C. in a pre-heated oil bath for 3 hours, after which it was cooled to room temperature. The reaction was diluted with water and the precipitate was collected, rinsed with water and diethyl ether providing after drying N-(2-chloro-5-(3-chlorobenzylcarbamoyl)phenyl)-2-((2,2-dimethyl-1,3-dioxolan-4-yl)methylamino)-7-oxo-7,8-dihydropyrido[2,3-d]pyrimidine-6-carboxamide. (Yield 65 mg, 59%).

HR-MS (ES⁺) m/z Calculated for C₂₈H₂₇Cl₂N₆O₅ ([M+H]⁺): 597.1415. Found: 597.1411.

### Example 125

### N-(2-Chloro-5-(3-chlorobenzylcarbamoyl)phenyl)-7-oxo-2-(piperidin-4-ylamino)-7,8-dihydropyrido[2,3-d]pyrimidine-6-carboxamide hydrochloride

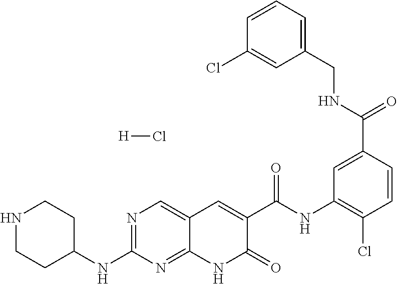

To a disposable tube with stir bar was added tert-butyl 4-(6-(2-chloro-5-(3-chlorobenzyl-carbamoyl)phenylcarbamoyl)-7-oxo-7,8-dihydropyrido[2,3-d]pyrimidin-2-ylamino)piperidine-1-carboxylate (65 mg, 97.5 μmol, Eq: 1.00) (from Example 91 supra), 1,4-dioxane (2 mL) and 4M hydrochloric acid in 1,4-dioxane (8.00 mmol, Eq: 82.0). The reaction was stirred at room temperature overnight, after which it was concentrated under reduced pressure to provide after drying N-(2-chloro-5-(3-chlorobenzylcarbamoyl)phenyl)-7-oxo-2-(piperidin-4-ylamino)-7,8-dihydropyrido[2,3-d]pyrimidine-6-carboxamide hydrochloride. (Yield 45 mg, 77%). LR-MS [M+H]⁺: 566.

### Example 126

### N-(2-Chloro-5-(3-chlorobenzylcarbamoyl)phenyl)-2-(2,3-dihydroxypropylamino)-7-oxo-7,8-dihydropyrido[2,3-d]pyrimidine-6-carboxamide

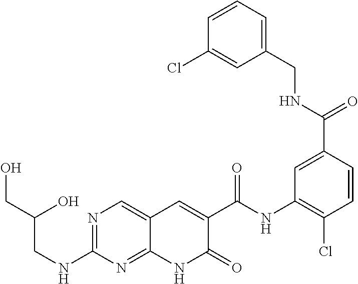

To a 25 mL round bottom flask was added N-(2-chloro-5-(3-chlorobenzyl-carbamoyl)phenyl)-2-((2,2-dimethyl-1,3-dioxolan-4-yl)methylamino)-7-oxo-7,8-dihydropyrido[2,3-d]pyrimidine-6-carboxamide (70 mg, 117 μmol, Eq: 1.00) (from Example 124 supra), THF (6 mL) and 3M hydrochloric acid (aqueous) (18.0 mmol, Eq: 154). The mixture was stirred at room temperature for 3 hours, after which the reaction was concentrated under reduced pressure to provide after drying N-(2-chloro-5-(3-chlorobenzylcarbamoyl)phenyl)-2-(2,3-dihydroxypropylamino)-7-oxo-7,8-dihydropyrido[2,3-d]pyrimidine-6-carboxamide. (Yield 55 mg, 85%).

HR-MS (ES⁺) m/z Calculated for C₂₅H₂₃Cl₂N₆O₅ ([M+H]⁺): 557.1102. Found: 557.1099.

### Example 127

### N-(2-Chloro-5-(3-chlorobenzylcarbamoyl)phenyl)-2-(2-hydroxy-2-methylpropylamino)-7-oxo-7,8-dihydropyrido[2,3-d]pyrimidine-6-carboxamide

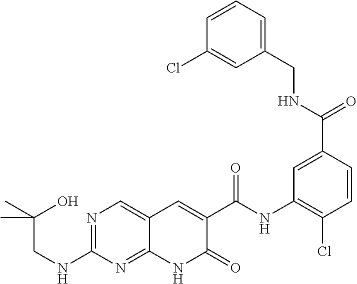

To a disposable tube with stir bar was added 2-methanesulfonyl-7-oxo-7,8-dihydro-pyrido[2,3-d]pyrimidine-6-carboxylic acid [2-chloro-5-(3-chloro-benzylcarbamoyl)-phenyl]-amide (150 mg, 275 μmol, Eq: 1.00) (from Example 75 supra), DMF (0.5 mL) and 1-amino-2-methylpropan-2-ol (1.22 g, 13.7 mmol, Eq: 50). The reaction was warmed at 75° C. in a pre-heated oil bath overnight, after which the reaction was diluted with water. The precipitated material was collected by filtration, rinsed with water and diethyl ether to provide after drying N-(2-chloro-5-(3-chlorobenzylcarbamoyl)phenyl)-2-(2-hydroxy-2-methylpropylamino)-7-oxo-7,8-dihydropyrido[2,3-d]pyrimidine-6-carboxamide. (Yield 110 mg, 72%).

HR-MS (ES⁺) m/z Calculated for C₂₆H₂₅Cl₂N₆O₄ ([M+H]⁺): 555.1309. Found: 555.1305.

### Example 128

### tert-Butyl 3-(4-chloro-3-(2-(2-(4-methylpiperazin-1-yl)ethylamino)-7-oxo-7,8-dihydropyrido[2,3-d]pyrimidine-6-carboxamido)benzamido)-3-phenylpropylcarbamate

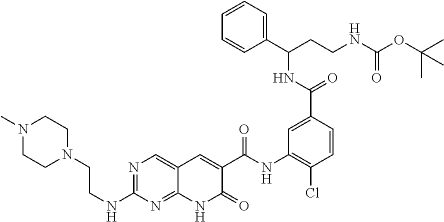

To a disposable tube with stir bar was added tert-butyl 3-(4-chloro-3-(2-(methylsulfonyl)-7-oxo-7,8-dihydropyrido[2,3-d]pyrimidine-6-carboxamido)benzamido)-3-phenylpropylcarbamate (100 mg, 153 μmol, Eq: 1.00) (from Example 116 supra), DMF (0.5 mL) and then 2-(4-methylpiperazin-1-yl)ethanamine (437 mg, 3.05 mmol, Eq: 20). The reaction was heated at 75° C. in a pre-heated oil bath for 3 hours, after which it was cooled to room temperature. The reaction was then diluted with water, the solid was filtered off and then rinsed with water and then diethyl ether to provide after drying tert-butyl 3-(4-chloro-3-(2-(2-(4-methylpiperazin-1-yl)ethylamino)-7-oxo-7,8-dihydropyrido[2,3-d]pyrimidine-6-carboxamido)benzamido)-3-phenylpropyl-carbamate. (Yield 74 mg, 68%).

LR-MS [M+H]⁺: 718.

### Example 129

### (1-{6-[5-(3-tert-Butoxycarbonylamino-1-phenyl-propylcarbamoyl)-2-chloro-phenylcarbamoyl]-7-oxo-7,8-dihydro-pyrido[2,3-d]pyrimidin-2-yl}-piperidin-4-yl)-carbamic acid tert-butyl ester

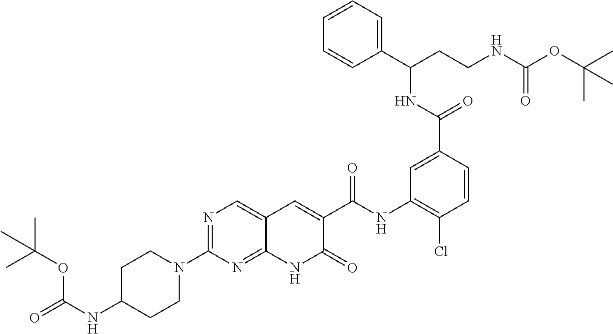

To a disposable tube with stir bar were added tert-butyl 3-(4-chloro-3-(2-(methylsulfonyl)-7-oxo-7,8-dihydropyrido[2,3-d]pyrimidine-6-carboxamido)benzamido)-3-phenylpropylcarbamate (100 mg, 153 μmol, Eq: 1.00) (from Example 116 supra), tert-butyl piperidin-4-ylcarbamate (611 mg, 3.05 mmol, Eq: 20) and then DMF (1 mL). The reaction was warmed at 75° C. in a pre-heated oil bath for 3 hours, after which it was cooled to room temperature and diluted with water. The precipitated solid was filtered and rinsed with water and ether to provide after drying (1-{6-[5-(3-tert-butoxycarbonylamino-1-phenyl-propylcarbamoyl)-2-chloro-phenylcarbamoyl]-7-oxo-7,8-dihydro-pyrido[2,3-d]pyrimidin-2-yl}-piperidin-4-yl)-carbamic acid tert-butyl ester. (Yield 85 mg, 72%).

LR-MS: [M−H]⁺ 773.

### Example 130

### (1-{6-[5-(2-tert-Butoxycarbonylamino-1-phenyl-ethylcarbamoyl)-2-chloro-phenylcarbamoyl]-7-oxo-7,8-dihydro-pyrido[2,3-d]pyrimidin-2-yl}-piperidin-4-yl)-carbamic acid tert-butyl ester

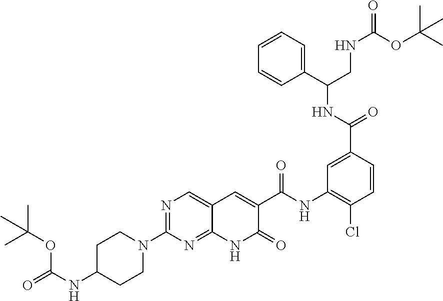

To a disposable tube with stir bar were added tert-butyl 2-(4-chloro-3-(2-(methylsulfonyl)-7-oxo-7,8-dihydropyrido[2,3-d]pyrimidine-6-carboxamido)benzamido)-2-phenylethylcarbamate (95 mg, 148 μmol, Eq: 1.00) (from Example 117 supra), tert-butyl piperidin-4-ylcarbamate (594 mg, 2.96 mmol, Eq: 20) and DMF (1 mL). The reaction was warmed at 75° C. in a pre-heated oil bath for 3 hours, after which it was cooled to room temperature and diluted with water. The precipitated solid was filtered off and rinsed with water, diethyl ether, water, ethyl acetate, water and finally diethyl ether to provide after drying (1-{6-[5-(2-tert-butoxycarbonylamino-1-phenyl-ethylcarbamoyl)-2-chloro-phenylcarbamoyl]-7-oxo-7,8-dihydro-pyrido[2,3-d]pyrimidin-2-yl}-piperidin-4-yl)-carbamic acid tert-butyl ester. (Yield 103 mg, 91%). LR-MS [M−H]⁺ 759.

### Example 131

### tert-Butyl 2-(4-chloro-3-(2-(2-(4-methylpiperazin-1-yl)ethylamino)-7-oxo-7,8-dihydropyrido[2,3-d]pyrimidine-6-carboxamido)benzamido)-2-phenylethylcarbamate

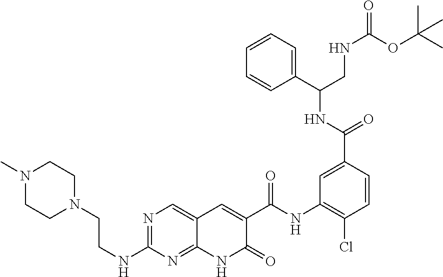

To a disposable tube with stir bar were added tert-butyl 2-(4-chloro-3-(2-(methylsulfonyl)-7-oxo-7,8-dihydropyrido[2,3-d]pyrimidine-6-carboxamido)benzamido)-2-phenylethylcarbamate (80 mg, 125 μmol, Eq: 1.00) (from Example 117 supra), DMF (0.5 mL) and 2-(4-methylpiperazin-1-yl)ethanamine (357 mg, 2.5 mmol, Eq: 20). The reaction was warmed at 75° C. in a pre-heated oil bath, after which it was cooled and diluted with water. The emulsion was filtered, rinsed with water and diethyl ether to provide after drying tert-butyl 2-(4-chloro-3-(2-(2-(4-methylpiperazin-1-yl)ethylamino)-7-oxo-7,8-dihydropyrido[2,3-d]pyrimidine-6-carbox-amido)benzamido)-2-phenylethylcarbamate. (Yield 71 mg, 81%).

LR-MS [M+H]⁺: 704.

### Example 132

### N-(5-(3-Amino-1-phenylpropylcarbamoyl)-2-chlorophenyl)-2-(2-(4-methylpiperazin-1-yl)ethylamino)-7-oxo-7,8-dihydropyrido[2,3-d]pyrimidine-6-carboxamide hydrochloride

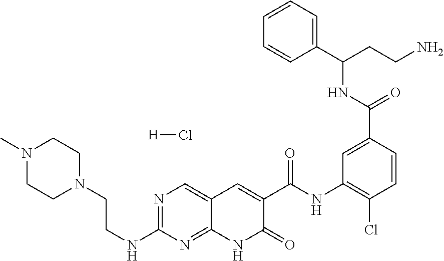

To a disposable tube with stir bar was added tert-butyl 3-(4-chloro-3-(2-(2-(4-methylpiperazin-1-yl)ethylamino)-7-oxo-7,8-dihydropyrido[2,3-d]pyrimidine-6-carboxamido)benzamido)-3-phenylpropylcarbamate (50 mg, 69.6 μmol, Eq: 1.00) (from Example 128 supra), 1,4-dioxane (1 mL) and 4M hydrochloric acid in 1,4-dioxane (4.00 mmol, Eq: 57.5) and the solution was allowed to stir at room temperature overnight. The reaction was concentrated under reduced pressure to provide after drying N-(5-(3-amino-1-phenylpropylcarbamoyl)-2-chlorophenyl)-2-(2-(4-methylpiperazin-1-yl)ethylamino)-7-oxo-7,8-dihydropyrido[2,3-d]pyrimidine-6-carboxamide hydrochloride. (Yield 40 mg, 79%).

HR-MS (ES⁺) m/z Calculated for C₃₁H₃₇ClN₉O₃ ([M+H]⁺): 618.2703. Found: 618.2703.

### Example 133

### (S)—N-(5-(2-Amino-1-phenylethylcarbamoyl)-2-chlorophenyl)-2-methoxy-7-oxo-7,8-dihydropyrido[2,3-d]pyrimidine-6-carboxamide

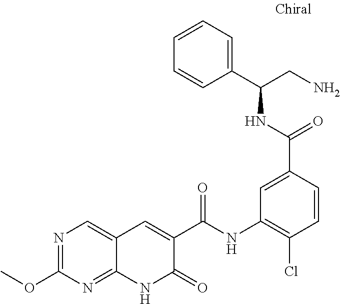

To a solution of ((S)-2-{4-chloro-3-[(2-methoxy-7-oxo-7,8-dihydro-pyrido[2,3-d]pyrimidine-6-carbonyl)-amino]-benzoylamino}-2-phenyl-ethyl)-carbamic acid tert-butyl ester (0.11 g, 0.27 mmol) (from Example 115 supra) in dichloromethane (5 mL) was added a mixture of 2,2,2-trifluoroacetic acid (2.5 mL) and dichloromethane (2.5 mL). The mixture was stirred at room temperature for 18 hours. The mixture was concentrated and then dissolved into methanol. The resulting solution was treated with saturated sodium carbonate solution (0.5 mL). The precipitate was collected by filtration and washed with methanol and water and dried in vacuum oven to give (S)—N-(5-(2-amino-1-phenylethylcarbamoyl)-2-chlorophenyl)-2-methoxy-7-oxo-7,8-dihydropyrido[2,3-d]pyrimidine-6-carboxamide. (Yield 0.082 g, 90.1%).

HR-MS (ES⁺) m/z Calculated for C₂₄H₂₂ClN₆O₄ ([M+H]⁺): 493.1387. Found: 493.1386.

### Example 134

### 2-Methoxy-7-oxo-7,8-dihydro-pyrido[2,3-d]pyrimidine-6-carboxylic acid {5-[(R)-2-amino-1-(3-chloro-phenyl)-ethylcarbamoyl]-2-chloro-phenyl}-amide

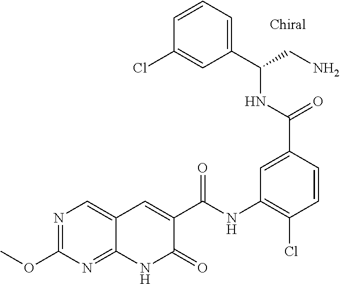

### Step A

### ((R)-2-{4-Chloro-3-[(2-methoxy-7-oxo-7,8-dihydro-pyrido[2,3-d]pyrimidine-6-carbonyl)-amino]-benzoylamino}-2-(3-chloro-phenyl-ethyl)-carbamic acid tert-butyl ester

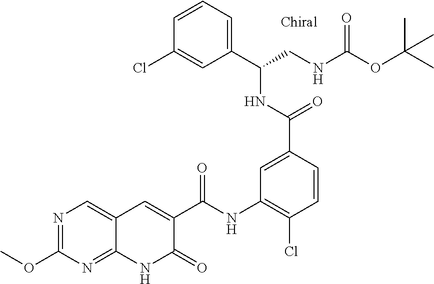

Triethylamine (0.042 mL, 0.30 mmol) was added to a mixture of 4-chloro-3-[(2-methoxy-7-oxo-7,8-dihydro-pyrido[2,3-d]pyrimidine-6-carbonyl)-amino]-benzoic acid (0.10 g, 0.27 mmol) (from Example 22 supra) and HATU (0.11 g, 0.30 mmol) (Aldrich) in DMF (4.0 mL). The mixture was stirred at room temperature for 30 minutes. [(R)-2-Amino-2-(3-chloro-phenyl)-ethyl]-carbamic acid tert-butyl ester (86.7 mg, 0.32 mmol) (from Example 12 supra) was added. The mixture was stirred for 18 hours and then partitioned between ethyl acetate and water. The precipitate was collected by filtration and washed with ethyl acetate and water and dried in vacuum oven to give ((R)-2-{4-chloro-3-[(2-methoxy-7-oxo-7,8-dihydro-pyrido[2,3-d]pyrimidine-6-carbonyl)-amino]-benzoylamino}-2-(3-chloro-phenyl-ethyl)-carbamic acid tert-butyl ester. (Yield 0.14 g, 83.6%).

### Step B

### 2-Methoxy-7-oxo-7,8-dihydro-pyrido[2,3-d]pyrimidine-6-carboxylic acid {5-[(R)-2-amino-1-(3-chloro-phenyl)-ethylcarbamoyl]-2-chloro-phenyl}-amide

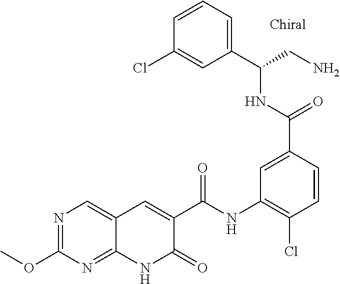

To a solution of ((R)-2-{4-chloro-3-[(2-methoxy-7-oxo-7,8-dihydro-pyrido[2,3-d]pyrimidine-6-carbonyl)-amino]-benzoylamino}-2-(3-chloro-phenyl-ethyl)-carbamic acid tert-butyl ester (0.14 g, 0.22 mmol)) in dichloromethane (8 mL) was added 2,2,2-trifluoroacetic acid (0.25 mL). The mixture was stirred at room temperature for 18 hours. The mixture was concentrated and then dissolved in methanol. The resulting solution was treated with saturated sodium carbonate solution (0.5 mL). The precipitate was collected by filtration and washed with methanol and water and dried in vacuum oven to give (R)—N-(5-(2-amino-1-(3-chlorophenyl)ethylcarbamoyl)-2-chlorophenyl)-2-methoxy-7-oxo-7,8-dihydropyrido[2,3-d]pyrimidine-6-carboxamide. (Yield 0.102 g, 86.4%).

HR-MS (ES⁺) m/z Calculated for C₂₄H₂₁Cl₂N₆O₄ ([M+H]⁺): 527.0996. Found: 527.0995.

### Example 135

### 2-Methoxy-7-oxo-7,8-dihydro-pyrido[2,3-d]pyrimidine-6-carboxylic acid {5-[(S)-2-amino-1-(3-chloro-phenyl)-ethylcarbamoyl]-2-chloro-phenyl}-amide

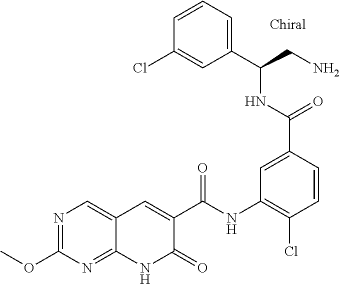

### Step A

### (S)-2-{4-Chloro-3-[(2-methoxy-7-oxo-7,8-dihydro-pyrido[2,3-d]pyrimidine-6-carbonyl)-amino]-benzoylamino}-2-(3-chloro-phenyl-ethyl)-carbamic acid tert-butyl ester

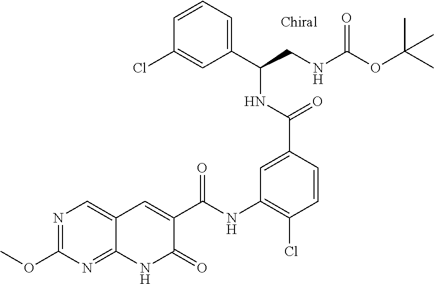

Triethylamine (0.042 mL, 0.30 mmol) was added to a mixture of 4-chloro-3-[(2-methoxy-7-oxo-7,8-dihydro-pyrido[2,3-d]pyrimidine-6-carbonyl)-amino]-benzoic acid (0.10 g, 0.27 mmol) (from Example 22 supra) and HATU (0.11 g, 0.30 mmol) (Aldrich) in DMF (4.0 mL). The mixture was stirred at room temperature for 30 minutes. [(S)-2-Amino-2-(3-chloro-phenyl)-ethyl]-carbamic acid tert-butyl ester (86.7 mg, 0.32 mmol) (from Example 13 supra) was added. Mixture was stirred for 18 hours and then partitioned between ethyl acetate and water. The precipitate was collected by filtration and washed with ethyl acetate and water and dried in vacuum oven to give (S)-2-{4-chloro-3-[(2-methoxy-7-oxo-7,8-dihydro-pyrido[2,3-d]pyrimidine-6-carbonyl)-amino]-benzoylamino}-2-(3-chloro-phenyl-ethyl)-carbamic acid tert-butyl ester. (Yield 0.13 g, 77.6%).

### Step B

### 2-Methoxy-7-oxo-7,8-dihydro-pyrido[2,3-d]pyrimidine-6-carboxylic acid {5-[(S)-2-amino-1-(3-chloro-phenyl)-ethylcarbamoyl]-2-chloro-phenyl}-amide

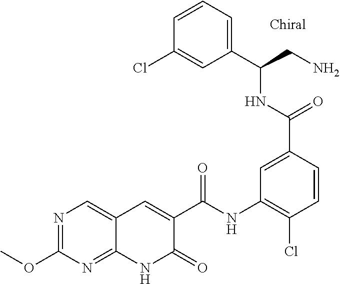

To a solution of (S)-2-{4-chloro-3-[(2-methoxy-7-oxo-7,8-dihydro-pyrido[2,3-d]pyrimidine-6-carbonyl)-amino]-benzoylamino}-2-(3-chloro-phenyl-ethyl)-carbamic acid tert-butyl ester (0.13 g, 0.21 mmol)) in dichloromethane (8 mL) was added 2,2,2-trifluoroacetic acid (0.25 mL). The mixture was stirred at room temperature for 18 hours. The mixture was concentrated and then dissolved in methanol. The resulting solution was treated with saturated sodium carbonate solution (0.5 mL). The precipitate was collected by filtration and washed with methanol and water and dried in vacuum oven to give 2-methoxy-7-oxo-7,8-dihydro-pyrido[2,3-d]pyrimidine-6-carboxylic acid {5-[(S)-2-amino-1-(3-chloro-phenyl)-ethylcarbamoyl]-2-chloro-phenyl}-amide. (Yield 0.082 g, 75.1%).

HR-MS (ES⁺) m/z Calculated for C₂₄H₂₁Cl₂N₆O₄ ([M+H]⁺): 527.0996. Found: 527.0995.

### Example 136

### 2-Methoxy-7-oxo-7,8-dihydro-pyrido[2,3-d]pyrimidine-6-carboxylic acid {5-[(S)-3-amino-1-(3-chloro-phenyl)-propylcarbamoyl]-2-chloro-phenyl}-amide

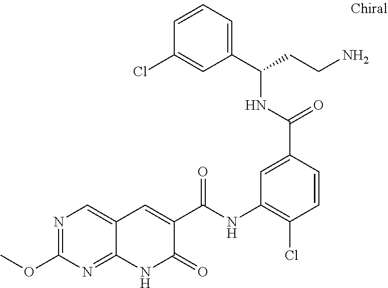

### Step A

### (S)-3-{4-Chloro-3-[(2-methoxy-7-oxo-7,8-dihydro-pyrido[2,3-d]pyrimidine-6-carbonyl)-amino]-benzoylamino}-3-(3-chloro-phenyl-propyl)-carbamic acid tert-butyl ester

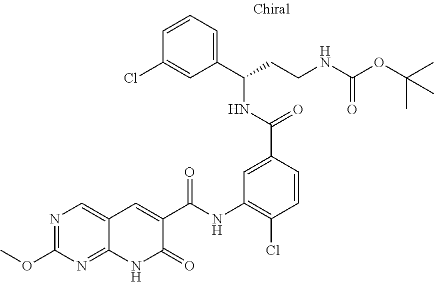

Triethylamine (0.042 mL, 0.30 mmol) was added to a mixture of 4-chloro-3-[(2-methoxy-7-oxo-7,8-dihydro-pyrido[2,3-d]pyrimidine-6-carbonyl)-amino]-benzoic acid (0.10 g, 0.27 mmol) (from Example 22 supra) and HATU (0.11 g, 0.30 mmol) (Aldrich) in DMF (4.0 mL). The mixture was stirred at room temperature for 30 minutes. [(S)-3-Amino-3-(3-chloro-phenyl)-propyl]-carbamic acid tert-butyl ester (91.2 mg, 0.32 mmol) (from Example 10 supra) was added. The mixture was stirred for 18 hours and then partitioned between ethyl acetate and water. The aqueous phase was extracted with ethyl acetate (3×). The combined organic phase was washed with water and brine, dried (magnesium sulfate) and concentrated. The residue was purified by flash chromatography eluting with 20-100% ethyl acetate in dichloromethane to give (S)-3-{4-chloro-3-[(2-methoxy-7-oxo-7,8-dihydro-pyrido[2,3-d]pyrimidine-6-carbonyl)-amino]-benzoylamino}-3-(3-chloro-phenyl-propyl)-carbamic acid tert-butyl ester. (Yield 0.15 g, 87.6%).

### Step B

### 2-Methoxy-7-oxo-7,8-dihydro-pyrido[2,3-d]pyrimidine-6-carboxylic acid {5-[(S)-3-amino-1-(3-chloro-phenyl)-propylcarbamoyl]-2-chloro-phenyl}-amide

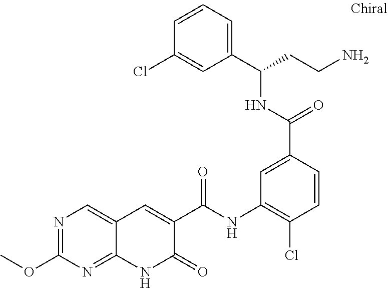

To a solution of (S)-3-{4-chloro-3-[(2-methoxy-7-oxo-7,8-dihydro-pyrido[2,3-d]pyrimidine-6-carbonyl)-amino]-benzoylamino}-3-(3-chloro-phenyl-propyl)-carbamic acid tert-butyl ester (0.15 g, 0.23 mmol)) in dichloromethane (8 mL) was added 2,2,2-trifluoroacetic acid (0.26 mL). The mixture was stirred at room temperature for 18 hours. The mixture was concentrated and then dissolved in methanol. The resulting solution was treated with saturated sodium carbonate solution (0.5 mL). The precipitate was collected by filtration and washed with methanol and water and dried in vacuum oven to give (S)—N-(5-(3-amino-1-(3-chlorophenyl)propylcarbamo-yl)-2-chlorophenyl)-2-methoxy-7-oxo-7,8-dihydropyrido[2,3-d]pyrimidine-6-carboxamide. (Yield 0.041 g, 32.4%).

HR-MS (ES⁺) m/z Calculated for C₂₅H₂₂Cl₂N₆O₄ ([M+H]⁺): 541.1153. Found: 541.1151.

### Example 137

### N-(5-(3-Amino-1-phenylpropylcarbamoyl)-2-chlorophenyl)-2-(4-aminopiperidin-1-yl)-7-oxo-7,8-dihydropyrido[2,3-d]pyrimidine-6-carboxamide hydrochloride

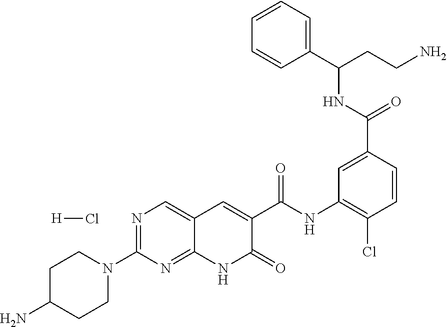

To a disposable vial with stir bar were added (1-{6-[5-(3-tert-butoxycarbonylamino-1-phenyl-propylcarbamoyl)-2-chloro-phenylcarbamoyl]-7-oxo-7,8-dihydro-pyrido[2,3-d]pyrimidin-2-yl}-piperidin-4-yl)-carbamic acid tert-butyl ester (100 mg, 129 μmol, Eq: 1.00) (from Example 129 supra), 1,4-dioxane (1.5 mL) and 4M hydrochloric acid in 1,4-dioxane (4.00 mmol, Eq: 31.0). The reaction was stirred at room temperature for 2 hours, after which it was concentrated under reduced pressure. The crude material was suspended in ethyl acetate, filtered and rinsed with ethyl acetate and diethyl ether to provide after drying N-(5-(3-amino-1-phenylpropylcarbamoyl)-2-chlorophenyl)-2-(4-aminopiperidin-1-yl)-7-oxo-7,8-dihydropyrido[2,3-d]pyrimidine-6-carboxamide hydrochloride. (Yield 71 mg, 85%).

HR-MS (ES⁺) m/z Calculated for C₂₉H₃₂ClN₈O₃ ([M+H]⁺): 575.2281. Found: 575.2279.

### Example 138

### N-(5-(2-Amino-1-phenylethylcarbamoyl)-2-chlorophenyl)-2-(4-aminopiperidin-1-yl)-7-oxo-7,8-dihydropyrido[2,3-d]pyrimidine-6-carboxamide hydrochloride

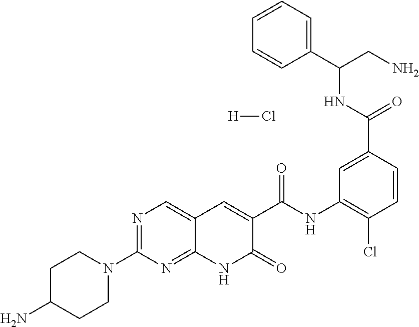

To a disposable vial with stir bar were added (1-{6-[5-(2-tert-butoxycarbonylamino-1-phenyl-ethylcarbamoyl)-2-chloro-phenylcarbamoyl]-7-oxo-7,8-dihydro-pyrido[2,3-d]pyrimidin-2-yl}-piperidin-4-yl)-carbamic acid tert-butyl ester (60 mg, 78.8 μmol, Eq: 1.00) (from Example 130 supra), 1,4-dioxane (1.5 mL) and 4M hydrochloric acid in 1,4-dioxane (4.00 mmol, Eq: 50.8). The reaction was allowed to stir at room temperature for 2 hours, after which it was concentrated under reduced pressure to remove the solvents. The crude material was suspended in ethyl acetate and solid was filtered and rinsed with ethyl acetate and diethyl ether to provide after drying N-(5-(2-amino-1-phenylethylcarbamoyl)-2-chlorophenyl)-2-(4-aminopiperidin-1-yl)-7-oxo-7,8-dihydropyrido[2,3-d]pyrimidine-6-carboxamide hydrochloride. (Yield 43 mg, 86%).

LR-MS [M+H]⁺: 561.

### Example 139

### N-(5-(2-Amino-1-phenylethylcarbamoyl)-2-chlorophenyl)-2-(2-(4-methylpiperazin-1-yl)ethylamino)-7-oxo-7,8-dihydropyrido[2,3-d]pyrimidine-6-carboxamide hydrochloride

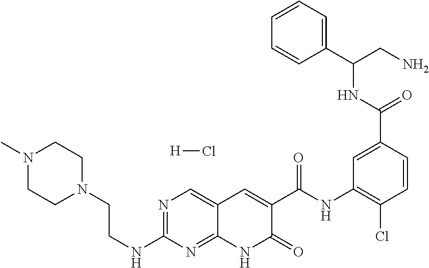

To a disposable tube with stir bar were added tert-butyl 2-(4-chloro-3-(2-(2-(4-methylpiperazin-1-yl)ethylamino)-7-oxo-7,8-dihydropyrido[2,3-d]pyrimidine-6-carboxamido)benzamido)-2-phenylethylcarbamate (53 mg, 75.3 μmol, Eq: 1.00) (from Example 131 supra), 1,4-dioxane (2 mL) and 4M hydrochloric acid in 1,4-dioxane (4.00 mmol, Eq: 53.1). The reaction was stirred at room temperature for 2 hours, after which it was concentrated under reduced pressure to remove the solvents. The crude material was suspended in ethyl acetate, filtered and rinsed with ethyl acetate and diethyl ether to provide after drying N-(5-(2-amino-1-phenylethylcarbamoyl)-2-chlorophenyl)-2-(2-(4-methylpiperazin-1-yl)ethylamino)-7-oxo-7,8-dihydropyrido[2,3-d]-pyrimidine-6-carboxamide hydrochloride. (Yield 48 mg, 89%).

HR-MS (ES⁺) m/z Calculated for C₃₀H₃₅ClN₉O₃ ([M+H]⁺): 604.2546. Found: 604.2548.

### Example 140

### N-(2-Chloro-5-(2-hydroxy-1-phenylethylcarbamoyl)phenyl)-2-(methylthio)-7-oxo-7,8-dihydropyrido[2,3-d]pyrimidine-6-carboxamide

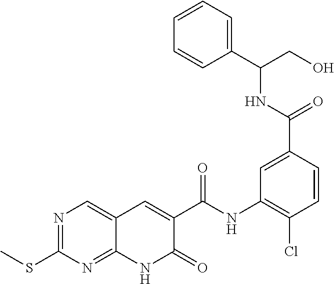

To a 25 mL round bottom flask with stir bar were added 4-chloro-3-[(2-methylsulfanyl-7-oxo-7,8-dihydro-pyrido[2,3-d]pyrimidine-6-carbonyl)-amino]-benzoic acid (390 mg, 998 μmol, Eq: 1.00) (from Example 98 supra), DMF (4.99 mL), 2-amino-2-phenylethanol (151 mg, 1.1 mmol, Eq: 1.1), triethylamine (505 mg, 4.99 mmol, Eq: 5) and HATU (455 mg, 1.2 mmol, Eq: 1.2). The reaction was allowed to stir at room temperature for 18 hours, after which it was diluted with water and a solid began to precipitate out of solution. The solid was filtered and rinsed with water, ethyl acetate and then diethyl ether to provide after drying N-(2-chloro-5-(2-hydroxy-1-phenylethylcarbamoyl)phenyl)-2-(methylthio)-7-oxo-7,8-dihydropyrido[2,3-d]pyrimidine-6-carboxamide. (Yield 350 mg, 69%).

HR-MS (ES⁺) m/z Calculated for C₂₄H₂₁ClN₅O₄S ([M+H]⁺): 510.0998. Found: 510.0996.

### Example 141

### N-(2-Chloro-5-(3-hydroxy-1-phenylpropylcarbamoyl)phenyl)-2-(methylthio)-7-oxo-7,8-dihydropyrido[2,3-d]pyrimidine-6-carboxamide

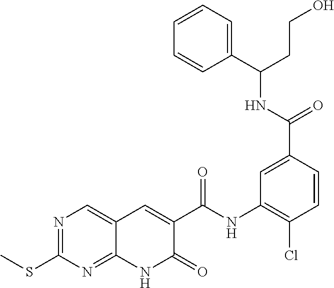

To a 25 mL pear shaped flask were added 4-chloro-3-[(2-methylsulfanyl-7-oxo-7,8-dihydro-pyrido[2,3-d]pyrimidine-6-carbonyl)-amino]-benzoic acid (390 mg, 998 μmol, Eq: 1.00) (from Example 98 supra), 3-amino-3-phenylpropan-1-ol (181 mg, 1.2 mmol, Eq: 1.2), DMF (4.99 mL), triethylamine (505 mg, 4.99 mmol, Eq: 5) and then HATU (455 mg, 1.2 mmol, Eq: 1.2). The reaction was stirred at room temperature for 18 hours, after which it was quenched with water. The resultant solid was filtered and rinsed with water, ethyl acetate and then diethyl ether, then dried in an oven vaccuum at 65° C. overnight to provide N-(2-chloro-5-(3-hydroxy-1-phenyl-propylcarbamoyl)phenyl)-2-(methylthio)-7-oxo-7,8-dihydropyrido[2,3-d]pyrimidine-6-carboxamide. (Yield 440 mg, 84%).

HR-MS (ES⁺) m/z Calculated for C₂₅H₂₂ClN₅NaO₄S ([M+Na]⁺): 546.0973. Found: 546.0971.

### Example 142

### 2-Morpholin-4-yl-7-oxo-7,8-dihydro-pyrido[2,3-d]pyrimidine-6-carboxylic acid [2-chloro-5-(2,2-dimethyl-propylcarbamoyl)-phenyl]-amide

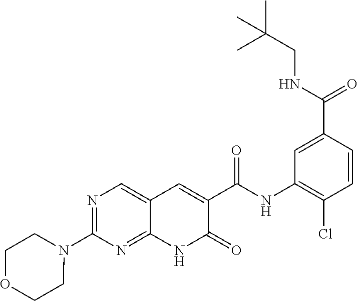

### Step A

### Ethyl 4-amino-2-morpholinopyrimidine-5-carboxylate

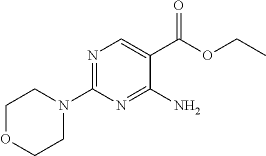

A mixture of ethyl 4-amino-2-mercaptopyrimidine-5-carboxylate (30 g, 151 mmol) and morpholine (100 mL) was heated at 90° C. overnight under argon. The mixture was then cooled and concentrated under reduced pressure. The residue was taken up in dichloromethane (300 mL), washed successively with water (200 mL), brine (150 mL), dried over anhydrous sodium sulfate, filtered and concentrated under reduced pressure to give ethyl 4-amino-2-morpholinopyrimidine-5-carboxylate as a white solid. (Yield 30 g, 78.8%).

### Step B

### (4-Amino-2-morpholinopyrimidin-5-yl)methanol

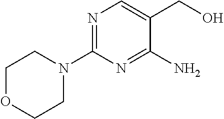

To a 2 M solution of lithium aluminum hydride in tetrahydrofuran (67 mL), cooled in an ice bath, was added ethyl 4-amino-2-morpholinopyrimidine-5-carboxylate (26 g, 103 mmol) in anhydrous tetrahydrofuran (400 mL). The mixture was stirred 60 minutes at room temperature, then cooled in an ice bath and quenched by the dropwise addition of 1 M sodium hydroxide (30 mL) and water (30 mL). The solids were removed by filtration, washing the solids with 100 mL of ethyl acetate. The filtrate was concentrated under reduced pressure to give (4-amino-2-morpholinopyrimidin-5-yl)methanol as a white solid. (Yield 19.8 g, 91.4%).

### Step C

### 4-Amino-2-morpholinopyrimidine-5-carbaldehyde

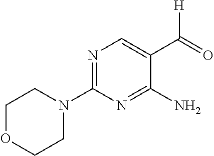

A mixture of (4-amino-2-morpholinopyrimidin-5-yl)methanol (19.8 g, 94.2 mmol), manganese dioxide (40.9 g, 471 mmol), in dichloromethane (800 mL) was stirred 24 hours under an atmosphere of nitrogen. The suspension was filtered through Celite, washing the filter cake extensively with hot acetone. The filtrate was concentrated under reduced pressure to give 4-amino-2-morpholinopyrimidine-5-carbaldehyde as a white solid. (Yield 18.5 g, 94.3%).

### Step D Methyl 2-morpholino-7-oxo-7,8-dihydropyrido[2,3-d]pyrimidine-6-carboxylate

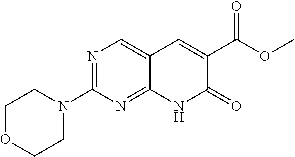

A mixture of 4-amino-2-morpholinopyrimidine-5-carbaldehyde (8 g, 38.4 mmol), dimethylmalonate (6.75 g, 51.1 mmol), sodium methoxide (2.49 g, 46.1 mmol) and methanol (187 mL) was heated at 80° C. overnight under an atmosphere of nitrogen. The mixture was cooled, acidified by the addition of acetic acid and diluted with ethyl acetate (200 mL). The precipitate was collected by suction filtration, washing with water. The filtrate was extracted twice with ethyl acetate (200 mL). The combined ethyl acetate layers were washed once with brine (200 mL), dried over anhydrous sodium sulfate, filtered and concentrated under reduced pressure to ca 100 mL volume. The resulting precipitate was collected by filtration, washing with hexanes-ethyl acetate (100 mL, 1:1). The solids were combined to give methyl 2-morpholino-7-oxo-7,8-dihydropyrido[2,3-d]pyrimidine-6-carboxylate. (Yield 5.8 g, 52%).

### Step E

### 2-Morpholino-7-oxo-7,8-dihydropyrido[2,3-d]pyrimidine-6-carboxylic acid

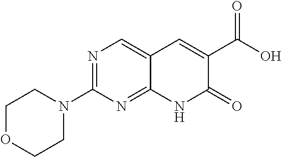

A mixture of methyl 2-morpholino-7-oxo-7,8-dihydropyrido[2,3-d]pyrimidine-6-carboxylate (4.0 g, 13.8 mmol) and 1 M sodium hydroxide (in water-methanol 1:1, 1080 mL) was heated at 75° C. overnight. The mixture was then concentrated under reduced pressure to remove the methanol. The mixture was acidified by the addition of 2M hydrochloric acid. The precipitate was collected by filtration, washing the solid with water, to give 2-morpholino-7-oxo-7,8-dihydropyrido[2,3-d]pyrimidine-6-carboxylic acid as a light yellow solid. (Yield 3.6 g, 94.4%).

### Step F

### 1H-Benzo[d][1,2,3]triazol-1-yl 3-amino-4-chlorobenzoate

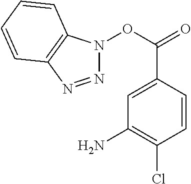

A mixture of BOP (5.16 g 11.7 mmol), 3-amino-4-chlorobenzoic acid (2 g, 11.7 mmol) (Aldrich), triethylamine (2.95 g, 29.1 mmol) and dimethylformamide (20 mL) was stirred at 0° C. for 2 hours then diluted with water (60 mL). The mixture was stirred for 20 minutes, then the solid was collected by filtration washing with water (100 mL). The solid was air dried overnight to give 1H-benzo[d][1,2,3]triazol-1-yl 3-amino-4-chlorobenzoate as an off-white solid. (Yield 2.6 g, 77%).

### Step G

### 3-Amino-4-chloro-N-neopentylbenzamide

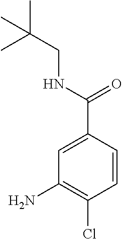

A mixture of 1H-benzo[d][1,2,3]triazol-1-yl 3-amino-4-chlorobenzoate (0.700 g, 2.42 mmol), 2,2-dimethylpropan-1-amine (0.211 g, 2.42 mmol), triethylamine (1.23 g, 12.1 mmol) and dimethylformamide (5 mL) was stirred at room temperature. After the reaction was complete, the mixture was diluted with dichloromethane (50 mL) and washed successively twice with water (50 mL) and then brine (50 mL). The dichloromethane layer was dried over anhydrous sodium sulfate, filtered and concentrated under reduced pressure. The residue was purified by chromatography on silica gel eluting with hexanes-ethyl acetate (gradient 70:30-30:70) to give 3-amino-4-chloro-N-neopentylbenzamide as a white solid. (Yield 0.420 g, 72.1%).

### Step H

### 2-Morpholin-4-yl-7-oxo-7,8-dihydro-pyrido[2,3-d]pyrimidine-6-carboxylic acid [2-chloro-5-(2,2-dimethyl-propylcarbamoyl)-phenyl]-amide

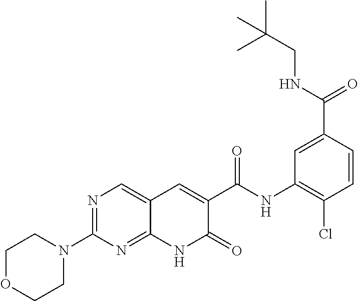

A mixture of HATU (0.248 g, 0.65 mmol), triethylamine (0.176 g, 1.74 mmol), 2-morpholino-7-oxo-7,8-dihydropyrido[2,3-d]pyrimidine-6-carboxylic acid (0.120 g, 0.434 mmol) and dimethylformamide (10 mL) was stirred at room temperature for 15 minutes, then 3-amino-4-chloro-N-neopentylbenzamide (0.125 g, 0.521 mmol) was added. The mixture was stirred overnight at room temperature, then taken up in dichloromethane (100 mL), washed successively with water (100 mL) and brine (50 mL). The dichloromethane layer was dried over anhydrous sodium sulfate, filtered and concentrated under reduced pressure. The residue was purified by chromatrography on silica gel, eluting with hexanes-ethyl acetate (1:1), to give 2-morpholin-4-yl-7-oxo-7,8-dihydro-pyrido[2,3-d]pyrimidine-6-carboxylic acid [2-chloro-5-(2,2-dimethyl-propylcarbamoyl)-phenyl]-amide as a white solid. (Yield 0.070 g, 32.3%).

HR-MS (ES⁺) m/z Calculated for C₂₄H₂₈ClN₆O₄ ([M+H]⁺): 499.1855. Found: 499.1855.

### Example 143

### 2-Morpholin-4-yl-7-oxo-7,8-dihydro-pyrido[2,3-d]pyrimidine-6-carboxylic acid {2-chloro-5-[1-(3-chloro-phenyl)-ethylcarbamoyl]-phenyl}-amide

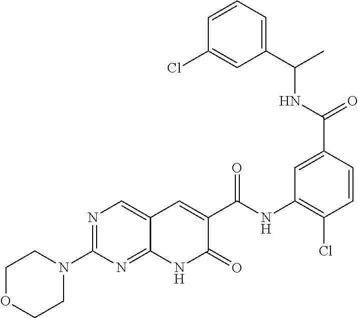

### Step A

### (rac)-3-Amino-4-chloro-N-(1-(3-chlorophenyl)ethyl)benzamide

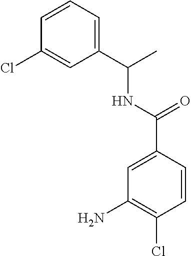

A mixture of 1H-benzo[d][1,2,3]triazol-1-yl 3-amino-4-chlorobenzoate (0.730 g, 2.53 mmol) (from Example 142 supra), (rac)-1-(3-chlorophenyl)ethanamine (0.394 g, 2.53 mmol), triethylamine (1.28 g, 12.6 mmol) and dimethylformamide (10 mL) was stirred at room temperature overnight. The mixture was diluted with dichloromethane (50 mL) and washed successively twice with water (50 mL) and then brine (50 mL). The dichloromethane layer was dried over anhydrous sodium sulfate, filtered and concentrated under reduced pressure. The residue was purified by chromatography on silica gel eluting with hexanes-ethyl acetate (gradient 70:30-30:70) to give (rac)-3-amino-4-chloro-N-(1-(3-chlorophenyl)ethyl)benzamide as a white solid. (Yield 0.600 g, 76.7%).

### Step B

### 2-Morpholin-4-yl-7-oxo-7,8-dihydro-pyrido[2,3-d]pyrimidine-6-carboxylic acid {2-chloro-5-[1-(3-chloro-phenyl)-ethylcarbamoyl]-phenyl}-amide

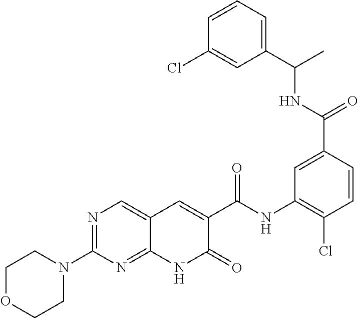

A mixture of HATU (0.303 g, 0.79 mmol), triethylamine (0.161 g, 1.59 mmol), 2-morpholino-7-oxo-7,8-dihydropyrido[2,3-d]pyrimidine-6-carboxylic acid (0.110 g, 0.398 mmol) (from Example 142 supra) and dimethylformamide (12.5 mL) was stirred at room temperature for 15 minutes, then (rac)-3-amino-4-chloro-N-(1-(3-chlorophenyl)ethyl)benzamide (0.123 g, 0.398 mmol) was added. The mixture was stirred overnight at room temperature, then taken up in dichloromethane (100 mL), washed successively with water (100 mL) and brine (50 mL). The dichloromethane layer was dried over anhydrous sodium sulfate, filtered and concentrated under reduced pressure. The residue was purified by chromatrography on silica gel, eluting with hexanes-ethyl acetate (1:1), to give (rac)-2-morpholin-4-yl-7-oxo-7,8-dihydro-pyrido[2,3-d]pyrimidine-6-carboxylic acid {2-chloro-5-[1-(3-chloro-phenyl)-ethylcarbamoyl]-phenyl}-amide as an off-white solid. (Yield 0.060 g, 26.6%).

HR-MS (ES⁺) m/z Calculated for C₂₇H₂₅Cl₂N₆O₄ ([M+H]⁺): 567.1309. Found: 567.1306.

### Example 144

### 2-Morpholin-4-yl-7-oxo-7,8-dihydro-pyrido[2,3-d]pyrimidine-6-carboxylic acid [2-chloro-5-(3-chloro-benzylcarbamoyl)-4-fluoro-phenyl]-amide

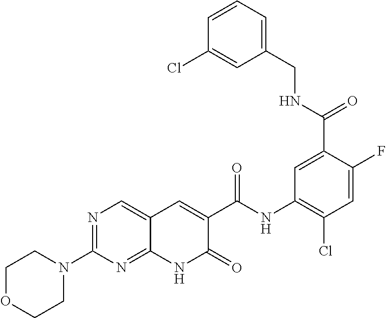

### Step A

### 1H-Benzo[d][1,2,3]triazol-1-yl 5-amino-4-chloro-2-fluorobenzoate

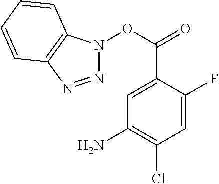

A mixture of BOP (2.33 g, 5.28 mmol), 5-amino-4-chloro-2-fluorobenzoic acid (1 g, 5.28 mmol), triethylamine (1.33 g, 13.2 mmol) and dimethylformamide (10 mL) was stirred at 0° C. for 3 hours then diluted with water (60 mL). The mixture was stirred for 20 minutes, then the solid was collected by filtration, washing with water (100 mL). The solid was air dried overnight to give 1H-benzo[d][1,2,3]triazol-1-yl 5-amino-4-chloro-2-fluorobenzoate as an off-white solid. (Yield 1.6 g, 98.8%).

### Step B

### 5-Amino-4-chloro-N-(3-chlorobenzyl)-2-fluorobenzamide

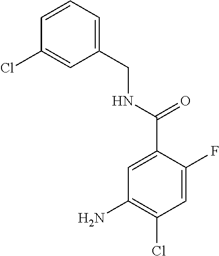

A mixture of 1H-benzo[d][1,2,3]triazol-1-yl 5-amino-4-chloro-2-fluorobenzoate (0.800 g, 2.61 mmol), (3-chlorophenyl)methanamine (0.369 g, 2.61 mmol), triethylamine (1.32 g, 13 mmol) and dimethylformamide (5.71 mL) was stirred at room temperature overnight. The mixture was diluted with dichloromethane (50 mL) and washed successively twice with water (50 mL) and then brine (50 mL). The dichloromethane layer was dried over anhydrous sodium sulfate, filtered and concentrated under reduced pressure. The residue was purified by chromatography on silica gel eluting with hexanes-ethyl acetate (gradient 70:30-30:70) to give 5-amino-4-chloro-N-(3-chlorobenzyl)-2-fluorobenzamide as a white solid. (Yield 0.260 g, 31.8%).

### Step C

### 2-Morpholin-4-yl-7-oxo-7,8-dihydro-pyrido[2,3-d]pyrimidine-6-carboxylic acid [2-chloro-5-(3-chloro-benzylcarbamoyl)-4-fluoro-phenyl]-amide

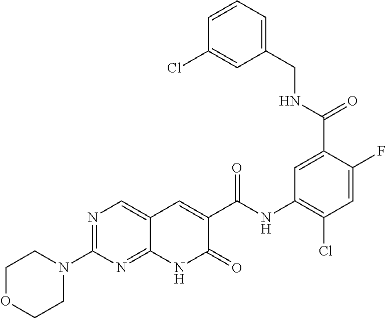

A mixture of (0.656 g, 1.72 mmol) HATU (0.656 g, 1.72 mmol), triethylamine (0.233 g, 2.3 mmol), 2-morpholino-7-oxo-7,8-dihydropyrido[2,3-d]pyrimidine-6-carboxylic acid (0.318 g, 1.15 mmol) (from Example 142 supra) and dimethylformamide (20.5 mL) was stirred at room temperature for 15 minutes, then 5-amino-4-chloro-N-(3-chlorobenzyl)-2-fluorobenzamide (0.180 g, 0.580 mmol) was added. The mixture was stirred overnight at room temperature, then taken up in (100 mL) dichloromethane (100 mL), washed successively with water (100 mL) and brine (50 mL). The dichloromethane layer was dried over anhydrous sodium sulfate, filtered and concentrated under reduced pressure. The residue was purified by chromatrography on silica gel, eluting with hexanes-ethyl acetate (1:1), to give 2-morpholin-4-yl-7-oxo-7,8-dihydro-pyrido[2,3-d]pyrimidine-6-carboxylic acid [2-chloro-5-(3-chloro-benzylcarbamoyl)-4-fluoro-phenyl]-amide as a light yellow solid. (Yield 0.080 g, 44%).

LR-MS [M+H]⁺: 571.

### Example 145

### tert-Butyl 1-(6-(2-chloro-5-(2-hydroxy-1-phenylethylcarbamoyl)phenylcarbamoyl)-7-oxo-7,8-dihydropyrido[2,3-d]pyrimidin-2-yl)piperidin-4-ylcarbamate

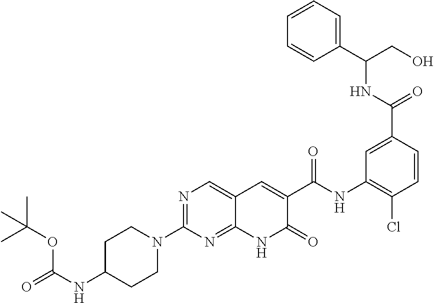

### Step A

### N-(2-Chloro-5-(2-hydroxy-1-phenylethylcarbamoyl)phenyl)-2-(methylsulfonyl)-7-oxo-7,8-dihydropyrido[2,3-d]pyrimidine-6-carboxamide

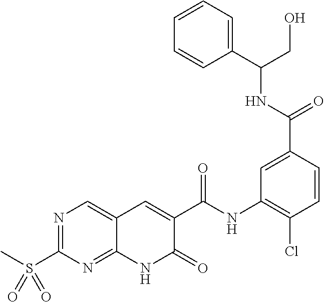

To a 25 mL round bottom flask with stir bar were added N-(2-chloro-5-(2-hydroxy-1-phenylethyl-carbamoyl)phenyl)-2-(methylthio)-7-oxo-7,8-dihydropyrido[2,3-d]pyrimidine-6-carboxamide (300 mg, 588 μmol, Eq: 1.00) (from Example 140 supra), water (7 mL), methanol (8 mL), THF (7.00 mL) and then Oxone (904 mg, 1.47 mmol, Eq: 2.5). The mixture was stirred at room temperature. After 2 hours, another equivalent of oxone was added and the reaction was allowed to stir at room temperature for 96 hours. The solvent was partially concentrated and the solid was filtered off, rinsed with water and then Et₂O. The solid was then dried in a vacuum oven overnight at 65° C. to provide N-(2-chloro-5-(2-hydroxy-1-phenylethylcarbamoyl)phenyl)-2-(methylsulfonyl)-7-oxo-7,8-dihydropyrido[2,3-d]pyrimidine-6-carboxamide as a solid. (Yield 227 mg, 71%).

### Step B

### tert-Butyl 1-(6-(2-chloro-5-(2-hydroxy-1-phenylethylcarbamoyl)phenylcarbamoyl)-7-oxo-7,8-dihydropyrido[2,3-d]pyrimidin-2-yl)piperidin-4-ylcarbamate

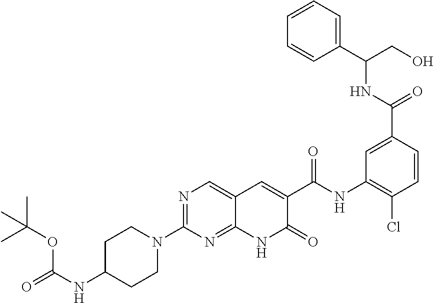

To a disposable tube with stir bar were added N-(2-chloro-5-(2-hydroxy-1-phenylethylcarb-amoyl)phenyl)-2-(methylsulfonyl)-7-oxo-7,8-dihydropyrido[2,3-d]pyrimidine-6-carboxamide (100 mg, 185 μmol, Eq: 1.00), tert-butyl piperidin-4-ylcarbamate (370 mg, 1.85 mmol, Eq: 10) and then DMF (1 mL). The mixture was heated at 65° C. in a pre-heated oil bath for 1.5 hours, after which it was cooled and then diluted with water. The resultant solid was collected by filtration and rinsed with water, diethyl ether, ethyl acetate, water and then diethyl ether. The solid was dried in an vacuum oven overnight at 65° C. to provide tert-butyl 1-(6-(2-chloro-5-(2-hydroxy-1-phenylethylcarbamoyl)phenylcarbamoyl)-7-oxo-7,8-dihydropyrido-[2,3-d]pyrimidin-2-yl)piperidin-4-ylcarbamate. (Yield 116 mg, 95%).

LR-MS [M−H]⁺ 660.

### Example 146

### tert-Butyl 1-(6-(2-chloro-5-(3-hydroxy-1-phenylpropylcarbamoyl)phenylcarbamoyl)-7-oxo-7,8-dihydropyrido[2,3-d]pyrimidin-2-yl)piperidin-4-ylcarbamate

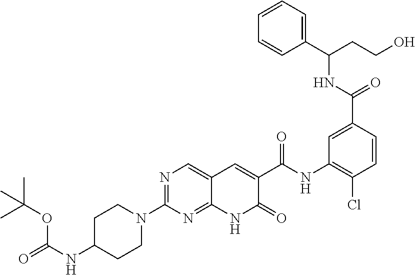

### Step A

### N-(2-Chloro-5-(3-hydroxy-1-phenylpropylcarbamoyl)phenyl)-2-(methylsulfonyl)-7-oxo-7,8-dihydropyrido[2,3-d]pyrimidine-6-carboxamide

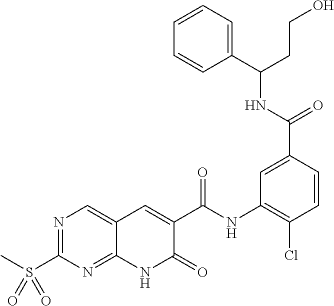

To a 25 mL round bottom flask with stir bar were added N-(2-chloro-5-(3-hydroxy-1-phenylpropylcarbamoyl)phenyl)-2-(methylthio)-7-oxo-7,8-dihydropyrido[2,3-d]pyrimidine-6-carboxamide (400 mg, 763 μmol, Eq: 1.00) (from Example 141 supra), MeOH (7 mL), THF (7.00 mL), water (7.00 mL) and then Oxone (1.17 g, 1.91 mmol, Eq: 2.5). The mixture was stirred at room temperature. After 2 hours, more water and another 2.5 equivalents of oxone were added. The reaction was allowed to stir at room temperature for 96 hours, after which a solid was filtered off, rinsed with sufficient water and then Et₂O. The solid was dried in a vacuum oven at 65° C. providing N-(2-chloro-5-(3-hydroxy-1-phenylpropylcarbamoyl)phenyl)-2-(methyl-sulfonyl)-7-oxo-7,8-dihydropyrido[2,3-d]pyrimidine-6-carboxamide. (Yield 270 mg, 64%).

### Step B

### tert-Butyl 1-(6-(2-chloro-5-(3-hydroxy-1-phenylpropylcarbamoyl)phenylcarbamoyl)-7-oxo-7,8-dihydropyrido[2,3-d]pyrimidin-2-yl)piperidin-4-ylcarbamate

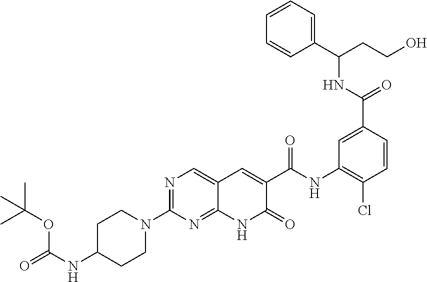

To a disposable tube with stir bar were added N-(2-chloro-5-(3-hydroxy-1-phenylpropylcarb-amoyl)phenyl)-2-(methylsulfonyl)-7-oxo-7,8-dihydropyrido[2,3-d]pyrimidine-6-carboxamide (100 mg, 180 μmol, Eq: 1.00), tert-butyl piperidin-4-ylcarbamate (360 mg, 1.8 mmol, Eq: 10) and DMF (1 mL). The mixture was heated at 65° C. in a pre-heated oil bath for 1.5 hours, after which, it was diluted with water. The solid was then filtered off and rinsed with water, ethyl acetate, diethyl ether, water and then diethyl ether. The solid was dried in vacuum oven overnight at 65° C. to provide tert-butyl 1-(6-(2-chloro-5-(3-hydroxy-1-phenylpropyl-carbamoyl)phenylcarbamoyl)-7-oxo-7,8-dihydropyrido[2,3-d]pyrimidin-2-yl)piperidin-4-ylcarbamate. (Yield 118 mg, 97%). LR-MS [M+H]⁺: 676.

### Example 147

### 2-(4-Aminopiperidin-1-yl)-N-(2-chloro-5-(2-hydroxy-1-phenylethylcarbamoyl)phenyl)-7-oxo-7,8-dihydropyrido[2,3-d]pyrimidine-6-carboxamide hydrochloride

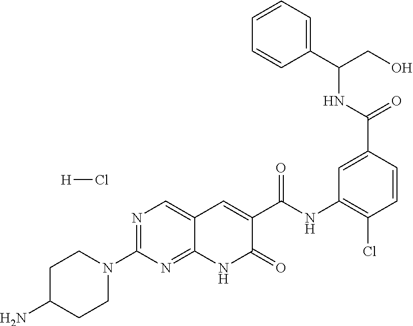

To a disposable vial with stir bar were added tert-butyl 1-(6-(2-chloro-5-(2-hydroxy-1-phenylethylcarbamoyl)phenylcarbamoyl)-7-oxo-7,8-dihydropyrido[2,3-d]pyrimidin-2-yl)piperidin-4-ylcarbamate (85 mg, 128 μmol, Eq: 1.00) (from Example 145 supra), 1,4-dioxane (1 mL) and then 4M hydrochloric acid in 1,4-dioxane (1 mL, 4.00 mmol, Eq: 31.2). The reaction was allowed to stir at room temperature for 17 hours, after which it was concentrated to remove solvents to provide 2-(4-aminopiperidin-1-yl)-N-(2-chloro-5-(2-hydroxy-1-phenylethyl-carbamoyl)phenyl)-7-oxo-7,8-dihydropyrido[2,3-d]pyrimidine-6-carboxamide hydrochloride. (Yield 65 mg, 84%). HR-MS (ES⁺) m/z Calculated for C₂₈H₂₉ClN₇O₄ ([M+H]⁺): 562.1964. Found: 562.1961.

### Example 148

### 2-(4-Aminopiperidin-1-yl)-N-(2-chloro-5-(3-hydroxy-1-phenylpropylcarbamoyl)phenyl)-7-oxo-7,8-dihydropyrido[2,3-d]pyrimidine-6-carboxamide hydrochloride

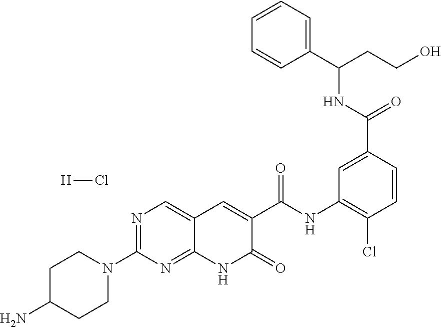

To a disposable tube with stir bar were added tert-butyl 1-(6-(2-chloro-5-(3-hydroxy-1-phenylpropylcarbamoyl)phenylcarbamoyl)-7-oxo-7,8-dihydropyrido[2,3-d]pyrimidin-2-yl)piperidin-4-ylcarbamate (85 mg, 126 μmol, Eq: 1.00) (from Example 146 supra), 1,4-dioxane (1 mL) and 4M hydrochloric acid in 1,4-dioxane (1 mL, 4.00 mmol, Eq: 31.8). The reaction was allowed to stir at room temperature overnight, after which it was concentrated under reduced pressure to remove solvents. The crude material was dissolved in water and MeOH and concentrated to dryness, providing 2-(4-aminopiperidin-1-yl)-N-(2-chloro-5-(3-hydroxy-1-phenylpropylcarbamoyl)phenyl)-7-oxo-7,8-dihydropyrido[2,3-d]pyrimidine-6-carboxamide hydrochloride. (Yield 68 mg, 88%).

HR-MS (ES⁺) m/z Calculated for C₂₉H₃₁ClN₇O₄ ([M+H]⁺): 576.2121. Found: 576.2123.

### Example 149

### tert-Butyl 2-(4-chloro-3-(7-oxo-7,8-dihydropyrido[2,3-d]pyrimidine-6-carboxamido)benzamido)-2-phenylethylcarbamate

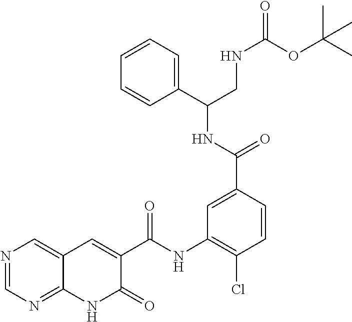

### Step A

### 4-Aminopyrimidine-5-carbonitrile

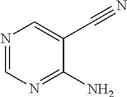

Aminomethaniminium acetate (4.37 g, 41.9 mmol) and malononitrile (1.385 g, 21.0 mmol) were suspended in methanol (5.0 mL). Sodium methoxide (0.5 M in methanol; 84 mL, 42.0 mmol) was added dropwise over 1 hour. Solid went into solution as methoxide was added and reaction was stirred at room temperature. After stirring overnight a large amount of solid had precipitated out of solution. The mixture was concentrated to about half of the original and then stirring continued at room temperature for another 24 hours. The precipitated solid was collected by filtration, washed with cold methanol and then air-dried with suction under house vacuum to give 4-aminopyrimidine-5-carbonitrile. (Yield 1.59 g, 11.9 mmol, 56.8%).

### Step B

### 4-Aminopyrimidine-5-carbaldehyde

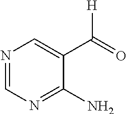

4-Aminopyrimidine-5-carbonitrile (0.945 g, 7.87 mmol) was suspended in water (9.50 mL) and mixture was chilled briefly. Sulfuric acid (1.90 mL) was added. Solid went into solution, resulting in a clear yellow solution. Palladium on carbon (10%; 143 mg, 134 μmol) was added and solution was hydrogenated (RT; 18-30 psi) for 90 minutes. The mixture was filtered through a bed of Celite to remove the catalyst. The clear, pale yellow filtrate was treated with concentrated ammonium hydroxide to neutralize the acid. After chilling, the solid was collected, washed with cold water and air-dried with suction under house vacuum to give 4-amino-pyrimidine-5-carbaldehyde. (Yield 0.654 g, 5.31 mmol, 67.5%).

### Step C

### Ethyl 7-oxo-7,8-dihydropyrido[2,3-d]pyrimidine-6-carboxylate

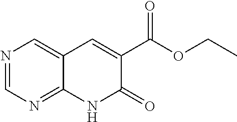

4-Aminopyrimidine-5-carbaldehyde (0.257 g, 2.09 mmol) was dissolved in DMF (9.1 mL). Diethyl malonate (670 mg, 4.18 mmol), potassium carbonate (430 mg, 3.11 mmol) and triethylamine (211 mg, 2.08 mmol) were added and the reaction mixture was heated at 85° C. After 30 minutes solid began to precipitate out of solution. The thick mixture was cooled to room temperature after 14 hours and diluted with EtOAc. The solid was collected and washed with EtOAc to give ethyl 7-oxo-7,8-dihydropyrido[2,3-d]pyrimidine-6-carboxylate. (Yield 910 mg, (Purity about 30%), 1.25 mmol, 59.7%). The material contained inorganic salt impurities but was used as is in the next step.

### Step D

### 7-Oxo-7,8-dihydropyrido[2,3-d]pyrimidine-6-carboxylic acid

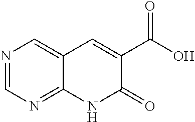

Ethyl 7-oxo-7,8-dihydropyrido[2,3-d]pyrimidine-6-carboxylate (0.73 g [estimated to be 30% pure], 0.999 mmol) was suspended in methanol (4.0 mL)/water (2.0 mL). Lithium hydroxide (65.2 mg, 2.72 mmol) was added. Clear solution was not seen and more solid began to precipitate out of solution. The mixture was heated in an oil bath at 65° C. for 3 hours and then removed from the bath and concentrated. The residue was suspended in water and the mixture was acidified with 6N HCl. The solid was collected, washed with water and dried to give 7-oxo-7,8-dihydropyrido[2,3-d]pyrimidine-6-carboxylic acid. (Yield 225 mg, 1.18 mmol, 118%; the yield was greater than theoretical indicating the purity of the starting material was likely greater than the estimated 30%).

### Step E

### Methyl 4-chloro-3-(7-oxo-7,8-dihydropyrido[2,3-d]pyrimidine-6-carboxamido)benzoate

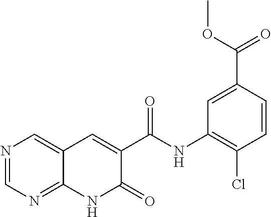

7-Oxo-7,8-dihydropyrido[2,3-d]pyrimidine-6-carboxylic acid (0.275 g, 1.44 mmol) and methyl 3-amino-4-chlorobenzoate (401 mg, 2.16 mmol) was suspended in DMF (4.5 mL). Triethylamine (728 mg, 7.19 mmol) was added, resulting in a clear, pale yellow solution. HATU (821 mg, 2.16 mmol) was then added. Solid began to precipitate out of solution again after 30 minutes. After stirring at room temperature for 6 hours the thick mixture was diluted with EtOAc. The solid was collected and dried to give methyl 4-chloro-3-(7-oxo-7,8-dihydropyrido[2,3-d]pyrimidine-6-carboxamido)benzoate. (Yield 0.295 g, 822 μmol, 57.2%).

### Step F

### 4-Chloro-3-(7-oxo-7,8-dihydropyrido[2,3-d]pyrimidine-6-carboxamido)benzoic acid

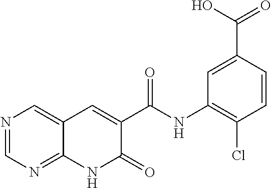

Methyl 4-chloro-3-(7-oxo-7,8-dihydropyrido[2,3-d]pyrimidine-6-carboxamido)benzoate (0.309 g, 861 μmol) was suspended in tetrahydrofuran (3.0 mL), methanol (3.0 mL) and water (1.5 mL). Lithium hydroxide (51.6 mg, 2.15 mmol) was added and reaction was heated in an oil bath at 60° C. The solid initially went into solution and then a new solid began to precipitate out of solution. The reaction was diluted with additional THF-MeOH-water (1:1:0.5) (2.50 mL) to improve stirring. The reaction was concentrated to near dryness after 2 hours. The residue was diluted with water and treated with 6N HCl. The product precipitated out as a gelatinous solid. EtOAC was added and the two-phased system was stirred for 15-20 minutes. The material changed in consistency and was then collected by filtration, washed with water and dried under house vacuum to give 4-chloro-3-(7-oxo-7,8-dihydropyrido[2,3-d]pyrimidine-6-carboxamido)-benzoic acid. (Yield 293 mg, 850 μmol, 98.7%).

### Step G

### tert-Butyl 2-(4-chloro-3-(7-oxo-7,8-dihydropyrido[2,3-d]pyrimidine-6-carboxamido)-benzamido)-2-phenylethylcarbamate

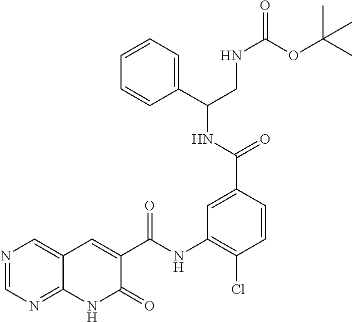

4-Chloro-3-(7-oxo-7,8-dihydropyrido[2,3-d]pyrimidine-6-carboxamido)benzoic acid (129.0 mg, 374 μmol), tert-butyl 2-amino-2-phenylethylcarbamate (97.3 mg, 412 μmol) (from Example 4 supra) and triethylamine (189 mg, 261 μl, 1.87 mmol) were combined in DMF (1.9 mL). HATU (164 mg, 430 μmol) was added and the mixture was stirred at room temperature for 4 hours. The reaction mixture was then diluted with water (˜7 mL). Material precipitated out of solution as a gelatinous emulsion that would not filter. EtOAc was added and the two-phased mixture was stirred until the material became filterable. The solid was collected, washed with water and EtOAc and dried under house vacuum to give tert-butyl 2-(4-chloro-3-(7-oxo-7,8-dihydropyrido[2,3-d]pyrimidine-6-carboxamido)benzamido)-2-phenylethylcarbamate. (Yield 144.8 mg, 231 μmol, 61.9%).

HR-MS (ES⁺) m/z Calculated for C₂₈H₂₈ClN₆O₅ ([M+H]⁺): 563.1804. Found: 563.1802.

### Example 150

### tert-Butyl 3-(4-chloro-3-(7-oxo-7,8-dihydropyrido[2,3-d]pyrimidine-6-carboxamido)benzamido)-3-phenylpropylcarbamate

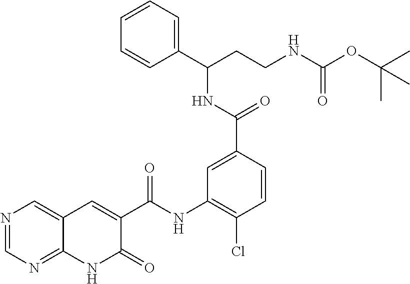

4-Chloro-3-(7-oxo-7,8-dihydropyrido[2,3-d]pyrimidine-6-carboxamido)benzoic acid (159.8 mg, 464 μmol) (from Example 149 supra) and tert-butyl 3-amino-3-phenyl-propylcarbamate (128 mg, 510 μmol) (from Example 6 supra) were combined in DMF (2.32 mL). Material remained insoluble. Triethylamine was added (232 mg, 320 μl, 2.3 mmol) and most of the material went into solution. HATU (203 mg, 533 μmol) was then added and mixture was stirred at room temperature for 4 hours. The reaction was diluted with EtOAc and water. The product initially oiled out of solution and then went to a solid after additional stirring. The solid was collected by filtration and washed with water and EtOAc to give tert-butyl 3-(4-chloro-3-(7-oxo-7,8-dihydropyrido[2,3-d]pyrimidine-6-carboxamido)benzamido)-3-phenylpropylcarbamate. (Yield 215.8 mg, 374 μmol, 80.7%).

HR-MS (ES⁺) m/z Calculated for C₂₉H₃₀ClN₆O₅ ([M+H]⁺): 577.1961. Found: 577.1959.

### Example 151

### N-(5-(2-Amino-1-phenylethylcarbamoyl)-2-chlorophenyl)-7-oxo-7,8-dihydropyrido[2,3-d]pyrimidine-6-carboxamide hydrochloride

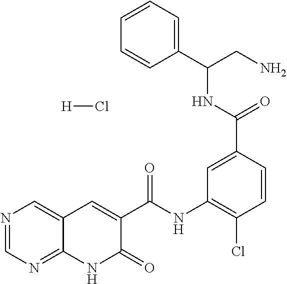

tert-Butyl 2-(4-chloro-3-(7-oxo-7,8-dihydropyrido[2,3-d]pyrimidine-6-carboxamido)-benzamido)-2-phenylethylcarbamate (130.1 mg, 231 μmol) (from Example 149 supra) was suspended in dioxane (2.30 mL). A solution of hydrogen chloride in dioxane (4.0M, 2.76 g, 2.30 mL, 9.2 mmol) was added dropwise. The pyrido-pyrimidine was seen to be going into solution, but a clear solution was not obtained before the product began to precipitate out of solution. The reaction mixture was concentrated after stirring at room temperature for 2 hours to a smaller volume and was then diluted with EtOAc and stirred 15-20 minutes. The solid was collected by filtration, washed with EtOAc and dried briefly under suction. This solid was dissolved in water, filtered to remove any insoluble material/particles and freeze-dried to give N-(5-(2-amino-1-phenylethylcarbamoyl)-2-chlorophenyl)-7-oxo-7,8-dihydropyrido[2,3-d]pyrimidine-6-carboxamide hydrochloride. (Yield 101.5 mg, 203 μmol, 88.0%).

HR-MS (ES⁺) m/z Calculated for C₂₃H₂₀ClN₆O₃ ([M+H]⁺): 463.1280. Found: 463.1278.

### Example 152

### N-(5-(3-Amino-1-phenylpropylcarbamoyl)-2-chlorophenyl)-7-oxo-7,8-dihydropyrido[2,3-d]pyrimidine-6-carboxamide hydrochloride

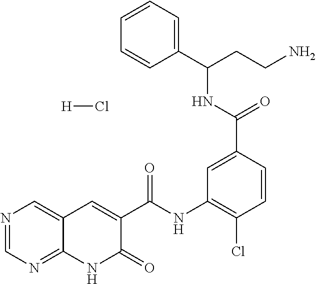

tert-Butyl 3-(4-chloro-3-(7-oxo-7,8-dihydropyrido[2,3-d]pyrimidine-6-carboxamido)-benzamido)-3-phenylpropylcarbamate (197.3 mg, 325 μmol) (from Example 150 supra) was suspended in dioxane (3.25 mL). A solution of hydrogen chloride in dioxane (4.0M, 3.25 mL, 13.0 mmol) was added in a slow stream. A clear yellow solution resulted briefly before the product began to precipitate out of solution. After stirring at room temperature for 2 hours the reaction mixture was concentrated to a smaller volume, diluted with EtOAc and stirred about 5 minutes. The solid was collected by filtration and washed with EtOAc. This material was dissolved in water and filtered through a cotton plug to remove any insoluble material and freeze-dried to give N-(5-(3-amino-1-phenylpropylcarbamoyl)-2-chlorophenyl)-7-oxo-7,8-dihydropyrido[2,3-d]pyrimidine-6-carboxamide hydrochloride. (Yield 171.9 mg, 318 μmol, 97.9%). HR-MS (ES⁺) m/z Calculated for C₂₄H₂₂ClN₆O₃ ([M+H]⁺): 477.1437. Found: 477.1434.

### Example 153

### N-(2-Chloro-5-(2-hydroxy-1-phenylethylcarbamoyl)phenyl)-2-(2-hydroxy-2-methylpropylamino)-7-oxo-7,8-dihydropyrido[2,3-d]pyrimidine-6-carboxamide

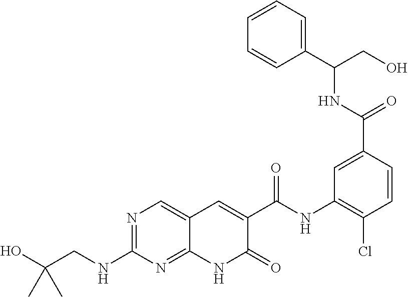

To a disposable tube with stir bar were added N-(2-chloro-5-(2-hydroxy-1-phenylethylcarb-amoyl)phenyl)-2-(methylsulfonyl)-7-oxo-7,8-dihydropyrido[2,3-d]pyrimidine-6-carboxamide (50 mg, 92.3 μmol, Eq: 1.00) (from Example 145 supra), DMF (0.5 mL) and 1-amino-2-methylpropan-2-ol (82.2 mg, 923 μmol, Eq: 10). The reaction was heated at 80° C. in a pre-heated oil bath for 3 hours, after which it was cooled to room temperature and then quenched with water. The precipitated solid was filtered off and rinsed with water, diethyl ether, ethyl acetate, diethyl ether, water and then diethyl ether. The solid was dried in vacuum oven overnight at 65° C., providing N-(2-chloro-5-(2-hydroxy-1-phenylethylcarbamoyl)phenyl)-2-(2-hydroxy-2-methylpropylamino)-7-oxo-7,8-dihydropyrido[2,3-d]pyrimidine-6-carboxamide. (Yield 42 mg, 83%). HR-MS (ES⁺) m/z Calculated for C₂₇H₂₈ClN₆O₅ ([M+H]⁺): δ 551.1804. Found: 551.1804.

### Example 154

### N-(2-Chloro-5-(3-hydroxy-1-phenylpropylcarbamoyl)phenyl)-2-(2-hydroxy-2-methylpropylamino)-7-oxo-7,8-dihydropyrido[2,3-d]pyrimidine-6-carboxamide

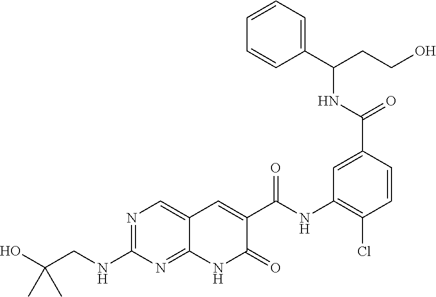

To a disposable tube with stir bar were added N-(2-chloro-5-(3-hydroxy-1-phenylpropylcarb-amoyl)phenyl)-2-(methylsulfonyl)-7-oxo-7,8-dihydropyrido[2,3-d]pyrimidine-6-carboxamide (50 mg, 89.9 μmol, Eq: 1.00) (from Example 146 supra), DMF (0.5 mL) and 1-amino-2-methylpropan-2-ol (80.2 mg, 899 μmol, Eq: 10) and the reaction was heated at 80° C. in a pre-heated oil bath for 3 hours, after which it was cooled to room temperature. The reaction was quenched with water and the precipitated solid was filtered off and rinsed with water, diethyl ether, water and then diethyl ether. The solid was dried in a vacuum oven overnight at 65° C., providing N-(2-chloro-5-(3-hydroxy-1-phenylpropylcarbamoyl)phenyl)-2-(2-hydroxy-2-methylpropylamino)-7-oxo-7,8-dihydropyrido[2,3-d]pyrimidine-6-carboxamide. (Yield 36 mg, 71%). HR-MS (ES⁺) m/z

Calculated for C₂₈H₃₀ClN₆O₅ ([M+H]⁺): 565.1961. Found: 565.1961.

### Example 155

### N-(2-Chloro-5-(3-chlorobenzylcarbamoyl)phenyl)-2-(dimethylamino)-7-oxo-7,8-dihydropyrido[2,3-d]pyrimidine-6-carboxamide

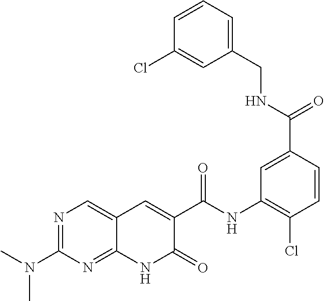

To a disposable tube with stir bar was added 2-methanesulfonyl-7-oxo-7,8-dihydro-pyrido[2,3-d]pyrimidine-6-carboxylic acid [2-chloro-5-(3-chloro-benzylcarbamoyl)-phenyl]-amide (100 mg, 183 μmol, Eq: 1.00) (from Example 75 supra), dimethylamine hydrochloride (500 mg, 6.13 mmol, Eq: 33.5), N,N-diisopropylethylamine (224 mg, 300 μL, 1.73 mmol, Eq: 9.47), and DMF (0.5 mL). The reaction was heated at 80° C. in a pre-heated oil bath for 2 hours, after which it was cooled to room temperature. The precipitated solid was filtered off and then rinsed with water, diethyl ether, ethyl acetate, water and then diethyl ether. The solid was dried in vacuum oven overnight at 65° C., providing N-(2-chloro-5-(3-chlorobenzylcarbamoyl)phenyl)-2-(dimethylamino)-7-oxo-7,8-dihydropyrido[2,3-d]pyrimidine-6-carboxamide. (Yield 79 mg, 84%). HR-MS (ES⁺) m/z Calculated for C₂₄H₂₁Cl₂N₆O₃ ([M+H]⁺): 511.1047. Found: 511.1047.

### Example 156

### 2-Morpholin-4-yl-7-oxo-7,8-dihydro-pyrido[2,3-d]pyrimidine-6-carboxylic acid [2-chloro-5-((R)-1-phenyl-ethylcarbamoyl)-phenyl]-amide

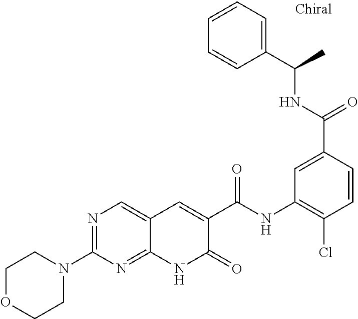

### Step A

### 3-Amino-4-chloro-N—((R)-1-phenyl-ethyl)-benzamide

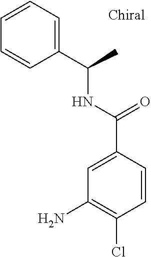

A mixture of 1H-benzo[d][1,2,3]triazol-1-yl 3-amino-4-chlorobenzoate (1.43 g, 4.95 mmol) (from Example 142 supra), (R)-1-phenylethanamine (0.5 g, 4.13 mmol), triethylamine (2.09 g, 20.6 mmol) and dimethylformamide (10 mL) was stirred overnight at room temperature. The mixture was then diluted with dichloromethane (50 mL) and washed twice with water (50 mL), once with brine (50 mL). The dichloromethane layer was dried over anhydrous sodium sulfate, filtered and concentrated under reduced pressure. The residue was purified by chromatography on silica gel eluting with hexanes-ethyl acetate (gradient 70:30-30:70) to give 3-amino-4-chloro-N—((R)-1-phenyl-ethyl)-benzamide as a white solid. (Yield 1.05 g, 92.5%).

### Step B

### N-[2-Chloro-5-((R)-1-phenyl-ethylcarbamoyl)-phenyl]-malonamic acid methyl ester

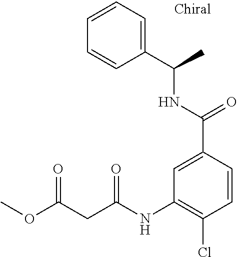

To an ice bath cooled mixture of 3-amino-4-chloro-N—((R)-1-phenyl-ethyl)-benzamide (1.05 g, 3.82 mmol), ethyl diisopropylamine (0.494 g, 3.82 mmol) and dichloromethane (60 mL) was added methyl 3-chloro-3-oxopropanoate (0.522 g, 3.82 mmol). The cooling bath was removed and the mixture stirred at room temperature overnight. The mixture was washed twice with water (50 mL) and once with brine (50 mL). The dichloromethane layer was dried over anhydrous sodium sulfate, filtered and concentrated under reduced pressure. The residue was purified by chromatography on silica gel eluting with hexanes-ethyl acetate (gradient 70:30-30:70) to give N-[2-chloro-5-((R)-1-phenyl-ethylcarbamoyl)-phenyl]-malonamic acid methyl ester as a white solid. (Yield 0.8 g, 51.2%).

### Step C

### 2-Morpholin-4-yl-7-oxo-7,8-dihydro-pyrido[2,3-d]pyrimidine-6-carboxylic acid [2-chloro-5-((R)-1-phenyl-ethylcarbamoyl)-phenyl]-amide

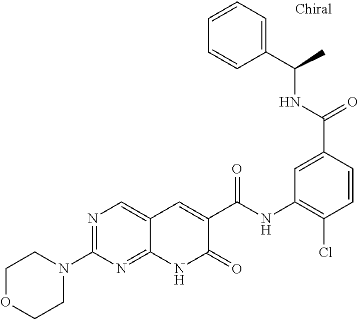

A mixture of 4-amino-2-morpholinopyrimidine-5-carbaldehyde (0.111 g, 0.534 mmol) (from Example 142 supra), N-[2-chloro-5-((R)-1-phenyl-ethylcarbamoyl)-phenyl]-malonamic acid methyl ester (0.20 g, 0.534 mmol), piperidine (0.0423 g, 0.497 mmol) and methanol (30 mL) was heated in a sealed tube at 85° C. overnight. The mixture was cooled and the solid was collected by suction filtration to give 2-morpholin-4-yl-7-oxo-7,8-dihydro-pyrido[2,3-d]pyrimidine-6-carboxylic acid [2-chloro-5-((R)-1-phenyl-ethylcarbamoyl)-phenyl]-amide as a white solid. (Yield 0.15 g, 52.7%). LC-MS: [M+H]⁺ 533.0.

### Example 157

### 2-Morpholin-4-yl-7-oxo-7,8-dihydro-pyrido[2,3-d]pyrimidine-6-carboxylic acid [2-chloro-5-((S)-1-phenyl-ethylcarbamoyl)-phenyl]-amide

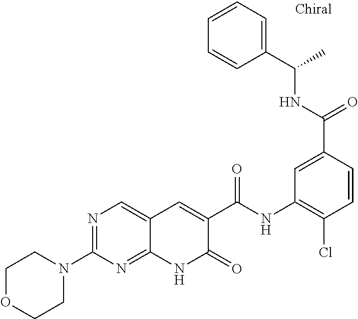

### Step A

### 3-Amino-4-chloro-N—((S)-1-phenyl-ethyl)-benzamide

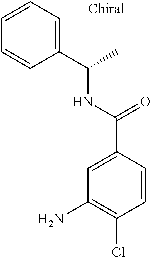

A mixture of 1H-benzo[d][1,2,3]triazol-1-yl 3-amino-4-chlorobenzoate (1.19 g, 4.13 mmol) (from Example 142 supra), (S)-1-phenylethanamine (0.5 g, 4.13 mmol), ethyldiisopropyl-amine (1.07 g, 8.25 mmol) and dimethylformamide (10 mL) was stirred overnight at room temperature. The mixture was then diluted with dichloromethane (50 mL) and washed twice with water (50 mL), once with brine (50 mL). The dichloromethane layer was dried over anhydrous sodium sulfate, filtered and concentrated under reduced pressure. The residue was purified by chromatography on silica gel eluting with hexanes-ethyl acetate (gradient 70:30-30:70) to give 3-amino-4-chloro-N—((S)-1-phenyl-ethyl)-benzamide as a white solid. (Yield 1.0 g, 88.1%).

### Step B

### N-[2-Chloro-5-((S)-1-phenyl-ethylcarbamoyl)-phenyl]malonamic acid methyl ester

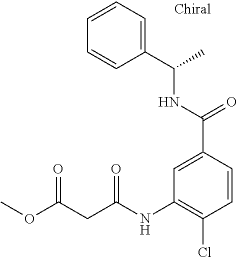

To an ice bath-cooled mixture of 3-amino-4-chloro-N—((S)-1-phenyl-ethyl)-benzamide (1.08 g, 3.93 mmol), ethyl diisopropylamine (1.02 g, 7.86 mmol) and dichloromethane (50 mL) was added a solution of methyl 3-chloro-3-oxopropanoate (0.805 g, 5.9 mmol) in dichloromethane (5 mL). The mixture was stirred for 5 minutes, then the cooling bath was removed and the mixture stirred at room temperature overnight. The mixture was diluted with dichloromethane (20 mL), washed twice with water (50 mL) and once with brine (50 mL). The dichloromethane layer was dried over anhydrous sodium sulfate, filtered and concentrated under reduced pressure. The residue was purified by chromatography on silica gel eluting with hexanes-ethyl acetate (gradient 70:30-30:70) to give N-[2-chloro-5-((S)-1-phenyl-ethylcarbamoyl)-phenyl]-malonamic acid methyl ester as a white solid. (Yield 0.7 g, 47.5%).

### Step C

### 2-Morpholin-4-yl-7-oxo-7,8-dihydro-pyrido[2,3-d]pyrimidine-6-carboxylic acid [2-chloro-5-((S)-1-phenyl-ethylcarbamoyl)-phenyl]-amide

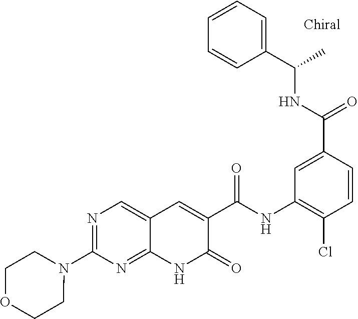

A mixture of 4-amino-2-morpholinopyrimidine-5-carbaldehyde (0.111 g, 0.534 mmol) (from Example 142 supra), N-[2-chloro-5-((S)-1-phenyl-ethylcarbamoyl)-phenyl]-malonamic acid methyl ester (0.20 g, 0.534 mmol), piperidine (0.0423 g, 0.497 mmol) and methanol (30 mL) was heated in a sealed tube at 85° C. overnight. The mixture was cooled and the solid was collected by suction filtration to give 2-morpholin-4-yl-7-oxo-7,8-dihydro-pyrido[2,3-d]pyrimidine-6-carboxylic acid [2-chloro-5-((S)-1-phenyl-ethylcarbamoyl)-phenyl]-amide as a white solid. (Yield 0.170 g, 59.7%). LC-MS: [M+H]⁺ 533.1.

### Example 158

### (R)—N-(2-Chloro-5-(3-hydroxy-1-phenylpropylcarbamoyl)phenyl)-2-methoxy-7-oxo-7,8-dihydropyrido[2,3-d]pyrimidine-6-carboxamide

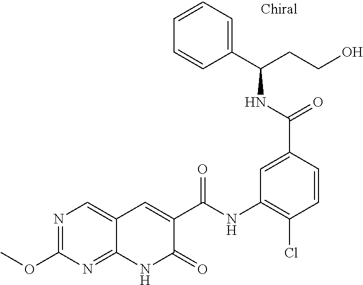

Triethylamine (0.021 mL, 0.15 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.057 g, 0.15 mmol) (Aldrich) in DMF (2.0 mL). The resultant mixture was stirred at room temperature for 30 minutes. (R)-3-Amino-3-phenylpropan-1-ol (24.2 mg, 0.16 mmol) (Aldrich) was added. The mixture was stirred for 18 hours and then partitioned between ethyl acetate and water. The precipitate was collected by filtration and washed with ethyl acetate and water and dried in vacuum oven to give (R)—N-(2-chloro-5-(3-hydroxy-1-phenylpropylcarbamoyl)phenyl)-2-methoxy-7-oxo-7,8-dihydropyrido[2,3-d]pyrimidine-6-carboxamide. (Yield 0.06 g, 88.5%).

HR-MS (ES⁺) m/z Calculated for C₂₅H₂₃ClN₅O₅ ([M+H]⁺): 508.1382. Found: 508.1383.

### Example 159

(S)—N-(2-Chloro-5-(3-hydroxy-1-phenylpropylcarbamoyl)phenyl)-2-methoxy-7-oxo-7,8-dihydropyrido[2,3-d]pyrimidine-6-carboxamide

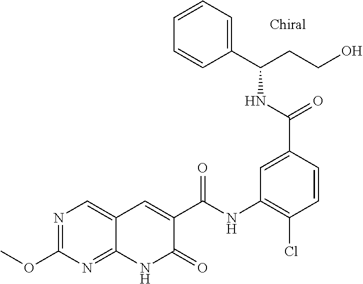

Triethylamine (0.021 mL, 0.15 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.057 g, 0.15 mmol) (Aldrich) in DMF (2.0 mL). The resultant mixture was stirred at room temperature for 30 minutes. (S)-3-Amino-3-phenylpropan-1-ol (24.2 mg, 0.16 mmol) (Aldrich) was added. The mixture was stirred for 18 hours and then partitioned between ethyl acetate and water. The precipitate was collected by filtration and washed with ethyl acetate and water and dried in vacuum oven to give (S)—N-(2-chloro-5-(3-hydroxy-1-phenylpropylcarbamoyl)phenyl)-2-methoxy-7-oxo-7,8-dihydropyrido[2,3-d]pyrimidine-6-carboxamide. (Yield 0.032 g, 47.2%).

HR-MS (ES⁺) m/z Calculated for C₂₅H₂₃ClN₅O₅ ([M+H]⁺): 508.1382. Found: 508.1384.

### Example 160

### 2-Morpholin-4-yl-7-oxo-7,8-dihydro-pyrido[2,3-d]pyrimidine-6-carboxylic acid {5-[3-amino-1-(3-chloro-phenyl)-propylcarbamoyl]-2-chloro-4-fluoro-phenyl}-amide

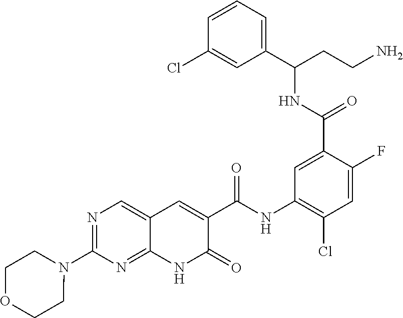

### Step A

### racemic-[3-(5-Amino-4-chloro-2-fluoro-benzoylamino)-3-(3-chloro-phenyl)-propyl]-carbamic acid tert-butyl ester

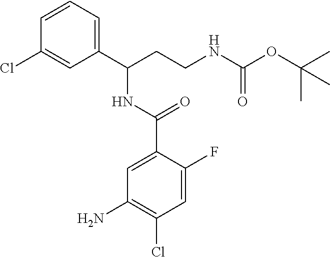

A mixture of 2-fluoro-4-chloro-5-amino-benzoic acid hydrochloride (0.141 g, 0.6 mmol) (CombiBlock), HBTU (0.250 g, 0.66 mmol), ethyl diisopropylamine (0.310 g, 2.4 mmol) and dimethylformamide (1 mL) was stirred for 15 minutes, and then racemic-[3-amino-3-(3-chloro-phenyl)-propyl]-carbamic acid tert-butyl ester (0.124 g, 0.6 mmol) (from Example 9 supra) in dimethylformamide (0.7 mL) was added. The mixture was stirred for 2 hours, then diluted with ethyl acetate (250 mL) and washed successively with 0.5 M sodium carbonate (50 mL), brine (50 mL), dried over anhydrous sodium sulfate, filtered and concentrated under reduced pressure. The residue was purified by chromatography on silica gel eluting with hexanes-ethyl acetate

(60:40) to give racemic-[3-(5-amino-4-chloro-2-fluoro-benzoylamino)-3-(3-chloro-phenyl)-propyl]-carbamic acid tert-butyl ester as a glass. (Yield 0.234 g, 85.5%).

### Step B

### N-{5-[3-tert-Butoxycarbonylamino-1-(3-chloro-phenyl)-propylcarbamoyl]-2-chloro-4-fluoro-phenyl}-malonamic acid methyl ester

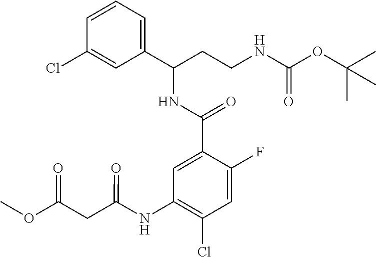

To an ice bath-cooled solution of racemic-[3-(5-amino-4-chloro-2-fluoro-benzoylamino)-3-(3-chloro-phenyl)-propyl]-carbamic acid tert-butyl ester (0.233 g, 0.51 mmol) and ethyldiisopropyl amine (0.132 g, 1 mmol) in dichloromethane (4 mL), was added over 1 minute a solution of methyl 3-chloro-3-oxopropanoate (0.104 g, 0.76 mmol) (Aldrich) in dichloromethane (1 mL). The resultant mixture was stirred 5 minutes, the cooling bath was removed and the mixture was stirred for another 2 hours and then quenched by the addition of saturated sodium bicarbonate solution (1 mL). The mixture was taken up in ethyl acetate (250 mL) and washed with brine (50 mL), dried over anhydrous sodium sulfate, filtered and concentrated under reduced pressure. The residue was purified by chromatography on silica gel eluting with hexanes-ethyl acetate (1:1) to give racemic-N-{5-[3-tert-butoxycarbonylamino-1-(3-chloro-phenyl)-propylcarbamo-yl]-2-chloro-4-fluoro-phenyl}-malonamic acid methyl ester as a glass. (Yield 0.211 g, 74.4%).

### Step C

### [3-{4-Chloro-2-fluoro-5-[(2-morpholin-4-yl-7-oxo-7,8-dihydro-pyrido[2,3-d]pyrimidine-6-carbonyl)-amino]-benzoylamino}-3-(3-chloro-phenyl)-propyl]-carbamic acid tert-butyl ester

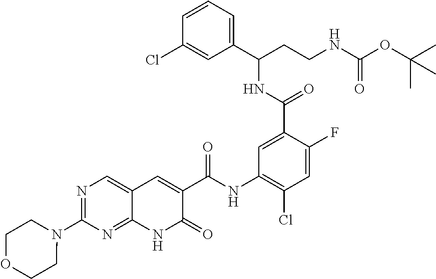

A mixture of 4-amino-2-morpholin-4-yl-pyrimidine-5-carbaldehyde (0.078 g, 0.37 mmol) (from Example 142 supra), racemic-N-{5-[3-tert-butoxycarbonylamino-1-(3-chloro-phenyl)-propylcarbamoyl]-2-chloro-4-fluoro-phenyl}-malonamic acid methyl ester (0.208 g, 0.37 mmol), piperidine (0.032 g, 0.38 mmol) and methanol (3 mL) was heated in a sealed tube at 80° C. for 4 hours. The mixture was then concentrated under reduced pressure. The residue was boiled in methanol (10 mL), cooled and the solid collected by suction filtration to give [3-{4-chloro-2-fluoro-5-[(2-morpholin-4-yl-7-oxo-7,8-dihydro-pyrido[2,3-d]pyrimidine-6-carbonyl)-amino]-benzoylamino}-3-(3-chloro-phenyl)-propyl]-carbamic acid tert-butyl ester as light yellow solid. (Yield 0.1501 g, 56.7%).

### Step D

### 2-Morpholin-4-yl-7-oxo-7,8-dihydro-pyrido[2,3-d]pyrimidine-6-carboxylic acid {5-[3-amino-1-(3-chloro-phenyl)-propylcarbamoyl]-2-chloro-4-fluoro-phenyl}-amide

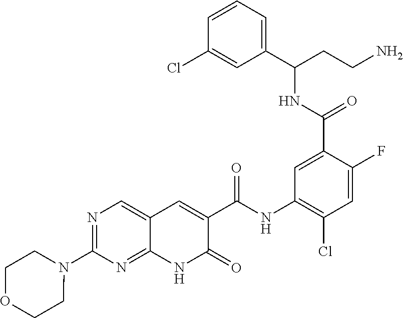

A mixture of [3-{4-chloro-2-fluoro-5-[(2-morpholin-4-yl-7-oxo-7,8-dihydro-pyrido[2,3-d]pyrimidine-6-carbonyl)-amino]-benzoylamino}-3-(3-chloro-phenyl)-propyl]-carbamic acid tert-butyl ester (0.132 g, 0.18 mmol) and dichloromethane-trifluoroacetic acid (2 mL, 1:1) was stirred for 2 hours and concentrated under reduced pressure. The residue was transferred to a separatory funnel containing ethyl acetate (250 mL) and 1 M aqueous sodium hydroxide solution (25 mL). The ethyl acetate layer was dried over anhydrous sodium sulfate, filtered and concentrated under reduced pressure. The residue was boiled in methanol, cooled and the solid collected by suction filtration to give 2-morpholin-4-yl-7-oxo-7,8-dihydro-pyrido[2,3-d]pyrimidine-6-carboxylic acid {5-[3-amino-1-(3-chloro-phenyl)-propylcarbamoyl]-2-chloro-4-fluoro-phenyl}-amide as a white solid. (Yield 0.059 g, 53.3%). LC-MS: [M+H]⁺ 614.1.

### Example 161

### 2-Morpholin-4-yl-7-oxo-7,8-dihydro-pyrido[2,3-d]pyrimidine-6-carboxylic acid [2-chloro-5-(3-fluoro-benzylcarbamoyl)-phenyl]-amide

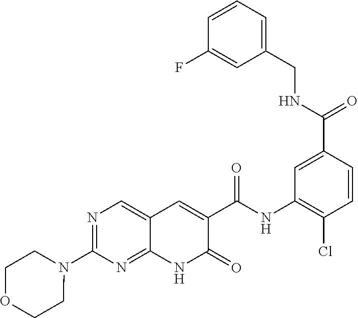

### Step A

### 3-Amino-4-chloro-N-(3-fluoro-benzyl)-benzamide

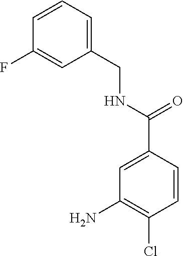

A mixture of 3-amino-4-chloro-benzoic acid HCL salt (0.686 g, 4 mmol) (Alfa), HBTU (1.668 g, 4.4 mmol), ethyl diisopropylamine (1.551 g, 12 mmol) and dimethylformamide (5 mL) was stirred for 15 minutes and then 3-fluorobenzylamine (0.501 g, 4 mmol) (Aldrich) in dimethylformamide (2 mL) was added. The mixture stirred at room temperature for 2 hours, and then taken up in ethyl acetate (250 mL), washed with 0.5 M sodium carbonate (50 mL), brine (50 mL), dried over anhydrous sodium sulfate, filtered and concentrated under reduced pressure. The residue was purified by chromatography on silica gel eluting with hexanes-ethyl acetate (60:40) to give 3-amino-4-chloro-N-(3-fluoro-benzyl)-benzamide. (Yield 0.902 g, 80.9%).

### Step B

### N-[2-Chloro-5-(3-fluoro-benzylcarbamoyl)-phenyl]-malonamic acid methyl ester

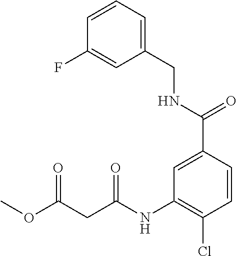

To an ice bath-cooled solution of 3-amino-4-chloro-N-(3-fluoro-benzyl)-benzamide (0.697 g, 2.5 mmol), ethyldiisopropyl amine (0.646 g, 5 mmol) in dichloromethane (7 mL) was added over 2.5 minutes, a solution of methyl 3-chloro-3-oxopropanoate (0.512 g, 3.8 mmol) (Aldrich) in dichloromethane (2.5 mL). The mixture was stirred 5 minutes, the cooling bath was removed and the mixture was stirred for 1 hour and then an additional portion of methyl 3-chloro-3-oxopropanoate (0.256 g, 1.7 mmol) in dichloromethane (1 mL) was added. After 20 minutes, the mixture was quenched by the addition of saturated sodium bicarbonate solution (2 mL). The mixture was taken up in ethyl acetate (250 mL) and washed with brine (50 mL), dried over anhydrous sodium sulfate, filtered and concentrated under reduced pressure. The residue was purified by chromatography on silica gel eluting with hexanes-ethyl acetate (1:1) to give N-[2-chloro-5-(3-fluoro-benzylcarbamoyl)-phenyl]-malonamic acid methyl ester as a white solid. (Yield 0.744 g, 78.6%).

### Step C

### 2-Morpholin-4-yl-7-oxo-7,8-dihydro-pyrido[2,3-d]pyrimidine-6-carboxylic acid [2-chloro-5-(3-fluoro-benzylcarbamoyl)-phenyl]-amide

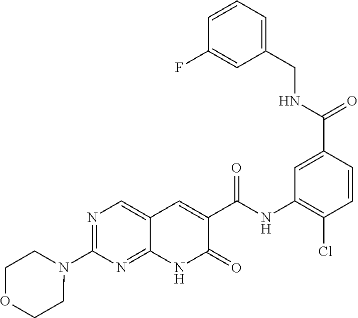

A mixture of 4-amino-2-morpholin-4-yl-pyrimidine-5-carbaldehyde (0.083 g, 0.4 mmol) (from Example 142 supra), N-[2-chloro-5-(3-fluoro-benzylcarbamoyl)-phenyl]-malonamic acid methyl ester (0.151 g, 0.4 mmol), piperidine (0.034 g, 0.4 mmol) and methanol (4 mL) was heated in a sealed tube at 80° C. for 6.5 hours. The mixture was then concentrated under reduced pressure. The residue was recrystallized from methanol to give 2-morpholin-4-yl-7-oxo-7,8-dihydro-pyrido[2,3-d]pyrimidine-6-carboxylic acid [2-chloro-5-(3-fluoro-benzylcarbamoyl)-phenyl]-amide as a yellow solid. (Yield 0.126 g, 58.7%).

LC-MS: [M+H]⁺ 537.3.

### Example 162

### 2-Morpholin-4-yl-7-oxo-7,8-dihydro-pyrido[2,3-d]pyrimidine-6-carboxylic acid [2-chloro-4-fluoro-5-(3-fluoro-benzylcarbamoyl)-phenyl]-amide

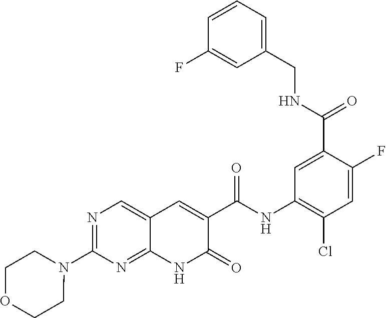

### Step A

### 5-Amino-4-chloro-2-fluoro-N-(3-fluoro-benzyl)-benzamide

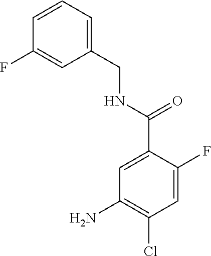

A mixture of 5-amino-4-chloro-2-fluoro-benzoic acid HCL salt (0.940 g, 4 mmol) (CombiBlock), HBTU (1.668 g, 4.4 mmol), ethyl diisopropylamine (2.07 g, 16 mmol) and dimethylformamide (5 mL) was stirred for 15 minutes and then 3-fluorobenzylamine (0.501 g, 4 mmol) (Aldrich) in dimethylformamide (2 mL) was added. The mixture stirred at room temperature for 2 hours, and then taken up in ethyl acetate (250 mL), washed with 0.5 M sodium carbonate solution (50 mL), brine (50 mL), dried over anhydrous sodium sulfate, filtered and concentrated under reduced pressure. The residue was purified by chromatography on silica gel eluting with hexanes-ethyl acetate (60:40) to give 5-amino-4-chloro-2-fluoro-N-(3-fluoro-benzyl)-benzamide as a white solid. (Yield 0.999 g, 84.2%).

### Step B

### N-[2-Chloro-4-fluoro-5-(3-fluoro-benzylcarbamoyl)-phenyl]-malonamic acid methyl ester

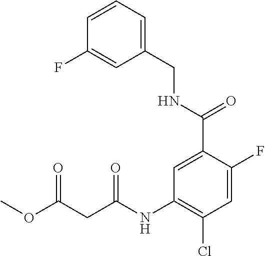

To an ice bath-cooled solution of 5-amino-4-chloro-2-fluoro-N-(3-fluoro-benzyl)-benzamide (0.742 g, 2.5 mmol) and ethyldiisopropyl amine (0.646 g, 5.8 mmol) in dichloromethane (7 mL), was added over 3 minutes a solution of methyl 3-chloro-3-oxopropanoate (0.785 g, 5.8 mmol) (Aldrich) in dichloromethane (3 mL). The mixture was stirred 2 minutes, the cooling bath was removed and the mixture was stirred for 1 hour and then the mixture was quenched by the addition of aqueous saturated sodium bicarbonate solution (3 mL). The mixture was taken up in ethyl acetate (250 mL) and washed with brine (50 mL), dried over anhydrous sodium sulfate, filtered and concentrated under reduced pressure. The residue was purified by chromatography on silica gel eluting with hexanes-ethyl acetate (1:1) to give N-[2-chloro-4-fluoro-5-(3-fluoro-benzylcarbamoyl)-phenyl]-malonamic acid methyl ester as a white solid. (Yield 0.698 g, 70.4%).

### Step C

### 2-Morpholin-4-yl-7-oxo-7,8-dihydro-pyrido[2,3-d]pyrimidine-6-carboxylic acid [2-chloro-4-fluoro-5-(3-fluoro-benzylcarbamoyl)-phenyl]-amide

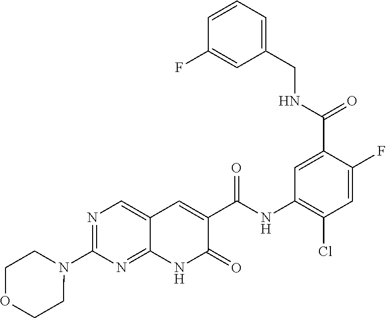

A mixture of 4-amino-2-morpholin-4-yl-pyrimidine-5-carbaldehyde (0.083 g, 0.4 mmol) (from Example 142 supra), N-[2-chloro-4-fluoro-5-(3-fluoro-benzylcarbamoyl)-phenyl]-malonamic acid methyl ester (0.159 g, 0.4 mmol), piperidine (0.034 g, 0.4 mmol) and methanol (4 mL) was heated in a sealed tube at 80° C. for 11 hours. The mixture was then concentrated under reduced pressure. The residue was recrystallized from methanol to give 2-morpholin-4-yl-7-oxo-7,8-dihydro-pyrido[2,3-d]pyrimidine-6-carboxylic acid [2-chloro-4-fluoro-5-(3-fluoro-benzylcarbamoyl)-phenyl]-amide as a light yellow solid. (Yield 0.158 g, 71.2%).

LC-MS: [M+H]⁺ 555.3.

### Example 163

### tert-Butyl 3-(4-chloro-3-(2-methoxy-7-oxo-7,8-dihydropyrido[2,3-d]pyrimidine-6-carboxamido)benzamido)propylcarbamate

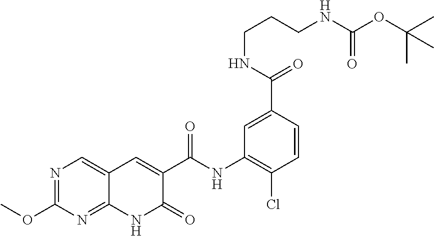

Triethylamine (0.042 mL, 0.30 mmol) was added to a mixture of 4-chloro-3-[(2-methoxy-7-oxo-7,8-dihydro-pyrido[2,3-d]pyrimidine-6-carbonyl)-amino]-benzoic acid (0.10 g, 0.27 mmol) (from Example 22 supra) and HATU (0.11 g, 0.30 mmol) (Aldrich) in DMF (4.0 mL). The mixture was stirred at room temperature for 30 minutes. tert-Butyl 3-aminopropylcarbamate (55.8 mg, 0.32 mmol) (Oakwood) was added. The mixture was stirred for 18 hours and then partitioned between ethyl acetate and water. The precipitate was collected by filtration and washed with ethyl acetate and water and dried in vacuum oven to give tert-butyl 3-(4-chloro-3-(2-methoxy-7-oxo-7,8-dihydropyrido[2,3-d]pyrimidine-6-carboxamido)benzamido)propyl-carbamate. (Yield 0.10 g, 70.6%).

HR-MS (ES⁺) m/z Calculated for C₂₄H₂₈ClN₆O₆ ([M+H]⁺): 553.1573. Found: 553.1573.

### Example 164

### N-(5-(3-Aminopropylcarbamoyl)-2-chlorophenyl)-2-methoxy-7-oxo-7,8-dihydropyrido[2,3-d]pyrimidine-6-carboxamide

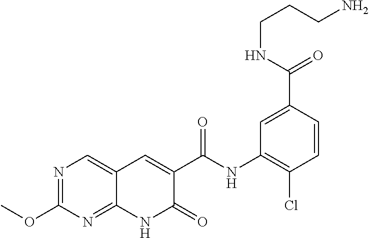

To a solution of tert-butyl 3-(4-chloro-3-(2-methoxy-7-oxo-7,8-dihydropyrido[2,3-d]pyrimidine-6-carboxamido)benzamido)propylcarbamate (0.08 g, 0.15 mmol) (from Example 22 supra) in dichloromethane (8 mL) was added 2,2,2-trifluoroacetic acid (0.5 mL, 6.73 mmol). The mixture was stirred at room temperature for 18 hours. The mixture was concentrated and then partitioned between dichloromethane and saturated sodium carbonate solution. The precipitate was collected by filtration and washed with dichloromethane and water and dried in vacuum oven to give N-(5-(3-aminopropylcarbamoyl)-2-chlorophenyl)-2-methoxy-7-oxo-7,8-dihydropyrido[2,3-d]pyrimidine-6-carboxamide. (Yield 0.053 g, 81.6%).

HR-MS (ES⁺) m/z Calculated for C₁₉H₂₀ClN₆O₄ ([M+H]⁺): 431.1229. Found: 431.1230.

### Example 165

### tert-Butyl 4-(4-chloro-3-(2-methoxy-7-oxo-7,8-dihydropyrido[2,3-d]pyrimidine-6-carboxamido)benzamido)-4-phenylpiperidine-1-carboxylate

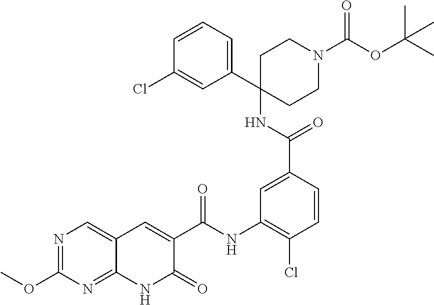

### Step A

### 4-Carbamoyl-4-phenyl-piperidine-1-carboxylic acid tert-butyl ester

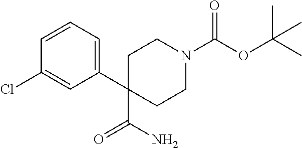

N-ethyl-N-isopropylpropan-2-amine (0.43 mL, 0.25 mmol) was added to a mixture of 1-(tert-butoxycarbonyl)-4-phenylpiperidine-4-carboxylic acid (0.5 g, 1.64 mmol) (Neosystem-T) and HATU (0.93 g, 2.46 mmol) (Aldrich) in DMF (10.0 mL). The mixture was stirred at room temperature for 30 minutes. 7N NH₃ in methanol solution (5.0 mL) (Aldrich) was added. The mixture was stirred for 18 hours and then partitioned between ethyl acetate and water. The aqueous phase was extracted with ethyl acetate (2×). The combined organic phase was washed with water and brine, dried (magnesium sulfate), filtered, and concentrated. The residue was collected by filtration and washed with ether and dried in vacuum oven to give 4-carbamoyl-4-phenyl-piperidine-1-carboxylic acid tert-butyl ester. (Yield 0.48 g, 96.3%).

### Step B

### tert-Butyl 4-amino-4-phenylpiperidine-1-carboxylate

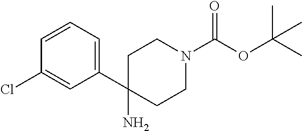

A mixture of 4-carbamoyl-4-phenyl-piperidine-1-carboxylic acid tert-butyl ester (0.2 g, 0.66 mmol) and [bis(tirifluoroaceoxy)iodo]benzene (0.29 g, 0.67 mmol) (Aldrich) in acetonitrile/water (1:1, 8 mL) was stirred at room temperature for 18 hours and then partitioned between ethyl acetate and water. The aqueous phase was extraced with ethyl acetate (2×) and the combined organic phase was washed with saturated sodium bicarbonate solution and brine, dried (magnesium sulfate), filtered, and concentrated to give crude tert-butyl 4-amino-4-phenylpiperidine-1-carboxylate. (Yield 0.15 g, 82.6%).

### Step C

### tert-Butyl 4-(4-chloro-3-(2-methoxy-7-oxo-7,8-dihydropyrido[2,3-d]pyrimidine-6-carboxamido)benzamido)-4-phenylpiperidine-1-carboxylate

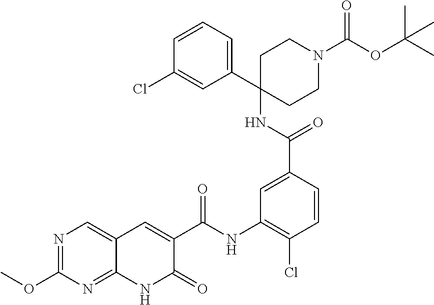

Triethylamine (0.063 mL, 0.45 mmol) was added to a mixture of 4-chloro-3-[(2-methoxy-7-oxo-7,8-dihydro-pyrido[2,3-d]pyrimidine-6-carbonyl)-amino]-benzoic acid (0.14 g, 0.37 mmol) (from Example 22 supra) and HATU (0.17 g, 0.45 mmol) (Aldrich) in DMF (6.0 mL). The resultant mixture was stirred at room temperature for 30 minutes. tert-Butyl 4-amino-4-phenylpiperidine-1-carboxylate (0.15 g, 0.56 mmol) was added. The mixture was stirred for 18 hours and then partitioned between ethyl acetate and water. The aqueous phase was extraced with ethyl acetate (2×) and the combined organic phase was washed with saturated sodium bicarbonate solution and brine, dried (magnesium sulfate), filtered, and concentrated. The residue was recrystallized from DMF/ethyl acetate to give tert-butyl 4-(4-chloro-3-(2-methoxy-7-oxo-7,8-dihydropyrido[2,3-d]pyrimidine-6-carboxamido)benzamido)-4-phenylpiperidine-1-carboxylate. (Yield 0.053 g, 22.4%). HR-MS (ES⁺) m/z Calculated for C₃₂H₃₃ClN₆O₆ ([M+H]⁺): 655.2042. Found: 655.2043.

### Example 166

### N-(2-Chloro-5-(4-phenylpiperidin-4-ylcarbamoyl)phenyl)-2-methoxy-7-oxo-7,8-dihydropyrido[2,3-d]pyrimidine-6-carboxamide

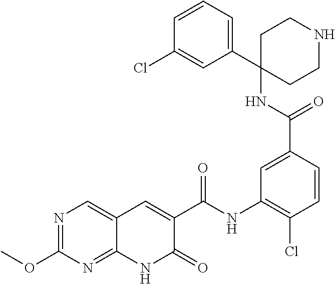

To a solution of tert-butyl 4-(4-chloro-3-(2-methoxy-7-oxo-7,8-dihydropyrido[2,3-d]pyrimidine-6-carboxamido)benzamido)-4-phenylpiperidine-1-carboxylate (0.053 g, 0.084 mmol) (from Example 165 supra) in dichloromethane (4 mL) was added a mixture of 2,2,2-trifluoroacetic acid (0.4 mL). The mixture was stirred at room temperature for 18 hours. The mixture was concentrated and then diluted with dichloromethane and saturated sodium carbonate solution. The precipitate was collected by filtration and washed with dichloromethane and water and dried in vacuum oven to give N-(2-chloro-5-(4-phenylpiperidin-4-ylcarbamoyl)phenyl)-2-methoxy-7-oxo-7,8-dihydropyrido[2,3-d]pyrimidine-6-carboxamide. (Yield 0.038 g, 80.7%).

HR-MS (ES⁺) m/z Calculated for C₂₇H₂₆ClN₆O₄ ([M+H]⁺): 533.1699. Found: 533.1702.

### Example 167

### N-(5-(3-Amino-3-oxopropylcarbamoyl)-2-chlorophenyl)-2-methoxy-7-oxo-7,8-dihydropyrido[2,3-d]pyrimidine-6-carboxamide

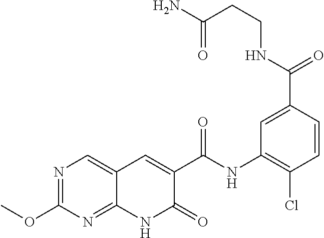

Triethylamine (0.028 ml, 0.20 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.06 g, 0.15 mmol) (Aldrich) in DMF (2.0 mL). Mixture was stirred at room temperature for 30 minutes. H-Beta-Ala-NH₂HCl (0.02 g, 0.16 mmol) was added. Mixture was stirred for 18 hours and then partitioned between ethyl acetate and water. The precipitate was collected by filtration and washed with ethyl acetate and water and dried in vacuum oven. The crude material was purified by HPLC eluting with MeCN/H₂O to give N-(5-(3-amino-3-oxopropylcarbamoyl)-2-chlorophenyl)-2-methoxy-7-oxo-7,8-dihydropyrido[2,3-d]pyrimidine-6-carboxamide. (Yield 0.010 g, 16.8%).

HR-MS (ES⁺) m/z Calculated for C₁₉H₁₈ClN₆O₅ ([M+H]⁺): 445.1022. Found: 445.1023.

### Example 168

### (R)—N-(5-(3-Amino-1-phenylpropylcarbamoyl)-2-chlorophenyl)-2-methyl-7-oxo-7,8-dihydropyrido[2,3-d]pyrimidine-6-carboxamide hydrochloride

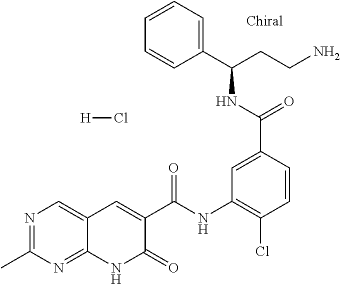

### Step A

### (R)-tert-Butyl 3-(4-chloro-3-(2-methyl-7-oxo-7,8-dihydropyrido[2,3-d]pyrimidine-6-carboxamido)benzamido)-3-phenylpropylcarbamate

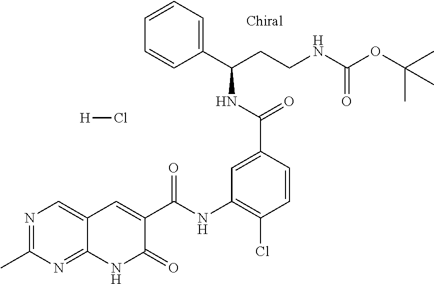

In a 25 mL pear-shaped flask, 4-chloro-3-(2-methyl-7-oxo-7,8-dihydropyrido[2,3-d]pyrimidine-6-carboxamido)benzoic acid (29.7 mg, 82.8 μmol) (from Example 111 supra), (R)-tert-butyl 3-amino-3-phenylpropyl-carbamate (24 mg, 95.9 μmol) (from Example 8 supra) and triethylamine (41.4 mg, 57 μL, 409 μmol) were combined with DMF (420 μL) to give a red solution. HATU (37.0 mg, 97.3 μmol) was added and the reaction was stirred at room temperature under an argon atmosphere. After 2 hours the reaction was diluted with water. A precipitate dropped out of solution. EtOAc was added and the mixture stirred. The solid was then collected by filtration, washed with water and EtOAc and dried under house vacuum to give (R)-tert-butyl 3-(4-chloro-3-(2-methyl-7-oxo-7,8-dihydropyrido[2,3-d]pyrimidine-6-carboxamido)benzamido)-3-phenylpropylcarbamate. (Yield 25.6 mg, 43.3 μmol, 52.3%).

### Step B

### (R)—N-(5-(3-Amino-1-phenylpropylcarbamoyl)-2-chlorophenyl)-2-methyl-7-oxo-7,8-dihydropyrido[2,3-d]pyrimidine-6-carboxamide hydrochloride

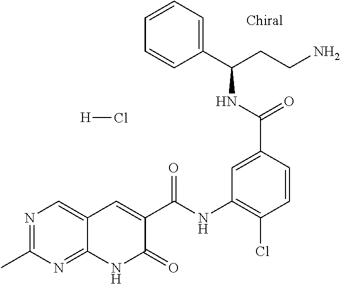

(R)-tert-Butyl 3-(4-chloro-3-(2-methyl-7-oxo-7,8-dihydropyrido[2,3-d]pyrimidine-6-carboxamido)benzamido)-3-phenylpropylcarbamate (22 mg, 37.2 μmol) was suspended in dioxane (0.37 mL). A solution of hydrogen chloride in dioxane (4.0M, 444 mg, 0.37 mL, 1.48 mmol) was added and the reaction was stirred at room temperature. After 2 hours the reaction was concentrated to a smaller volume and diluted with EtOAc. The solid was collected by filtration and washed with EtOAc. The yellow solid was dissolved in water and filtered through a cotton plug to remove any insoluble particles and freeze-dried to give (R)—N-(5-(3-amino-1-phenylpropylcarbamoyl)-2-chlorophenyl)-2-methyl-7-oxo-7,8-dihydropyrido[2,3-d]pyrimidine-6-carboxamide hydrochloride. (Yield 17.9 mg, 33.9 μmol, 91.2%).

HR-MS (ES⁺) m/z Calculated for C₂₅H₂₄ClN₆O₃ ([M+H]⁺): 491.1593. Found: 491.1593.

### Example 169

### N-(5-(3-Amino-1-phenylpropylcarbamoyl)-2-chlorophenyl)-2-methoxy-4-methyl-7-oxo-7,8-dihydropyrido[2,3-d]pyrimidine-6-carboxamide2,2,2-trifluoroacetate

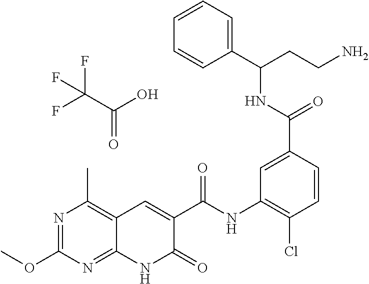

### Step A

### 4-Amino-2-methoxy-6-methylpyrimidine-5-carbonitrile

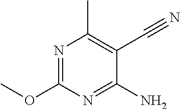

Methyl carbamimidate hydrochloride (2.21 g, 20.0 mmol) was suspended in ethanol (5.0 mL) and sodium ethoxide (6.48 g, 7.47 mL, 20.0 mmol) was added. A clear solution never resulted before additional solid began to precipitate out of solution. The mixture was stirred at room temperature for about 45 minutes and then was filtered and washed with ethanol. The filtrate (clear and yellow in color) was treated with 2-(1-ethoxyethylidene)malononitrile (1.36 g, 10 mmol). The malononitrile did not completely dissolve before a new solid began to precipitate out of solution. The mixture was stirred at room temperature for about 1 hour. The solid was then collected, washed with ethanol and dried to give 4-amino-2-methoxy-6-methylpyrimidine-5-carbonitrile. (Yield 1.50 g, 9.14 mmol, 91.4%).

### Step B

### 4-Amino-2-methoxy-6-methylpyrimidine-5-carbaldehyde

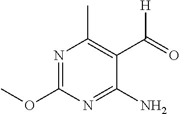

4-Amino-2-methoxy-6-methylpyrimidine-5-carbonitrile (420 mg, 2.56 mmol) was suspended in water (3.0 mL). Sulfuric acid (600 μL) was added which resulted in a clear, colorless solution. Palladium on carbon (10%; 58.8 mg, 55.3 μmol) was added and the mixture was hydrogenated with the Parr shaker (5-14 psi) for 1 hour. The reaction mixture was filtered through a bed of Celite. The filtrate was cooled in an ice-water bath and then neutralized with concentrated ammonium hydroxide. The solid was collected, washed with water and dried to give 4-amino-2-methoxy-6-methylpyrimidine-5-carbaldehyde. (Yield 326 mg, 1.95 mmol, 76.2%).

### Step C

### Ethyl 2-methoxy-4-methyl-7-oxo-7,8-dihydropyrido[2,3-d]pyrimidine-6-carboxylate

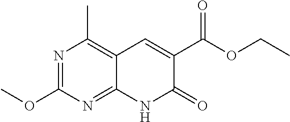

4-Amino-2-methoxy-6-methylpyrimidine-5-carbaldehyde (323 mg, 1.93 mmol) was dissolved in DMF (8.6 mL), resulting in a slightly cloudy, colorless solution. Diethyl malonate (617 mg, 585 μL, 3.85 mmol), potassium carbonate (401 mg, 2.9 mmol) and triethylamine (196 mg, 270 μL, 1.94 mmol) were added and the mixture was heated in an oil bath at 85° C. for 16 hours. After cooling to room temperature, the solid was collected by filtration, washed with EtOAc and dried to give ethyl 2-methoxy-4-methyl-7-oxo-7,8-dihydropyrido[2,3-d]pyrimidine-6-carboxylate. (About 50% pure. Yield 666 mg, 1.26 mmol, 65.5%).

### Step D

### 2-Methoxy-4-methyl-7-oxo-7,8-dihydropyrido[2,3-d]pyrimidine-6-carboxylic acid

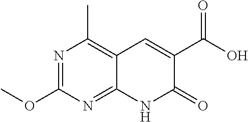

Ethyl 2-methoxy-4-methyl-7-oxo-7,8-dihydropyrido[2,3-d]pyrimidine-6-carboxylate (0.66 g [50% purity], 1.00 mmol) was suspended in methanol (3.2 mL) and water (1.6 mL). Lithium hydroxide (60.0 mg, 2.51 mmol) was added and the mixture was heated in an oil bath at 65° C. The mixture was cooled to room temperature after about 3 hours and concentrated. The residue was diluted with water. Most of the material went into solution. The aqueous solution was washed with EtOAc and then acidified with 6N HCl, resulting in the precipitation of a solid. The solid was collected by filtration, washed with water and dried to give 2-methoxy-4-methyl-7-oxo-7,8-dihydropyrido[2,3-d]pyrimidine-6-carboxylic acid. (Yield 131 mg, 557 μmol, 55.5%).

### Step E

### Methyl 4-chloro-3-(2-methoxy-4-methyl-7-oxo-7,8-dihydropyrido[2,3-d]pyrimidine-6-carboxamido)benzoate

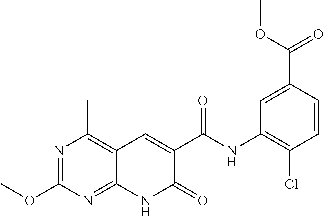

2-Methoxy-4-methyl-7-oxo-7,8-dihydropyrido[2,3-d]pyrimidine-6-carboxylic acid (131 mg, 557 μmol) was suspended in DMF (1.70 mL). Methyl 3-amino-4-chlorobenzoate (155 mg, 835 μmol) was added, followed by triethylamine (283 mg, 390 μL, 2.8 mmol). HATU (318 mg, 835 μmol) was then added. Solution was initially clear but solid soon began to precipitate out of solution. After stirring at room temperature for 16 hours the reaction mixture was diluted with EtOAc (1 mL). The solid was then collected by filtration, washed with EtOAc and dried to give methyl 4-chloro-3-(2-methoxy-4-methyl-7-oxo-7,8-dihydropyrido[2,3-d]pyrimidine-6-carboxamido)-benzoate. (Yield 108 mg, 244 μmol, 43.8%).

### Step F

### 4-Chloro-3-(2-methoxy-4-methyl-7-oxo-7,8-dihydropyrido[2,3-d]pyrimidine-6-carboxamido)-benzoic acid

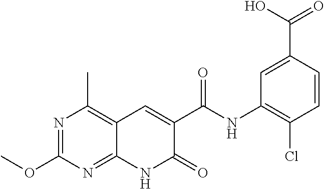

Methyl 4-chloro-3-(2-methoxy-4-methyl-7-oxo-7,8-dihydropyrido[2,3-d]pyrimidine-6-carboxamido)benzoate (105 mg, 261 μmol) was suspended in methanol (850 μL) and water (425 μL). Lithium hydroxide (17.2 mg, 717 μmol) was added and the mixture was heated in an oil bath at 65° C. Additional lithium hydroxide (4 to 5 mg) was added after 3 hours when the reaction was found to be incomplete. The mixture was heated for another 1.5 hours and then was cooled to room temperature and concentrated to near dryness. The residue was taken up in 8 to 9 mL water and heated to dissolve. Aqueous hydrochloric acid was added resulting in the precipitation of product. The mixture was chilled and then the solid was collected, washed with water and dried to give 4-chloro-3-(2-methoxy-4-methyl-7-oxo-7,8-dihydropyrido[2,3-d]pyrimidine-6-carboxamido)benzoic acid. (Yield 86.6 mg, 223 μmol, 85.5%).

### Step G

### tert-Butyl 3-(4-chloro-3-(2-methoxy-4-methyl-7-oxo-7,8-dihydropyrido[2,3-d]pyrimidine-6-carboxamido)benzamido)-3-phenylpropylcarbamate

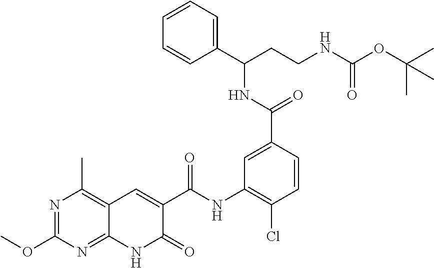

4-Chloro-3-(2-methoxy-4-methyl-7-oxo-7,8-dihydropyrido[2,3-d]pyrimidine-6-carboxamido)-benzoic acid (41.7 mg, 107 μmol) and tert-butyl 3-amino-3-phenylpropylcarbamate (29.5 mg, 118 μmol) (from Example 6 supra) were combined in DMF (540 μL). Triethylamine (54.3 mg, 74.8 μL, 536 μmol) was added followed by HATU (48.9 mg, 129 μmol). A clear solution was never seen. Stirred at room temperature for 5 hours. After workup, the reaction was found to be incomplete. The material was again taken up in DMF (450 μL) and treated with additional amine (10.6 mg), triethylamine (75 μL) and HATU (27.6 mg). Following stirring at room temperature for 2.5 hours the reaction was diluted with water. Solid precipitated out but then gummed up. The mixture was diluted with EtOAc and stirred. A filterable solid slowly formed which was collected, giving tert-butyl 3-(4-chloro-3-(2-methoxy-4-methyl-7-oxo-7,8-dihydropyrido[2,3-d]pyrimidine-6-carboxamido)benzamido)-3-phenylpropylcarbamate. (Yield 24.8 mg, 37.9 μmol, 35.4%).

### Step H

### N-(5-(3-Amino-1-phenylpropylcarbamoyl)-2-chlorophenyl)-2-methoxy-4-methyl-7-oxo-7,8-dihydropyrido[2,3-d]pyrimidine-6-carboxamide 2,2,2-trifluoroacetate

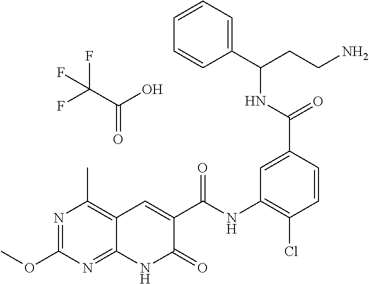

tert-Butyl 3-(4-chloro-3-(2-methoxy-4-methyl-7-oxo-7,8-dihydropyrido[2,3-d]pyrimidine-6-carboxamido)benzamido)-3-phenylpropylcarbamate (24.5 mg, 39.4 μmol) was suspended in dioxane (400 μL). A hydrogen chloride solution in dioxane (4M, 480 mg, 400 μL, 1.6 mmol) was added and the mixture was stirred at room temperature. After 2 hours the reaction mixture was diluted with EtOAc. The solid was collected and washed with EtOAc. The crude material was purified by flash chromatography (reverse phase, 50 g Polaris C18, 10% to 100% acetonitrile in water (each containing 0.1% TFA)) in several runs. The product-containing fractions from each run were combined and concentrated. The residue was dissolved in water, filtered and freeze-dried to give N-(5-(3-amino-1-phenylpropylcarbamoyl)-2-chlorophenyl)-2-methoxy-4-methyl-7-oxo-7,8-dihydropyrido[2,3-d]pyrimidine-6-carboxamide 2,2,2-trifluoroacetate. (Yield 8.4 mg, 13.2 μmol, 33.5%).

HR-MS (ES⁺) m/z Calculated for C₂₆H₂₆ClN₆O₄ ([M+H]⁺): 521.1699. Found: 521.1699.

### Example 170

### N-(5-(2-Amino-1-phenylethylcarbamoyl)-2-chlorophenyl)-2-methoxy-4-methyl-7-oxo-7,8-dihydropyrido[2,3-d]pyrimidine-6-carboxamide 2,2,2-trifluoroacetate

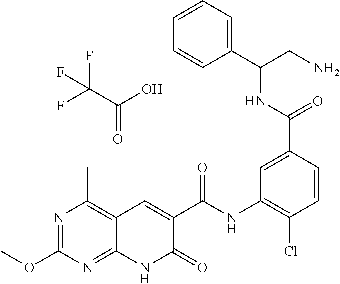

### Step A

### tert-Butyl 2-(4-chloro-3-(2-methoxy-4-methyl-7-oxo-7,8-dihydropyrido[2,3-d]pyrimidine-6-carboxamido)benzamido)-2-phenylethylcarbamate

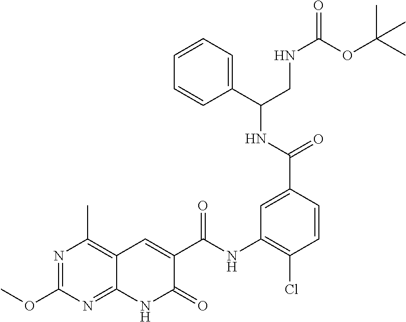

4-Chloro-3-(2-methoxy-4-methyl-7-oxo-7,8-dihydropyrido[2,3-d]pyrimidine-6-carboxamido)-benzoic acid (41.3 mg, 106 μmol) (from Example 169 supra) and tert-butyl 2-amino-2-phenylethylcarbamate (27.6 mg, 117 μmol) (from Example 4 supra) were combined in DMF (540 μL). Most of the material remained insoluble. Triethylamine (54.4 mg, 75.0 μL, 538 μmol) was added followed by HATU (48.5 mg, 127 μmol) and the thick mixture was stirred at room temperature. The reaction was found to be incomplete after 2.5 hours and additional HATU (10 mg) was added. After 5.5 hours, the reaction was diluted with water and filtered. The solid was washed with DMF-water (1×), water (1×) and EtOAc (2×) and then dried to give tert-butyl 2-(4-chloro-3-(2-methoxy-4-methyl-7-oxo-7,8-dihydropyrido[2,3-d]pyrimidine-6-carboxamido)benzamido)-2-phenylethylcarbamate. (Yield 37 mg, 57.9 μmol, 54.5%).

### Step B

### N-(5-(2-Amino-1-phenylethylcarbamoyl)-2-chlorophenyl)-2-methoxy-4-methyl-7-oxo-7,8-dihydropyrido[2,3-d]pyrimidine-6-carboxamide 2,2,2-trifluoroacetate

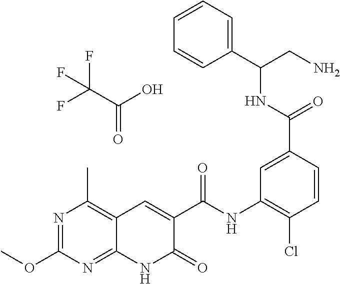

tert-Butyl 2-(4-chloro-3-(2-methoxy-4-methyl-7-oxo-7,8-dihydropyrido[2,3-d]pyrimidine-6-carboxamido)benzamido)-2-phenylethylcarbamate (33.5 mg, 55.2 μmol) was suspended in dioxane (550 μL). Hydrochloric acid (4M in dioxane, 660 mg, 550 μL, 2.2 mmol) was added and the reaction was stirred at room temperature for 2 hours. The reaction was diluted with EtOAc and stirred. The solid was collected, washed with EtOAc and dried briefly. The crude material was purified by flash chromatography (reverse phase, 50 g Polaris C18, 10% to 100% acetonitrile in water (each containing 0.1% TFA)) in several runs. The product-containing fractions from each run were combined and concentrated. The residue was dissolved in water and freeze-dried to give N-(5-(2-amino-1-phenylethylcarbamoyl)-2-chlorophenyl)-2-methoxy-4-methyl-7-oxo-7,8-dihydropyrido[2,3-d]pyrimidine-6-carboxamide 2,2,2-trifluoroacetate. (Yield 16.3 mg, 26.2 μmol, 47.6%).

HR-MS (ES⁺) m/z Calculated for C₂₅H₂₄ClN₆O₄ ([M+H]⁺): 507.1542. Found: 507.1543.

### Example 171

### (R)—N-(5-(3-Amino-1-phenylpropylcarbamoyl)-2-chlorophenyl)-2-methoxy-7-oxo-7,8-dihydropyrido[2,3-d]pyrimidine-6-carboxamide

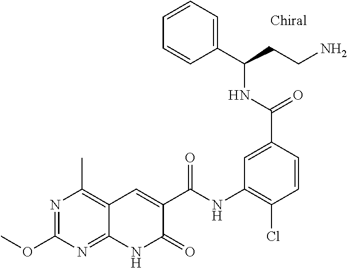

### Step A

### (R)-3-{4-Chloro-3-[(2-methoxy-7-oxo-7,8-dihydro-pyrido[2,3-d]pyrimidine-6-carbonyl)-amino]-benzoylamino}-3-phenyl-propyl)-carbamic acid tert-butyl ester

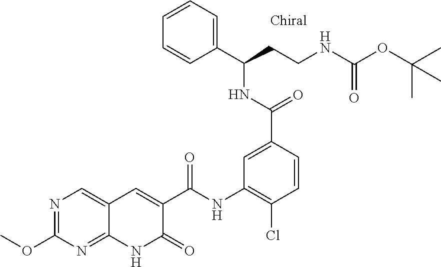

Triethylamine (0.024 mL, 1.72 mmol) was added to a mixture of 4-chloro-3-[(2-methoxy-7-oxo-7,8-dihydro-pyrido[2,3-d]pyrimidine-6-carbonyl)-amino]-benzoic acid (0.58 g, 1.54 mmol) (from Example 22 supra) and HATU (0.66 g, 1.72 mmol) (Aldrich) in DMF (20.0 mL). The mixture was stirred at room temperature for 30 minutes. (R)-tert-Butyl 3-Amino-3-phenylpropylcarbamate (0.46 g, 1.84 mmol) (from Example 8 supra) was added. The mixture was stirred for 18 hours and then partitioned between ethyl acetate and water. The aqueous phase was extraced with ethyl acetate (3×) and the combined organic phase was washed with water and brine, dried (magnesium sulfate), filtered, and concentrated. The precipitate was collected by filtration and washed with hot dichloromethane and ethyl acetate and dried in vacuum oven to give (R)-3-{4-chloro-3-[(2-methoxy-7-oxo-7,8-dihydro-pyrido[2,3-d]pyrimidine-6-carbonyl)-amino]-benzoylamino}-3-phenyl-propyl)-carbamic acid tert-butyl ester (0.62 g). The filtrate was concentrated. The residue was purified by flash chromatography eluting with 20-50% ethyl acetate in dichloromethane to give second crop of product (0.1 g). (Total yield 0.72 g, 77.2%).

### Step B

### (R)—N-(5-(3-Amino-1-phenylpropylcarbamoyl)-2-chlorophenyl)-2-methoxy-7-oxo-7,8-dihydropyrido[2,3-d]pyrimidine-6-carboxamide

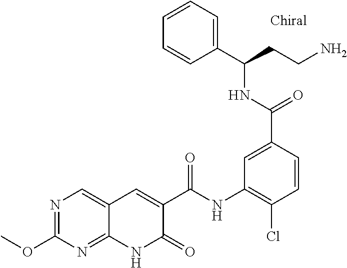

To a solution of (R)-3-{4-chloro-3-[(2-methoxy-7-oxo-7,8-dihydro-pyrido[2,3-d]pyrimidine-6-carbonyl)-amino]-benzoylamino}-3-phenyl-propyl)-carbamic acid tert-butyl ester (0.62 g, 1.02 mmol) in dichloromethane (20 mL) was added 2,2,2-trifluoroacetic acid (4.0 mL). The mixture was stirred at room temperature for 18 hours. The mixture was concentrated and then diluted with dichloromethane and saturated sodium carbonate solution. The precipitate was collected by filtration and washed with dichloromethane and water and dried in vacuum oven to give (R)—N-(5-(3-amino-1-phenylpropylcarbamoyl)-2-chlorophenyl)-2-methoxy-7-oxo-7,8-dihydropyrido-[2,3-d]pyrimidine-6-carboxamide. (Yield 0.4 g, 77.3%).

HR-MS (ES⁺) m/z Calculated for C₂₅H₂₄ClN₆O₄ ([M+H]⁺): 507.1542. Found: 507.1542.

### Example 172

### 2-(4-Methyl-piperazin-1-yl)-7-oxo-7,8-dihydro-pyrido[2,3-d]pyrimidine-6-carboxylic acid (5-benzylcarbamoyl-2-chloro-phenyl)-amide

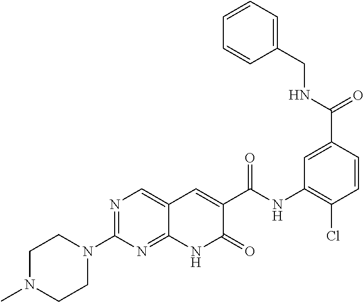

### Step A

### 4-Amino-2-(4-methyl-piperazin-1-yl)-pyrimidine-5-carboxylic acid ethyl ester

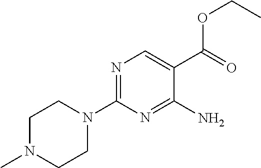

A mixture of ethyl 4-amino-2-mercaptopyrimidine-5-carboxylate (6 g, 30.1 mmol) and 1-methyl piperazine (20 mL) was heated at 90° C. overnight. The mixture was concentrated under reduced pressure. The residue was taken up in dichloromethane (50 mL) and washed once with water (50 mL), once with brine (50 mL), dried over anhydrous sodium sulfate, filtered and concentrated under reduced pressure. The residue was triturated with hexanes-ethyl acetate (9:1) to give 4-amino-2-(4-methyl-piperazin-1-yl)-pyrimidine-5-carboxylic acid ethyl ester as a yellow solid. (Yield 6.2 g, 77.6%).

### Step B

### [4-Amino-2-(4-methyl-piperazin-1-yl)-pyrimidin-5-yl]-methanol

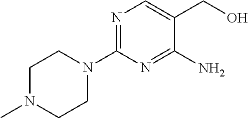

To an ice cooled solution of 2M lithium aluminum hydride in tetrahydrofuran (15 mL) was added, over 30 minutes, a solution of 4-amino-2-(4-methyl-piperazin-1-yl)-pyrimidine-5-carboxylic acid ethyl ester (6.2 g, 23.4 mmol) in tetrahydrofuran (60 mL). The mixture was stirred at room temperature for 1 hour, then cooled in an ice bath and quenched by the cautious addition of 1M sodium hydroxide solution (3 mL), and water (3 mL). Solids were removed by suction filtration, washing the filter cake with ethyl acetate (100 mL). The filtrate was concentrated under reduced pressure to give [4-amino-2-(4-methyl-piperazin-1-yl)-pyrimidin-5-yl]-methanol, which was used without further purification. (Yield 4.2 g, 80.4%).

### Step C

### 4-Amino-2-(4-methyl-piperazin-1-yl)-pyrimidine-5-carbaldehyde

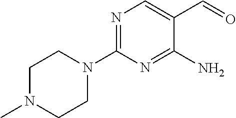

A mixture of [4-amino-2-(4-methyl-piperazin-1-yl)-pyrimidin-5-yl]-methanol (4.2 g, 18.8 mmol), manganese dioxide (8.18 g, 94.1 mmol) and dichloromethane (100 mL) was stirred at room temperature for 24 hours. The solids were removed by filtration through Celite, washing the filter pad with hot acetone. The filtrate was concentrated under reduced pressure to give 4-amino-2-(4-methyl-piperazin-1-yl)-pyrimidine-5-carbaldehyde as a white solid. (Yield 2.1 g, 50.5%).

### Step D

### N-(5-Benzylcarbamoyl-2-chloro-phenyl)-malonamic acid methyl ester

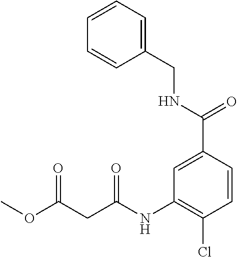

To an ice bath cooled mixture of 3-amino-N-benzyl-4-chloro-benzamide (0.90 g, 3.45 mmol) (from Example 2 supra), ethyl diisopropylamine (0.892 g, 6.9 mmol) and dichloromethane (50 mL) was added a solution of methyl 3-chloro-3-oxopropanoate (0.707 g, 5.18 mmol) in dichloromethane (5 mL). The mixture was stirred for 5 minutes, then the cooling bath was removed and the mixture stirred at room temperature overnight. The mixture was diluted with dichloromethane (20 mL), washed with water (20 mL). The dichloromethane layer was dried over anhydrous sodium sulfate, filtered and concentrated under reduced pressure. The residue was purified by chromatography on silica gel eluting with hexanes-ethyl acetate (70:30) to give N-(5-benzylcarbamoyl-2-chloro-phenyl)-malonamic acid methyl ester as white solid. (Yield 0.55 g, 44.2%).

### Step E

### 2-(4-Methyl-piperazin-1-yl)-7-oxo-7,8-dihydro-pyrido[2,3-d]pyrimidine-6-carboxylic acid (5-benzylcarbamoyl-2-chloro-phenyl)-amide

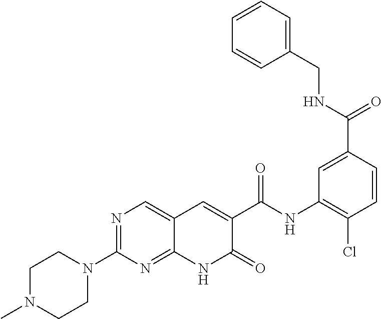

A mixture of 4-amino-2-(4-methyl-piperazin-1-yl)-pyrimidine-5-carbaldehyde (0.147 g, 0.67 mmol), N-(5-benzylcarbamoyl-2-chloro-phenyl)-malonamic acid methyl ester (0.20 g, 0.55 mmol), piperidine (0.047 g, 0.55 mmol) and methanol (30 mL) was heated in a sealed tube at 85° C. overnight. The mixture was cooled and the solid was collected by suction filtration to give 2-(4-methyl-piperazin-1-yl)-7-oxo-7,8-dihydro-pyrido[2,3-d]pyrimidine-6-carboxylic acid (5-benzylcarbamoyl-2-chloro-phenyl)-amide as a white solid. (Yield 0.150 g, 51.3%).

LC-MS: [M+H]⁺ 532.4.

### Example 173

### 2-(4-Methyl-piperazin-1-yl)-7-oxo-7,8-dihydro-pyrido[2,3-d]pyrimidine-6-carboxylic acid [2-chloro-5-((S)-1-phenyl-ethylcarbamoyl)-phenyl]-amide

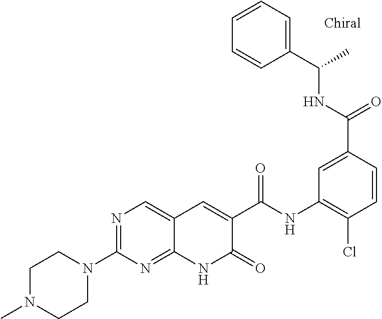

A mixture (0.106 g, 0.48 mmol) of 4-amino-2-(4-methyl-piperazin-1-yl)-pyrimidine-5-carbaldehyde (from Example 172 supra), (0.150 g, 0.4 mmol) of N-[2-chloro-5-((S)-1-phenyl-ethylcarbamoyl)-phenyl]-malonamic acid methyl ester (from Example 157 supra), (0.034 g, 0.4 mmol) of piperidine and methanol (30 mL) was heated in a sealed tube at 85° C. overnight. The mixture was cooled and the solid was collected by suction filtration to give 2-(4-methyl-piperazin-1-yl)-7-oxo-7,8-dihydro-pyrido[2,3-d]pyrimidine-6-carboxylic acid [2-chloro-5-((S)-1-phenyl-ethylcarbamoyl)-phenyl]-amide as a white solid. (Yield 0.120 g, 54.9%).

LC-MS: [M+H]⁺ 546.4.

### Example 174

### 2-(4-Methyl-piperazin-1-yl)-7-oxo-7,8-dihydro-pyrido[2,3-d]pyrimidine-6-carboxylic acid [2-chloro-5-((R)-1-phenyl-ethylcarbamoyl)-phenyl]-amide

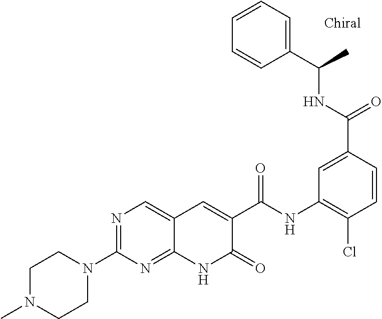

A mixture (0.089 g, 0.4 mmol) of 4-amino-2-(4-methyl-piperazin-1-yl)-pyrimidine-5-carbaldehyde (from Example 172 supra), (0.150 g, 0.4 mmol) of N-[2-chloro-5-((R)-1-phenyl-ethylcarbamoyl)-phenyl]-malonamic acid methyl ester (from Example 156 supra), (0.034 g, 0.4 mmol) piperidine and methanol (30 mL) was heated in a sealed tube at 85° C. overnight. The mixture was cooled and the solid was collected by suction filtration to give 2-(4-methyl-piperazin-1-yl)-7-oxo-7,8-dihydro-pyrido[2,3-d]pyrimidine-6-carboxylic acid [2-chloro-5-((R)-1-phenyl-ethylcarbamoyl)-phenyl]-amide as a light yellow solid. (Yield 0.044 g, 20.1%).

LC-MS: [M+H]⁺ 546.4.

### Example 175

### (S)-tert-Butyl 3-(4-chloro-3-(2-methoxy-7-oxo-7,8-dihydropyrido[2,3-d]pyrimidine-6-carboxamido)benzamido)-3-phenylpropylcarbamate

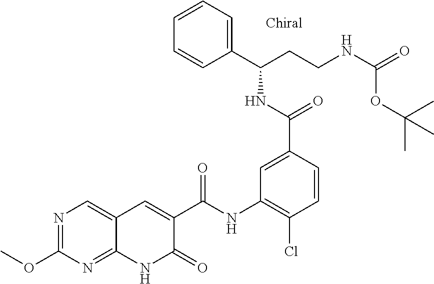

Triethylamine (0.08 mL, 0.6 mmol) was added to a mixture of 4-chloro-3-[(2-methoxy-7-oxo-7,8-dihydro-pyrido[2,3-d]pyrimidine-6-carbonyl)-amino]-benzoic acid (0.20 g, 0.53 mmol) (from Example 22 supra) and HATU (0.23 g, 0.6 mmol) (Aldrich) in DMF (10.0 mL). The resultant mixture was stirred at room temperature for 30 minutes. (S)-tert-Butyl 3-amino-3-phenylpropyl-carbamate (0.16 g, 0.64 mmol) (from Example 7 supra) was added. The mixture was stirred for 18 hours and then partitioned between ethyl acetate and water. The aqueous phase was extraced with ethyl acetate (3×) and the combined organic phase was washed with water and brine, dried (magnesium sulfate), filtered, and concentrated. The precipitate was collected by filtration and washed with hot ethyl acetate and dried in vacuum oven to give (S)-tert-butyl 3-(4-chloro-3-(2-methoxy-7-oxo-7,8-dihydropyrido[2,3-d]pyrimidine-6-carboxamido)benzamido)-3-phenylpropylcarbamate. (Yield 0.32 g, 98.8%).

HR-MS (ES⁺) m/z Calculated for C₃₀H₃₂ClN₆O₆ ([M+H]⁺): 629.1886. Found: 629.1888.

### Example 176

### (S)—N-(5-(3-Amino-1-phenylpropylcarbamoyl)-2-chlorophenyl)-2-methoxy-7-oxo-7,8-dihydropyrido[2,3-d]pyrimidine-6-carboxamide

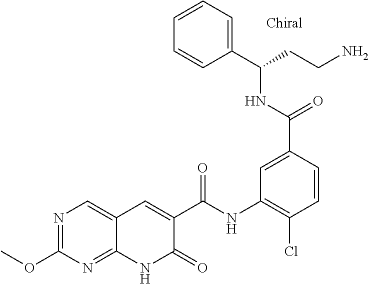

To a solution of (S)-tert-butyl 3-(4-chloro-3-(2-methoxy-7-oxo-7,8-dihydropyrido[2,3-d]pyrimidine-6-carboxamido)benzamido)-3-phenylpropylcarbamate (0.30 g, 0.49 mmol) (from Example 175 supra) in dichloromethane (10 mL) was added 2,2,2-trifluoroacetic acid (4.0 mL). The mixture was stirred at room temperature for 18 hours. The mixture was concentrated and then diluted with dichloromethane and saturated sodium carbonate solution. The precipitate was collected by filtration and washed with dichloromethane, methanol and water and dried in vacuum oven to give (S)—N-(5-(3-amino-1-phenylpropylcarbamoyl)-2-chlorophenyl)-2-methoxy-7-oxo-7,8-dihydropyrido[2,3-d]pyrimidine-6-carboxamide. (Yield 0.1 g, 39.9%).

HR-MS (ES⁺) m/z Calculated for C₂₅H₂₄ClN₆O₄ ([M+H]⁺): 507.1542. Found: 507.1539.

### Example 177

### 2-(4-Methyl-piperazin-1-yl)-7-oxo-7,8-dihydro-pyrido[2,3-d]pyrimidine-6-carboxylic acid [2-chloro-5-(3-fluoro-benzylcarbamoyl)-phenyl]amide

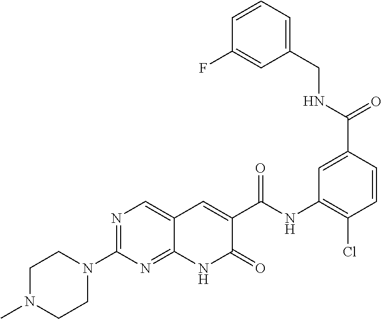

### Step A

### 3-Amino-4-chloro-N-(3-fluoro-benzyl)-benzamide

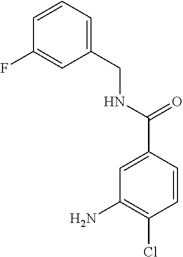

A mixture of 1H-benzo[d][1,2,3]triazol-1-yl 3-amino-4-chlorobenzoate (1 g, 3.46 mmol) (from Example 142 supra), 3-fluoro-benzylamine (0.433 g, 3.46 mmol), triethylamine (1.75 g, 17.3 mmol) and dimethylformamide (10 mL) was stirred at room temperature for 3 hours. The mixture was diluted with dichloromethane (50 mL) and washed successively twice with water (50 mL) and then brine (50 mL). The dichloromethane layer was dried over anhydrous sodium sulfate, filtered and concentrated under reduced pressure. The residue was purified by chromatography on silica gel eluting with hexanes-ethyl acetate (70:30) to give 3-amino-4-chloro-N-(3-fluoro-benzyl)-benzamide as a white solid. (Yield 0.81 g, 84%).

### Step B

### N-[2-Chloro-5-(3-fluoro-benzylcarbamoyl)-phenyl]-malonamic acid methyl ester

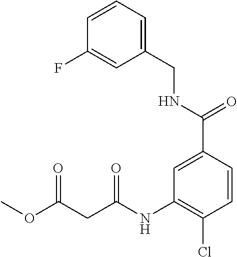

To an ice bath-cooled mixture of 3-amino-4-chloro-N-(3-fluoro-benzyl)-benzamide (0.81 g, 2.91 mmol), ethyl diisopropylamine (0.751 g, 5.81 mmol) and dichloromethane (50 mL) was added a solution of methyl 3-chloro-3-oxopropanoate (0.595 g, 4.36 mmol) in dichloromethane (5 mL). The mixture was stirred for 5 minutes, then the cooling bath was removed and the mixture stirred at room temperature overnight. The mixture was diluted with dichloromethane (20 mL), washed with water (20 mL). The dichloromethane layer was dried over anhydrous sodium sulfate, filtered and concentrated under reduced pressure. The residue was purified by chromatography on silica gel eluting with hexanes-ethyl acetate (60:40) to give N-[2-chloro-5-(3-fluoro-benzylcarbamoyl)-phenyl]-malonamic acid methyl ester as white solid. (Yield 0.56 g, 50.8%).

### Step C

### 2-(4-Methyl-piperazin-1-yl)-7-oxo-7,8-dihydro-pyrido[2,3-d]pyrimidine-6-carboxylic acid [2-chloro-5-(3-fluoro-benzylcarbamoyl)-phenyl]-amide

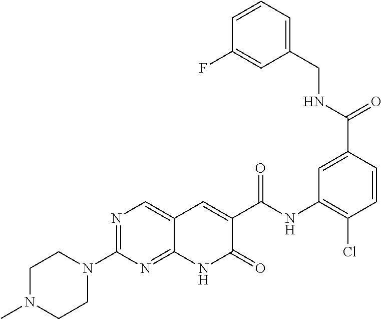

A mixture of 4-amino-2-(4-methyl-piperazin-1-yl)-pyrimidine-5-carbaldehyde (0.105 g, 0.48 mmol) (from Example 172 supra), N-[2-chloro-5-(3-fluoro-benzylcarbamoyl)-phenyl]-malonamic acid methyl ester (0.15 g, 0.4 mmol), piperidine (0.034 g, 0.4 mmol) and methanol (30 mL) was heated in a sealed tube at 85° C. overnight. The mixture was cooled and the solid was collected by suction filtration to give 2-(4-methyl-piperazin-1-yl)-7-oxo-7,8-dihydro-pyrido[2,3-d]pyrimidine-6-carboxylic acid [2-chloro-5-(3-fluoro-benzylcarbamoyl)-phenyl]-amide as a white solid. (Yield 0.150 g, 68.2%). LC-MS: [M+H]⁺ 550.2.

### Example 178

### 2-(4-Methyl-piperazin-1-yl)-7-oxo-7,8-dihydro-pyrido[2,3-d]pyrimidine-6-carboxylic acid [2-chloro-5-(2,5-difluoro-benzylcarbamoyl)-phenyl]amide

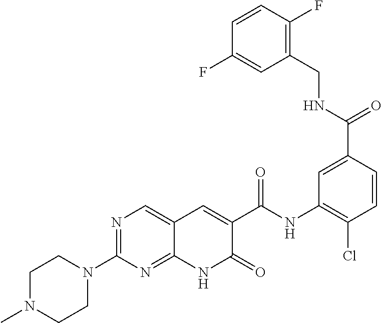

### Step A

### 3-Amino-4-chloro-N-(2,5-difluoro-benzyl)-benzamide

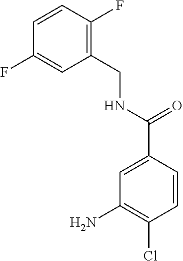

A mixture of 1H-benzo[d][1,2,3]triazol-1-yl 3-amino-4-chlorobenzoate (1.5 g, 5.2 mmol) (from Example 172 supra), 2,5-difluoro-benzylamine (0.744 g, 5.2 mmol), triethylamine (2.63 g, 26 mmol) and (15 mL) dimethylformamide (15 mL) was stirred at room temperature for 3 hours. The mixture was diluted with dichloromethane (50 mL) and washed successively twice with water (50 mL) and then brine (50 mL). The dichloromethane layer was dried over anhydrous sodium sulfate, filtered and concentrated under reduced pressure. The residue was purified by chromatography on silica gel eluting with hexanes-ethyl acetate (70:30) to give 3-amino-4-chloro-N-(2,5-difluoro-benzyl)-benzamide as a white solid. (Yield 1.2 g, 77.8%).

### Step B

### N-[2-Chloro-5-(2,5-difluoro-benzylcarbamoyl)-phenyl]-malonamic acid methyl ester

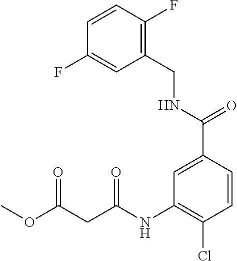

To an ice bath-cooled mixture of 3-amino-4-chloro-N-(2,5-difluoro-benzyl)-benzamide (1.2 g, 4.04 mmol), ethyl diisopropylamine (1.05 g, 8.09 mmol) and dichloromethane (50 mL) was added a solution of methyl 3-chloro-3-oxopropanoate (0.828 g, 6.07 mmol) in dichloromethane (5 mL). The mixture was stirred for 5 minutes, then the cooling bath was removed and the mixture stirred at room temperature overnight. The mixture was diluted with dichloromethane (20 mL), washed with water (20 mL). The dichloromethane layer was dried over anhydrous sodium sulfate, filtered and concentrated under reduced pressure. The residue was purified by chromatography on silica gel eluting with hexanes-ethyl acetate (60:40) to give N-[2-chloro-5-(2,5-difluoro-benzylcarbamoyl)-phenyl]-malonamic acid methyl ester as white solid. (Yield 0.75 g, 46.8%).

### Step C

### 2-(4-Methyl-piperazin-1-yl)-7-oxo-7,8-dihydro-pyrido[2,3-d]pyrimidine-6-carboxylic acid [2-chloro-5-(2,5-difluoro-benzylcarbamoyl)-phenyl]-amide

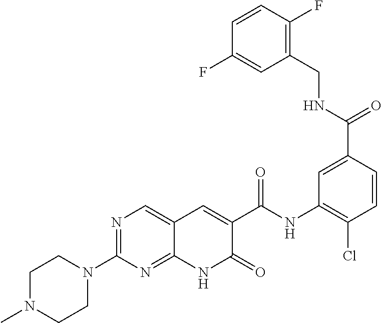

A mixture of 4-amino-2-(4-methyl-piperazin-1-yl)-pyrimidine-5-carbaldehyde (0.10 g, 0.45 mmol) (from Example 172 supra), N-[2-chloro-5-(2,5-difluoro-benzylcarbamoyl)-phenyl]-malonamic acid methyl ester (0.15 g, 0.38 mmol), piperidine (0.032 g, 0.38 mmol) and methanol (30 mL) was heated in a sealed tube at 85° C. overnight. The mixture was cooled and the solid was collected by suction filtration to give 2-(4-methyl-piperazin-1-yl)-7-oxo-7,8-dihydro-pyrido[2,3-d]pyrimidine-6-carboxylic acid [2-chloro-5-(2,5-difluoro-benzylcarbamoyl)-phenyl]-amide as a white solid. (Yield 0.160 g, 74.1%).

LC-MS: [M+H]⁺ 568.2.

### Example 179

### 2-(1R,5S)-8-Oxa-3-aza-bicyclo [3.2.1]oct-3-yl-7-oxo-7,8-dihydro-pyrido[2,3-d]pyrimidine-6-carboxylic acid [2-chloro-5-(3-chloro-benzylcarbamoyl)-phenyl]amide

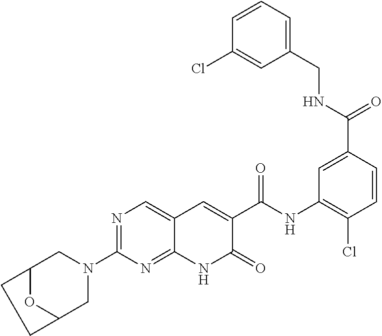

To a disposable tube with stir bar and equipped with a Teflon-lined screw cap was added 2-methanesulfonyl-7-oxo-7,8-dihydro-pyrido[2,3-d]pyrimidine-6-carboxylic acid [2-chloro-5-(3-chloro-benzylcarbamoyl)-phenyl]-amide (150 mg, 275 μmol, Eq: 1.00) (from Example 75 supra), (1R,5S)-8-oxa-3-azabicyclo[3.2.1]octane hydrochloride (218 mg, 1.46 mmol, Eq: 5.31) (from Example 15 supra), N,N-dimethylformamide (500 μL) and triethylamine (218 mg, 300 μL, 2.15 mmol, Eq: 7.84). The reaction was heated at 85-90° C. in a pre-heated oil bath for 18 hours, after which it was cooled to room temperature and quenched with water. The precipitated solid was filtered off, rinsed with water, diethyl ether, ethyl acetate, water and then diethyl ether and then dried under air aspiration. The solid was freeze dried from acetonitrile/water overnight to provide 2-(1R,5S)-8-oxa-3-aza-bicyclo[3.2.1]oct-3-yl-7-oxo-7,8-dihydro-pyrido[2,3-d]pyrimidine-6-carboxylic acid [2-chloro-5-(3-chloro-benzylcarbamoyl)-phenyl]-amide. (Yield 77 mg, 48%).

LR-MS [M+H]⁺: 579.

### Example 180

### (3-{4-Chloro-3-[((1R,5S)-2-8-oxa-3-aza-bicyclo[3.2.1]oct-3-yl-7-oxo-7,8-dihydro-pyrido[2,3-d]pyrimidine-6-carbonyl)-amino]-benzoylamino}-3-phenyl-propyl)-carbamic acid tert-butyl ester

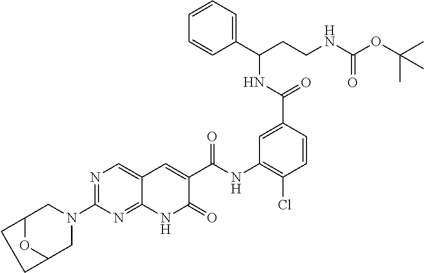

To a disposable tube with stir bar was added tert-butyl 3-(4-chloro-3-(2-(methylsulfonyl)-7-oxo-7,8-dihydropyrido[2,3-d]pyrimidine-6-carboxamido)benzamido)-3-phenylpropylcarbamate (60 mg, 91.6 μmol, Eq: 1.00) (from Example 116 supra), (1R,5S)-8-oxa-3-azabicyclo[3.2.1]octane hydrochloride (180 mg, 1.2 mmol, Eq: 13.1) (from Example 15 supra), DMF (0.3 mL) and triethylamine (218 mg, 300 μL, 2.15 mmol, Eq: 23.5). The reaction was then heated at 85° C. in a pre-heated oil bath for 18 hours, after which it was quenched with water. The precipitated solid was collected, rinsed with water, diethyl ether, ethyl acetate, water and diethyl ether and then dried under aspiration. The solid was then freeze dried from acetonitrile/water overnight to provide (3-{4-chloro-3-[((1R,5S)-2-8-oxa-3-aza-bicyclo[3.2.1]oct-3-yl-7-oxo-7,8-dihydro-pyrido[2,3-d]pyrimidine-6-carbonyl)-amino]-benzoylamino}-3-phenyl-propyl)-carbamic acid tert-butyl ester. (Yield 55 mg, 87%).

LR-MS [M+H]⁺: 688.

### Example 181

### 2-(1R,5S)-8-Oxa-3-aza-bicyclo [3.2.1]oct-3-yl-7-oxo-7,8-dihydro-pyrido[2,3-d]pyrimidine-6-carboxylic acid [2-chloro-5-(2-hydroxy-1-phenyl-ethylcarbamoyl)-phenyl]amide

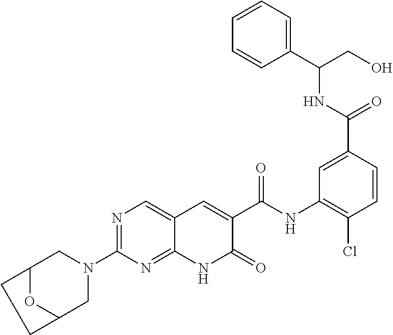

To a disposable tube with stir bar were added N-(2-chloro-5-(2-hydroxy-1-phenylethylcarb-amoyl)phenyl)-2-(methylsulfonyl)-7-oxo-7,8-dihydropyrido[2,3-d]pyrimidine-6-carboxamide (75 mg, 138 μmol, Eq: 1.00) (from Example 145 supra), (1R,5S)-8-oxa-3-azabicyclo-[3.2.1]octane hydrochloride (175 mg, 1.17 mmol, Eq: 8.45) (from Example 15 supra), DMF (300 μL) and then triethylamine (182 mg, 250 μL, 1.79 mmol, Eq: 13.0). The reaction was heated at 90° C. in a pre-heated oil bath for 18 hours, after which it was cooled to room temperature and quenched with water. The precipitated solid was filtered off, rinsed with water, diethyl ether, ethyl acetate and then diethyl ether. The solid was freeze dried from CH₃CN/H₂O to provide 2-(1R,5S)-8-oxa-3-aza-bicyclo[3.2.1]oct-3-yl-7-oxo-7,8-dihydro-pyrido[2,3-d]pyrimidine-6-carboxylic acid [2-chloro-5-(2-hydroxy-1-phenyl-ethylcarbamoyl)-phenyl]-amide. (Yield 42 mg, 53%). LR-MS [M+H]⁺: 575.

### Example 182

### 2-(1R,5S)-8-Oxa-3-aza-bicyclo [3.2.1]oct-3-yl-7-oxo-7,8-dihydro-pyrido[2,3-d]pyrimidine-6-carboxylic acid [2-chloro-5-(3-hydroxy-1-phenyl-propylcarbamoyl)-phenyl]amide

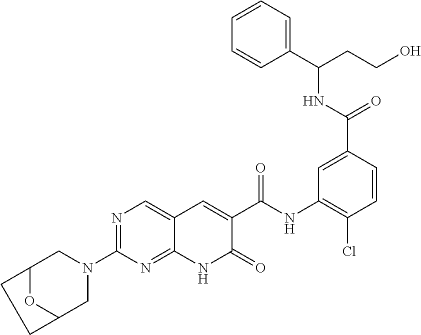

To a disposable tube with stir bar were added N-(2-chloro-5-(3-hydroxy-1-phenylpropylcarb-amoyl)phenyl)-2-(methylsulfonyl)-7-oxo-7,8-dihydropyrido[2,3-d]pyrimidine-6-carboxamide (75 mg, 135 μmol, Eq: 1.00) (from Example 146 supra), (1R,5S)-8-oxa-3-azabicyclo-[3.2.1]octane hydrochloride (175 mg, 1.17 mmol, Eq: 8.67) (from Example 15 supra), DMF (300 μL) and then triethylamine (182 mg, 250 μL, 1.79 mmol, Eq: 13.3). The reaction was heated at 90° C. in a pre-heated oil bath for 18 hours, after which it was cooled to room temperature and quenched with water. The precipitated solid was collected by filtration, rinsed with water, diethyl ether, water, ethyl acetate and then diethyl ether. The solid was freeze dried from acetonitrile/water to provide 2-(1R,5S)-8-oxa-3-aza-bicyclo[3.2.1]oct-3-yl-7-oxo-7,8-dihydro-pyrido[2,3-d]pyrimidine-6-carboxylic acid [2-chloro-5-(3-hydroxy-1-phenyl-propylcarbamoyl)-phenyl]-amide. (Yield 38 mg, 48%). LR-MS [M+H]⁺: 589.

### Example 183

### 2-(1R,5S)-8-Oxa-3-aza-bicyclo [3.2.1]oct-3-yl-7-oxo-7,8-dihydro-pyrido[2,3-d]pyrimidine-6-carboxylic acid [5-(3-amino-1-phenyl-propylcarbamoyl)-2-chloro-phenyl]amide; hydrochloride

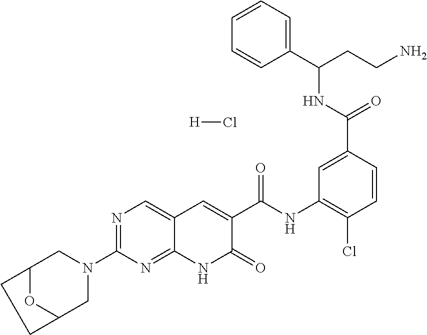

To a 10 mL round bottom flask with stir bar was added tert-butyl 3-(3-(2-((1R,5S)-8-oxa-3-azabicyclo[3.2.1]octan-3-yl)-7-oxo-7,8-dihydropyrido[2,3-d]pyrimidine-6-carboxamido)-4-chlorobenzamido)-3-phenylpropylcarbamate (25 mg, 36.3 μmol, Eq: 1.00) (from Example 180 supra) 1,4-dioxane (500 μL) and then 4M hydrochloric acid in 1,4-dioxane (500 μL, 2.00 mmol, Eq: 55.1). The reaction was allowed to stir at room temperature overnight. The solution was concentrated under reduced pressure to remove the solvents. The crude material was dissolved in water and then freeze dried to provide 2-(1R,5S)-8-oxa-3-aza-bicyclo[3.2.1]oct-3-yl-7-oxo-7,8-dihydro-pyrido[2,3-d]pyrimidine-6-carboxylic acid [5-(3-amino-1-phenyl-propylcarbamoyl)-2-chloro-phenyl]-amide; hydrochloride. (Yield 20 mg, 88%).

LR-MS [M+H]⁺: 588.

### Example 184

### (R)—N-(5-(3-Amino-3-phenylpropylcarbamoyl)-2-chlorophenyl)-2-methoxy-7-oxo-7,8-dihydropyrido[2,3-d]pyrimidine-6-carboxamide

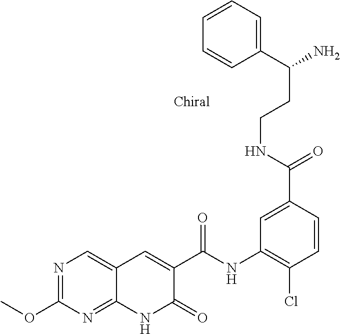

### Step A

### ((R)-2-(3-Hydroxy-1-phenylpropyl)isoindoline-1,3-dione

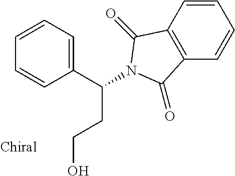

Ethyl 1,3-dioxoisoindoline-2-carboxylate (17.3 g, 79.1 mmol) (Aldrich) was added to a mixture of (R)-3-amino-3-phenylpropan-1-ol (9.97 g, 65.9 mmol) and sodium carbonate (10.6 g, 98.9 mmol) in a 1:1 mixture of THF-H₂O (200 mL). Mixture was stirred at room temperature for 18 hours and then diluted with ethyl acetate. The aqueous phase was extraced with ethyl acetate (2×) and the combined organic phase was washed with water and brine, dried (magnesium sulfate), filtered, and concentrated. The residue was purified by flash chromatography eluting with 0-40% ethyl acetate in hexanes to give ((R)-2-(3-hydroxy-1-phenylpropyl)isoindoline-1,3-dione. (Yield 11.99 g, 64.6%).

### Step B

### (R)-3-(1,3-Dioxoisoindolin-2-yl)-3-phenylpropyl 4-methylbenzenesulfonate

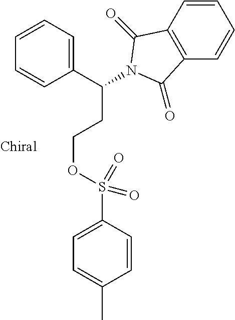

4-Methylbenzene-1-sulfonyl chloride (11.0 g, 57.8 mmol) (Aldrich) was added to a mixture of ((R)-2-(3-hydroxy-1-phenylpropyl)isoindoline-1,3-dione (8.13 g, 28.9 mmol) and pyridine (26 mL) in dichloromethane (100 mL) at 0° C. The mixture was stirred at room temperature for 18 hours and then partitioned between dichloromethane and water. The organic phase was washed with 2N HCl solution and brine, dried (magnesium sulfate), filtered, and concentrated. The residue was purified by flash chromatography eluting with 0-40% ethyl acetate in hexanes to give (R)-3-(1,3-dioxoisoindolin-2-yl)-3-phenylpropyl 4-methylbenzenesulfonate. (Yield 9.42 g, 74.8%).

### Step C

### (R)-2-(3-Azido-1-phenylpropyl)isoindoline-1,3-dione

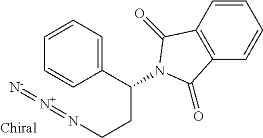

A mixture of (R)-3-(1,3-dioxoisoindolin-2-yl)-3-phenylpropyl 4-methyl-benzenesulfonate (9.42 g, 21.6 mmol) and sodium azide (2.81 g, 43.3 mmol) in DMSO (30 mL) was heated at 80° C. for 2 hours. After cooling, the mixture was partitioned between ethyl acetate and water. The aqueous phase was extracted with ethyl acetate (2×). The combined organic phase was washed with brine, dried (magnesium sulfate), filtered, and concentrated. The residue was purified by flash chromatography eluting with 0-40% ethyl acetate in hexanes to give (R)-2-(3-azido-1-phenylpropyl)isoindoline-1,3-dione. (Yield 5.95 g, 89.8%).

### Step D

### (R)-2-(3-Amino-3-phenylpropyl) isoindoline-1,3-dione

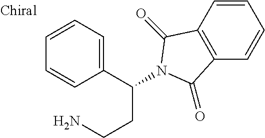

A mixture of (R)-2-(3-azido-1-phenylpropyl)isoindoline-1,3-dione (5.95 g, 19.4 mmol) and 10% Pd/C (0.6 g) in methanol (100 mL) was hydrogenated on a Parr hydrogenator (50 psi) for 6 hours. The mixture was filtered though a Celite pad and then concentrated to give crude (R)-2-(3-amino-3-phenylpropyl) isoindoline-1,3-dione. (Yield 5.23 g, 96.1%).

### Step E

### (R)-tert-Butyl 3-(1,3-dioxoisoindolin-2-yl)-1-phenylpropylcarbamate

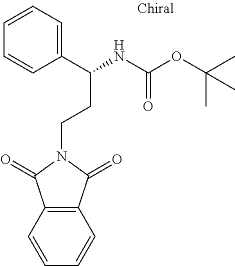

A mixture of (R)-2-(3-amino-3-phenylpropyl)isoindoline-1,3-dione (5.23 g, 18.7 mmol) and di-tert-butyl dicarbonate (6.11 g, 28.0 mmol) in THF (80 mL) was stirred at room temperature for 18 hours. The mixture was concentrate. The residue was purified by flash chromatography eluting with 20-100% ethyl acetate in hexanes to give (R)-tert-butyl 3-(1,3-dioxoisoindolin-2-yl)-1-phenylpropylcarbamate. (Yield 5.23 g, 73.8%).

### Step F

### (R)-tert-Butyl 3-amino-1-phenylpropylcarbamate

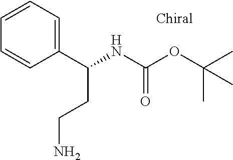

A solution of (R)-tert-butyl 3-(1,3-dioxoisoindolin-2-yl)-1-phenylpropylcarbamate (5.24 g, 13.8 mmol) and hydrazine (4.3 mL, 0.14 mmol) in methanol (100 mL) was heated at reflux for 5 hours. Solid was filtered and washed with methanol. The solution was concentrated. The residue was diluted with dichloromethane and washed with 1N NaOH solution, dried (magnesium sulfate+several pellets of solid sodium hydroxide), filtered, and concentrated. The residue was purified by flash chromatography eluting with 0-7% methanol in dichloromethane (with 1% NH₄OH) to give (R)-tert-butyl 3-amino-1-phenylpropylcarbamate. (Yield 3.4 g, 98.6%).

### Step G

### (R)-tert-Butyl 3-(4-chloro-3-(2-methoxy-7-oxo-7,8-dihydropyrido[2,3-d]pyrimidine-6-carboxamido)benzamido)-1-phenylpropylcarbamate

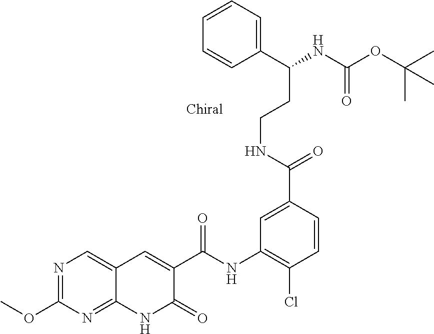

Triethylamine (0.029 mL, 0.21 mmol) was added to a mixture of 4-chloro-3-[(2-methoxy-7-oxo-7,8-dihydro-pyrido[2,3-d]pyrimidine-6-carbonyl)-amino]-benzoic acid (0.07 g, 0.19 mmol) (from Example 22 supra) and HATU (0.08 g, 0.21 mmol) (Aldrich) in DMF (2.0 mL). Mixture was stirred at room temperature for 30 minutes. (R)-tert-Butyl 3-amino-1-phenyl-propylcarbamate (0.06 g, 0.24 mmol) was added. Mixture was stirred for 1 day, and then partitioned between ethyl acetate and water. The aqueous phase was extraced with ethyl acetate (3×) and the combined organic phase was washed with water and brine, dried (magnesium sulfate), filtered, and concentrated. The residue was purified by HPLC eluting with MeCN—H₂O to give (R)-tert-butyl 3-(4-chloro-3-(2-methoxy-7-oxo-7,8-dihydropyrido[2,3-d]pyrimidine-6-carboxamido)benzamido)-1-phenylpropylcarbamate. (Yield 0.048 g, 42.3%).

### Step H

### (R)—N-(5-(3-Amino-3-phenylpropylcarbamoyl)-2-chlorophenyl)-2-methoxy-7-oxo-7,8-dihydropyrido[2,3-d]pyrimidine-6-carboxamide

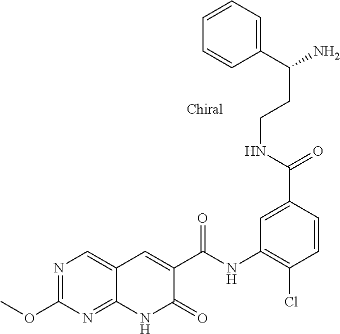

To a solution of (R)-tert-butyl 3-(4-chloro-3-(2-methoxy-7-oxo-7,8-dihydropyrido[2,3-d]pyrimidine-6-carboxamido)benzamido)-1-phenylpropylcarbamate (0.048 g, 0.79 mmol) in dichloromethane (5 mL) was added 2,2,2-trifluoroacetic acid (0.5 mL). The mixture was stirred at room temperature for 5 hours. The mixture was concentrated and then diluted with dichloromethane and saturated sodium carbonate solution. The precipitate was collected by filtration and washed with dichloromethane and water and dried in vacuum oven to give (R)—N-(5-(3-amino-3-phenylpropylcarbamoyl)-2-chlorophenyl)-2-methoxy-7-oxo-7,8-dihydropyrido[2,3-d]pyrimidine-6-carboxamide. (Yield 0.038 g, 95.0%).

HR-MS (ES⁺) m/z Calculated for C₂₅H₂₄ClN₆O₄ ([M+H]⁺): 507.1542. Found: 507.1542.

### Example 185

### tert-Butyl 5-(4-chloro-3-(2-methoxy-7-oxo-7,8-dihydropyrido[2,3-d]pyrimidine-6-carboxamido)benzamido)-5-phenylpentylcarbamate

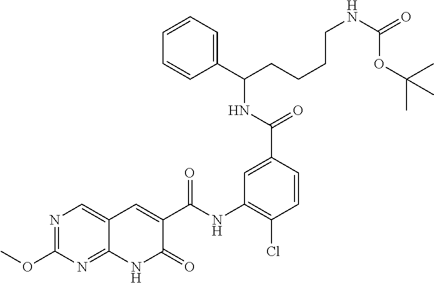

### Step A

### tert-Butyl 5-oxo-5-phenylpentylcarbamate

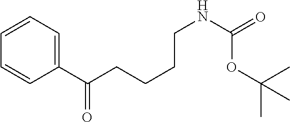

Phenylmagnesium bromide 1M in THF solution (28.9 mL, 28.9 mmol) (Aldrich) was added to a solution of tert-butyl 2-oxopiperidine-1-carboxylate (5.0 g, 25.1 mmol) (Aldrich) in THF (100 mL) at −78° C. Mixture was stirred at −78° C. for 40 minutes, and then poured into brine. The aqueous phase was extraced with ethyl acetate (2×) and the combined organic phase was dried (magnesium sulfate), filtered, and concentrated. The residue was purified by flash chromatography eluting with 5-40% ethyl acetate in hexanes to give tert-butyl 5-oxo-5-phenylpentylcarbamate. (Yield 4.97 g, 71.4%).

### Step B

### (E)-tert-Butyl 5-(hydroxyimino)-5-phenylpentyl-carbamate

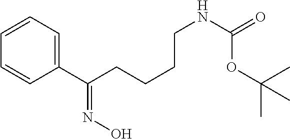

A stirred suspension of tert-butyl 5-oxo-5-phenylpentylcarbamate (4.97 g, 17.9 mmol) in ethanol (24 mL) was treated with a mixture of hydroxylamine hydrochloride (2.49 g, 35.8 mmol) and sodium acetate (3.09 g, 37.6 mmol) in water (24 mL). The mixture was heated at reflux for 5 hours. After cooling to room temperature, water was added and the mixture was extracted with dichloromethane (2×). The combined organic phase was washed with brine, dried (magnesium sulfate), filtered, and concentrated to give crude (E)-tert-butyl 5-(hydroxyimino)-5-phenylpentyl-carbamate. (Yield 5.06 g, 96.6%).

### Step C

### tert-Butyl 5-amino-5-phenylpentylcarbamate

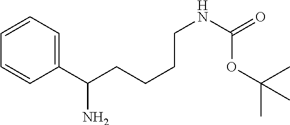

A mixture of (E)-tert-butyl 5-(hydroxyimino)-5-phenylpentylcarbamate (0.63 g, 2.15 mmol) and 10% Pd/C (0.6 g) in methanol (20 mL) was hydrogenated on a Parr hydrogenator (10 psi) for 18 hours. The mixture was filtered though a Celite pad and then concentrated to give crude tert-butyl 5-amino-5-phenylpentylcarbamate. (Yield 0.5 g, 83.4%).

### Step D

### tert-Butyl 5-(4-chloro-3-(2-methoxy-7-oxo-7,8-dihydropyrido[2,3-d]pyrimidine-6-carboxamido)benzamido)-5-phenylpentylcarbamate

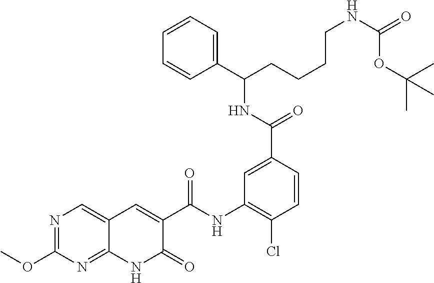

Triethylamine (0.022 mL, 0.16 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.06 g, 0.16 mmol) (Aldrich) in DMF (2.0 mL). Mixture was stirred at room temperature for 30 minutes. tert-Butyl 5-amino-5-phenylpentyl carbamate (0.09 g, 0.33 mmol) was added. Mixture was stirred at room temperature for 18 hours and then partitioned between ethyl acetate and water. The aqueous phase was extraced with ethyl acetate (3×) and the combined organic phase was washed with saturated sodium bicarbonate solution and brine, dried (magnesium sulfate), filtered, and concentrated. The residue was re-crystallized from dichloromethane-ethyl acetate to give tert-butyl 5-(4-chloro-3-(2-methoxy-7-oxo-7,8-dihydropyrido[2,3-d]pyrimidine-6-carboxamido)benzamido)-5-phenylpentylcarbamate (Yield 0.054 g, 63.7%).

HR-MS (ES⁺) m/z Calculated for C₃₂H₃₅ClN₆NaO₆ ([M+Na]⁺): 657.2199. Found: 657.2198.

### Example 186

### N-(5-(5-Amino-1-phenylpentylcarbamoyl)-2-chlorophenyl)-2-methoxy-7-oxo-7,8-dihydropyrido[2,3-d]pyrimidine-6-carboxamide

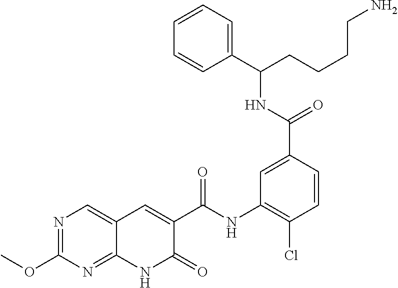

To a solution of tert-butyl 5-(4-chloro-3-(2-methoxy-7-oxo-7,8-dihydropyrido[2,3-d]pyrimidine-6-carboxamido)benzamido)-5-phenylpentylcarbamate (0.049 g, 0.077 mmol) (from Example 185 supra) in dichloromethane (5 mL) was added 2,2,2-trifluoroacetic acid (0.5 mL). The mixture was stirred at room temperature for 18 hours. The mixture was concentrated and then diluted with dichloromethane and saturated sodium carbonate solution. The precipitate was collected by filtration and washed with dichloromethane and water and dried in vacuum oven to give N-(5-(5-amino-1-phenyl-pentylcarbamoyl)-2-chlorophenyl)-2-methoxy-7-oxo-7,8-dihydropyrido[2,3-d]pyrimidine-6-carboxamide. (Yield 0.038 g, 92.1%).

HR-MS (ES⁺) m/z Calculated for C₂₇H₂₈ClN₆O₄ ([M+H]⁺): 535.1855. Found: 535.1857.

### Example 187

### 2-(4-Methyl-piperazin-1-yl)-7-oxo-7,8-dihydro-pyrido[2,3-d]pyrimidine-6-carboxylic acid {5-[(S)-1-aminomethyl-2-(3-fluoro-phenyl)-ethylcarbamoyl]-2-chloro-phenyl}-amide

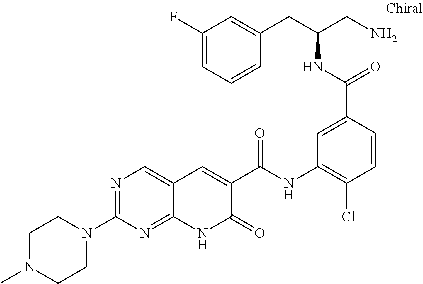

### Step A

### [(S)-1-(1,3-Dioxo-1,3-dihydro-isoindol-2-ylmethyl)-2-(3-fluoro-phenyl)-ethyl]-carbamic acid tert-butyl ester

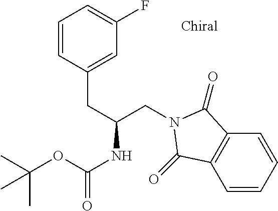

To an ice cooled mixture of [(S)-2-(3-fluoro-phenyl)-1-hydroxymethyl-ethyl]carbamic acid tert-butyl ester (1.214 g, 4.5 mmol), triphenyl phosphine (1.359 g, 5.18 mmol), phthalimide (0.762 g, 5.18 mmol) and tetrahydrofuran (15 mL) was added a solution of diisopropyldiazodicarboxylate (1.048 g, 5.18 mmol) in tetrahydrofuran (5 mL). The mixture was stirred for 30 minutes at room temperature and then concentrated under reduced pressure. The residue was purified by chromatography on silica gel, eluting with dichloromethane-ethyl acetate (90:10) to give [(S)-1-(1,3-dioxo-1,3-dihydro-isoindol-2-ylmethyl)-2-(3-fluoro-phenyl)-ethyl]carbamic acid tert-butyl ester as a white solid. (Yield 1.205 g, 67.2%).

### Step B

### 2-[(S)-2-Amino-3-(3-fluoro-phenyl)-propyl]isoindole-1,3-dione trifluoroacetic acid salt

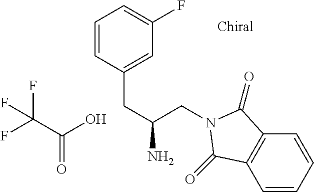

A mixture [(S)-1-(1,3-dioxo-1,3-dihydro-isoindol-2-ylmethyl)-2-(3-fluoro-phenyl)-ethyl]carbamic acid tert-butyl ester (1.203 g, 3 mmol) and trifluoroacetic acid-dichloromethane (15 mL, 2:1) was stirred at room temperature for 1 hour and then concentrated under reduced pressure to give crude 2-[(S)-2-amino-3-(3-fluoro-phenyl)-propyl]-isoindole-1,3-dione trifluoroacetic acid salt. (Yield 1.516, 123%).

### Step C

### 3-Amino-4-chloro-N-[(S)-1-(1,3-dioxo-1,3-dihydro-isoindol-2-ylmethyl)-2-(3-fluoro-phenyl)-ethyl]-benzamide

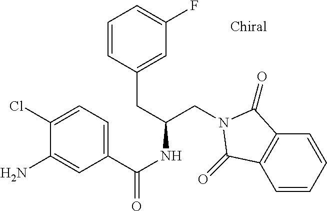

A mixture of 3-amino-4-chloro-benzoic acid (0.464 g, 2.7 mmol), HBTU (1.128 g), ethyl diisopropylamine (1.852 g) and dimethylformamide (5 mL) was stirred for 15 minutes, then added to a flask containing 2-[(S)-2-amino-3-(3-fluoro-phenyl)-propyl]isoindole-1,3-dione trifluoroacetic acid salt (1.516 g, 3 mmol). After stirring for 2 hours, the mixture was diluted with ethyl acetate (300 mL) and washed successively with 0.5M sodium carbonate (50 mL), water (50 mL), brine (50 mL), dried over anhydrous sodium sulfate, filtered and concentrated under reduced pressure. This material was used without further purification.

### Step D

### 3-Amino-N—[(S)-1-aminomethyl-2-(3-fluoro-phenyl)-ethyl]-4-chloro-benzamide

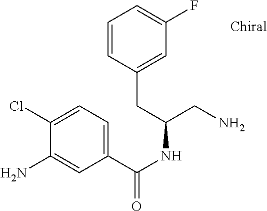

A mixture of the 3-amino-4-chloro-N—[(S)-1-(1,3-dioxo-1,3-dihydro-isoindol-2-ylmethyl)-2-(3-fluoro-phenyl)-ethyl]-benzamide prepared above, methanol (20 mL) and hydrazine (0.85 g) was heated at reflux for 2 hours. The mixture was taken up in dichloromethane (300 mL), washed with 1 M sodium hydroxide (75 mL), dried over anhydrous sodium sulfate, filtered and concentrated under reduced pressure to give crude 3-amino-N—[(S)-1-aminomethyl-2-(3-fluoro-phenyl)-ethyl]-4-chloro-benzamide as a white solid. (Yield 0.946 g, 109%).

### Step E

### [(S)-2-(3-Amino-4-chloro-benzoylamino)-3-(3-fluoro-phenyl)-propyl]-carbamic acid tert-butyl ester

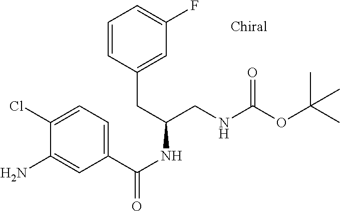

A mixture of 3-amino-N—[(S)-1-aminomethyl-2-(3-fluoro-phenyl)-ethyl]-4-chloro-benzamide (0.943 g, 2.9 mmol), Boc anhydride (0.831 g, 3.8 mmol), ethyl diisopropylamine (1.136 g, 8.8 mmol) and tetrahydrofuran (10 mL) was stirred for 30 minutes then concentrated under reduced pressure. The residue was triturated with ether to give crude [(S)-2-(3-amino-4-chloro-benzoylamino)-3-(3-fluoro-phenyl)-propyl]-carbamic acid tert-butyl ester as a white solid. (Yield 1.418 g, 116%).

### Step F

### N-{5-[(S)-1-(tert-Butoxycarbonylamino-methyl)-2-(3-fluoro-phenyl)-ethylcarbamoyl]-2-chloro-phenyl}-malonamic acid methyl ester

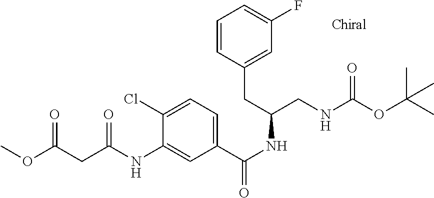

To an ice bath cooled mixture of [(S)-2-(3-amino-4-chloro-benzoylamino)-3-(3-fluoro-phenyl)-propyl]-carbamic acid tert-butyl ester (0.738 g, 1.75 mmol), ethyl diisopropylamine (0.452 g, 3.5 mmol) and dichloromethane (7 mL) was added a solution methyl 3-chloro-3-oxopropanoate (0.418 g, 3.06 mmol) in dichloromethane (3 mL). The mixture was stirred for 5 minutes, then the cooling bath was removed and the mixture stirred at room temperature for 30 minutes and then quenched by the addition of saturated sodium bicarbonate (3 mL). The mixture was diluted with dichloromethane (300 mL), washed with brine (50 mL), dried over anhydrous sodium sulfate, filtered and concentrated under reduced pressure. The residue was purified by chromatography on silica gel, eluting with hexanes-ethyl acetate (1:1), to give N-{5-[(S)-1-(tert-butoxycarbonylamino-methyl)-2-(3-fluoro-phenyl)-ethylcarbamoyl]-2-chloro-phenyl}-malonamic acid methyl ester as a white solid. (Yield 0.685 g, 75%).

### Step G

### [(S)-2-(4-Chloro-3-{[2-(4-methyl-piperazin-1-yl)-7-oxo-7,8-dihydro-pyrido[2,3-d]pyrimidine-6-carbonyl]amino}-benzoylamino)-3-(3-fluoro-phenyl)-propyl]-carbamic acid tert-butyl ester

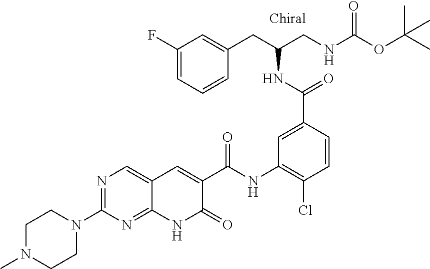

A mixture of 4-amino-2-(4-methyl-piperazin-1-yl)-pyrimidine-5-carbaldehyde (0.086 g, 0.39 mmol) (from Example 172 supra), [(S)-2-(4-chloro-3-{[2-(4-methyl-piperazin-1-yl)-7-oxo-7,8-dihydro-pyrido[2,3-d]pyrimidine-6-carbonyl]-amino}-benzoylamino)-3-(3-fluoro-phenyl)-propyl]-carbamic acid tert-butyl ester (0.204 g, 0.39 mmol), piperidine (0.033 g, 0.39 mmol) and methanol (3 mL) was heated in a sealed tube at 85° C. for 24 hours. The mixture was cooled and the solid was collected by suction filtration to give [(S)-2-(4-chloro-3-{[2-(4-methyl-piperazin-1-yl)-7-oxo-7,8-dihydro-pyrido[2,3-d]pyrimidine-6-carbonyl]-amino}-benzoylamino)-3-(3-fluoro-phenyl)-propyl]-carbamic acid tert-butyl ester as a light yellow solid. (Yield 0.202 g, 74.7%).

### Step H

### 2-(4-Methyl-piperazin-1-yl)-7-oxo-7,8-dihydro-pyrido[2,3-d]pyrimidine-6-carboxylic acid {5-[(S)-1-aminomethyl-2-(3-fluoro-phenyl)-ethylcarbamoyl]-2-chloro-phenyl}-amide

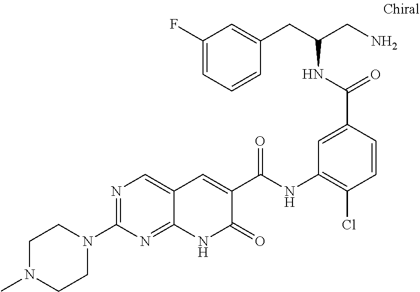

A mixture of [(S)-2-(4-chloro-3-{[2-(4-methyl-piperazin-1-yl)-7-oxo-7,8-dihydro-pyrido[2,3-d]pyrimidine-6-carbonyl]-amino}-benzoylamino)-3-(3-fluoro-phenyl)-propyl]-carbamic acid tert-butyl ester (0.2 g, 0.29 mmol) and dichloromethane-trifluoroacetic acid (3 mL, 1:1) was stirred for 2 hours and then concentrated under reduced pressure. The residue was partitioned between ethyl acetate and 1 M sodium hydroxide saturated with sodium chloride. The precipitate which formed was collected by suction filtration to give 2-(4-methyl-piperazin-1-yl)-7-oxo-7,8-dihydro-pyrido[2,3-d]pyrimidine-6-carboxylic acid {5-[(S)-1-aminomethyl-2-(3-fluoro-phenyl)-ethylcarbamoyl]-2-chloro-phenyl}-amide as a light yellow solid. (Yield 0.055 g, 32%). LC-MS: [M+H]⁺ 548.0.

### Example 188

### tert-Butyl 4-(4-chloro-3-(2-methoxy-7-oxo-7,8-dihydropyrido[2,3-d]pyrimidine-6-carboxamido)benzamido)-4-phenylbutylcarbamate

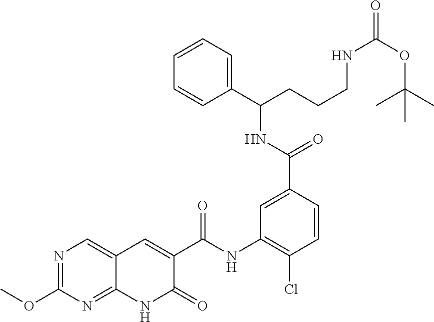

### Step A

### tert-Butyl 4-oxo-4-phenylbutylcarbamate

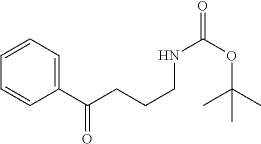

Phenylmagnesium bromide 1M in THF solution (31.9 mL, 31.0 mmol) (Aldrich) was added to a solution of tert-butyl 2-oxopyrrolidine-1-carboxylate (5.0 g, 24.3 mmol) (Aldrich) in THF (100 mL) at −78° C. Mixture was stirred at −78° C. for 40 minutes and then poured into brine. The aqueous phase was extraced with ethyl acetate (2×) and the combined organic phase was dried (magnesium sulfate), filtered, and concentrated. The residue was purified by flash chromatography eluting with 5-40% ethyl acetate in hexanes to give tert-butyl 4-oxo-4-phenylbutylcarbamate. (Yield 4.31 g, 60.6%).

### Step B

### (E)-tert-Butyl 4-(hydroxyimino)-4-phenylbutylcarbamate

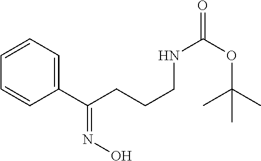

A stirred suspension of tert-butyl 4-oxo-4-phenylbutylcarbamate (4.31 g, 16.4 mmol) in ethanol (24 mL) was treated with a mixture of hydroxylamine hydrochloride (2.27 g, 32.7 mmol) and sodium acetate (2.82 g, 34.4 mmol) in water (24 mL). The mixture was heated at reflux for 5 hours. After cooling to room temperature, water was added and the mixture was extracted with dichloromethane (2×). The combined organic phase was washed with brine, dried (magnesium sulfate), filtered, and concentrated to give (E)-tert-butyl 4-(hydroxyimino)-4-phenylbutyl carbamate. (Yield 4.03 g, 88.5%).

### Step C

### tert-Butyl 4-amino-4-phenylbutylcarbamate

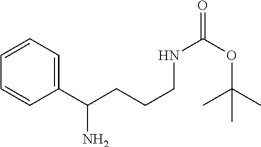

A mixture of (E)-tert-butyl 4-(hydroxyimino)-4-phenylbutylcarbamate (0.77 g, 2.77 mmol) and 10% Pd/C (0.08 g) in methanol (15 mL) was hydrogenated on a Parr hydrogenator (10 psi) for 1 day. The mixture was passed though a Celite pad and then concentrated to give crude tert-butyl 4-amino-4-phenylbutylcarbamate. (Yield 0.71 g, 97.1%).

### Step D

### tert-Butyl 4-(4-chloro-3-(2-methoxy-7-oxo-7,8-dihydropyrido[2,3-d]pyrimidine-6-carboxamido)benzamido)-4-phenylbutylcarbamate

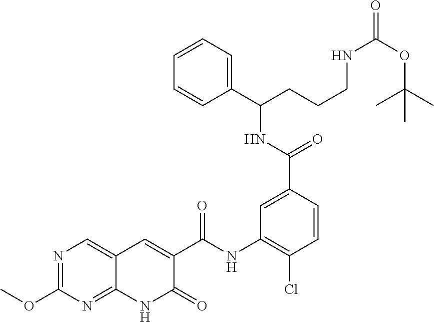

Triethylamine (0.022 mL, 0.16 mmol) was added to a mixture of 4-chloro-3-[(2-methoxy-7-oxo-7,8-dihydro-pyrido[2,3-d]pyrimidine-6-carbonyl)-amino]-benzoic acid (0.05 g, 0.13 mmol) (from Example 22 supra) and HATU (0.06 g, 0.16 mmol) (Aldrich) in DMF (2.0 mL). Mixture was stirred at room temperature for 30 minutes. tert-Butyl 4-amino-4-phenylbutyl carbamate (0.09 g, 0.33 mmol) was added. Mixture was stirred at room temperature for 18 hours and then partitioned between ethyl acetate and water. The precipitate was collected by filtration and washed with ethyl acetate and water and dried in vacuum oven to give N-(5-(5-amino-1-phenylpentylcarbamoyl)-2-chlorophenyl)-2-methoxy-7-oxo-7,8-dihydropyrido[2,3-d]pyrimidine-6-carboxamide. (Yield 0.038 g, 92.1%).

HR-MS (ES⁺) m/z Calculated for C₃₁H₃₄ClN₆O₆ ([M+H]⁺): 621.2223. Found: 621.2220.

### Example 189

### N-(5-(4-Amino-1-phenylbutylcarbamoyl)-2-chlorophenyl)-2-methoxy-7-oxo-7,8-dihydropyrido[2,3-d]pyrimidine-6-carboxamide

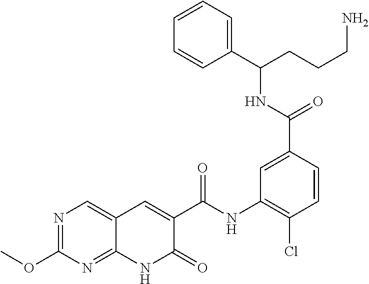

To a solution of tert-butyl 4-(4-chloro-3-(2-methoxy-7-oxo-7,8-dihydropyrido[2,3-d]pyrimidine-6-carboxamido)benzamido)-4-phenylbutylcarbamate (0.040 g, 0.064 mmol) (from Example 188 supra) in dichloromethane (5 mL) was added 2,2,2-trifluoroacetic acid (0.5 mL). The mixture was stirred at room temperature for 18 hours. The mixture was concentrated and then diluted with dichloromethane and saturated sodium carbonate solution. The precipitate was collected by filtration and washed with dichloromethane and water and dried in vacuum oven to give N-(5-(4-amino-1-phenylbutyl-carbamoyl)-2-chlorophenyl)-2-methoxy-7-oxo-7,8-dihydropyrido[2,3-d]pyrimidine-6-carboxamide. (Yield 0.029 g, 86.4%).

HR-MS (ES⁺) m/z Calculated for C₂₆H₂₆ClN₆O₄ ([M+H]⁺): 521.1699. Found: 521.1698.

### Example 190

### tert-Butyl 2-(4-chloro-3-(2-methoxy-7-oxo-7,8-dihydropyrido[2,3-d]pyrimidine-6-carboxamido)benzamido)-2-cyclohexylethylcarbamate

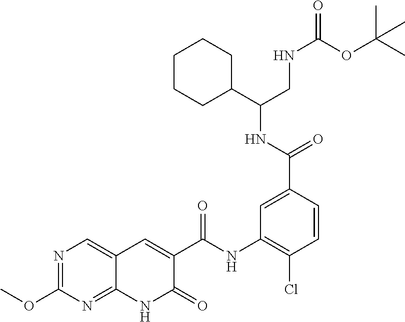

### Step A

### 2-Cyclohexyl-2-hydroxyacetonitrile

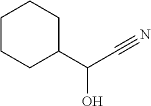

To a suspension of potassium cyanide (13.9 g, 0.21 mmol) in anhydrous ether (200 mL) at 5° C. was added dropwise a solution of cyclohexanecarbaldehyde (10 g, 89.2 mmol) (Aldrich) in conc. acetic acid (12 mL). The mixture was stirred at room temperature for 18 hours. The potassium acetate was filtered and the solution was concentrated to give crude 2-cyclohexyl-2-hydroxyacetonitrile. (Yield 9.46 g, 76.2%).

### Step B

### 2-Amino-1-cyclohexylethanol

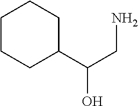

To a 1M solution of Lithium aluminum hydride in THF (80 mL) at 0° C. was added crude 2-cyclohexyl-2-hydroxyacetonitrile (9.42 g, 67.7 mmol) in THF (30 mL). The mixture was stirred at room temperature for 18 hours. The reaction was quenched with sequential addition of water (3.5 mL), 2N NaOH (9 mL) and water (13 mL). The aluminum salt was filtered off and the filtrate was concentrated to crude 2-amino-1-cyclohexylethanol. (Yield 7.15 g, 73.8%).

### Step C

### tert-Butyl 2-cyclohexyl-2-hydroxyethylcarbamate

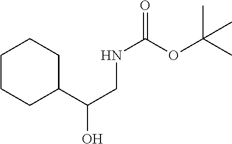

To a solution of crude 2-amino-1-cyclohexylethanol (7.15 g, 49.9 mmol) in THF (100 mL) was added di-tert-butyl dicarbonate (16.3 g, 74.9 mmol) (Aldrich) at room temperature with magnetic stirring. After 18 hours, the mixture was concentrated and the residue was purified by flash chromatography eluting with 5-30% ethyl acetate in hexanes to give tert-butyl 2-cyclohexyl-2-hydroxyethylcarbamate. (Yield 6.11 g, 50.3%).

### Step D

### tert-Butyl 2-cyclohexyl-2-(1,3-dioxoisoindolin-2-yl)ethylcarbamate

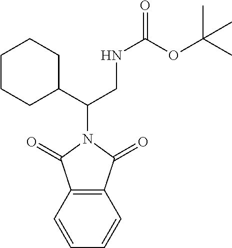

To a solution of tert-butyl 2-cyclohexyl-2-hydroxyethylcarbamate (6.11 g, 25.1 mmol), triphenylphosphine (7.57 g, 28.9 mmol) (Aldrich) and phthalimide (3.69 g, 25.1 mmol) (Aldrich) in THF (100 mL) was added diisopropylazo-dicarboxylate (5.69 mL, 28.9 mmol) (Aldrich) dropwise at room temperature with magnetic stirring. After 18 hours, mixture was concentrated and the residue was purified by flash chromatography eluting with 2-5% ethyl acetate in dichloromethane to give tert-butyl 2-cyclohexyl-2-(1,3-dioxoisoindolin-2-yl)ethylcarbamate. (Yield 7.37 g, 78.8%).

### Step E

### tert-Butyl 2-amino-2-cyclohexylethylcarbamate

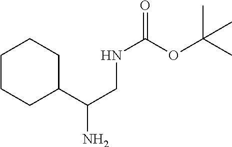

A solution of tert-butyl 2-cyclohexyl-2-(1,3-dioxoisoindolin-2-yl)ethylcarbamate (7.37 g, 19.8 mmol) and anhydrous hydrazine (6.14 mL, 198 mmol) in methanol (100 mL) was heated at reflux for 8 hours. Solid was filtered and washed with methanol. The filtrate was concentrated. The residue was diluted with dichloromethane and 1N NaOH solution. The aqueous phase was extracted with dichloromethane (1×). The combined organic phase was washed with brine, dried (magnesium sulfate), filtered, and concentrated to give crude tert-butyl 2-amino-2-cyclohexylethylcarbamate. (Yield 4.8 g, 100.0%).

### Step F

### tert-Butyl 2-(4-chloro-3-(2-methoxy-7-oxo-7,8-dihydropyrido[2,3-d]pyrimidine-6-carboxamido)benzamido)-2-cyclohexylethylcarbamate

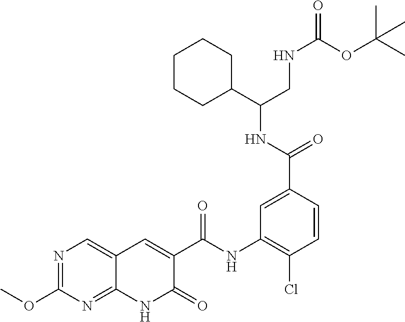

Triethylamine (0.042 mL, 0.30 mmol) was added to a mixture of 4-chloro-3-[(2-methoxy-7-oxo-7,8-dihydro-pyrido[2,3-d]pyrimidine-6-carbonyl)-amino]-benzoic acid (0.10 g, 0.27 mmol) (from Example 22 supra) and HATU (0.11 g, 0.30 mmol) (Aldrich) in DMF (5.0 mL). Mixture was stirred at room temperature for 30 minutes. Crude tert-butyl 2-amino-2-cyclohexylethylcarbamate (0.13 g, 0.53 mmol) was added. Mixture was stirred at room temperature for 1 day and then partitioned between ethyl acetate and water. The aqueous phase was extracted with ethyl acetate (3×). The combined organic phase was washed with water and brine, dried (magnesium sulfate), filtered, and concentrated. The residue was washed with ethyl acetate—water to give tert-butyl 2-(4-choro-3-(2-methoxy-7-oxo-7,8-dihydropyrido[2,3-d]pyrimidine-6-carboxamido)benzamido)-2-cyclohexylethyl-carbamate. (Yield 0.055 g, 34.4%).

HR-MS (ES⁺) m/z Calculated for C₂₉H₃₆ClN₆O₆ ([M+H]⁺): 599.2380. Found: 599.2376.

### Example 191

### tert-Butyl 3-(4-chloro-3-(2-methoxy-7-oxo-7,8-dihydropyrido[2,3-d]pyrimidine-6-carboxamido)benzamido)-3-cyclohexylpropylcarbamate

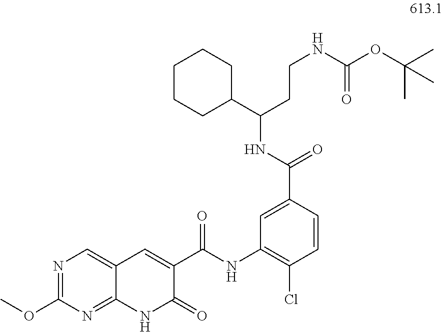

### Step A

### 3-Cyclohexyl-3-oxopropanenitrile

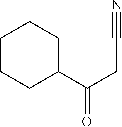

A suspension of sodium hydride (60%, 3.1 g, 77.4 mmol) in THF (70 mL) was heated at 75° C. with magnetic stirring for 15 minutes. A mixture of methyl cyclohexanecarboxylate (10 g, 70.3 mmol) (Aldrich) and MeCN (4.8 mL, 91.4 mmol) in THF (70 mL) was added dropwise. The mixture was stirred for an additional 4 hours and then partitioned between ethyl acetate and 1N HCl solution. The organic phase was separated, dried (magnesium sulfate), filtered, and concentrated. The residue was purified by flash chromatography eluting with 0-30% ethyl acetate in hexanes to give 3-cyclohexyl-3-oxopropanenitrile. (Yield 4.04 g, 38.0%).

### Step B

### 3-Amino-1-cyclohexylpropan-1-ol

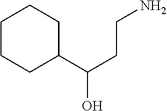

To a suspension of lithium aluminum hydride (2.27 g, 61.5 mmol) in THF (30 mL) at 0° C. was added 3-cyclohexyl-3-oxopropanenitrile (4.04 g, 26.7 mmol) in THF (30 mL). The mixture was stirred at room temperature for 18 hours. The reaction was quenched with sequential addition of water (3.0 mL), 2N NaOH (6 mL) and water (10 mL). The aluminum salt was filtered and the filtrate was concentrated to give crude 3-amino-1-cyclohexylpropan-1-ol. (Yield 4.03 g, 95.5%).

### Step C

### tert-Butyl 3-cyclohexyl-3-hydroxypropylcarbamate

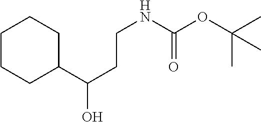

To a solution of crude 3-amino-1-cyclohexylpropan-1-ol (4.03 g, 25.6 mmol) in THF (100 mL) was added di-tert-butyl dicarbonate (8.39 g, 38.4 mmol) (Aldrich) at room temperature with magnetic stirring. After 18 hours, the mixture was concentrated and the residue was purified by flash chromatography eluting with 5-30% ethyl acetate in hexanes to give tert-butyl 3-cyclohexyl-3-hydroxypropylcarbamate. (Yield 4.07 g, 61.7%).

### Step D

### tert-Butyl 3-cyclohexyl-3-(1,3-dioxoisoindolin-2-yl)propylcarbamate

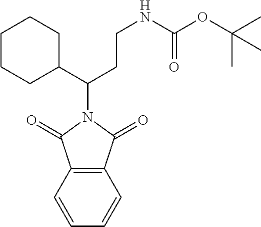

To a solution of tert-butyl 3-cyclohexyl-3-hydroxypropylcarbamate (4.07 g, 15.8 mmol), triphenylphosphine (4.77 g, 18.2 mmol) (Aldrich) and phthalimide (2.33 g, 15.8 mmol) (Aldrich) in THF (80 mL) was added diisopropylazo-dicarboxylate (3.58 mL, 18.2 mmol) (Aldrich) dropwise at room temperature with magnetic stirring. After 18 hours, mixture was concentrated and the residue was purified by flash chromatography eluting with 2-5% ethyl acetate in dichloromethane to give tert-butyl 3-cyclohexyl-3-(1,3-dioxoisoindolin-2-yl)propylcarbamate. (Yield 2.13 g, 34.9%).

### Step E

### tert-Butyl 3-amino-3-cyclohexylpropyl-carbamate

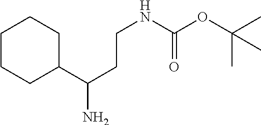

A solution of tert-butyl 3-cyclohexyl-3-(1,3-dioxoisoindolin-2-yl)propylcarbamate (2.13 g, 5.51 mmol) and anhydrous hydrazine (1.71 mL, 55.1 mmol) in methanol (50 mL) was heated at reflux for 8 hours. Solid was filtered and washed with methanol. The filtrate was concentrate. The residue was diluted with dichloromethane and 1N NaOH solution. The aqueous phase was extracted with dichloromethane (1×). The combined organic phase was washed with brine, dried (magnesium sulfate), filtered, and concentrated. The residue was purified by flash chromatography eluting with 0-10% methanol in dichloromethane to give tert-butyl 3-amino-3-cyclohexylpropyl-carbamate. (Yield 0.59 g, 41.8%).

### Step F

### tert-Butyl 3-(4-chloro-3-(2-methoxy-7-oxo-7,8-dihydropyrido[2,3-d]pyrimidine-6-carboxamido)benzamido)-3-cyclohexylpropylcarbamate

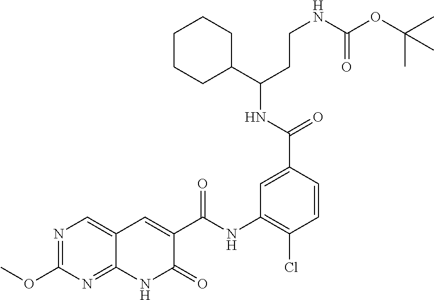

Triethylamine (0.033 mL, 0.24 mmol) was added to a mixture of 4-chloro-3-[(2-methoxy-7-oxo-7,8-dihydro-pyrido[2,3-d]pyrimidine-6-carbonyl)-amino]-benzoic acid (0.08 g, 0.21 mmol) (from Example 22 supra) and HATU (0.09 g, 0.24 mmol) (Aldrich) in DMF (5.0 mL). Mixture was stirred at room temperature for 30 minutes. tert-Butyl 3-amino-3-cyclohexylpropyl-carbamate (0.08 g, 0.32 mmol) was added. Mixture was stirred at room temperature for 18 hours and then partitioned between ethyl acetate and water. The aqueous phase was extracted with ethyl acetate (3×). The combined organic phase was washed with water and brine, dried (magnesium sulfate), filtered, and concentrated. The residue was recrystallized from hexanes-ethyl acetate to give tert-butyl 3-(4-choro-3-(2-methoxy-7-oxo-7,8-dihydropyrido[2,3-d]pyrimidine-6-carboxamido)benzamido)-3-cyclohexylpropylcarbamate. (Yield 0.106 g, 81.0%).

HR-MS (ES⁺) m/z Calculated for C₃₀H₃₈ClN₆O₆ ([M+H]⁺): 613.2536. Found: 613.2533.

### Example 192

### N-(5-(2-Amino-1-cyclohexylethylcarbamoyl)-2-chlorophenyl)-2-methoxy-7-oxo-7,8-dihydropyrido[2,3-d]pyrimidine-6-carboxamide

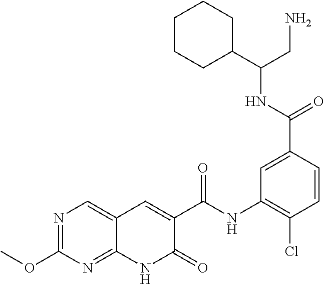

To a solution of tert-butyl 2-(4-chloro-3-(2-methoxy-7-oxo-7,8-dihydropyrido[2,3-d]pyrimidine-6-carboxamido)benzamido)-2-cyclohexylethylcarbamate (0.047 g, 0.079 mmol) (from Example 190 supra) in dichloromethane (5 mL) was added 2,2,2-trifluoroacetic acid (0.5 mL). The mixture was stirred at room temperature for 18 hours. The mixture was concentrated and then diluted with dichloromethane and saturated sodium carbonate solution. The precipitate was collected by filtration and washed with dichloromethane and water and dried in vacuum oven to give N-(5-(2-amino-1-cyclohexylethylcarbamoyl)-2-chlorophenyl)-2-methoxy-7-oxo-7,8-dihydropyrido[2,3-d]pyrimidine-6-carboxamide. (Yield 0.022 g, 56.2%).

HR-MS (ES⁺) m/z Calculated for C₂₄H₂₈ClN₆O₄ ([M+H]⁺): 499.1855. Found: 499.1853.

### Example 193

### N-(5-(3-Amino-1-cyclohexylpropylcarbamoyl)-2-chlorophenyl)-2-meth oxy-7-oxo-7,8-dihydropyrido[2,3-d]pyrimidine-6-carboxamide 2,2,2-trifluoroacetate

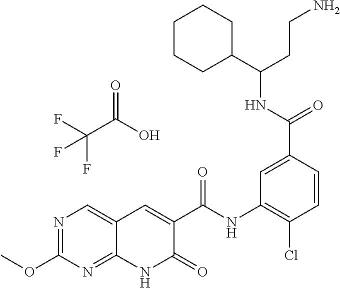

To a solution of tert-butyl 3-(4-chloro-3-(2-methoxy-7-oxo-7,8-dihydropyrido[2,3-d]pyrimidine-6-carboxamido)benzamido)-3-cyclohexylpropylcarbamate (0.104 g, 0.17 mmol) (from Example 191 supra) in dichloromethane (5 mL) was added 2,2,2-trifluoroacetic acid (0.5 mL). The mixture was stirred at room temperature for 18 hours. The mixture was concentrated and then diluted with dichloromethane and saturated sodium carbonate solution. The precipitate was collected by filtration and washed with dichloromethane and water and dried in vacuum oven. The crude material was purified by HPLC eluting with MeCN—H₂O to give N-(5-(3-amino-1-cyclohexylpropylcarbamoyl)-2-chlorophenyl)-2-methoxy-7-oxo-7,8-dihydropyrido[2,3-d]pyrimidine-6-carboxamide 2,2,2-trifluoroacetate. (Yield 0.055 g, 51.7%).

HR-MS (ES⁺) m/z Calculated for C₂₅H₃₀ClN₆O₄ ([M+H]⁺): 513.2012. Found: 513.2008.

### Example 194

### (3-Amino-2,2-difluoro-3-phenyl-propyl)-carbamic acid tert-butyl ester

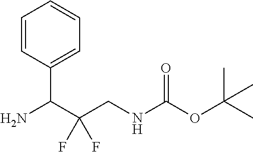

### Step A

### (4-Methoxy-benzyl)-[1-phenyl-meth-(E)-ylidene]-amine

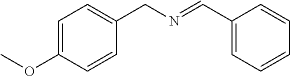

To a stirred solution of 4-methoxy-benzylamine (30.00 g, 218.69 mmol) in dichloromethane (300 mL) was added benzaldehyde (23.18 g, 218.69 mmol) and MgSO₄ (46.30 g, 382.70 mmol) and the resulting mixture was stirred at RT for 12 hours (silica TLC; EtOAc-hexanes 1:9 R_{f}=0.8). The reaction mass was filtered through a Celite bed and washed with dichloromethane (˜50 mL). Collected filtrate was concentrated under reduced pressure to give (4-methoxy-benzyl)-[1-phenyl-meth-(E)-ylidene]-amine as a colorless oil. (Yield 45.0 g, 91.8%).

### Step B

### 3,3-Difluoro-1-(4-methoxy-benzyl)-4-phenyl-azetidin-2-one

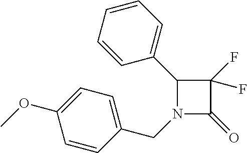

To a refluxing suspension of freshly activated Zn dust (5.80 g, 77.77 mmol) in dry THF (50 mL) was added (4-methoxy-benzyl)-[1-phenyl-meth-(E)-ylidene]-amine (5.00 g, 22.22 mmol) and a solution of ethyl bromodifluoro acetate (5.80 g, 88.88 mmol) in THF (5 mL) simultaneously at reflux temperature under nitrogen. The reaction mixture was heated at reflux for 1 hour (silica gel TLC; EtOAc-hexanes 1:9; R_{f}=0.6). After cooling to room temperature, the mixture was quenched with saturated NH₄Cl solution (50 mL), filtered through a Celite bed and the filtrate was extracted with dichloromethane (2×50 mL). The collected organic parts were washed with water, brine, dried, filtered and concentrated under reduced pressure to obtain a crude mass, which was purified by column chromatography (silica gel, using ethyl acetate-hexanes, 1:9) to give pure 3,3-difluoro-1-(4-methoxy-benzyl)-4-phenyl-azetidin-2-one. (Yield 3.20 g, 47.8%).

### Step C

### 2,2-Difluoro-3-(4-methoxy-benzylamino)-3-phenyl-propionamide

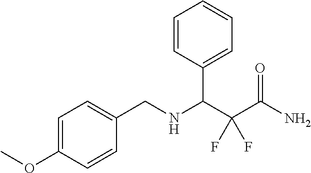

A solution of 3,3-difluoro-1-(4-methoxy-benzyl)-4-phenyl-azetidin-2-one (44.00 g, 148.99 mmol) in 10% ammonia in methanol (350 mL) was stirred at 0° C. for 4 hours (silica gel TLC; EtOAc-hexanes 2:3; R_{f}=0.4). Reaction mixture was concentrated under reduced pressure to obtain crude mass that was triturated with hexanes to give 2,2-difluoro-3-(4-methoxy-benzylamino)-3-phenyl-propionamide as off white solid. (Yield 42.00 g, 90.4%).

### Step D

### 2,2-Difluoro-N1-(4-methoxy-benzyl)-1-phenyl-propane-1,3-diamine

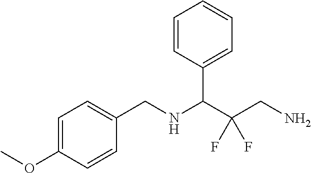

BH₃—(CH₃)₂S complex (32.9 mL, 342.60 mmol) was added to a stirred solution of 2,2-difluoro-3-(4-methoxy-benzylamino)-3-phenyl-propionamide (42.00 g, 137.07 mmol) in dry THF (500 mL) at room temperature under nitrogen and the reaction mixture was heated at 80-90° C. for 4 hours (silica gel TLC; EtOAc-hexanes 1:1; R_{f}=0.5). Reaction was quenched by addition of 3N HCl (75 mL) solution and was washed with ethyl acetate (to get rid of organic impurities). Organic part was separated out, pH of the aqueous part was adjusted to about 12 with the addition of aqueous NaOH [10% (w/w)] solution and was extracted with ethyl acetate (2×100 mL). The organic parts were washed with water, brine, dried, filtered, and concentrated under reduced pressure to give a crude mass, which was purified by column chromatography (silica gel, ethyl acetate-hexanes, 1:1 as eluting solvent) to give 2,2-difluoro-N1-(4-methoxy-benzyl)-1-phenyl-propane-1,3-diamine as a light brown liquid. (Yield 24.0 g, 60%).

### Step E

### [2,2-Difluoro-3-(4-methoxy-benzylamino)-3-phenyl-propyl]-carbamic acid tert-butyl ester

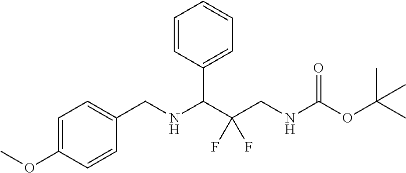

To a stirred solution of 2,2-difluoro-N-1-(4-methoxy-benzyl)-1-phenyl-propane-1,3-diamine (25.00 g, 81.69 mmol) in dry dichloromethane (500 mL) were add TEA (11.38 mL, 81.69 mmol) and (Boc)₂O (17.0 mL, 81.69 mmol) simultaneously at 0° C. under nitrogen. The reaction mixture was stirred for 16 hours at room temperature (silica gel TLC; EtOAc-hexanes 1:5; R_{f}=0.8). Reaction mixture was concentrated under reduced pressure and the obtained crude mass was purified by column chromatography (silica gel, ethyl acetate-hexanes 1:5 as eluting solvent) to give [2,2-difluoro-3-(4-methoxy-benzylamino)-3-phenyl-propyl]-carbamic acid tert-butyl ester as a light yellow liquid. (Yield 26.2 g, 84.6%).

### Step F

### (3-Amino-2,2-difluoro-3-phenyl-propyl)-carbamic acid tert-butyl ester

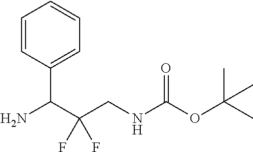

To a deoxygenated solution (argon was purged through the solvent for 10 minutes) of [2,2-difluoro-3-(4-methoxy-benzylamino)-3-phenyl-propyl]-carbamic acid tert-butyl ester (26.00 g, 64.03 mmol) in methanol (250 mL) was add Pd(OH)₂—C (1.1 g/1 g, 28.6 g) at room temperature and was hydrogenated under balloon pressure (about 20 psi) for 5 hours (silica gel TLC; EtOAc-hexanes 1:1; R_{f}=0.5). The reaction mixture was filtered through a Celite bed, residue was washed with methanol (50 mL) and the combined filtrates was concentrated under reduced pressure to give crude mass, which was purified by column chromatography (silica gel, ethyl acetate-hexanes, 1:1 as eluting solvent) to give (3-amino-2,2-difluoro-3-phenyl-propyl)-carbamic acid tert-butyl ester as a white solid. (Yield 13.5 g, 74.2%). LR-MS (ES⁺) [M+H]⁺ 287.

### Example 195

### tert-Butyl 3-(4-chloro-3-(2-methoxy-7-oxo-7,8-dihydropyrido[2,3-d]pyrimidine-6-carboxamido)benzamido)-2,2-difluoro-3-phenylpropylcarbamate

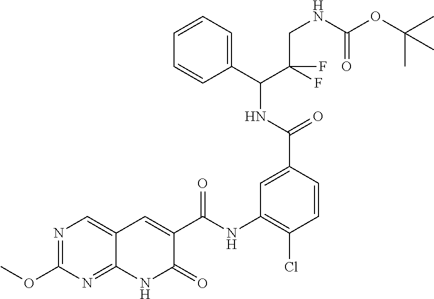

Triethylamine (0.042 mL, 0.30 mmol) was added to a mixture of 4-chloro-3-[(2-methoxy-7-oxo-7,8-dihydro-pyrido[2,3-d]pyrimidine-6-carbonyl)-amino]-benzoic acid (0.10 g, 0.27 mmol) (from Example 22 supra) and HATU (0.11 g, 0.30 mmol) (Oakwood) in DMF (5.0 mL). Mixture was stirred at room temperature for 30 minutes. (3-Amino-2,2-difluoro-3-phenyl-propyl)-carbamic acid tert-butyl ester (0.10 g, 0.35 mmol) (from Example 194 supra) was added. Mixture was stirred at room temperature for 18 hours and then partitioned between ethyl acetate and water. The aqueous phase was extracted with ethyl acetate (3×). The combined organic phase was washed with water and brine, dried (magnesium sulfate), filtered, and concentrated. The residue was washed with ethyl acetate and water to give tert-butyl 3-(4-choro-3-(2-methoxy-7-oxo-7,8-dihydropyrido[2,3-d]pyrimidine-6-carboxamido)-2,2-difluoro-3-phenylpropylcarbamate. (Yield 0.12 g, 69.9%). HR-MS (ES⁺) m/z Calculated for C₃₀H₃₀ClF₂N₆O₆ ([M+H]⁺): 643.1878. Found: 643.1874.

### Example 196

### N-(5-(3-amino-2,2-difluoro-1-phenylpropylcarbamoyl)-2-chlorophenyl)-2-methoxy-7-oxo-7,8-dihydropyrido[2,3-d]pyrimidine-6-carboxamide

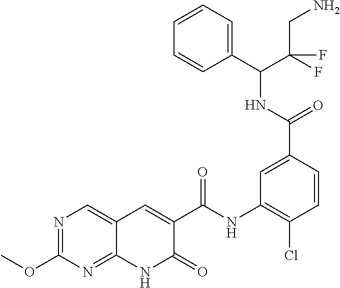

To a solution of tert-butyl 3-(4-chloro-3-(2-methoxy-7-oxo-7,8-dihydropyrido[2,3-d]pyrimidine-6-carboxamido)benzamido)-2,2-difluoro-3-phenylpropy-lcarbamate (0.12 g, 0.19 mmol) (from Example 195 supra) in dichloromethane (10 mL) was added 2,2,2-trifluoroacetic acid (1.5 mL). The mixture was stirred at room temperature for 18 hours. The mixture was concentrated and then diluted with dichloromethane and saturated sodium carbonate solution. The precipitate was collected by filtration and washed with dichloromethane and water and dried in vacuum oven to give N-(5-(3-amino-2,2-difloro-1-phenylpropylcarbamoyl)-2-chlorophenyl)-2-methoxy-7-oxo-7,8-dihydropyrido[2,3-d]pyrimidine-6-carboxamide. (Yield 0.085 g, 83.9%).

HR-MS (ES⁺) m/z Calculated for C₂₅H₂₂ClF₂N₆O₄ ([M+H]⁺): 543.1354. Found: 543.1350.

### Example 197

### tert-Butyl 3-(4-chloro-3-(2-methoxy-7-oxo-7,8-dihydropyrido[2,3-d]pyrimidine-6-carboxamido)benzamido)-3-cyclopentylpropylcarbamate

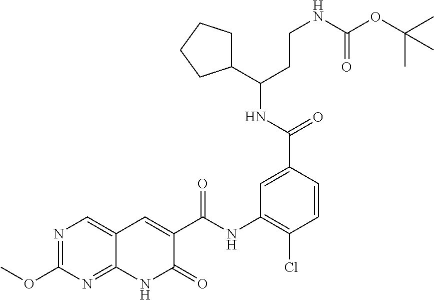

### Step A

### 3-Cyclopentyl-3-oxopropanenitrile

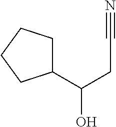

A suspension of sodium hydride (60%, 3.43 g, 85.8 mmol) in THF (70 mL) was heated at 75° C. with magnetic stirring for 15 minutes. A mixture of methyl cyclopentanecarboxylate (10 g, 78.0 mmol) (Aldrich) and MeCN (5.34 mL, 0.1 mmol) in THF (70 mL) was added dropwise. The mixture was stirred for an additional 4 hours and then partitioned between ethyl acetate and 1N HCl solution. The organic phase was separated, dried (magnesium sulfate), filtered, and concentrated. The residue was purified by flash chromatography eluting with 0-30% ethyl acetate in hexanes to give 3-cyclopentyl-3-oxopropanenitrile (Yield 1.48 g, 13.8%).

### Step B

### 3-Amino-1-cyclopentylpropan-1-ol

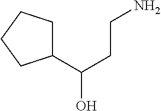

To a suspension of lithium aluminum hydride (0.88 g, 23.1 mmol) in THF (15 mL) at 0° C. was added 3-cyclopentyl-3-oxopropanenitrile (1.38 g, 10.1 mmol) in THF (15 mL). The mixture was stirred at room temperature for 18 hours. The reaction was quenched with sequential addition of water (1.0 mL), 2N NaOH (3 mL) and water (3 mL). The aluminum salt was filtered and the filtrate was concentrated to give crude 3-amino-1-cyclopentylpropan-1-ol. (Yield 1.40 g, 97.2%).

### Step C

### tert-Butyl 3-cyclopentyl-3-hydroxypropylcarbamate

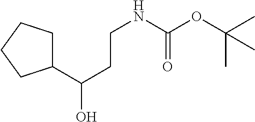

To the solution of crude 3-amino-1-cyclopentylpropan-1-ol (1.4 g, 9.77 mmol) in THF (20 mL) was added di-tert-butyl dicarbonate (3.2 g, 14.7 mmol) (Aldrich) at room temperature with magnetic stirring. After 18 hours, the mixture was concentrated and the residue was purified by flash chromatography eluting with 5-30% ethyl acetate in hexanes to give tert-butyl 3-cyclopentyl-3-hydroxypropylcarbamate. (Yield 0.86 g, 36.2%).

### Step D

### tert-Butyl 3-cyclopentyl-3-(1,3-dioxoisoindolin-2-yl)propylcarbamate

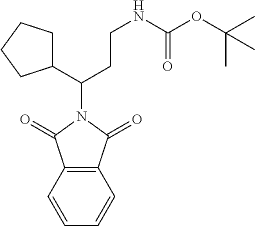

To a solution of tert-butyl 3-cyclopentyl-3-hydroxypropylcarbamate (0.86 g, 3.53 mmol), triphenylphosphine (1.07 g, 4.06 mmol) (Aldrich) and phthalimide (0.52 g, 3.53 mmol) (Aldrich) in THF (20 mL) was added diisopropylazo-dicarboxylate (0.8 mL, 4.06 mmol) (Aldrich) dropwise at room temperature with magnetic stirring. After 18 hours, mixture was concentrated and the residue was purified by flash chromatography eluting with 2-5% ethyl acetate in dichloromethane to give tert-butyl 3-cyclopentyl-3-(1,3-dioxoisoindolin-2-yl)propylcarbamate. (Yield 0.38 g, 28.9%).

### Step E

### tert-Butyl 3-amino-3-cyclohexylpropyl-carbamate

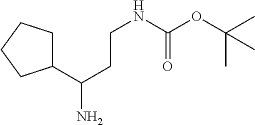

A solution of tert-butyl 3-cyclopentyl-3-(1,3-dioxoisoindolin-2-yl)propylcarbamate (0.38 g, 1.02 mmol) and anhydrous hydrazine (0.03 mL, 10.2 mmol) in methanol (10 mL) was heated at reflux for 8 hours. Solid was filtered and washed with methanol. The filtrate was concentrate. The residue was diluted with dichloromethane and 1N NaOH solution. The aqueous phase was extracted with dichloromethane (1×). The combined organic phase was washed with brine, dried (magnesium sulfate), filtered, and concentrated. The residue was purified by flash chromatography eluting with 0-10% methanol in dichloromethane to give tert-butyl 3-amino-3-cyclohexylpropyl-carbamate. (Yield 0.22 g, 89.0%).

### Step F

### tert-Butyl 3-(4-chloro-3-(2-methoxy-7-oxo-7,8-dihydropyrido[2,3-d]pyrimidine-6-carboxamido)benzamido)-3-cyclopentylpropylcarbamate

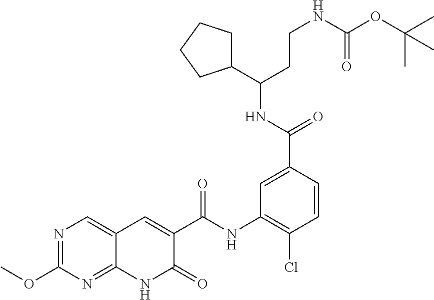

Triethylamine (0.042 mL, 0.30 mmol) was added to a mixture of 4-chloro-3-[(2-methoxy-7-oxo-7,8-dihydro-pyrido[2,3-d]pyrimidine-6-carbonyl)-amino]-benzoic acid (0.10 g, 0.27 mmol) (from Example 22 supra) and HATU (0.11 g, 0.30 mmol) (Aldrich) in DMF (5.0 mL). Mixture was stirred at room temperature for 30 minutes. tert-Butyl 3-amino-3-cyclohexylpropyl-carbamate (0.10 g, 0.4 mmol) was added. Mixture was stirred at room temperature for 1 day and then partitioned between ethyl acetate and water. The aqueous phase was extracted with ethyl acetate (3×). The combined organic phase was washed with water and brine, dried (magnesium sulfate), filtered, and concentrated. The residue was collected by filtration and washed with ethyl acetate, water and dried in vacuum oven to give tert-butyl 3-(4-choro-3-(2-methoxy-7-oxo-7,8-dihydropyrido[2,3-d]pyrimidine-6-carboxamido)benzamido)-3-cyclopentylpropylcarbamate. (Yield 0.117 g, 73.2%).

HR-MS (ES⁺) m/z Calculated for C₂₉H₃₅ClN₆NaO₆ ([M+Na]⁺): 621.2199. Found: 621.2199.

### Example 198

### 2-Methoxy-7-oxo-7,8-dihydro-pyrido[2,3-d]pyrimidine-6-carboxylic acid [5-(3-amino-1-cyclopentyl-propylcarbamoyl)-2-chloro-phenyl]-amide

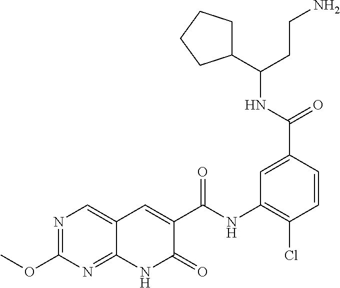

To a solution of tert-butyl 3-(4-chloro-3-(2-methoxy-7-oxo-7,8-dihydropyrido[2,3-d]pyrimidine-6-carboxamido)benzamido)-3-cyclopentylpropylcarbamate (0.10 g, 0.167 mmol) (from Example 197 supra) in dichloromethane (5 mL) was added 2,2,2-trifluoroacetic acid (0.5 mL). The mixture was stirred at room temperature for 18 hours. The mixture was concentrated and then diluted with methanol and saturated sodium carbonate solution. The precipitate was collected by filtration and washed with water and dried in vacuum oven to give N-(5-(3-amino-1-cyclopentylpropylcarbamoyl)-2-chlorophenyl)-2-methoxy-7-oxo-7,8-dihydropyrido[2,3-d]pyrimidine-6-carboxamide. (Yield 0.082 g, 98.5%).

HR-MS (ES⁺) m/z Calculated for C₂₄H₂₈ClN₆O₄ ([M+H]⁺): 499.1855. Found: 499.1852.

### Example 199

### (2-{4-Chloro-3-[(2-methoxy-7-oxo-7,8-dihydro-pyrido[2,3-d]pyrimidine-6-carbonyl)-amino]-benzoylamino}-2-cyclopentyl-ethyl)-carbamic acid tert-butyl ester

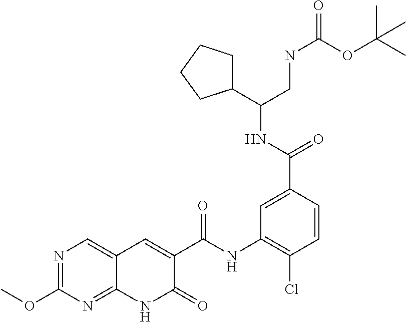

### Step A

### 2-Cyclopentyl-2-hydroxyacetonitrile

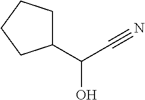

To a suspension of potassium cyanide (4.65 g, 0.21 mmol) in anhydrous ether (100 mL) at 5° C. was added dropwise a solution of cyclopentanecarbaldehyde (5.0 g, 50.9 mmol) (Aldrich) in conc. acetic acid (6 mL). The mixture was stirred at room temperature for 18 hours. Potassium acetate was filtered and the filtrate was concentrated to give crude 2-cyclopentyl-2-hydroxyacetonitrile. (Yield 6.38 g, 100%).

### Step B

### 2-Amino-1-cyclopentylethanol

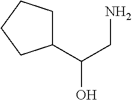

To a solution of lithium aluminum hydride (2.26 g, 61.2 mmol) in THF (80 mL) at 0° C. was added crude 2-cyclopentyl-2-hydroxyacetonitrile (6.38 g, 51.0 mmol) in THF (30 mL). The mixture was stirred at room temperature for 18 hours. The reaction was quenched with sequential addition of water (3.5 mL), 2N NaOH (9 mL) and water (13 mL). The aluminum salt was filtered and the filtrate was concentrated to give crude 2-amino-1-cyclopentylethanol. (Yield 4.67 g, 70.9%).

### Step C

### tert-Butyl 2-cyclopentyl-2-hydroxyethylcarbamate

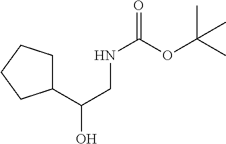

To a solution of crude 2-amino-1-cyclopentylethanol (4.67 g, 36.1 mmol) in THF (100 mL) was added di-tert-butyl dicarbonate (10.3 g, 47.0 mmol) (Aldrich) at room temperature with magnetic stirring. After 18 hours, the mixture was concentrated and the residue was purified by flash chromatography eluting with 5-30% ethyl acetate in hexanes to give tert-butyl 2-cyclopentyl-2-hydroxyethylcarbamate. (Yield 6.11 g, 73.3%).

### Step D

### tert-Butyl 2-cyclohexyl-2-(1,3-dioxoisoindolin-2-yl)ethylcarbamate

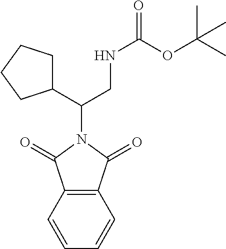

To a solution of tert-butyl 2-cyclopentyl-2-hydroxyethylcarbamate (6.11 g, 26.6 mmol), triphenylphosphine (8.04 g, 30.6 mmol) (Aldrich) and phthalimide (3.92 g, 26.6 mmol) (Aldrich) in THF (100 mL) was added diisopropylazo-dicarboxylate (6.35 mL, 30.6 mmol) (Aldrich) dropwise at room temperature with magnetic stirring. After 18 hours, mixture was concentrated and the residue was purified by flash chromatography eluting with 2-5% ethyl acetate in dichloromethane to give tert-butyl 2-cyclohexyl-2-(1,3-dioxoisoindolin-2-yl)ethylcarbamate. (Yield 1.97 g, 20.6%).

### Step E

### tert-Butyl 2-amino-2-cyclopentylethylcarbamate

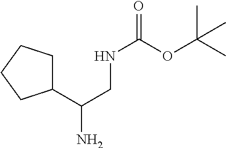

A solution of tert-butyl 2-cyclopentyl-2-(1,3-dioxoisoindolin-2-yl)ethylcarbamate (1.97 g, 5.5 mmol) and anhydrous hydrazine (1.71 mL, 55 mmol) in methanol (50 mL) was heated at reflux for 18 hours. Solid was filtered and washed with methanol. The filtrate was concentrated. The residue was diluted with dichloromethane and 1N NaOH solution. The aqueous phase was extracted with dichloromethane (1×). The combined organic phase was washed with brine, dried (magnesium sulfate), filtered, and concentrated. The residue was purified by flash chromatography eluting with 0-10% methanol in dichloromethane to give tert-butyl 2-amino-2-cyclopentylethylcarbamate. (Yield 1.24 g, 98.8%).

### Step F

### (2-{4-Chloro-3-[(2-methoxy-7-oxo-7,8-dihydro-pyrido[2,3-d]pyrimidine-6-carbonyl)-amino]-benzoylamino}-2-cyclopentyl-ethyl)-carbamic acid tert-butyl ester

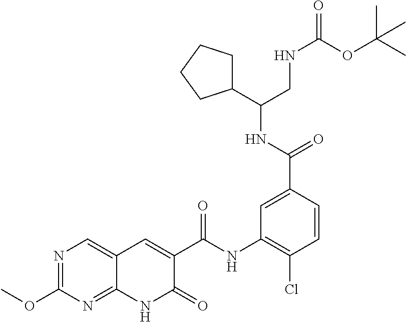

Triethylamine (0.042 mL, 0.30 mmol) was added to a mixture of 4-chloro-3-[(2-methoxy-7-oxo-7,8-dihydro-pyrido[2,3-d]pyrimidine-6-carbonyl)-amino]-benzoic acid (0.10 g, 0.27 mmol) (from Example 22 supra) and HATU (0.11 g, 0.30 mmol) (Aldrich) in DMF (5.0 mL). Mixture was stirred at room temperature for 30 minutes. tert-Butyl 2-amino-2-cyclopentylethyl-carbamate (0.12 g, 0.53 mmol) was added. Mixture was stirred at room temperature for 1 day and then partitioned between ethyl acetate and water. The aqueous phase was extracted with ethyl acetate (3×). The combined organic phase was washed with water and brine, dried (magnesium sulfate), filtered, and concentrated. The residue was collected by filtration and washed with hot ethyl acetate and dried in vacuum oven to give tert-butyl 2-(4-choro-3-(2-methoxy-7-oxo-7,8-dihydropyrido[2,3-d]pyrimidine-6-carboxamido)benzamido)-2-cyclopentylethylcarbamate. (Yield 0.104 g, 66.6%).

HR-MS (ES⁺) m/z Calculated for C₂₈H₃₄ClN₆O₆ ([M+H]⁺): 585.2223. Found: 585.2220.

### Example 200

### 2-Methoxy-7-oxo-7,8-dihydro-pyrido[2,3-d]pyrimidine-6-carboxylic acid [5-(2-amino-1-cyclopentyl-ethylcarbamoyl)-2-chloro-phenyl]-amide

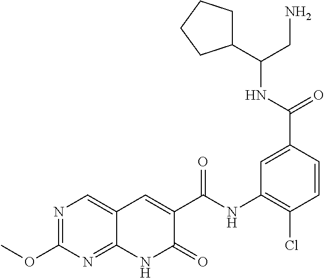

To a solution of 2-(4-chloro-3-(2-methoxy-7-oxo-7,8-dihydropyrido[2,3-d]pyrimidine-6-carboxamido)benzamido)-2-cyclopentylethylcarbamate (0.094 g, 0.161 mmol) (from Example 199 supra) in dichloromethane (5 mL) was added 2,2,2-trifluoroacetic acid (0.5 mL). The mixture was stirred at room temperature for 18 hours. The mixture was concentrated and then diluted with methanol and saturated sodium carbonate solution. The precipitate was collected by filtration and washed with water and dried in vacuum oven to give N-(5-(2-amino-1-cyclopentyl-ethylcarbamoyl)-2-chlorophenyl)-2-methoxy-7-oxo-7,8-dihydropyrido[2,3-d]pyrimidine-6-carboxamide. (Yield 0.077 g, 98.8%). HR-MS (ES⁺) m/z Calculated for C₂₃H₂₅ClN₆O₄ ([M+H]⁺): 485.1699. Found: 485.1699.

The pharmacological properties of the compounds of this invention may be confirmed by a number of pharmacological assays. The exemplified antiproliferative activity assays which follow have been carried out with the compounds according to the invention.

If test compounds were assessed in multiple runs of the same assay, the activities reported in Table I below are the averages of the results obtained from the multiple runs of the assay.

### Example 201

### DYRK1B Kinase TR-FRET (IMAP-Tb) Assay

### Assay Principle

The kinase TR-FRET (IMAP-Tb) assay uses a fluorescence labeled substrate peptide in the kinase reaction. Upon phosphorylation by the kinase, phosphopeptide is produced, which will be detected by the binding solution provided in IMAP TR-FRET binding kit. After the completion of the kinase reaction, the reaction will be stopped by adding the binding solution containing terbium tracer. This tracer is immobilized on the surface of the IMAP beads, which also contain metal ions on the beads that bind to the phospho-groups of the products. Thus the phosphorylated product of the reaction can enter into close proximity to the tracer, producing resonance energy transfer. Due to the long lifetime of terbium (Tb) fluorescence the detection can be run in time resolved mode, which virtually eliminates fluorescence interference from assay components or compounds.

The TR-FRET signal measurement from this assay, given as an IC₅₀ measurement, is a measure of a test compound's ability to interfere with the phosphorylation of the peptide substrate, that is it inhibits the phosphorylation of the substrate peptide by DYRK1B, and is thus a measure of the test compound's ability to inhibit the activity of DYRK1B. IC₅₀ is the amount of test compound that inhibits 50% of the activity of DYRK1B in this assay. In some cases where the IC₅₀ was not determined, then the % inhibition at 10 μM test compound concentration was reported. The results of this assay for sample compounds of the invention are provided in Table I below.

### Materials and Reagents

1. Human DYRK1B: from Invitrogen. Part #PR8350B (former PV4649)
2. Substrate Peptide: in-house synthesized: RRRFRPASPLRGPPK
3. IMAP TR-FRET IPP Explorer Kit: from Molecular Devices. Part #R8157
4. Kinase Assay Buffer (KAB): 10 mM HEPES pH 7.0, 50 mM NaCl, 5 mM MgCl₂, 1 mM DTT, 1 mM NaVO₄, 200 μg/mL BSA (0.02%)
5. Assay Plate: Remp polypropylene clear 384-well microplate. Cat #23490-102
6. Detection Plate: Costa black 384-well microplate. Cat #3710

### Assay Procedure: This Assay was Performed as Follows:

1. Transfer 1.5 μL of 20× compound solution to each well of an assay plate.
2. Add to each well 22.5 μL of KAB Buffer.
3. Add to each well 3 μL of the solution of DYRK1B and ATP. The final concentration of DYRK1B is 1.25 nM and ATP concentration is 70 μM (3 times of Km of ATP, which is 23.3 μM)
4. Add to each well 3 μL of the substrate peptide. The assay concentration is 1.0 μM
5. Incubate the assay plates at 37° C. for 60 minutes.
6. Add 18 μL of Detection Solution (1:800 diluted Progress bead stock, 1:400 diluted Tb stock, 80% Buffer A, and 20% Buffer B) into each well of detection plates.
7. Transfer 6 μL of assay solution from the assay plate to the detection plate.
8. Shake detection plates for 30 minutes.
9. Read plates in Envision with wavelength set at excitation 340 nm for Tb, emission 490 nm, and excitation 520 nm.
10. Calculation:

TR-FRET Signal=(Reading at 520 nM/Reading at 490 nM)×2000000

### Example 202

### DYRK1A Kinase TR-FRET (IMAP-Tb) Assay

### Assay Principle

The kinase TR-FRET (IMAP-Tb) assay uses a fluorescence labeled substrate peptide in the kinase reaction. Upon phosphorylation by the kinase, phosphopeptide is produced, which will be detected by the binding solution provided in IMAP TR-FRET binding kit. After the completion of the kinase reaction, the reaction will be stopped by adding the binding solution containing terbium tracer. This tracer is immobilized on the surface of the IMAP beads, which also contain metal ions on the beads that bind to the phospho-groups of the products. Thus the phosphorylated product of the reaction can enter into close proximity to the tracer, producing resonance energy transfer. Due to the long lifetime of terbium (Tb) fluorescence the detection can be run in time resolved mode, which virtually eliminates fluorescence interference from assay components or compounds.

The TR-FRET signal measurement from this assay, given as an IC₅₀ measurement, is a measure of a test compound's ability to interfere with the phosphorylation of the peptide substrate, that is it inhibits the phosphorylation of the substrate peptide by DYRK1A, and is thus a measure of the test compound's ability to inhibit the activity of DYRK1A. IC₅₀ is the amount of test compound that inhibits 50% of the activity of DYRK1A in this assay. In some cases where the IC₅₀ was not determined, then the % inhibition at 10 μM test compound concentration was reported. The results of this assay for sample compounds of the invention are provided in Table I below.

### Materials and Reagents

1. Human DYRK1A: from Invitrogen. Part #PV3997
2. Substrate Peptide: RRRFRPASPLRGPPK
3. IMAP TR-FRET IPP Explorer Kit: from Molecular Devices. Part #R8157
4. Kinase Assay Buffer (KAB): 10 mM HEPES pH 7.0, 50 mM NaCl, 5 mM MgCl₂, 1 mM DTT, 1 mM NaVO₄, 200 μg/mL BSA (0.02%)
5. Assay Plate: Remp polypropylene clear 384-well microplate. Cat#23490-102
6. Detection Plate: Costa black 384-well microplate. Cat #3710

### Assay Procedure: This Assay was Performed as Follows:

1. Transfer 1.5 μL of 20× compound solution to each well of an assay plate.
2. Add to each well 22.5 μL of KAB Buffer.
3. Add to each well 3 μL of the solution of DYRK1A and ATP. The final concentration of DYRK1A is 1.25 nM and ATP concentration is 70 μM (3 times of Km of ATP, which is 23.3 μM)
4. Add to each well 3 μL of the substrate peptide. The assay concentration is 1.0 μM
5. Incubate the assay plates at 37° C. for 60 minutes.
6. Add 18 μL of Detection Solution (1:800 diluted Progress bead stock, 1:400 diluted Tb stock, 80% Buffer A, and 20% Buffer B) into each well of detection plates.
7. Transfer 6 μL of assay solution from the assay plate to the detection plate.
8. Shake detection plates for 30 minutes.
9. Read plates in Envision with wavelength set at excitation 340 nm for Tb, emission 490 nm, and excitation 520 nm.
10. Calculation:

TR-FRET Signal=(Reading at 520 nM/Reading at 490 nM)×2000000

### Example 203

### SW620 Cell Viability Assay

1. Cell Plate Preparation: SW620 human colon cancer cells (known to express DYRK1B), obtained from ATCC, were seeded into 96-well plates at 3×10³ cells/well in 50 μL of media.
Harvested the required number of cells (counts & viability determined by Guava Viacount).
Centrifuge cells to pellet and removed supernant.
Resuspended in growth media (50 μL/well) & pipetted thoroughly to break up clumps.
Setup for ˜100 wells/plate, therefore, V_{T}=5 mL/plate @ 1.2×10⁵ cells/mL.
   1A. Some cells (S—) were allowed to attach for 24 hrs, serum starved for 48 hrs, and then followed with drug treatments
   Test Compound Solutions were Prepared in Regular Corresponding Media Supplemented with Serum
   2. Test Compound Preparation: Test compounds were solubilized in either DMSO or media and prepared at various stock concentrations.
All compounds were incubated at 37° C. for 30 minutes and vortexed.
   (5 mM stocks of test compound were prepared for non-soluble drugs)

### 3. Test Compound Plate Preparation:

The 10 mM drug stock was diluted to a concentration of 100× the final Cₘₐₓ concentration.
Then the test compound stock is diluted 50-fold in media and/or second drug for a final Cₘₐₓ concentration in the test compound plate.
The Cmax in the test compound plate (2% DMSO) is 2-fold higher than the final Cₘₐₓ in the cell plate (60 uL titrations were 1:3).
   4. Viability Assay: This assay was performed as follows:
Transfer 50 μL of test compound solution per well from the test compound plate onto the cell plate prepared in Step #1.
Mix the plate with treated cells by pipetting up and down three times with 200 μL multi-channel pipette.
Incubate the cells in 5% CO₂ incubator @ 37° C. for 4 days.
Run the CellTiter-Glo™ Luminescent Cell Viability Assay.

The results of this assay, given as EC₅₀ values, indicate the concentration of test compound that inhibits tumor cell proliferation by 50%. The results of this assay for sample compounds of the invention are provided in Table I below.

TABLE 1
Kinase enzyme and cellular activity

| | Enzyme | Enzyme | Cellular |
|---|---|---|---|
| | IC₅₀ (μM) | IC₅₀ (μM) | EC₅₀ (μM) |
| Example | DYRK1B | DYRK1A | SW620 |
| 19 | 0.252 | 0.116 | 92.5 |
| 21 | 0.135 | 0.097 | 3.34 |
| 22 | 2.73 | 0.601 | |
| 26 | 0.496 | 0.323 | |
| 27 | 2 | 0.376 | |
| 29 | 0.12 | 0.0859 | |
| 31 | 0.048 | 0.018 | >100 |
| 33 | 0.128 | 0.095 | |
| 34 | 0.06 | 0.05 | |
| 35 | 0.031 | 0.0046 | 7.049 |
| 37 | 0.405 | 0.468 | |
| 38 | 0.08 | 0.0385 | >100 |
| 39 | 0.0248 | 0.0128 | 2.29 |
| 40 | 0.22 | 0.208 | |
| 41 | 0.07 | 0.0796 | |
| 42 | 39% | 60% | |
| 43 | 41% | 9.8 | |
| 45 | 0.85 | 0.729 | |
| 46 | 0.192 | 1.155 | |
| 47 | 0.0908 | 0.0343 | |
| 48 | 0.217 | 0.205 | |
| 49 | 2.97 | 0.777 | |
| 50 | 3% | 30% | |
| 52 | 5.25 | 1.19 | |
| 53 | >10 | 32% | |
| 54 | 0.0677 | 0.0215 | 1.93 |
| 55 | 1.368 | 4.098 | |
| 57 | 0.096 | 0.041 | 2.41 |
| 58 | 0.04 | 0.03 | 1.733 |
| 59 | 0.222 | 0.0723 | |
| 60 | 0.357 | 0.561 | |
| 62 | 0.437 | 0.119 | |
| 63 | 28% | 4.584 | |
| 64 | 46.6% | 0.903 | |
| 65 | 0.586 | 0.305 | |
| 66 | 0.282 | 0.16 | |
| 67 | 0.669 | 0.263 | |
| 68 | 0.261 | 0.251 | |
| 70 | 0.204 | 0.352 | 2.3 |
| 71 | 0.135 | 0.342 | 2.66 |
| 72 | 0.00935 | 0.00835 | 0.828 |
| 74 | 0.0553 | 0.0256 | 1.066 |
| 75 | 0.103 | 0.0211 | 0.563 |
| 76 | 0.0128 | 0.0088 | 0.657 |
| 78 | 0.248 | 0.107 | 2.52 |
| 79 | 1.518 | 0.779 | |
| 80 | 0.231 | 0.0046 | 0.325 |
| 81 | 0.128 | 0.0313 | 0.651 |
| 82 | 1.11 | 0.463 | |
| 84 | 0.0108 | 0.0124 | 0.235 |
| 85 | 0.029 | 0.03 | 0.524 |
| 86 | 3.248 | 0.07 | >10 |
| 87 | 0.0859 | 0.061 | 0.186 |
| 88 | 0.0749 | 0.0466 | >10 |
| 89 | 0.023 | 0.0189 | 0.192 |
| 90 | 0.0523 | 0.0113 | 0.3125 |
| 92 | 0.035 | 0.0202 | |
| 94 | 0.0622 | 0.0245 | 1.133 |
| 95 | 0.012 | 0.0257 | 0.406 |
| 96 | 0.233 | 0.156 | 1.973 |
| 97 | 0.0819 | 0.0448 | 0.0662 |
| 99 | 0.0347 | 0.0202 | 0.0276 |
| 100 | 0.142 | 0.0381 | 0.068 |
| 101 | 0.0221 | 0.0263 | 0.0663 |
| 102 | 0.047 | 0.0356 | 0.0531 |
| 103 | 0.0291 | 0.00974 | >10 |
| 104 | 0.0613 | 0.0226 | 0.0787 |
| 105 | 0.0099 | 0.0096 | 0.296 |
| 107 | 0.0105 | 0.00467 | 0.236 |
| 108 | 0.056 | 0.114 | 0.528 |
| 112 | 0.018 | 0.037 | 0.89 |
| 114 | 0.0146 | 0.0373 | 1.32 |
| 118 | 0.0767 | 0.0569 | 0.438 |
| 119 | 0.135 | 0.044 | 0.152 |
| 120 | <0.0046 | 0.006 | 0.18 |
| 121 | 0.0137 | 0.0114 | 0.157 |
| 122 | 0.009 | 0.012 | 0.359 |
| 123 | 0.0082 | 0.0067 | 0.319 |
| 124 | 0.107 | 0.113 | >10 |
| 125 | 0.206 | 0.0303 | 1.496 |
| 126 | 0.059 | 0.0197 | >10 |
| 127 | 0.086 | 0.0223 | 0.165 |
| 132 | 0.0306 | 0.0128 | 0.228 |
| 133 | 0.0365 | 0.0054 | 0.7377 |
| 134 | 1.977 | 0.481 | 7.57 |
| 135 | 0.0457 | 0.0109 | 0.523 |
| 136 | 2.548 | 1.056 | |
| 137 | 0.0258 | 0.0091 | 0.659 |
| 138 | 0.0319 | 0.0184 | 2.902 |
| 139 | 0.019 | 0.006 | 0.396 |
| 140 | 0.17 | 0.073 | 3.067 |
| 141 | 0.12 | 0.101 | 2.388 |
| 142 | 44% | 2.43 | |
| 143 | 24% | 57% | |
| 144 | 23% | 0.205 | |
| 147 | 0.483 | 0.194 | 1.44 |
| 148 | 0.321 | 0.15 | 0.069 |
| 151 | 0.096 | 0.061 | 4.26 |
| 152 | 0.033 | 0.066 | 2.68 |
| 153 | 0.185 | 0.0931 | |
| 154 | 0.078 | 0.075 | |
| 155 | 0.0923 | 0.0485 | |
| 156 | 0.586 | 0.189 | |
| 157 | >10 | >10 | |
| 158 | 0.082 | 0.037 | 1.71 |
| 159 | 22% | 3.878 | |
| 160 | 0.0932 | 0.0532 | 0.219 |
| 161 | 0.0472 | 0.0462 | >10 |
| 162 | 0.135 | 0.0432 | >10 |
| 164 | 0.774 | 1.611 | >10 |
| 166 | 1.894 | 1.229 | >10 |
| 167 | 0.769 | 0.338 | >10 |
| 168 | <0.0046 | 0.0047 | 0.83 |
| 169 | 0.101 | 0.0516 | 3.13 |
| 170 | 0.117 | 0.0164 | 3.21 |
| 171 | 0.017 | 0.0093 | 0.446 |
| 172 | 0.208 | 0.109 | 0.49 |
| 173 | >10 | >10 | |
| 174 | 0.207 | 0.0873 | 0.512 |
| 176 | 1.15 | 1.03 | >1 |
| 177 | 0.176 | 0.0715 | |
| 178 | 0.356 | 0.0748 | |
| 179 | 0.131 | 0.0369 | 0.367 |
| 181 | 0.148 | 0.063 | 1.88 |
| 182 | 0.081 | 0.083 | 1.117 |
| 183 | 0.0077 | 0.0046 | 0.027 |
| 184 | 0.691 | 0.331 | 5.09 |
| 186 | 1.005 | 0.797 | |
| 187 | 0.202 | 0.13 | |
| 189 | 0.0214 | 0.0154 | |
| 192 | 0.047 | 0.036 | |
| 193 | 0.615 | 0.301 | |
| 196 | 0.339 | 0.175 | |
| 198 | 0.36 | 0.47 | |
| 200 | 0.47 | 0.54 | |

What is claimed:

1. A compound of formula I 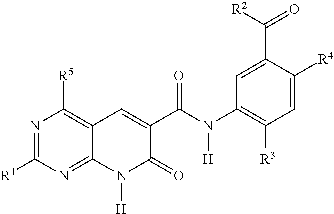
wherein
R¹ is selected from the group
(a) H,
(b) C₁₋₄ alkyl,
(c) C₁₋₄ alkyl substituted with up to 3 groups selected from cycloalkyl, heterocycle, OR⁶, NR⁶R⁷, and CN,
(d) OR⁶,
(e) NR⁶R⁷,
(f) heterocycle,
(g) heterocycle substituted with up to three groups selected from C₁₋₄ alkyl, OR⁸, NR⁸R⁹, CN and C(O)C₁₋₄ alkyl, and
(h) SR⁶;
R² is selected from the group
(a) NR¹⁰R¹¹, and
(b) OR¹²;
R³ is selected from the group
(a) H,
(b) CH₃,
(c) F,
(d) Cl, and
(f) Br;
R⁴ is selected from the group
(a) H, and
(b) F;
R⁵ is selected from the group
(a) H, and
(b) C₁₋₄ alkyl;
R⁶ and R⁷ are independently selected from the group
(a) H,
(b) C₁₋₄ alkyl,
(c) C₁₋₄ alkyl substituted with up to 3 groups selected from
OH,
OC₁₋₄ alkyl,
NR⁸R⁹,
CN,
heterocycle,
heterocycle substituted with up to three C₁₋₄ alkyl groups,
cycloalkyl, and cycloalkyl substituted with up to 3 groups selected from OH, NH₂ and C₁₋₄ alkyl,
(d) cycloalkyl,
(e) cycloalkyl substituted with up to 3 groups selected from OH, NR⁸R⁹ and C₁₋₄ alkyl,
(f) heterocycle, and
(g) heterocycle substituted with up to three C₁₋₄ alkyl groups;
R⁸ and R⁹ are independently selected from the group
(a) H, and
(b) C₁₋₄ alkyl;
R¹⁰ and R¹¹ are independently selected from the group
(a) H,
(b) C₁₋₆ alkyl,
(c) C₁₋₆ alkyl substituted with up to 4 groups selected from
aryl,
aryl substituted with Cl, F, CH₃, or CF₃
heteroaryl,
cycloalkyl,
heterocycle,
F, provided that no more than two substituents are F at the same time,
OH,
OC₁₋₄ alkyl,
NR⁸R⁹,
CN, and
CONR⁸R⁹,
(d) heterocycle substituted with aryl that optionally is substituted with halogen or C₁₋₄ alkyl, and
(e) aryl optionally substituted with Cl, F or CH₃; and
R¹² is selected from the group
(a) C₁₋₆ alkyl,
(b) C₁₋₆ alkyl substituted with up to 4 groups selected from
aryl,
aryl substituted with Cl, F, CH₃, or CF₃
heteroaryl,
cycloalkyl,
heterocycle,
F, provided that no more than two substituents are F at the same time,
OH,
OC₁₋₄ alkyl,
NR⁸R⁹,
CN, and
CONR⁸R⁹,
(c) heterocycle substituted with aryl that is optionally substituted with halogen or C₁₋₄ alkyl, and
(d) aryl optionally substituted with Cl, F or CH₃;
or a pharmaceutically acceptable salt thereof.

2. The compound of claim 1 wherein R¹ is H, or a pharmaceutically acceptable salt thereof.

3. The compound of claim 1 wherein R¹ is OR⁶ and R⁶ is C₁₋₄ alkyl optionally substituted with OC₁₋₄ alkyl or heterocycle, or a pharmaceutically acceptable salt thereof.

4. The compound of claim 3 wherein OR⁶ is OCH₃.

5. The compound of claim 1 wherein R¹ is SR⁶ and R⁶ is C₁₋₄ alkyl, or a pharmaceutically acceptable salt thereof.

6. The compound of claim 5 wherein SR⁶ is SCH₃, or a pharmaceutically acceptable salt thereof.

7. The compound of claim 1 wherein R¹ is heterocycle optionally substituted with C₁₋₄ alkyl, NR⁸R⁹ or OR⁸, and R⁸ and R⁹ are independently selected from H and methyl, or a pharmaceutically acceptable salt thereof.

8. The compound of claim 7 wherein R¹ is heterocycle optionally substituted with CH₃, NH₂ or OH.

9. The compound of claim 8 wherein the heterocycle is 8-oxa-3-aza-bycyclo[3.2.1]octane.

10. The compound of claim 7 wherein R¹ is selected from piperidinyl, piperazinyl and morpholinyl each of which optionally may be substituted with CH₃.

11. The compound of claim 1 wherein R¹ is NR⁶R⁷ and R⁶ and R⁷ are independently selected from H, C₁₋₄ alkyl that is optionally substituted with OH or OC₁₋₄ alkyl, N-ethyl, heterocycle or cycloalkyl, and wherein each of the heterocycle and cycloalkyl may optionally be substituted with CH₃, OH, NH₂, or a pharmaceutically acceptable salt thereof.

12. The compound of claim 11 wherein NR⁶R⁷ is C₁₋₄ alkyl that is substituted with a heterocycle selected from piperidinyl, piperazinyl and morpholinyl, each of which may optionally be substituted with CH₃, or a pharmaceutically acceptable salt thereof.

13. The compound of claim 1, wherein R² is OR¹² and R¹² is C₁₋₄ alkyl optionally substituted with aryl, or a pharmaceutically acceptable salt thereof.

14. The compound of claim 13, wherein R¹² is methyl or ethyl.

15. The compound of claim 13 wherein R¹² is C₁₋₄ substituted with benzene which optionally is substituted with Cl.

16. The compound of claim 1, wherein R² is NR¹⁰R¹¹ and each of R¹⁰ and R¹¹ are independently selected from H and C₁₋₆ alkyl that is optionally substituted with NH₂, OH, CONH₂, cycloalkyl, heterocycle, heteroaryl or aryl that is optionally substituted with Cl, F or CF₃, or a pharmaceutically acceptable salt thereof.

17. The compound of claim 16 wherein both R¹⁰ and R¹¹ are methyl.

18. The compound of claim 1, wherein one of R¹⁰ or R¹¹ is heterocycle optionally substituted with aryl that is optionally substituted with Cl.

19. The compound of claim 1, wherein R³ is Cl, or a pharmaceutically acceptable salt thereof.

20. The compound of claim 1, wherein R³ is CH₃, or a pharmaceutically acceptable salt thereof.

21. The compound of claim 1, wherein R³ is H, Br or F, or a pharmaceutically acceptable salt thereof.

22. The compound of claim 1, wherein R⁴ is H, or a pharmaceutically acceptable salt thereof.

23. The compound of claim 1, wherein R⁴ is F, or a pharmaceutically acceptable salt thereof.

24. The compound of claim 1, wherein R⁵ is H, or a pharmaceutically acceptable salt thereof.

25. The compound of claim 1, wherein R⁵ is CH₃, or a pharmaceutically acceptable salt thereof.

26. The compound of claim 1, wherein R⁶ and R⁷ are independently selected from H and C₁₋₄ alkyl that optionally is substituted with OH, OC₁₋₄ alkyl and N-ethyl.

27. The compound of claim 1, wherein R⁶ and R⁷ are independently selected from heterocycle and cycloalkyl, wherein the cycloalkyl may be substituted with CH₃, OH, NH₂, and C₁₋₄ alkyl, and the heterocyle optionally may be substituted with C₁₋₄ alkyl, or a pharmaceutically acceptable salt thereof.

28. The compound of claim 1, wherein R⁸ and R⁹ are independently H or CH₃, or a pharmaceutically acceptable salt thereof.

29. The compound of claim 1, wherein R¹⁰ and R¹¹ are independently selected from H and C₁₋₆ alkyl that optionally is substituted with aryl that optionally is substituted with Cl, F or CH₃, or a pharmaceutically acceptable salt thereof.

30. The compound of claim 1, wherein R¹⁰ and R¹¹ are independently selected from H and C₁₋₆ alkyl that optionally is substituted with heterocycle that optionally is substituted with CH₃, or a pharmaceutically acceptable salt thereof.

31. The compound of claim 1, wherein R¹⁰ and R¹¹ are independently selected from H, OH, OCH₃, NH₂ and CONH₂, or a pharmaceutically acceptable salt thereof.

32. The compound of claim 1, wherein R¹⁰ and R¹¹ are independently selected from H and aryl that is optionally substituted with Cl, or a pharmaceutically acceptable salt thereof.

33. The compound of claim 1, wherein R¹⁰ is H and R¹¹ is C₁₋₆ alkyl that is substituted with up to three substituents selected from heteroaryl, NR⁸R⁹ and aryl which itself is optionally substituted with Cl, or a pharmaceutically acceptable salt thereof.

34. The compound of claim 1, wherein R¹² is selected from C₁₋₄ alkyl optionally substituted with aryl that optionally is substituted with Cl, or a pharmaceutically acceptable salt thereof.

35. The compound of claim 1 selected from the group consisting of:
3-[(2-Methoxy-7-oxo-7,8-dihydro-pyrido[2,3-d]pyrimidine-6-carbonyl)-amino]-4-methyl-benzoic acid methyl ester;
4-Chloro-3-[(2-methoxy-7-oxo-7,8-dihydro-pyrido[2,3-d]pyrimidine-6-carbonyl)-amino]-benzoic acid methyl ester;
4-Chloro-3-[(2-methoxy-7-oxo-7,8-dihydro-pyrido[2,3-d]pyrimidine-6-carbonyl)-amino]-benzoic acid;
4-Methyl-3-[(2-methylsulfanyl-7-oxo-7,8-dihydro-pyrido[2,3-d]pyrimidine-6-carbonyl)-amino]-benzoic acid methyl ester;
3-[(2-Methoxy-7-oxo-7,8-dihydro-pyrido[2,3-d]pyrimidine-6-carbonyl)-amino]-4-methyl-benzoic acid benzyl ester;
2-Methoxy-7-oxo-7,8-dihydro-pyrido[2,3-d]pyrimidine-6-carboxylic acid (5-benzylcarbamoyl-2-methyl-phenyl)-amide;
4-Methyl-3-[(2-morpholin-4-yl-7-oxo-7,8-dihydro-pyrido[2,3-d]pyrimidine-6-carbonyl)-amino]-benzoic acid methyl ester;
4-Chloro-3-[(2-methylsulfanyl-7-oxo-7,8-dihydro-pyrido[2,3-d]pyrimidine-6-carbonyl)-amino]-benzoic acid methyl ester;
2-Methoxy-7-oxo-7,8-dihydro-pyrido[2,3-d]pyrimidine-6-carboxylic acid (5-benzylcarbamoyl-2-chloro-phenyl)-amide;
4-Chloro-3-[(2-morpholin-4-yl-7-oxo-7,8-dihydro-pyrido[2,3-d]pyrimidine-6-carbonyl)-amino]-benzoic acid methyl ester;
4-Chloro-3-[(7-oxo-2-piperidin-1-yl-7,8-dihydro-pyrido[2,3-d]pyrimidine-6-carbonyl)-amino]-benzoic acid methyl ester;
4-Chloro-3-{[2-(4-methyl-piperazin-1-yl)-7-oxo-7,8-dihydro-pyrido[2,3-d]pyrimidine-6-carbonyl]amino}-benzoic acid methyl ester;
4-Chloro-3-[(7-oxo-2-piperazin-1-yl-7,8-dihydro-pyrido[2,3-d]pyrimidine-6-carbonyl)-amino]-benzoic acid methyl ester;
4-Chloro-3-{[2-(2,3-dihydroxy-propylamino)-7-oxo-7,8-dihydro-pyrido[2,3-d]pyrimidine-6-carbonyl]-amino}-benzoic acid methyl ester; and
3-({2-[Bis-(2-hydroxy-ethyl)-amino]-7-oxo-7,8-dihydro-pyrido[2,3-d]pyrimidine-6-carbonyl}-amino)-4-chloro-benzoic acid methyl ester;
or a pharmaceutically acceptable salt of the foregoing compounds.

36. The compound of claim 1 selected from the group consisting of
3-({2-[Bis-(2-ethoxy-ethyl)-amino]-7-oxo-7,8-dihydro-pyrido[2,3-d]pyrimidine-6-carbonyl}-amino)-4-chloro-benzoic acid methyl ester;
4-Chloro-3-{[2-(2-hydroxy-ethylamino)-7-oxo-7,8-dihydro-pyrido[2,3-d]pyrimidine-6-carbonyl]amino}-benzoic acid methyl ester;
4-Chloro-3-{[2-(2-ethoxy-ethylamino)-7-oxo-7,8-dihydro-pyrido[2,3-d]pyrimidine-6-carbonyl]amino}-benzoic acid methyl ester;
4-Fluoro-3-[(2-methoxy-7-oxo-7,8-dihydro-pyrido[2,3-d]pyrimidine-6-carbonyl)-amino]-benzoic acid methyl ester;
4-Chloro-3-{[2-(2-ethoxy-ethoxy)-7-oxo-7,8-dihydro-pyrido[2,3-d]pyrimidine-6-carbonyl]amino}-benzoic acid methyl ester;
4-Chloro-3-{[2-(2-diethylamino-ethoxy)-7-oxo-7,8-dihydro-pyrido[2,3-d]pyrimidine-6-carbonyl]amino}-benzoic acid methyl ester;
2-Methoxy-7-oxo-7,8-dihydro-pyrido[2,3-d]pyrimidine-6-carboxylic acid [2-chloro-5-(4-chloro-benzylcarbamoyl)-phenyl]-amide;
2-Methoxy-7-oxo-7,8-dihydro-pyrido[2,3-d]pyrimidine-6-carboxylic acid [2-chloro-5-(2-chloro-benzylcarbamoyl)-phenyl]-amide;
4-Bromo-3-[(2-methoxy-7-oxo-7,8-dihydro-pyrido[2,3-d]pyrimidine-6-carbonyl)-amino]-benzoic acid methyl ester;
2-Methoxy-7-oxo-7,8-dihydro-pyrido[2,3-d]pyrimidine-6-carboxylic acid (2-chloro-5-phenylcarbamoyl-phenyl)-amide;
4-Chloro-3-[(2-methoxy-7-oxo-7,8-dihydro-pyrido[2,3-d]pyrimidine-6-carbonyl)-amino]-benzoic acid benzyl ester;
4-Chloro-3-[(2-methoxy-7-oxo-7,8-dihydro-pyrido[2,3-d]pyrimidine-6-carbonyl)-amino]-benzoic acid 4-chloro-benzyl ester;
4-Chloro-3-[(2-methoxy-7-oxo-7,8-dihydro-pyrido[2,3-d]pyrimidine-6-carbonyl)-amino]-benzoic acid 2-chloro-benzyl ester;
4-Chloro-3-[(2-methoxy-7-oxo-7,8-dihydro-pyrido[2,3-d]pyrimidine-6-carbonyl)-amino]-benzoic acid 3-chloro-benzyl ester; and
2-Methoxy-7-oxo-7,8-dihydro-pyrido[2,3-d]pyrimidine-6-carboxylic acid [2-chloro-5-(3-chloro-benzylcarbamoyl)-phenyl]-amide;
or a pharmaceutically acceptable salt of the foregoing compounds.

37. The compound of claim 1 selected from the group consisting of
2-Methoxy-7-oxo-7,8-dihydro-pyrido[2,3-d]pyrimidine-6-carboxylic acid (2-chloro-5-dimethylcarbamoyl-phenyl)-amide;
2-Methoxy-7-oxo-7,8-dihydro-pyrido[2,3-d]pyrimidine-6-carboxylic acid {2-chloro-5-[(thiophen-2-ylmethyl)-carbamoyl]-phenyl}-amide;
2-Methoxy-7-oxo-7,8-dihydro-pyrido[2,3-d]pyrimidine-6-carboxylic acid {2-chloro-5-[(thiophen-3-ylmethyl)-carbamoyl]-phenyl}-amide;
2-Methoxy-7-oxo-7,8-dihydro-pyrido[2,3-d]pyrimidine-6-carboxylic acid [2-chloro-5-(3-trifluoromethyl-benzylcarbamoyl)-phenyl]-amide;
2-Methoxy-7-oxo-7,8-dihydro-pyrido[2,3-d]pyrimidine-6-carboxylic acid [5-(benzyl-methyl-carbamoyl)-2-chloro-phenyl]-amide;
2-Methoxy-7-oxo-7,8-dihydro-pyrido[2,3-d]pyrimidine-6-carboxylic acid [5-(3-chloro-benzylcarbamoyl)-2-methyl-phenyl]-amide;
2-Methoxy-7-oxo-7,8-dihydro-pyrido[2,3-d]pyrimidine-6-carboxylic acid {5-[(3-chloro-benzyl)-methyl-carbamoyl]-2-methyl-phenyl}-amide;
7-Oxo-2-piperidin-1-yl-7,8-dihydro-pyrido[2,3-d]pyrimidine-6-carboxylic acid {2-chloro-5-[(3-chloro-benzyl)-methyl-carbamoyl]-phenyl}-amide;
2-Methoxy-7-oxo-7,8-dihydro-pyrido[2,3-d]pyrimidine-6-carboxylic acid {2-chloro-5-[(3-chloro-benzyl)-methyl-carbamoyl]-phenyl}-amide;
2-Methoxy-7-oxo-7,8-dihydro-pyrido[2,3-d]pyrimidine-6-carboxylic acid {2-chloro-5-[(pyridin-2-ylmethyl)-carbamoyl]-phenyl}-amide;
2-Methoxy-7-oxo-7,8-dihydro-pyrido[2,3-d]pyrimidine-6-carboxylic acid {2-chloro-5-[(pyridin-3-ylmethyl)-carbamoyl]-phenyl}-amide;
2-Methoxy-7-oxo-7,8-dihydro-pyrido[2,3-d]pyrimidine-6-carboxylic acid {2-chloro-5-[(pyridin-4-ylmethyl)-carbamoyl]-phenyl}-amide;
2-Methoxy-7-oxo-7,8-dihydro-pyrido[2,3-d]pyrimidine-6-carboxylic acid (2-chloro-5-phenethylcarbamoyl-phenyl)-amide;
2-Methoxy-7-oxo-7,8-dihydro-pyrido[2,3-d]pyrimidine-6-carboxylic acid {2-chloro-5-[2-(3-chloro-phenyl)-ethylcarbamoyl]-phenyl}-amide; and
2-Methoxy-7-oxo-7,8-dihydro-pyrido[2,3-d]pyrimidine-6-carboxylic acid [5-(2-amino-1-phenyl-ethylcarbamoyl)-2-chloro-phenyl]-amide;
or a pharmaceutically acceptable salt of the foregoing compounds.

38. The compound of claim 1 selected from the group consisting of
2-Methylsulfanyl-7-oxo-7,8-dihydro-pyrido[2,3-d]pyrimidine-6-carboxylic acid [2-chloro-5-(3-chloro-benzylcarbamoyl)-phenyl]-amide;
2-(4-Methyl-piperazin-1-yl)-7-oxo-7,8-dihydro-pyrido[2,3-d]pyrimidine-6-carboxylic acid [2-chloro-5-(3-chloro-benzylcarbamoyl)-phenyl]amide;
2-Methoxy-7-oxo-7,8-dihydro-pyrido[2,3-d]pyrimidine-6-carboxylic acid [5-(3-amino-1-phenyl-propylcarbamoyl)-2-chloro-phenyl]-amide;
2-Methoxy-7-oxo-7,8-dihydro-pyrido[2,3-d]pyrimidine-6-carboxylic acid (2-chloro-5-methylcarbamoyl-phenyl)-amide;
2-Methoxy-7-oxo-7,8-dihydro-pyrido[2,3-d]pyrimidine-6-carboxylic acid (2-chloro-5-isobutylcarbamoyl-phenyl)-amide;
2-(3-Morpholin-4-yl-propoxy)-7-oxo-7,8-dihydro-pyrido[2,3-d]pyrimidine-6-carboxylic acid [2-chloro-5-(3-chloro-benzylcarbamoyl)-phenyl]amide;
2-(2-Morpholin-4-yl-ethoxy)-7-oxo-7,8-dihydro-pyrido[2,3-d]pyrimidine-6-carboxylic acid [2-chloro-5-(3-chloro-benzylcarbamoyl)-phenyl]amide;
4-Chloro-3-[(2-methoxy-7-oxo-7,8-dihydro-pyrido[2,3-d]pyrimidine-6-carbonyl)-amino]-benzoic acid ethyl ester;
2-Methoxy-7-oxo-7,8-dihydro-pyrido[2,3-d]pyrimidine-6-carboxylic acid {5-[3-amino-1-(3-chloro-phenyl)-propylcarbamoyl]-2-chloro-phenyl}-amide;
2-Methoxy-7-oxo-7,8-dihydro-pyrido[2,3-d]pyrimidine-6-carboxylic acid {5-[2-amino-1-(3-chloro-phenyl)-ethylcarbamoyl]-2-chloro-phenyl}-amide;
2-Morpholin-4-yl-7-oxo-7,8-dihydro-pyrido[2,3-d]pyrimidine-6-carboxylic acid [2-chloro-5-(3-chloro-benzylcarbamoyl)-phenyl]-amide;
2-[2-(4-Methyl-piperazin-1-yl)-ethoxy]-7-oxo-7,8-dihydro-pyrido[2,3-d]pyrimidine-6-carboxylic acid [2-chloro-5-(3-chloro-benzylcarbamoyl)-phenyl]amide;
2-Methoxy-7-oxo-7,8-dihydro-pyrido[2,3-d]pyrimidine-6-carboxylic acid (5-carbamoyl-2-chloro-phenyl)-amide;
Oxo-2-(2-pyrrolidin-1-yl-ethylamino)-7,8-dihydro-pyrido[2,3-d]pyrimidine-6-carboxylic acid [2-chloro-5-(3-chloro-benzylcarbamoyl)-phenyl]amide;
2-(3-Morpholin-4-yl-propylamino)-7-oxo-7,8-dihydro-pyrido[2,3-d]pyrimidine-6-carboxylic acid [2-chloro-5-(3-chloro-benzylcarbamoyl)-phenyl]-amide; and
2-(4-Hydroxy-piperidin-1-yl)-7-oxo-7,8-dihydro-pyrido[2,3-d]pyrimidine-6-carboxylic acid [2-chloro-5-(3-chloro-benzylcarbamoyl)-phenyl]amide;
or a pharmaceutically acceptable salt of the foregoing compounds.

39. The compound of claim 1 selected from the group consisting of
2-(4-Hydroxy-cyclohexylamino)-7-oxo-7,8-dihydro-pyrido[2,3-d]pyrimidine-6-carboxylic acid [2-chloro-5-(3-chloro-benzylcarbamoyl)-phenyl]amide;
2-(4-Hydroxy-cyclohexylamino)-7-oxo-7,8-dihydro-pyrido[2,3-d]pyrimidine-6-carboxylic acid [2-chloro-5-(3-chloro-benzylcarbamoyl)-phenyl]amide;
2-Methoxy-7-oxo-7,8-dihydro-pyrido[2,3-d]pyrimidine-6-carboxylic acid {2-chloro-5-[1-(3-chloro-phenyl)-3-dimethylamino-propylcarbamoyl]-phenyl}-amide;
2-(4-Amino-piperidin-1-yl)-7-oxo-7,8-dihydro-pyrido[2,3-d]pyrimidine-6-carboxylic acid [2-chloro-5-(3-chloro-benzylcarbamoyl)-phenyl]amide;
2-[2-(4-Methyl-piperazin-1-yl)-ethylamino]-7-oxo-7,8-dihydro-pyrido[2,3-d]pyrimidine-6-carboxylic acid [2-chloro-5-(3-chloro-benzylcarbamoyl)-phenyl]-amide;
N-(2-chloro-5-(3-chlorobenzylcarbamoyl)phenyl)-2-(2-(4-methylpiperazin-1-yl)ethylamino)-7-oxo-7,8-dihydropyrido[2,3-d]pyrimidine-6-carboxamide hydrochloride;
2-[3-(4-Methyl-piperazin-1-yl)-propylamino]-7-oxo-7,8-dihydro-pyrido[2,3-d]pyrimidine-6-carboxylic acid [2-chloro-5-(3-chloro-benzylcarbamoyl)-phenyl]amide;
2-[3-(4-Methyl-piperazin-1-yl)-propylamino]-7-oxo-7,8-dihydro-pyrido[2,3-d]pyrimidine-6-carboxylic acid [2-chloro-5-(3-chloro-benzylcarbamoyl)-phenyl]amide hydrochloride;
N-(2-chloro-5-(3-chlorobenzylcarbamoyl)phenyl)-2-(2-hydroxyethylamino)-7-oxo-7,8-dihydropyrido[2,3-d]pyrimidine-6-carboxamide;
N-(2-chloro-5-(3-chlorobenzylcarbamoyl)phenyl)-2-(2-(1-methylpiperidin-4-yl)ethylamino)-7-oxo-7,8-dihydropyrido[2,3-d]pyrimidine-6-carboxamide;
N-(5-(2-amino-1-phenylethylcarbamoyl)-2-chlorophenyl)-2-(methylthio)-7-oxo-7,8-dihydropyrido[2,3-d]pyrimidine-6-carboxamide;
2-Methylsulfanyl-7-oxo-7,8-dihydro-pyrido[2,3-d]pyrimidine-6-carboxylic acid [5-(3-amino-1-phenyl-propylcarbamoyl)-2-chloro-phenyl]-amide; hydrochloride;
2-(4-Acetylpiperazin-1-yl)-N-(2-chloro-5-(3-chlorobenzylcarbamoyl)phenyl)-7-oxo-7,8-dihydropyrido[2,3-d]pyrimidine-6-carboxamide;
N-(5-(3-Amino-1-phenylpropylcarbamoyl)-2-chlorophenyl)-2-methyl-7-oxo-7,8-dihydropyrido[2,3-d]pyrimidine-6-carboxamide hydrochloride; and
N-(5-(2-Amino-1-phenylethylcarbamoyl)-2-chlorophenyl)-2-methyl-7-oxo-7,8-dihydropyrido[2,3-d]pyrimidine-6-carboxamide hydrochloride;
or a pharmaceutically acceptable salt of the foregoing compounds.

40. The compound of claim 1 selected from the group consisting of
2-((1s,4s)-4-Aminocyclohexylamino)-N-(2-chloro-5-(3-chlorobenzylcarbamoyl)phenyl)-7-oxo-7,8-dihydropyrido[2,3-d]pyrimidine-6-carboxamide hydrochloride;
N-(2-Chloro-5-(3-chlorobenzylcarbamoyl)phenyl)-7-oxo-2-(piperazin-1-yl)-7,8-dihydropyrido[2,3-d]pyrimidine-6-carboxamide hydrochloride;
N-(5-(3-Amino-1-phenylpropylcarbamoyl)-2-chlorophenyl)-2-(3-(4-methylpiperazin-1-yl)propylamino)-7-oxo-7,8-dihydropyrido[2,3-d]pyrimidine-6-carboxamide;
N-(5-(3-Amino-1-phenylpropylcarbamoyl)-2-chlorophenyl)-2-(3-(4-methylpiperazin-1-yl)propylamino)-7-oxo-7,8-dihydropyrido[2,3-d]pyrimidine-6-carboxamide hydrochloride;
N-(5-(2-Amino-1-phenylethylcarbamoyl)-2-chlorophenyl)-2-(3-(4-methylpiperazin-1-yl)propylamino)-7-oxo-7,8-dihydropyrido[2,3-d]pyrimidine-6-carboxamide;
N-(5-(2-Amino-1-phenylethylcarbamoyl)-2-chlorophenyl)-2-(3-(4-methylpiperazin-1-yl)propylamino)-7-oxo-7,8-dihydropyrido[2,3-d]pyrimidine-6-carboxamide hydrochloride;
N-(2-Chloro-5-(3-chlorobenzylcarbamoyl)phenyl)-2-((2,2-dimethyl-1,3-dioxolan-4-yl)methylamino)-7-oxo-7,8-dihydropyrido[2,3-d]pyrimidine-6-carboxamide;
N-(2-Chloro-5-(3-chlorobenzylcarbamoyl)phenyl)-7-oxo-2-(piperidin-4-ylamino)-7,8-dihydropyrido[2,3-d]pyrimidine-6-carboxamide hydrochloride;
N-(2-Chloro-5-(3-chlorobenzylcarbamoyl)phenyl)-2-(2,3-dihydroxypropylamino)-7-oxo-7,8-dihydropyrido[2,3-d]pyrimidine-6-carboxamide;
N-(2-Chloro-5-(3-chlorobenzylcarbamoyl)phenyl)-2-(2-hydroxy-2-methylpropylamino)-7-oxo-7,8-dihydropyrido[2,3-d]pyrimidine-6-carboxamide;
N-(5-(3-Amino-1-phenylpropylcarbamoyl)-2-chlorophenyl)-2-(2-(4-methylpiperazin-1-yl)ethylamino)-7-oxo-7,8-dihydropyrido[2,3-d]pyrimidine-6-carboxamide hydrochloride;
(S)—N-(5-(2-Amino-1-phenylethylcarbamoyl)-2-chlorophenyl)-2-methoxy-7-oxo-7,8-dihydropyrido[2,3-d]pyrimidine-6-carboxamide;
2-Methoxy-7-oxo-7,8-dihydro-pyrido[2,3-d]pyrimidine-6-carboxylic acid {5-[(R)-2-amino-1-(3-chloro-phenyl)-ethylcarbamoyl]-2-chloro-phenyl}-amide;
2-Methoxy-7-oxo-7,8-dihydro-pyrido[2,3-d]pyrimidine-6-carboxylic acid {5-[(S)-2-amino-1-(3-chloro-phenyl)-ethylcarbamoyl]-2-chloro-phenyl}-amide; and
2-Methoxy-7-oxo-7,8-dihydro-pyrido[2,3-d]pyrimidine-6-carboxylic acid {5-[(S)-3-amino-1-(3-chloro-phenyl)-propylcarbamoyl]-2-chloro-phenyl}-amide;
or a pharmaceutically acceptable salt of the foregoing compounds.

41. The compound of claim 1 selected from the group consisting of
N-(5-(3-Amino-1-phenylpropylcarbamoyl)-2-chlorophenyl)-2-(4-aminopiperidin-1-yl)-7-oxo-7,8-dihydropyrido[2,3-d]pyrimidine-6-carboxamide hydrochloride;
N-(5-(2-Amino-1-phenylethylcarbamoyl)-2-chlorophenyl)-2-(4-aminopiperidin-1-yl)-7-oxo-7,8-dihydropyrido[2,3-d]pyrimidine-6-carboxamide hydrochloride;
N-(5-(2-Amino-1-phenylethylcarbamoyl)-2-chlorophenyl)-2-(2-(4-methylpiperazin-1-yl)ethylamino)-7-oxo-7,8-dihydropyrido[2,3-d]pyrimidine-6-carboxamide hydrochloride;
N-(2-Chloro-5-(2-hydroxy-1-phenylethylcarbamoyl)phenyl)-2-(methylthio)-7-oxo-7,8-dihydropyrido[2,3-d]pyrimidine-6-carboxamide;
N-(2-Chloro-5-(3-hydroxy-1-phenylpropylcarbamoyl)phenyl)-2-(methylthio)-7-oxo-7,8-dihydropyrido[2,3-d]pyrimidine-6-carboxamide;
2-Morpholin-4-yl-7-oxo-7,8-dihydro-pyrido[2,3-d]pyrimidine-6-carboxylic acid [2-chloro-5-(2,2-dimethyl-propylcarbamoyl)-phenyl]-amide;
2-Morpholin-4-yl-7-oxo-7,8-dihydro-pyrido[2,3-d]pyrimidine-6-carboxylic acid {2-chloro-5-[1-(3-chloro-phenyl)-ethylcarbamoyl]-phenyl}-amide;
2-Morpholin-4-yl-7-oxo-7,8-dihydro-pyrido[2,3-d]pyrimidine-6-carboxylic acid[2-chloro-5-(3-chloro-benzylcarbamoyl)-4-fluoro-phenyl]-amide;
2-(4-Aminopiperidin-1-yl)-N-(2-chloro-5-(2-hydroxy-1-phenylethylcarbamoyl)phenyl)-7-oxo-7,8-dihydropyrido[2,3-d]pyrimidine-6-carboxamide hydrochloride;
2-(4-Aminopiperidin-1-yl)-N-(2-chloro-5-(3-hydroxy-1-phenylpropylcarbamoyl)phenyl)-7-oxo-7,8-dihydropyrido[2,3-d]pyrimidine-6-carboxamide hydrochloride;
N-(5-(2-Amino-1-phenylethylcarbamoyl)-2-chlorophenyl)-7-oxo-7,8-dihydropyrido[2,3-d]pyrimidine-6-carboxamide hydrochloride;
N-(5-(3-Amino-1-phenylpropylcarbamoyl)-2-chlorophenyl)-7-oxo-7,8-dihydropyrido[2,3-d]pyrimidine-6-carboxamide hydrochloride;
N-(2-Chloro-5-(2-hydroxy-1-phenylethylcarbamoyl)phenyl)-2-(2-hydroxy-2-methylpropylamino)-7-oxo-7,8-dihydropyrido[2,3-d]pyrimidine-6-carboxamide;
N-(2-Chloro-5-(3-hydroxy-1-phenylpropylcarbamoyl)phenyl)-2-(2-hydroxy-2-methylpropylamino)-7-oxo-7,8-dihydropyrido[2,3-d]pyrimidine-6-carboxamide; and
N-(2-Chloro-5-(3-chlorobenzylcarbamoyl)phenyl)-2-(dimethylamino)-7-oxo-7,8-dihydropyrido[2,3-d]pyrimidine-6-carboxamide;
or a pharmaceutically acceptable salt of the foregoing compounds.

42. The compound of claim 1 selected from the group consisting of
2-Morpholin-4-yl-7-oxo-7,8-dihydro-pyrido[2,3-d]pyrimidine-6-carboxylic acid [2-chloro-5-((R)-1-phenyl-ethylcarbamoyl)-phenyl]-amide;
2-Morpholin-4-yl-7-oxo-7,8-dihydro-pyrido[2,3-d]pyrimidine-6-carboxylic acid [2-chloro-5-((S)-1-phenyl-ethylcarbamoyl)-phenyl]-amide;
(R)—N-(2-Chloro-5-(3-hydroxy-1-phenylpropylcarbamoyl)phenyl)-2-methoxy-7-oxo-7,8-dihydropyrido[2,3-d]pyrimidine-6-carboxamide;
(S)—N-(2-Chloro-5-(3-hydroxy-1-phenylpropylcarbamoyl)phenyl)-2-methoxy-7-oxo-7,8-dihydropyrido[2,3-d]pyrimidine-6-carboxamide;
2-Morpholin-4-yl-7-oxo-7,8-dihydro-pyrido[2,3-d]pyrimidine-6-carboxylic acid {5-[3-amino-1-(3-chloro-phenyl)-propylcarbamoyl]-2-chloro-4-fluoro-phenyl}-amide;
2-Morpholin-4-yl-7-oxo-7,8-dihydro-pyrido[2,3-d]pyrimidine-6-carboxylic acid [2-chloro-5-(3-fluoro-benzylcarbamoyl)-phenyl]-amide;
2-Morpholin-4-yl-7-oxo-7,8-dihydro-pyrido[2,3-d]pyrimidine-6-carboxylic acid [2-chloro-4-fluoro-5-(3-fluoro-benzylcarbamoyl)-phenyl]-amide;
N-(5-(3-Aminopropylcarbamoyl)-2-chlorophenyl)-2-methoxy-7-oxo-7,8-dihydropyrido[2,3-d]pyrimidine-6-carboxamide;
N-(2-Chloro-5-(4-phenylpiperidin-4-ylcarbamoyl)phenyl)-2-methoxy-7-oxo-7,8-dihydropyrido[2,3-d]pyrimidine-6-carboxamide;
N-(5-(3-Amino-3-oxopropylcarbamoyl)-2-chlorophenyl)-2-methoxy-7-oxo-7,8-dihydropyrido[2,3-d]pyrimidine-6-carboxamide;
(R)—N-(5-(3-Amino-1-phenylpropylcarbamoyl)-2-chlorophenyl)-2-methyl-7-oxo-7,8-dihydropyrido[2,3-d]pyrimidine-6-carboxamide hydrochloride;
N-(5-(3-Amino-1-phenylpropylcarbamoyl)-2-chlorophenyl)-2-methoxy-4-methyl-7-oxo-7,8-dihydropyrido[2,3-d]pyrimidine-6-carboxamide 2,2,2-trifluoroacetate;
N-(5-(2-Amino-1-phenylethylcarbamoyl)-2-chlorophenyl)-2-methoxy-4-methyl-7-oxo-7,8-dihydropyrido[2,3-d]pyrimidine-6-carboxamide 2,2,2-trifluoroacetate;
(R)—N-(5-(3-Amino-1-phenylpropylcarbamoyl)-2-chlorophenyl)-2-methoxy-7-oxo-7,8-dihydropyrido[2,3-d]pyrimidine-6-carboxamide; and
2-(4-Methyl-piperazin-1-yl)-7-oxo-7,8-dihydro-pyrido[2,3-d]pyrimidine-6-carboxylic acid (5-benzylcarbamoyl-2-chloro-phenyl)-amide;
or a pharmaceutically acceptable salt of the foregoing compounds.

43. The compound of claim 1 selected from the group consisting of
2-(4-Methyl-piperazin-1-yl)-7-oxo-7,8-dihydro-pyrido[2,3-d]pyrimidine-6-carboxylic acid [2-chloro-5-((S)-1-phenyl-ethylcarbamoyl)-phenyl]-amide;
2-(4-Methyl-piperazin-1-yl)-7-oxo-7,8-dihydro-pyrido[2,3-d]pyrimidine-6-carboxylic acid [2-chloro-5-((R)-1-phenyl-ethylcarbamoyl)-phenyl]amide;
(S)—N-(5-(3-Amino-1-phenylpropylcarbamoyl)-2-chlorophenyl)-2-methoxy-7-oxo-7,8-dihydropyrido[2,3-d]pyrimidine-6-carboxamide;
2-(4-Methyl-piperazin-1-yl)-7-oxo-7,8-dihydro-pyrido[2,3-d]pyrimidine-6-carboxylic acid [2-chloro-5-(3-fluoro-benzylcarbamoyl)-phenyl]-amide;
2-(4-Methyl-piperazin-1-yl)-7-oxo-7,8-dihydro-pyrido[2,3-d]pyrimidine-6-carboxylic acid [2-chloro-5-(2,5-difluoro-benzylcarbamoyl)-phenyl]-amide; 2-(1R,5S)-8-Oxa-3-aza-bicyclo[3.2.1]oct-3-yl-7-oxo-7,8-dihydro-pyrido[2,3-d]pyrimidine-6-carboxylic acid [2-chloro-5-(3-chloro-benzylcarbamoyl)-phenyl]amide;
2-(1R,5S)-8-Oxa-3-aza-bicyclo[3.2.1]oct-3-yl-7-oxo-7,8-dihydro-pyrido[2,3-d]pyrimidine-6-carboxylic acid [2-chloro-5-(2-hydroxy-1-phenyl-ethylcarbamoyl)-phenyl]-amide;
2-(1R,5S)-8-Oxa-3-aza-bicyclo[3.2.1]oct-3-yl-7-oxo-7,8-dihydro-pyrido[2,3-d]pyrimidine-6-carboxylic acid [2-chloro-5-(3-hydroxy-1-phenyl-propylcarbamoyl)-phenyl]-amide;
2-(1R,5S)-8-Oxa-3-aza-bicyclo[3.2.1]oct-3-yl-7-oxo-7,8-dihydro-pyrido[2,3-d]pyrimidine-6-carboxylic acid [5-(3-amino-1-phenyl-propylcarbamoyl)-2-chloro-phenyl]-amide; hydrochloride;
(R)—N-(5-(3-Amino-3-phenylpropylcarbamoyl)-2-chlorophenyl)-2-methoxy-7-oxo-7,8-dihydropyrido[2,3-d]pyrimidine-6-carboxamide;
N-(5-(5-Amino-1-phenylpentylcarbamoyl)-2-chlorophenyl)-2-methoxy-7-oxo-7,8-dihydropyrido[2,3-d]pyrimidine-6-carboxamide;
2-(4-Methyl-piperazin-1-yl)-7-oxo-7,8-dihydro-pyrido[2,3-d]pyrimidine-6-carboxylic acid {5-[(S)-1-aminomethyl-2-(3-fluoro-phenyl)-ethylcarbamoyl]-2-chloro-phenyl}-amide;
N-(5-(4-Amino-1-phenylbutylcarbamoyl)-2-chlorophenyl)-2-methoxy-7-oxo-7,8-dihydropyrido[2,3-d]pyrimidine-6-carboxamide;
N-(5-(2-Amino-1-cyclohexylethylcarbamoyl)-2-chlorophenyl)-2-methoxy-7-oxo-7,8-dihydropyrido[2,3-d]pyrimidine-6-carboxamide;
N-(5-(3-Amino-1-cyclohexylpropylcarbamoyl)-2-chlorophenyl)-2-methoxy-7-oxo-7,8-dihydropyrido[2,3-d]pyrimidine-6-carboxamide 2,2,2-trifluoroacetate;
N-(5-(3-amino-2,2-difluoro-1-phenylpropylcarbamoyl)-2-chlorophenyl)-2-methoxy-7-oxo-7,8-dihydropyrido[2,3-d]pyrimidine-6-carboxamide;
2-Methoxy-7-oxo-7,8-dihydro-pyrido[2,3-d]pyrimidine-6-carboxylic acid [5-(3-amino-1-cyclopentyl-propylcarbamoyl)-2-chloro-phenyl]-amide; and
2-Methoxy-7-oxo-7,8-dihydro-pyrido[2,3-d]pyrimidine-6-carboxylic acid [5-(2-amino-1-cyclopentyl-ethylcarbamoyl)-2-chloro-phenyl]-amide;
or a pharmaceutically acceptable salt of the foregoing compounds.

44. The compound 2-methoxy-7-oxo-7,8-dihydro-pyrido[2,3-d]pyrimidine-6-carboxylic acid [2-chloro-5-(3-chloro-benzylcarbamoyl)-phenyl]-amide, and pharmaceutically acceptable salts thereof.

45. A pharmaceutical composition comprising a compound of claim 1, or a pharmaceutically acceptable salt thereof, as an active ingredient and a pharmaceutically acceptable carrier or excipient.
